(19) United States
(12) Patent Application Publication
(10) Pub. No.: US 2010/0008977 A1
(43) Pub. Date: Jan. 14, 2010

(54) LIPOSOMALLY ENCAPSULATED HYBRID ADENOVIRUS-SEMLIKI FOREST VIRUS (SFV) VECTORS CARRYING RNAI CONSTRUCTS AND THERAPEUTIC GENES FOR USE AGAINST CANCER TARGETS AND OTHER DISEASES

(76) Inventors: Parthenios Boulikas, Attikis (GR); Michael Roberts, Attikis (GR)

Correspondence Address: BROOKS KUSHMAN P.C., 1000 TOWN CENTER, TWENTY-SECOND FLOOR, SOUTHFIELD, MI, 48075, US

(21) Appl. No.: 12/439,636

(22) PCT Filed: Sep. 3, 2007
(86) PCT No.: PCT/GR2007/000044

§ 371 Date: Sep. 11, 2009

(30) Foreign Application Priority Data
Sep. 1, 2006 (GR) 20060100496

(51) Int. Cl. A61K 9/127; C12N 15/00; A61K 35/76

(52) U.S. Cl. 424/450; 435/320.1; 424/93.2

(57) ABSTRACT

A hybrid adenovirus Semliki Forest Virus (SFV) vector includes 3′ and 5′ inverted terminal repeat (ITR) of adenovirus, the packaging signal of adenovirus, the structural genes encoding the adenovirus hexon and penton proteins, fiber and knob proteins and that may be deleted in the E4 region, E2 region or in the both the E2 and E4 regions. The adenovirus vector may not require a helper virus coinfection for propagation in producer cell lines. A hybrid vector includes a eukaryotic promoter controlling expression of the 42S genome of SFV comprising the nonstructural genes 1-4 or two point mutations thereof, and the therapeutic mRNA, in the cytoplasm. In use, the hybrid vector further comprises cDNA encoding for microRNA (miRNA) and hairpin loops of short interfering RNA (siRNA) or cDNA encoding for double-stranded RNA (dsRNA).

### FIELD OF THE INVENTION

a. Gene Therapy of Cancer

It is estimated that approximately one in three of us will contract some form of cancer during our lifetime and that a quarter of us will die from its effects. Cancer is a complex and multifactorial disease that arises after a series of genetic alterations occur in susceptible cells that results in their uncontrolled growth and proliferation, ultimately leading to their escape to distant sites where the malignant cells disrupt the normal function of various organs resulting in death. Presently, surgery, chemotherapy and radiation therapy are the best treatment options for affected patients, and although over the past few decades these types of treatment have saved many lives, more effective therapeutic strategies against cancer still have to be devised. One of the hopes of the successful cure of all cancers lies in the field of gene therapy. It is anticipated that by using efficient gene transfer techniques, we will be able to delivery therapeutic genetic material to cancerous lesions (including metastases) ultimately resulting in irreversible tumor regression. Indeed, almost 70% of all approved clinical trials using gene therapy protocols conducted to date are specifically-designed at combating cancer. A number of different virus types have been adapted to create replication-defective gene transfer vectors; this patent describes the amalgamation of two viruses, Semliki Forest Virus (SFV) and Adenovirus (Ad) to create a novel hybrid gene transfer viral vector capable of expressing large quantities of therapeutic RNA in infected cells.

b. SFV Vectors

The wild type Semliki Forest Virus (SFV) contains a single copy of a single stranded RNA genome encapsulated into a tetrameric assembly of 240 capsid proteins, which is encapsulated in a lipid bilayer also containing 240 trimeric spike proteins. The RNA genome is 5′-capped and 3′-polyadenylated and is some 11.4 kb in length. It has positive polarity, i.e. it functions as mRNA, and can start a productive infection as soon as it enters the cytoplasm of the cell. After cell entry, infection proceeds with the translation of the 5′ two-thirds of the genome into a polyprotein that is cleaved into the four non-structural proteins nsP1-4. Following their synthesis nsP1-4 controls the replication of the plus strand into multiple full-length copies to generate the minus strand, which then serve as templates for the production of new genomic RNAs. Additionally, the minus strands are also templates for the synthesis of shorter subgenomic RNA from the internal 26S promoter present in the full length (42S) minus strand, thus generating a shorter RNA species that is 4.0 kb in length and comprises the last one-third of the viral genome. The shorter 26S RNA codes for all the structural proteins, which are synthesized as a polyprotein that is self cleaved by a viral protease. After assembly of the RNA genome with the structural proteins, the viral particles are processed by extensive post-translational modifications through the endoplasmic reticulum and Golgi apparatus where they are released through a budding process so that the particles are surrounded by a lipid bilayer (for detailed review see Strauss and Strauss, 1994). The SFV has the advantage that genomic replication occurs in the cytoplasm, where the viral replicase transcribes and caps the subgenomes for production of the structural proteins. It would obviously be very valuable to include this feature in a cDNA expression cassette to eliminate the many problems that are encountered in the conventional nuclear cDNA expression systems such as mRNA splicing, limitations in transcription factors, problems with capping efficiency and MRNA transport. Moreover, the genome of the SFV has several features that make it an ideal choice for a gene transfer vehicle: (1) it has an RNA genome of positive polarity that functions like mRNA, (2) its RNA is efficiently replicated in the cytoplasm, (3) it has late onset of cytopathogenic effects, (4) it has a broad host range and (5) it is safe to work with. SFV-based expression vectors are based on a full-length cDNA clone from which the 26S structural genes are deleted and are replaced by the heterologous insert. For in vivo packaging of recombinant viral particles a second plasmid containing the 26S structural genes is required. Both plasmids are in vitro transcribed using SP6 polymerase and their RNA is transfected into a mammalian cell line, where it is translated in the cytoplasm and the SFV genome containing the foreign gene is packaged into replication-defective viral particles. This method of SFV production is costly and inefficient. To redress this issue a number of groups have constructed alphaviral producer cell lines that express the structural genes and allow high titre production of alphaviral vector upon transfection of a plasmid encoding the replicon with the exogenous gene of interest cloned downstream of the subgenomic promoter. In this patent we describe a hybrid adenoviral-SFV vector encoding the SFV replicons with the therapeutic gene/RNAi construct cloned downstream of the SFV subgenomic promoter. It has not escaped our notice that such a vector can be used to infect alphaviral vector producer cell lines to result in increased efficiency of recombinant SFV vector production from said producer cell lines.

c. Adenoviral Vectors

One of the major limitations in using SFV vectors is the expense of producing the components required for viral vector production, indeed the process of producing the RNA genome in the test tube and its subsequent transfection into mammalian cell cultures is an extremely unwieldy process and does not scale up well for pharmaceutical application. To address this problem a number of groups have demonstrated the feasibility of using cDNA expression cassettes to produce recombinant SFV vector from RNA polymerase II promoters in mammalian cell lines (Dubensky et al, 1996; DiCommo and Bremner, 1998). However, subsequent purification of SFV vector from these cell lines at a suitable grade for pharmaceutical application is also a major hurdle in bringing these vectors into the clinic. Therefore, ideally a method of producing these alphavirus vectors at the pathologic site is required. It has been previously demonstrated that the SFV structural genes were not required to produce efficient levels of heterologous transcript from the 42S recombinant genome when cDNA Sindbis virus plasmids were injected into rodent muscle (Dubensky et al, 1996). This study suggested that the replicon present on the 42S RNA genome of the recombinant Sinbis viral vector was sufficient to drive efficient replication and subsequent expression of the foreign gene (in this case LacZ) and implied that alphaviral vector production was not necessary in order for the viral replicon to mediate efficient gene expression. Therefore, in order to increase the delivery efficiency of the alphaviral 42S RNA genome containing the therapeutic message (siRNA) in vivo, we propose to utilize adenoviral vectors as the initial gene delivery vehicle.

Adenoviruses contain a single copy of 36 kb double-stranded DNA as their genome encapsulated in an icosahedral protein capsid entity, which contains a fibre/knob protein emanating from each vertice. Adenoviruses gain entry into the cell when the fibre/knob binds to the Coxsacchie/Adenovirus receptor, which is present on a broad collection of cell types, and the RGD motif on the capsid interacts with integrin-alphaV mediating endocytosis. The adenoviral genome is then transported to the nucleus where the DNA is transcribed in two phases; (1) Early, where genes E1 to E4, involved in viral replication, are transcribed, and (2) Late, where the structural genes are transcribed from the major late promoter. Adenoviruses are attractive candidate gene transfer vehicles as they infect a broad range of cells with very high efficiency, do not require replicating cells for a productive infection, can be propagated and purified to high viral titres at pharmaceutical grade and are present in the general population and are thus considered as safe gene transfer vehicles. When using adenoviruses as gene transfer agents the essential E1 region is deleted to make the vector replication defective and to provide extra space for cloning in the structural genes. Subsequent deletions in E2, E3 and E4 regions have generated adenoviral vectors with capacities of up to 10.5 kb, and by removing all the adenoviral genome, with exception of the essential ITR and PSI cis elements, to form gutted vectors it is possible to clone up to 36 kb into this vector (reviewed in Channon and George, 1997). Adenoviral vectors deleted in the E1 region can only propagate in E1-complementing cell lines (and removal of the E2 and E4 regions also requires vector propagation in E2- and E4- complementing cell lines, respectively). Gutted adenoviral vectors require the presence of a helper adenovirus in order to propagate and this feature limits the upscalability of these vectors, as it is often difficult to separate the helper virus from the recombinant viral vector. At present adenoviral vectors are the most widely applied gene transfer vehicles in the gene therapy field, accounting for a quarter of all studies in the clinic at present, see for example (http://www.wiley.co.uk/genetherapy/clinical/). Indeed, the first gene therapy drug for the treatment of cancer approved by the Chinese FDA is based on an E1-deleted adenoviral vector expressing wild-type p53 protein and has been successfully used in the treatment of head and neck cancer (Peng, 2005), Therefore, at present E1, E2, E3 and E4-delected helper-independent adenoviral vectors represent the most effective, standardized gene transfer vehicles used in human studies to date.

In this patent we describe the application of hybrid adenovirus/SFV viral vectors, containing the 42S SFV genome inserted into E1-, E3- with E2 or E4-deleted helper-independent adenoviral vectors and under the control of an inducible or tissue specific promoters, for the transcription of therapeutic genes or siRNA messages in the treatment of cancer.

In a previous study a hybrid virus based on an adenoviral and alphaviral vector has been used in the treatment of cancer (Guan et al, 2006). In this study a hybrid Adeno-SFV construct was designed to express IL-12 from the SFV replicon that was under control of an alpha-fetoprotein (AFP) promoter. In this study a helper-dependent adenoviral vector was used as the backbone adenovirus element and contained the 5′ and 3′ adenoviral ITRs and packaging signal and the SFV replicon with IL-12 under control of the subgenomic promoter was flanked by HPRT and C346 stuffer regions of DNA. The AFP promoter, which drove expression of the SFV replicon, ensured that the RNA synthesis only occurred in cancerous cells of hepatocellular carcinoma origin. The authors proposed that the combination of an enhanced immune response against the tumour mediated by IL-12 and apoptosis induced by SFV-mediated RNA replication would enhance tumour shrinkage. The SFV element was shown to enhance the expression of IL-12 when compared to normal cDNA expression cassettes and resulted in an enhanced anti-cancer therapeutic effect in established hepatocellular carcinoma tumours. This construct has been patented by the authors as document

The hybrid Ad-SFV vector presented in the current patent differs from the previous hybrid vector in that it is based on a helper-independent adenoviral vector that can be propagated more efficiently in producer cell lines, without the risk of contamination of non-therapeutic helper virus. Although, this reduces the size of therapeutic DNA that can be inserted into the hybrid vector to some 2.5 kb, this is still large enough to accommodate a number of cytotoxic genes and provides excess space for the insertion of cDNA this is used for RNAi by encoding shRNA molecules or other longer double stranded RNA molecules that will activate the RNAi pathway. The use of hybrid Ad-SFV vectors to mediate RNAi against oncogenes or essential cellular housekeeping genes (e.g. genes involved in essential cellular metabolism or DNA replication, etc) in an inducible manner, specifically in cancerous cells represents a substantial addition to the current state-of-the-art in cancer gene therapy and is the main focus of the current patent. As such, the hybrid Ad-SFV vector presented here provides an excellent vehicle with which RNAI can be introduced in vivo for the treatment of human malignancies.

d. RNAi

The use of RNA interference (RNAi) as a therapeutic agent is gaining momentum as it has been successfully employed as a strategy to silence cancer-associate genes in animal models and now awaits evaluation in the clinic (reviewed in Hede, 2005). For example, expression of short interfering RNA molecules targeting the c-myc oncogene from a plasmid-based RNA polymerase III promoter successfully reduced the growth rate of MCF-7 breast cancer cells, both in vitro and in vivo (Wang et al, 2004). In lung cancer, ASH1 (Osada et al, 2005), EGFR (Zhang et al, 2005), hTERT (Tian et al, 2005) and SKP2 (Sumimoto et al, 2004) have all been successfully targeted by RNAi-based strategies in order to reduce the rate of tumour cell growth and promote cell death. In colon cancer, RNAi-mediate inhibition of STAT6 results in inhibition of proliferation, G1/S-arrest and initiation of apoptosis (Zhang et al, 2005). Prostate cancer cells have also been shown to be sensitive to urokinase plasminogen activator and urokinase plasminogen activator receptor knock-down by siRNA (Pulukuri et al, 2005). Moreover, knockdown of VEGF (Wannenes et al, 2005) and Androgen receptor (Haag et al, 2005) also results in a reduction of tumor growth in the prostate. RNAi-mediated silencing of hTERT has also been shown to prevent bladder cancer growth (Zou et al, 2006), as has knockdown of the PLK1 gene (Nogawa et al, 2005). Therefore, although a number of different cancer indications have been targeted for RNAi-based therapeutic pre-clinical studies, we await the first clinical trial in this area. One of the major obstacles for progression of RNAi-based therapies into the clinic is the inefficiency of in vivo delivery vehicles required to express the short RNA sequences in tumor cells. It is widely accepted that delivery of small inhibitory RNA molecules, with or without liposomal encapsulation in vivo is an extremely inefficient strategy and for application in the clinic a number of cDNA expression cassettes have been designed that function by expressing hairpin RNA messages from eukaryotic promoters such as H1 (Brummelkamp et al, 2002). However, this restricted choice of RNA polymerase III promoters for in vivo delivery of siRNA is a major limitation on the progression of this field into the clinical setting. On the other hand, SFV vectors are an ideal RNA delivery tool that do not require transcription in the nucleus for efficient replication of their RNA message and can therefore be considered as optimal delivery vehicles for siRNA. Indeed, RNAi-mediated knockdown of the GATA factor (Attado et al, 2003) and the Broad-Complex (Uhlirova et al, 2003) have been successfully achieved using another alphavirus vector (Sindbis virus) in mosquito cells. Therefore, recombinant SFV-based vectors provide the perfect tool with which to deliver therapeutic RNAi in vivo, when considering the ultimate goal of using these vectors to deliver therapeutic short RNA messages into patients with a variety of different cancer indications. At present a number of different mechanisms can be employed to activate the RNAi pathways to facilitate specific knock-down of target genes. Traditionally, RNAi delivery using alphaviruses has employed longer knock-down target sequences that other vectors, for example the standard shRNA target size is between 20-24 bp with a 4-8 bp intervening sequence to establish the hairpin loop, Attardo et al (2003) designed an dsRNA target sequence of 300 bp with an intervening 80 bp intron to act as a spacer between the sense and antisense sequences. Uhlirova et al (2003) employed a different strategy to express a dsRNA molecule of to activate the RNAi pathway and specifically downregulate the target gene. A 705 bp antisense sequence of the BR-C gene was cloned downstream of the subgenomic promoter of the Sindbis virus vector. This generated dsRNA when only when the minus strand of the 26S RNA encoding sense BR-C was synthesised, which could then function to activate the RNAi machinery. Therefore, a number of strategies are used in this patent to facilitate RNAi against cancer targets: (1) shRNA, (2) miRNA, (3) long sequences of RNA complementary to the target gene that form dsRNA during secondary structure formation as described in Attardo et al (2003) and (4) antisense RNA that pairs with sense RNA once the minus strand of the 26S RNA is synthesised during the SFV lifecycle as described in Uhlirova et al (2003). For brevity in the Claims section below methods (3) and (4) above are referred to under the umbrella term dsRNA.

### SUMMARY OF THE INVENTION

The design of new efficient and safe vehicles for the delivery of therapeutic siRNA in humans is essential if this field of gene therapy is to progress. One approach to achieve this goal is to take the most advantageous elements from different viruses and combine them together to make recombinant hybrid viral vectors capable to expressing siRNA in cancer cells. In adopting this approach, the company Regulon Inc. has sought to construct a novel viral construct based on the Adenovirus (Ad) and the Semliki Forest Virus (SFV). Regulon Inc. has used recombinant Ad vectors deleted either in the E1, E2 and E3 (Hodges et al, 2000) or E1, E3 and E4 regions (He et al, 1998) and cloned into these vectors a conventional SFV vector genome and one that is mutated in the structural genes and displays a better safety profile (SFV.PD) (Lundstrom et al, 2003).

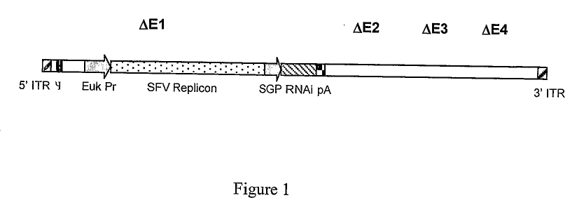

According to a first aspect of the invention, there is provided a hybrid adenovirus Semliki Forest Virus (SFV) vector comprising a structure as shown in FIG. 1. The vector may comprise the 3′ and 5′ inverted terminal repeat (ITR) of adenovirus. The hybrid vector may also comprise the packaging signal of adenovirus used to package the vector genome into the adenoviral capsid.

Suitably, the vector may comprise the structural genes encoding the adenovirus hexon and penton proteins, fiber and knob proteins. The vector may be deleted in the E4 region, in the E2 region, or in the both the E2 and E4 regions. The adenovirus vector may require a helper virus coinfection for propagation in producer cell lines. The hybrid vector may comprise a eukaryotic promoter controlling expression of the 42S genome of SFV comprising the nonstructural genes 1-4 endowed with enhanced cytotoxicity after infection of target cells and retaining the ability to replicate the 42S genome, which also comprises the therapeutic mRNA, in the cytoplasm. The hybrid vector may comprise a eukaryotic promoter controlling expression of the 42S genome of SFV comprising the nonstructural genes 1-4 containing two point mutations.

The hybrid may further comprise cDNA encoding for microRNA (miRNA) and hairpin loops of short interfering RNA (siRNA). Alternatively, the hybrid vector may further comprise cDNA encoding for double-stranded RNA (dsRNA). In another embodiment, the hybrid vector may further comprise cDNA encoding for microRNA (miRNA) and hairpin loops of short interfering RNA or dsRNA is directed against cyclin A mRNA which are placed downstream of the SFV 42S genome and under control of the SFV subgenomic promoter (SGP) for replication in the cytoplasm. In another alternative embodiment, the hybrid vector may further comprise cDNA encoding for microRNA (miRNA) and hairpin loops of short interfering RNA or dsRNA is directed against cyclin B mRNA which are placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA is directed against cyclin C mRNA which are placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA is directed against cyclin D mRNA are placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA is directed against cyclin E mRNA are placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA is directed against cyclin E mRNA which are placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA is directed against genes involved in DNA replication, e.g. DNA polymerases alpha, beta, gamma and delta, DNA ligases and topoisomerases.

The vector may further comprise cDNA encoding for microRNA (miRNA) and hairpin loops of short interfering RNA or dsRNA is directed against essential metabolic enzymes, e.g. ATPases or enzymes involved in glycolysis and the mitochondrial membrane electron transport chain.

The hybrid vector may further comprise cDNA encoding for microRNA (miRNA) and hairpin loops of short interfering RNA or dsRNA is directed against p53 mutants.

The hybrid vector may further comprise cDNA encoding for microRNA (miRNA) and hairpin loops of short interfering RNA or dsRNA is directed against aberrant signal transduction molecules, e.g. activated tyrosine kinases and tyrosine kinase receptors, EGFR, Ras, Raf, c-myc.

The hybrid vector may further comprise cDNA encoding for microRNA (miRNA) and hairpin loops of short interfering RNA or dsRNA is directed against drug resistance genes in order to convert drug-resistant tumors to chemotherapy-sensitive.

The hybrid vector may further comprise cDNA encoding for TNF-alpha, Interferon-gamma, for cancer immunotherapy is inserted into the hybrid adeno-SFV vector and specifically placed downstream of the SFV 42S genome and under control of the SFV subgenomic promoter (SGP) for replication in the cytoplasm.

The hybrid vector may further comprise cDNA encoding for wild type p53 to induce cancer cell-specific cell death is inserted into the hybrid adeno-SFV vector and specifically placed downstream of the SFV 42S genome and under control of the SFV subgenomic promoter (SGP) for replication in the cytoplasm.

The hybrid vector may further comprise cDNA encoding for wild type p53 mutagenized at 2-3 nucleotides to abort the PAX5 suppressive site and simultaneous insertion of the Pax5 cDNA whose expression product would suppress the endogenous mutated p53 are inserted into the hybrid adeno-SFV vector.

The hybrid vector may further comprise cDNA encoding for APIT to induce rapid cancer cell-specific cell death is inserted into the hybrid adeno-SFV vector and specifically placed downstream of the SFV 42S genome and under control of the SFV subgenomic promoter (S GP) for replication in the cytoplasm.

The hybrid vector may further comprise cDNA encoding for TRAIL to induce rapid programmed cell death is inserted into the hybrid adeno-SFV vector and specifically placed downstream of the SFV 42S genome and under control of the SFV subgenomic promoter (SGP) for replication in the cytoplasm.

The hybrid vector may further comprise cDNA encoding for Cip-1/Waf-1/p21, GADD45, cyclin G, mdm2, PCNA, muscle creatine kinase MCK, EGFR, Bax, and thrombospondin-1. Alternatively, it may further comprise cDNA encoding for the suicide genes, HSV-tk, CD, dCK, nitroreductase and PNP, or cDNA encoding for tumor suppressor genes Cip-1/Waf-1/p21, p16, RB, E1A, or cDNA encoding for TGF-β1, Interleukin-6 (IL-6), IL-2, interleukin-1 (IL-1), the tumor necrosis factor-oc (TNF-α), interferon (INF)-gamma, granulocyte macrophage colony stimulating factor (GM-CSF), or cDNA encoding for transcription factors E2F, RBF-1, ATF, AP-1, Sp1, NF-κB.

The hybrid vector may further comprise cDNA encoding for Bax, Bc1-2, Bc1-xs, Bc1-xL, c-Myc, Interleukin-1β converting enzyme (ICE), poly(ADP-ribose) polymerase (PARP), or cDNA encoding for ERK1, ERK2, MEK1, MEK2, MEK3, MEK4, MEK6 kinases, ceramide-activated kinase, IκB kinase, Raf-1, Jun N-terminal kinases or JNKs, p38/Mpk2), mitogen-activated protein kinase (MAPK)/extracellular signal-regulated kinase (ERK) kinase kinase 1 (MEKK1), or cDNA encoding for Adenosine deaminase (ADA) used for SCID (severe combined immunodeficiency), bc1-2 for cancer, Factor VIII for Hemophilia A, Factor IX for Hemophilia B, Growth hormone (human) for increase in growth, HSV-tk for proliferative vitreoretinopathy (PVR), IL-1 receptor antagonist (IL-IRa) for Rheumatoid arthritis (RA), LDL receptor for Familial hypercholesterolemia (FH), Nerve Growth Factor (NGF) for Alzheimer's disease and multiple sclerosis, XPD (ERCC2) for xeroderma pigmentosum (XP), TH (Tyrosine hydroxylase) for Parkinson's disease (PD). The hybrid vector may further comprise cDNA encoding for cyclin-dependent kinases (CDKs).

The hybrid vectors as described above in relation to any embodiment of the invention may further comprise an siRNA construct or a gene which is controlled by an origin of replication (ORIs).

According to a second aspect of the invention, there is provided a hybrid vector as described above wherein the hybrid adeno-SFV vector expressing a therapeutic constructs is used to infect an SFV producer cell line.

According to a third aspect of the invention, there is provided a hybrid vector as described above wherein the hybrid adeno-SFV virus is encapsulated into liposomes composed of DPPG, cholesterol, hydrogenated soy phosphatidylcholine or other lipids and coated with mPEG-DSPE.

The encapsulated virus may be targeted to tumors and metastases, to inflammatory areas in cardiovascular disease, to arthritic joints, to inflammatory bowel diseases and to other inflammatory areas in general after intravenous injection to animals and humans.

The encapsulated virus suitably may permit repetitive administrations to humans for therapy of disease without eliciting an immune reaction to the virus leading to its destruction as well as to complications to the patients such as allergic reaction, drop in blood pressure from hypotension, dyspnea, fever, rash, cardiac episodes and ultimately allergic shock.

The encapsulated virus may further comprise specific peptides with an affinity for cancer antigens selected from peptide ligand libraries where in said peptides are attached to the end of PEG-DSPE molecules in order to obtain liposomal viruses (lipoviruses) directed against specific types of tumors.

According to a fourth aspect of the invention, there is provided a hybrid vector of any one of claims 1 to 36 for use in medicine. In some embodiments of the invention, this aspect extends to the use of a hybrid vector of any one of claims 1 to 36 in the manufacture of a medicament for the treatment of tumors and/or metastases, inflammatory diseases, cardiovascular disease, arthritis, or inflammatory bowel disease. In other embodiments of the invention, this aspect extends to a method for the treatment of tumors and/or metastases, inflammatory diseases, cardiovascular disease, arthritis, or inflammatory bowel disease, comprising the step of administering a composition comprising a hybrid vector of claims 1 to 36 to a patient.

Said compositions may be formulated for administration by any suitable route such as intravenous, intraperitoneal, intrathecal, intramuscular, oral, topical, vaginal or rectal. The compositions may be formulated with any suitable pharmaceutically acceptable diluent, buffer and/or adjuvant as may be required.

The 3′ and 5′ ITR (inverted terminal repeat) of adenovirus may be used as a replication signal. The packaging signal of adenovirus may be used to package the vector genome into the adenoviral capsid. The structural genes encoding the adenovirus hexon and penton proteins, fiber and knob proteins may be used for capsid formation.

The vector may be deleted in the E4 region (in addition to deletion in the E1 and E3 regions) thus providing the capacity to accommodate the SFV elements; our hybrid vectors needs to be propagated in cell lines expressing the E1 and E4 region. The E4 region deletion is the most important because it provides a capacity to insert up to 10-kb of foreign DNA (SFV elements and therapeutic genes).

The vector may be is deleted in the E2 region (in addition to deletion in the E1 and E3 regions) thus providing the capacity to accommodate up to 9-kb of foreign DNA elements (SFV elements and therapeutic genes).

The vector may be deleted in the both the E2 and E4 regions (in addition to deletion in the E1 and E3 regions) thus providing the capacity to accommodate up to 12-kb of foreign DNA elements (SFV elements and therapeutic genes)

The adenovirus vector suitably does need need a helper virus coinfection for propagation in producer cell lines because the capsid proteins are being encoded by the adenoviral part of the vector

The vector may comprise a eukaryotic promoter controlling expression of the 42S genome of SFV comprising the nonstructural genes 1-4 contains two point mutations allowing for decreased cytotoxicity after infection of target cells and retaining the ability to replicate the 42S genome, which also comprises the therapeutic mRNA, in the cytoplasm.

The vector may further comprise cDNA encoding for microRNA (miRNA) and hairpin loops of short interfering RNA (siRNA) able to shut down the translation of specific cellular mRNAs encoding for proteins important for cellular functions that can be used against cancer, cardiovascular disease, arthritis, diabetes, dermaceutical disorders are inserted into the hybrid adeno-SFV vector and specifically placed downstream of the SFV 42S genome and under control of the SFV subgenomic promoter (SGP) for replication in the cytoplasm

The vector may further comprises cDNA encoding for double-stranded RNA (dsRNA) able to activate the RNAi/DICER pathway and shut down the translation of specific cellular mRNAs encoding for proteins important for cellular functions that can be used against cancer, cardiovascular disease, arthritis, diabetes, dermaceutical disorders are inserted into the hybrid adeno-SFV vector and specifically placed downstream of the SFV 42S genome and under control of the SFV subgenomic promoter (SGP) for replication in the cytoplasm.

The hybrid vector may further comprise cDNA encoding for microRNA (miRNA) and hairpin loops of short interfering RNA or dsRNA which is directed against genes involved in DNA replication, e.g. DNA polymerases alpha, beta, gamma and delta, DNA ligases and topoisomerases: these are proteins that control DNA replication and repair. Disruption of these proteins will prevent cell division and result in cell death.

The hybrid vector may further comprises cDNA encoding for microRNA (miRNA) and hairpin loops of short interfering RNA or dsRNA is directed against essential metabolic enzymes, e.g. ATPases or enzymes involved in glycolysis and the mitochondrial membrane electron transport chain: these enzymes regulate the essential energy metabolism of the cell. Disruption of these functions disrupt cell viability.

The hybrid vector may further comprise cDNA encoding for microRNA (miRNA) and hairpin loops of short interfering RNA or dsRNA which is directed against p53 mutants: specifically knock down p53 mutants and reexpression of wild type p53 will result in apoptosis only in cancer cells.

The hybrid vector may farther comprise cDNA encoding for microRNA (miRNA) and hairpin loops of short interfering RNA or dsRNA which is directed against aberrant signal transduction molecules, e.g. activated tyrosine kinases and tyrosine kinase receptors, EGFR, Ras, Raf, c-myc: these are oncoproteins that drive uncontrolled proliferation of the cell. Disruption of these proteins will reduce cell division and promote death of the cell.

The hybrid vector may further comprises cDNA encoding for microRNA (miRNA) and hairpin loops of short interfering RNA or dsRNA is directed against drug resistance genes in order to convert drug-resistant tumors to chemotherapy-sensitive.

| Drug resistance gene | Confers resistance to |
|---|---|
| MDR1 (multidrug resistance) | Daunomycin, doxorubicin, taxol |
| Mutant dihydrofolate reductase | Methotrexate (MTX) |
| Glutathione transferase | DNA alkylating agents |
| O6-methyl guanine transferase | Nitrosoureas |
| Cytidine deaminase | Cytosine arabinoside (Ara-C) |
| Aldehyde dehydrogenase | Cyclophosphamide |

The hybrid vector may further comprise cDNA encoding for Adenosine deaminase (ADA) used for SCID (severe combined immunodeficiency), bc1-2 for cancer, Factor VIII for Hemophilia A, Factor IX for Hemophilia B, Growth hormone (human) for increase in growth, HSV-tk for proliferative vitreoretinopathy (PVR), IL-1 receptor antagonist (IL-1Ra) for Rheumatoid arthritis (RA), LDL receptor for Familial hypercholesterolemia (FH), Nerve Growth Factor (NGF) for Alzheimer's disease and multiple sclerosis, XPD (ERCC2) for xeroderma pigmentosum (XP), TH (Tyrosine hydroxylase) for Parkinson's disease (PD) are inserted into the hybrid adeno-SFV vector.

| Gene target | Human disease |
|---|---|
| Adenosine deaminase (ADA) | SCID (severe combined |
| | immunodeficiency) |
| bcl-2 | cancer |
| Factor VIII | Hemophilia A |
| Factor IX | Hemophilia B |

| Gene target | Human disease |
|---|---|
| Growth hormone (human) | Increase in growth |
| HSV-tk | proliferative vitreoretinopathy (PVR) |
| IL-1 receptor antagonist (IL-1Ra) | Rheumatoid arthritis (RA) |
| LDL receptor | Familial hypercholesterolemia (FH) |
| Nerve Growth Factor (NGF) | Alzheimer's disease |
| XPD (ERCC2) | xeroderma pigmentosum (XP) |
| TH (Tyrosine hydroxylase) | Parkinson's disease (PD) |

The hybrid vector may further comprise an siRNA constructs or a gene which are controlled by origins of replication (ORIs) selected by the ORI TRAP method (U.S. Pat. No. 5,894,060; issued Apr. 13, 1999 to Boulikas and transferred to Regulon, Inc). Thus expression of the therapeutic genes can be achieved in specific tumors with much lower expression in other types of cells including normal cells. Basically the method can be used to isolate origins of replication from the human genome based on the matrix-attached regions (MARs) technology. ORIs are being used to drive the expression of therapeutic genes inside the cells' nucleus for months, compared to a few days (3-7 days) achieved with other existing technologies, in animal studies and clinical trials.

The hybrid vector may express a therapeutic constructs which is used to infect SFV producer cell lines (i.e. cell lines that express the SFV structural genes) and thereby introduction of the hybrid adeno-SFV vector will result in the production of high-titre SFV vector stock.

The hybrid vector virus may be encapsulated into liposomes (lipoviruses) composed of DPPG, cholesterol, hydrogenated soy phosphatidylcholine or other lipids and coated with mPEG-DSPE to generate nanoparticles carriers of sizes below 130 nm able to evade immune surveillance and protect their content from destruction at the macrophages of the liver. The method is described in U.S. Pat. No. 6,030,956 Issued Feb. 29, 2000 to Teni Boulikas and assigned to Regulon, Inc.

Such encapsulated viruses may be targeted to tumors and metastases, to inflammatory areas in cardiovascular disease, to arthritic joints, to inflammatory bowel diseases and to other inflammatory areas in general after intravenous injection to animals and humans by its long circulation as well as by its preferential extravasation through the compromised vasculature during neoangiogenesis, during arthritis and during inflammation

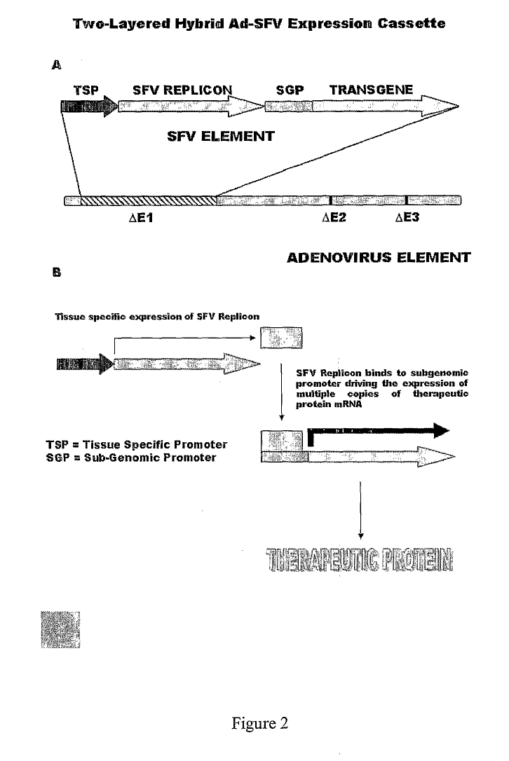

FIG. 1 illustrates the essential elements of the invention. The initial sequence comprises the 5′ ITR (inverted terminal repeat) of the adenoviral vector genome, which is immediately followed by the adenovirus packaging signal (□). A eukaryotic promoter (Euk Pr) is then inserted in the E1 region of the adenovirus immediately upstream of the SFV Replicon that initiates transcription in either an inducible, e.g. Tetracyclin-inducible, or in a tumour/tissue-specific manner, e.g. the PSA prostate-specific promoter. The cDNA encoding the active RNAi sequence (or therapeutic gene) is inserted downstream of the subgenomic promoter (SGP) that is located in the 3′ end of the SFV Replicon and to ensure efficient nuclear transcription of the entire expression cassette a poly adenylation signal (pA) is added to the 3′ end. The left-hand sequence of the adenoviral vector prior to the 3′ITR comprises deletions in the E2 and E3 regions, E3 and E4 regions or E2, E3 and E4 regions and the corresponding vector is thus propagated in the complementing producer cell line, without the need of a helper virus. In this system, the hybrid AdSFV vector produces therapeutic siRNA in a two step manner (FIG. 2):

(A) The hybrid virus first infects the cell and its DNA genome is transported to the nucleus where either an Inducible Promoter (IP) or a Tissue Specific Promoter (TSP) drives expression of the SFV RNA genome. (B) The RNA genome is transported to the cytoplasm where the SFV replicon components are expressed and assemble to drive replication of the SFV RNA. This then allows the therapeutic siRNA to be expressed from the Sub Genomic Promoter (SGP) of the SFV to high enough levels to elicit therapeutic benefit.

In short, The invention relates to a hybrid adenoviral-Semliki Forest Virus (SFV) gene expression vector which is characterised in that it comprises at least the following elements, oriented from 5′ to 3′, namely: (i) a first chain of adenoviral origin, which contains a first inverted terminal repeated sequence (ITR) and a signal sequence for packing the adenovirus; (ii) a sequence corresponding to a specific tissue or inducible promoter; (iv) an SFV-derived cDNA chain, the sequence of which is in part complementary to an SFV RNA that is mutated at two points in the nsP1-4 region to reduce toxicity, comprising at least one sequence coding for at least one exogenous hairpin loop of short interfering RNA; (v) a polyadenylation sequence; and (vi) a second adenoviral sequence deleted in the E3 and E2 or E4 regions to the 3′ adenoviral inverted terminal repeat sequence (ITR). The invention preferably relates to a hybrid adenoviral-SFV vector which comprises, by way of an exogenous hairpin loop of short interfering RNA, miRNA or dsRNA and more preferably still, to RNA interference sequences directed against genes encoding cyclins A, B, C, D & E, DNA polymerases alpha, beta, gamma & delta, DNA ligases and DNA topoisomerases, genes encoding essential elements of cellular metabolism ATPases, glycolytic enzymes and mitochondrial membrane electron transport chain components and towards oncogenes activated tyrosine kinases and tyrosine kinase receptors, EGFR, Ras, Raf, c-myc and mutant p53. A novel method is also described for construction of new adenoviruses by recombination of a shuttle plasmid with an adenoviral backbone plasmid deleted in E1, E3, E2 and E4.

Preferred features for the second and subsequent aspects of the invention are as for the first aspect mutatis mutandis.

### DETAILED DESCRIPTION OF THE INVENTION

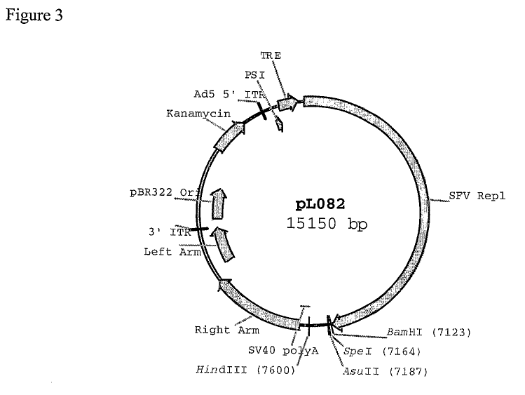

1. In order to construct this hybrid virus we have used the AdEasy system (He et al, 1998) for the generation of Ad vectors. In this system the SFV genome is first cloned into a shuttle plasmid that will be later used to fuse with the Ad genome and produce hybrid viral vectors in a complementing cell line (FIG. 3). The SFV (nsP1-4) is cloned in this shuttle plasmid in the context of an RNA polymerase II-based expression cassette and contains a Tetracyclin-Inducible Promoter and a SV40 polyadenylation signal:

Use of the Tetracycline controllable expression systems (the “Tet Technology”) is covered by a series of patents including U.S. Pat. Nos. 5,464,758 and 5,814,618, which are proprietary to TET Systems Holding GmbH & Co KG.

2. The therapeutic siRNA is then cloned into the Multiple Cloning Site present immediately after the SFV SGP.

3. The hybrid shuttle plasmid is then recombined with the Ad backbone by their co-transformation into a special strain of bacteria (He et al, 1998) and positive recombinants are selected for further analysis. Regulon has used three different Ad backbones, each with the standard E1 and E3 deletions for replication incompetence, but which differ by the introduction of an extra deletion at the E2 and/or E4 region. For propagation, E2-deleted viruses are grown in an E2-complementing cell line (Amalfitano et al, 1997), E4-deleted viruses are grown in 911E4 cells, an E4-complementing cell line and E2/E4-deleted viruses are grown in a proprietary E2/E4 complimentary cell line.

4. High titre stocks of hybrid AdSFV vectors expressing therapeutic genes or siRNA are prepared and used for increased delivery to the pathologic site, for instance by targeting using specially modified liposomes with tumour/specific peptides.

The sequence of each hybrid adenoviral-SFV construct is shown at the end of the present description.

The present invention will now be further described with reference to the following examples and drawings which are present for the purposes of illustration only and are not to be construed as being limiting on the invention.

FIG. 1 shows the essential elements of the hybrid Adenoviral-SFV Vector

FIG. 2 shows an overview of hybrid Adenoviral-SFV vector

FIG. 3 shows a schematic diagram of hybrid shuttle plasmid

At the end of the present description is showed a sequence of Hybrid Ad-SFV vector containing E1, E2 and E3 deletions from the adenovirus backbone.

### EXAMPLES

The examples describe the use of a hybrid Adenoviral-SFV vector for delivery of RNA interference constructs against the following targets:
1. Cyclin family of proteins, e.g. cyclin A, B, C, D and E: these are proteins that regulate the cell cycle. Disruption of these functions prevents cell division
2. Essential metabolic enzymes, e.g. ATPases or enzymes involved in glycolysis and the mitochondrial membrane electron transport chain: these enzymes regulate the essential energy metabolism of the cell. Disruption of these functions disrupts cell viability.
3. p53 mutants: specifically knock down p53 mutants and reexpression of wild type p53 will result in apoptosis only in cancer cells.
4. Aberrant signal transduction molecules e.g. activated tyrosine kinases and tyrosine kinase receptors, EGFR, Ras, Raf, c-myc: these are oncoproteins that drive uncontrolled proliferation of the cell. Disruption of these proteins will reduce cell division and promote death of the cell.
5. Genes involved in DNA replication, e.g. DNA polymerase family of enzymes—alpha, beta, gamma and delta, DNA ligases and topoisomerases: these are proteins that control DNA replication and repair. Disruption of these proteins will prevent cell division and result in cell death.
6. cDNA encoding for microRNA (miRNA) and hairpin loops of short interfering RNA or dsRNA is directed against drug resistance genes in order to convert drug-resistant tumors to chemotherapy-sensitive.

The examples also describes the use of the hybrid adenoviral-SFV vector for the delivery of the following genes for cancer and other diseases (as indicated):
1. TNF-alpha and Interferon-gamma for cancer immunotherapy
2. Wild type p53 to induce cancer cell-specific cell death. The ability of p53 to suppress neoplastic growth is lost by mutations on p53 that result in loss of its ability to bind to DNA or to interact with other transcription protein factors. Mutant p53 can transactivate genes that up-regulate cellular growth such as PCNA, EGFR, multiple drug resistance (MDR1), and human HSP70 in vivo supporting the idea for an oncogene function of the mutant p53 protein (for references see Boulikas, 1998, GTMB Vol 1, p54).
3. Wild type p53 mutagenized at 2-3 nucleotides to abort the PAX5 suppressive site and simultaneous insertion of the Pax5 cDNA whose expression product would suppress the endogenous mutated p53. Effective suppression of tumor growth with p53 vectors could be achieved by the simultaneous transfer of wt p53 plus Pax5 to cancer cells; Pax5 is a well established suppressor of the p53 gene; its effect is exerted via a direct interaction of Pax5 with a control element in the first exon of the p53 gene (Stuart et al, 1995). Pax5 is an homeotic protein, controlling the formation of body structures during development; Pax5 is expressed in early embryo stages to keep the levels of p53 low and allow rapid proliferation of embryonic tissues. Simultaneous transfer to solid tumors of a PAX5 and p53 genes in the same expression vector but with the wt p53 mutagenized at 2-3 nucleotides to abort the PAX5 suppressive site is a strategy previously patented to effectively suppress tumor cell proliferation (Boulikas, U.S. Pat. No. 6,030,956; issued Feb. 29, 2000).
4. TRAIL to induce rapid programmed cell death.
5. Cip-1/Waf-1/p21, GADD45, cyclin G, mdm2, PCNA, muscle creatine kinase MCK, EGFR, Bax, and thrombospondin-1. Expression of all these genes are upregulated by p53 protein and their upregulation can be applied to specific tumours to suppress tumour cell proliferation (for references see Boulikas, 1998, GTMB Vol 1, p52). The cDNAs. Gadd45 inhibits cell cycle progression. p21/CIP1/WAF1 and GADD45 interact with PCNA to inhibit its association with DNA polymerase 8 thus causing arrest in DNA replication. Mdm2 acts as a feedback loop for the biological functions of p53 apparently to moderate the G1/S arrest or apoptosis triggered by p53 following severe damage to DNA. Mdm2 protein associates with p53 causing p53 inactivation by preventing its sequence-specific binding to regulatory targets in DNA. Elevated levels of Mdm2 mimic the effect of T antigen, E1B of adenovirus, E6 of HPV, which also inactivate p53 in a similar manner; overexpression of Mdm2 can block the induction of apoptosis by p53. The PCNA promoter is up-regulated in the presence of moderate amounts of wt p53; however, at higher levels of wt p53 the PCNA promoter is inhibited whereas tumor-derived p53 mutants activate the PCNA promoter. PCNA is a protein auxiliary to DNA polymerase δ.
6. HSV-tk, CD, dCK, nitroreductase and PNP. These encode prokaryotic or viral enzymes able to convert nontoxic prodrugs into toxic derivatives. The toxic derivative produced in tumor cells which are infected can diffuse to surrounding cells causing their killing even in the absence of infection of these cells, a phenomenon known as “bystander effect”. Thymidine kinase from HSV uses the 9-{[2-hydroxy-1-(hydroxymethyl)-ethoxy]methyl}guanine or ganciclovir (GCV) and converts to GCV monophosphate for toxicity to cancer cells. Cytosine deaminase (CD) from E. coli uses 5-fluorocytosine (5FC) and converts to the toxic agent 5-fluorouracil (5FU). The E. coli (DeoD) gene encodes the purine nucleoside phosphorylase (PNP). The PNP gene product can convert the 6-methylpurine deoxyribose (MeP-dR) prodrug into the diffusible, toxic 6-methylpurine and can become a powerful suicide gene killing infected tumor cells. The method consists of infection of tumors with these Adeno-SFV constructs encoding PNP followed by treatment of the patients with MeP-dR. Purine nucleoside phosphorylase (PNP) from E. coli also uses Arabinofuranosyl-2-fluoroadenine monophosphate (F-araAMP) commercially known as fludarabine and converts it to a very toxic adenine analog. Human deoxycytidine kinase (dCK) uses Cytosine arabinoside (ara-C) and converts it to a toxic drug inducing lethal strand breaks in DNA. Nitroreductase from E. coli uses 5-(aziridin-1-yl)-2,4-dinitrobenzamide (CB1954) and converts it to a potent dysfunctional alkylating agent which crosslinks DNA.
7. Cip-1/Waf-1/p21, p16, RB and E1A. Introduction of p21 with adenoviral vectors into malignant cells completely suppressed their growth in vivo and also reduced the growth of established pre-existing tumours. One of the most frequent abnormalities in the progression of gliomas is the inactivation of the tumor-suppressor gene p16, suggesting that loss of p16 is associated with acquisition of malignant characteristics. Retinoblastoma (RB) protein is a transcription factor involved in the regulation of cell cycle progression genes. The role of RB on cell proliferation and tumor suppression arises (i) from its association with E2F, an association disrupted by RB phosphorylation at the G1/S checkpoint resulting in release of E2F and in the upregulation of a number of genes required for DNA replication; (ii) from the direct association of RB protein with a number of viral oncoproteins or key regulatory proteins including E1A of adenovirus, SV40 large T and the human papilloma virus E7 protein. RB also suppresses cell growth by directly repressing transcription of the rRNA and tRNA genes by blocking the activity of RNA polymerase I transcription factor UBF.
8. TGF-β1, Interleukin-6 (IL-6), IL-2, Interleukin-1(IL-1), The tumor necrosis factor-α (TNF-α), interferon (INF)-gamma, granulocyte macrophage colony stimulating factor (GM-CSF). Expression of these genes has pleiotropic effects on various tumors and normal cells and can also be used for cancer immunotherapy as well as against viral infections and other diseases.
9. Transcription factors E2F, RBF-1, ATF, AP-1, Sp1, NF-κB. Expression of these genes has pleiotropic effects on various tumors and normal cells and can also be used for triggering apoptosis of tumor cells and in other diseases.
10. Bax, Bc1-2, Bc1-xs, Bc1-xL, c-Myc, Interleukin-1β converting enzyme (ICE), poly(ADP-ribose) polymerase (PARP). Expression of these genes has pleiotropic effects on various tumors and normal cells and can also be used for triggering apoptosis of tumor cells, in autoimmune disease, in ischemic heart disease and in other diseases.
11. ERK1, ERK2, MEK1, MEK2, MEK3, MEK4, MEK6 kinases, ceramide-activated kinase, IκB kinase, Raf-1, Jun N-terminal kinases or JNKs, p38/Mpk2), mitogen-activated protein kinase (MAPK)/extracellular signal-regulated kinase (ERK) kinase kinase 1 (MEKK1) kinases. Expression of these genes has pleiotropic effects on various tumors and normal cells and can also be used for triggering apoptosis of tumor cells and in other diseases.
12. Adenosine deaminase (ADA) used for SCID (severe combined immunodeficiency), bc1-2 for cancer, Factor VIII for Hemophilia A, Factor IX for Hemophilia B, Growth hormone (human) for increase in growth, HSV-tk for proliferative vitreoretinopathy (PVR), IL-1 receptor antagonist (IL-1Ra) for Rheumatoid arthritis (RA), LDL receptor for Familial hypercholesterolemia (FH), Nerve Growth Factor (NGF) for Alzheimer's disease and multiple sclerosis, XPD (ERCC2) for xeroderma pigmentosum (XP), TH (Tyrosine hydroxylase) for Parkinson's disease (PD).
13. Cyclin-dependent kinases (CDKs). Overexpression of CDK proteins that enhance cell proliferation could have an advantage in combating viral infections, cahexia, malnutrition. CDK activity is essential for the phosphorylation of RB at the G1/S checkpoint of the cell cycle resulting in the release of E2F transcription factor from RB-E2F complexes and in the up-regulation by the released E2F of genes required for DNA synthesis. p21 levels are reduced considerably in tumor cells that have lost the p53 protein or contain a nonfunctional mutated form of p53.

### REFERENCES

Amalfitano A, Chamberlain J S. Isolation and characterization of packaging cell lines that coexpress the adenovirus E1, DNA polymerase, and preterminal proteins: implications for gene therapy. Gene Ther. 1997;4(3):258-63.

Attardo G M, Higgs S, Klingler K A, Vanlandingham D L, Raikhel A S. RNA interference-mediated knockdown of a GATA factor reveals a link to anautogeny in the mosquito Aedes aegypti. Proc Natl Acad Sci U S A. 2003;100(23):13374-9.

Brummelkamp T R, Bernards R, Agami R. A system for stable expression of short interfering RNAs in mammalian cells. Science. 2002;296(5567):550-3.

Channon K M, George S E. Improved adenoviral vectors: cautious optimism for gene therapy. QJM. 1997;90(2):105-9.

DiCiommo D P, Bremner R. Rapid, high level protein production using DNA-based Semliki Forest virus vectors. J Biol Chem. 1998;273(29):1 8060-6.

Dubensky T W Jr, Driver D A, Polo J M, Belli B A, Latham E M, Ibanez C E, Chada S, Brumm D, Banks T A, Mento S J, Jolly D J, Chang S M. Sindbis virus DNA-based expression vectors: utility for in vitro and in vivo gene transfer. J Virol. 1996;70(1):508-19.

Guan M, Rodriguez-Madoz J R, Alzuguren P, Gomar C, Kramer M G, Kochanek S, Prieto J, Smerdou C, Qian C. Increased efficacy and safety in the treatment of experimental liver cancer with a novel adenovirus-alphavirus hybrid vector. Cancer Res. 2006;66(3):1620-9.

Haag P, Bektic J, Bartsch G, Klocker H, Eder I E. Androgen receptor down regulation by small interference RNA induces cell growth inhibition in androgen sensitive as well as in androgen independent prostate cancer cells. J Steroid Biochem Mol Biol. 2005;96(3-4):251-8.

He T C, Zhou S, da Costa L T, Yu J, Kinzler K W, Vogelstein B. A simplified system for generating recombinant adenoviruses. Proc Natl Acad Sci U S A. 1998;95(5):2509-14.

Hodges B L, Serra D, Hu H, Begy C A, Chamberlain J S, Amalfitano A. Multiply deleted [E1, polymerase-, and pTP-] adenovirus vector persists despite deletion of the preterminal protein. J Gene Med. 2000;2(4):250-9.

Lundstrom K, Abenavoli A, Malgaroli A, Ehrengruber M U. Novel Semliki Forest virus vectors with reduced cytotoxicity and temperature sensitivity for long-term enhancement of transgene expression.

Mol Ther. 2003;7(2):202-9.

Nogawa M, Yuasa T, Kimura S, Tanaka M, Kuroda J, Sato K, Yokota A, Segawa H, Toda Y, Kageyama S, Yoshiki T, Okada Y, Maekawa T. Intravesical administration of small interfering RNA targeting PLK-1 successfully prevents the growth of bladder cancer. J Clin Invest. 2005;1 15(4):978-85.

Osada H, Tatematsu Y, Yatabe Y, Horio Y, Takahashi T. ASH1 gene is a specific therapeutic target for lung cancers with neuroendocrine features. Cancer Res. 2005;65(23): 10680-5.

Peng Z. Current Status of Gendicine in China: Recombinant Human Ad-p53 Agent for Treatment of Cancers. Hum. Gene Ther. 2005; 16:106-27.

Pulukuri S M, Gondi C S, Lakka S S, Jutla A, Estes N, Gujrati M, Rao J S. RNA interference-directed knockdown of urokinase plasminogen activator and urokinase plasminogen activator receptor inhibits prostate cancer cell invasion, survival, and tumorigenicity in vivo. J Biol Chem. 2005;280(43):36529-40.

Strauss J H, Strauss E G. The alphaviruses: gene expression, replication, and evolution. Microbiol. Rev. 1994; 58(3):491-562.

Sumimoto H, Yamagata S, Shimizu A, Miyoshi H, Mizuguchi H, Hayakawa T, Miyagishi M, Taira K, Kawakami Y. Gene therapy for human small-cell lung carcinoma by inactivation of Skp-2 with virally mediated RNA interference. Gene Ther. 2005;12(1):95-100.

Tian F J, Wang Z Y, Ma J Y, Zhao Y X, Lu W. [Inhibitory effect of hTERT dsRNA on telomerase activity in lung carcinoma cell line A549] Ai Zheng. 2005;24(3):257-61. Chinese.

Uhlirova M, Foy B D, Beaty B J, Olson K E, Riddiford L M, Jindra M. Use of Sindbis virus-mediated RNA interference to demonstrate a conserved role of Broad-Complex in insect metamorphosis. Proc Natl Acad Sci U S A. 2003;100(26):15607-12.

Wang Y H, Liu S, Zhang G, Zhou C Q, Zhu H X, Zhou X B, Quan L P, Bai J F, Xu N Z. Knockdown of c-Myc expression by RNAi inhibits MCF-7 breast tumor cells growth in vitro and in vivo. Breast Cancer Res. 2005;7(2):R220-8.

Wannenes F, Ciafre S A, Niola F, Frajese G, Farace M G. Vector-based RNA interference against vascular endothelial growth factor-A significantly limits vascularization and growth of prostate cancer in vivo. Cancer Gene Ther. 2005;12(12):926-34.

Zhang M, Zhang X, Bai C X, Song X R, Chen J, Gao L, Hu J, Hong Q Y, West M J, Wei M Q. Silencing the epidermal growth factor receptor gene with RNAi may be developed as a potential therapy for non small cell lung cancer. Genet Vaccines Ther. 2005;3:5.

Zhang M, Zhou Y, Xie C, Zhou F, Chen Y, Han G, Zhang W J. STAT6 specific shRNA inhibits proliferation and induces apoptosis in colon cancer HT-29 cells. Cancer Lett. 2005 Dec 29; [Epub ahead of print]

Zou L, Zhang P, Luo C, Tu Z. shRNA-targeted hTERT suppress cell proliferation of bladder cancer by inhibiting telomerase activity. Cancer Chemother Pharmacol. 2006;57(3):328-34.

Abbreviations:

Adenovirus (Ad), wild-type (wt), Semliki Forest Virus (SFV), HSV-tk (Herpes Simplex Virus thymidine kinase), IL (interleukin), GM-CSF (granulocyte macrophage colony-stimulating factor), xeroderma pigmentosum (XP), xeroderma pigmentosum complementation group D (XPD), cyclin-dependent kinases (CDKs), Cytosine deaminase (CD), Cytosine arabinoside (ara-C), Purine nucleoside phosphorylase (PNP), deoxycytidine kinase (dCK)

1. A hybrid adenovirus Semliki Forest Virus (SFV) vector comprising a structure as shown in FIG. 1.

2. The hybrid vector of claim 1 wherein the said vector comprises 3′ and 5′ inverted terminal repeat (ITR) of adenovirus.

3. The hybrid vector of claim 1 wherein the said vector comprises the packaging signal of adenovirus used to package the vector genome into the adenoviral capsid.

4. The hybrid vector of claim 1 wherein the said vector comprises the structural genes encoding the adenovirus hexon and penton proteins, fiber and knob proteins.

5. The hybrid vetor vector of claim 1 wherein the said vector is deleted in the E4 region.

6. The hybrid vector of claim 1 wherein the said vector is deleted in the E2 region.

7. The hybrid vector of claim 1 wherein the said vector is deleted in the both the E2 and E4 regions.

8. The hybrid vector of claim 1 wherein the said adenovirus vector does need a helper virus coinfection for propagation in producer cell lines.

9. The hybrid vector of claim 1 wherein the said vector comprises a eukaryotic promoter controlling expression of the 42S genome of SFV comprising the nonstructural genes 1-4 endowed with enhanced cytotoxicity after infection of target cells and retaining the ability to replicate the 42S genome, which also comprises the therapeutic mRNA, in the cytoplasm.

10. The hybrid vector of claim 1 wherein the said vector comprises a eukaryotic promoter controlling expression of the 42S genome of SFV comprising the nonstructural genes 1-4 containing two point mutations.

11. The hybrid vector of claim 1 wherein the vector further comprises cDNA encoding for microRNA (miRNA) and hairpin loops of short interfering RNA (siRNA).

12. The hybrid vector of claim 1 wherein the vector further comprises cDNA encoding for double-stranded RNA (dsRNA).

13. The hybrid vector of claim 1 wherein the vector further comprises cDNA encoding for microRNA (miRNA) and hairpin loops of short interfering RNA or dsRNA is directed against cyclin A mRNA which are placed downstream of the SFV 42S genome and under control of the SFV subgenomic promoter (SGP) for replication in the cytoplasm.

14. The hybrid vector of claim 1 wherein the vector further comprises cDNA encoding for microRNA (miRNA) and hairpin loops of short interfering RNA or dsRNA is directed against cyclin B mRNA which are placed downstream of the SFV 42S genome and under control of the SFV subgenomic promoter (SGP) for replication in the cytoplasm.

15. The hybrid vector of claim 1 wherein the vector further comprises cDNA encoding for microRNA (miRNA) and hairpin loops of short interfering RNA or dsRNA is directed against cyclin C mRNA which are placed downstream of the SFV 42S genome and under control of the SFV subgenomic promoter (SGP) for replication in the cytoplasm.

16. The hybrid vector of claim 1 wherein the vector further comprises cDNA encoding for microRNA (miRNA) and hairpin loops of short interfering RNA or dsRNA is directed against cyclin D mRNA are placed downstream of the SFV 42S genome and under control of the SFV subgenomic promoter (SGP) for replication in the cytoplasm.

17. The hybrid vector of claim 1 wherein the vector further comprises cDNA encoding for microRNA (miRNA) and hairpin loops of short interfering RNA or dsRNA is directed against cyclin E mRNA are placed downstream of the SFV 42S genome and under control of the SFV subgenomic promoter (SGP) for replication in the cytoplasm.

18. The hybrid vector of claim 1 wherein the vector further comprises cDNA encoding for microRNA (miRNA) and hairpin loops of short interfering RNA or dsRNA is directed against cyclin E mRNA which are placed downstream of the SFV 42S genome and under control of the SFV subgenomic promoter (SGP) for replication in the cytoplasm.

19. The hybrid vector of claim 1 where the vector further comprises cDNA encoding for microRNA (miRNA) and hairpin loops of short interfering RNA or dsRNA is directed against genes involved in DNA replication, e.g. DNA polymerases alpha, beta, gamma and delta, DNA ligases and topoisomerases.

20. The hybrid vector of claim 1 wherein the vector further comprises cDNA encoding for microRNA (miRNA) and hairpin loops of short interfering RNA or dsRNA is directed against essential metabolic enzymes, e.g. ATPases or enzymes involved in glycolysis and the mitochondrial membrane electron transport chain.

21. The hybrid vector of claim 1 where the vector further comprises cDNA encoding for microRNA (miRNA) and hairpin loops of short interfering RNA or dsRNA is directed against p53 mutants.

22. The hybrid vector of claim 1 where the vector further comprises cDNA encoding for microRNA (miRNA) and hairpin loops of short interfering RNA or dsRNA is directed against aberrant signal transduction molecules, e.g. activated tyrosine kinases and tyrosine kinase receptors, EGFR, Ras, Raf, c-myc.

22. The hybrid vector of claim 1 wherein the vector further comprises cDNA encoding for microRNA (miRNA) and hairpin loops of short interfering RNA or dsRNA is directed against drug resistance genes in order to convert drug-resistant tumors to chemotherapy-sensitive.

23. The hybrid vector of claim 1 wherein the vector further comprises cDNA encoding for TNF-alpha, Interferon-gamma, for cancer immunotherapy is inserted into the hybrid adeno-SFV vector and specifically placed downstream of the SFV 42S genome and under control of the SFV subgenomic promoter (SGP) for replication in the cytoplasm.

24. The hybrid vector of claim 1 wherein the vector further comprises cDNA encoding for wild type p53 to induce cancer cell-specific cell death is inserted into the hybrid adeno-SFV vector and specifically placed downstream of the SFV 42S genome and under control of the SFV subgenomic promoter (SGP) for replication in the cytoplasm.

25. The hybrid vector of claim 1 wherein the vector further comprises cDNA encoding for wild type p53 mutagenized at 2-3 nucleotides to abort the PAX5 suppressive site and simultaneous insertion of the Pax5 cDNA whose expression product would suppress the endogenous mutated p53 are inserted into the hybrid adeno-SFV vector.

26. The hybrid vector of claim 1 wherein the vector further comprises cDNA encoding for APIT to induce rapid cancer cell-specific cell death is inserted into the hybrid adeno-SFV vector and specifically placed downstream of the SFV 42S genome and under control of the SFV subgenomic promoter (SGP) for replication in the cytoplasm.

27. The hybrid vector of claim 1 wherein the vector further comprises cDNA encoding for TRAIL to induce rapid programmed cell death is inserted into the hybrid adeno-SFV vector and specifically placed downstream of the SFV 42S genome and under control of the SFV subgenomic promoter (SGP) for replication in the cytoplasm.

28. The hybrid vector of claim 1 wherein the vector further comprises cDNA encoding for Cip-1/Waf-1/p21, GADD45, cyclin G, mdm2, PCNA, muscle creatine kinase MCK, EGFR, Bax, and thrombospondin-1.

29. The hybrid vector of claim 1 wherein the vector further comprises cDNA encoding for the suicide genes, HSV-tk, CD, dCK, nitroreductase and PNP.

30. The hybrid vector of claim 1 wherein the vector further comprises cDNA encoding for tumor suppressor genes Cip-1/Waf-1/p21, p16, RB, E1A.

31. The hybrid vector of claim 1 wherein the vector further comprises cDNA encoding for TGF-β1, Interleukin-6 (IL-6), IL-2, Interleukin-1 (IL-1), the tumor necrosis factor-α (TNF-α ), interferon (INF)-gamma, granulocyte macrophage colony stimulating factor (GM-CSF).

32. The hybrid vector of claim 1 wherein the vector further comprises cDNA encoding for transcription factors E2F, RBF-1, ATF, AP-1, Sp1, NF-κB.

33. The hybrid vector of claim 1 wherein the vector further comprises cDNA encoding for Bax, Bc1-2, Bc1-xs, Bc1-xL, c-Myc, Interleukin-1β converting enzyme (ICE), poly(ADP-ribose) polymerase (PARP).

34. The hybrid vector of claim 1 wherein the vector further comprises cDNA encoding for ERK1, ERK2, MEK1, MEK2, MEK3, MEK4, MEK6 kinases, ceramide-activated kinase, IκB kinase, Raf-1, Jun N-terminal kinases or JNKs, p38/Mpk2), mitogen-activated protein kinase (MAPK)/extracellular signal-regulated kinase (ERK) kinase kinase 1 (MEKK1).

35. The hybrid vector of claim 1 wherein the vector further comprises cDNA encoding for Adenosine deaminase (ADA) used for SCID (severe combined immunodeficiency), bc1-2 for cancer, Factor VIII for Hemophilia A, Factor IX for Hemophilia B, Growth hormone (human) for increase in growth, HSV-tk for proliferative vitreoretinopathy (PVR), IL-1 receptor antagonist (IL-IRa) for Rheumatoid arthritis (RA), LDL receptor for Familial hypercholesterolemia (FH), Nerve Growth Factor (NGF) for Alzheimer's disease and multiple sclerosis, XPD (ERCC2) for xeroderma pigmentosum (XP), TH (Tyrosine hydroxylase) for Parkinson's disease (PD).

36. The hybrid vector of claim 1 wherein the vector further comprises cDNA encoding for cyclin-dependent kinases (CDKs).

37. The hybrid vector of claim 1 wherein the vector further comprises an siRNA construct or a gene which is controlled by origins of replication (ORIs).

38. The hybrid vector of claim 1 wherein the hybrid adeno-SFV vector expressing a therapeutic constructs is used to infect SFV producer cell lines.

39. The hybrid vector of claim 1 wherein the hybrid adeno-SFV virus is encapsulated into liposomes composed of DPPG, cholesterol, hydrogenated soy phosphatidylcholine or other lipids and coated with mPEG-DSPE.

40. The encapsulated virus of claim 39 wherein the encapsulated virus is targeted to tumors and metastases, to inflammatory areas in cardiovascular disease, to arthritic joints, to inflammatory bowel diseases and to other inflammatory areas in general after intravenous injection to animals and humans.

41. The encapsulated virus of claim 38 wherein the liposomal virus permits repetitive administrations to humans for therapy of disease without eliciting an immune reaction to the virus leading to its destruction as well as to complications to the patients such as allergic reaction, drop in blood pressure from hypotension, dyspnea, fever, rash, cardiac episodes and ultimately allergic shock.

42. The encapsulated virus of claim 38 wherein said virus further comprises specific peptides with an affinity for cancer antigens selected from peptide ligand libraries which are attached to the end of PEG-DSPE molecules in said viruses in order to obtain lipoviruses directed against specific types of tumors.

43. A hybrid vector of claim 1 for use in medicine.

44. The use of a hybrid vector claim 1 in the manufacture of a medicament for the treatment of tumors and/or metastases, inflammatory diseases, cardiovascular disease, arthritis, or inflammatory bowel disease.

45. A method for the treatment of tumors and/or metastases, inflammatory diseases, cardiovascular disease, arthritis, or inflammatory bowel disease, comprising the step of administering a composition comprising a hybrid vector of claim 1 to a patient.


SEQUENCE LISTING (3 sequences)

SEQ ID NO: 1 - DNA, 75350 bp, Artificial
  /note = Sequence of Hybrid Ad-SFV vector containing E1, E2 and E3 deletions from the adenovirus backbone
taacatcatcaataatataccttattttggattgaagccaatatgataatgagggggtggagtttgtgacgtggcgcggggcgtgggaacggggcgggtgattgtagtagttattatatggaataaaacctaacttcggttatactattactcccccacctcaaacactgcaccgcgccccgcacccttgccccgcccacacgtagtagtgtggcggaagtgtgatgttgcaagtgtggcggaacacatgtaagcgacggatgtggcaaaagtgacgtttttggtgtgcgccggtgtacatgcatcatcacaccgccttcacactacaacgttcacaccgccttgtgtacattcgctgcctacaccgttttcactgcaaaaaccacacgcggccacatgtcaggaagtgacaattttcgcgcggttttaggcggatgttgtagtaaatttgggcgtaaccgagtaagatttggccattttcgcgggaaaactgaataagagtccttcactgttaaaagcgcgccaaaatccgcctacaacatcatttaaacccgcattggctcattctaaaccggtaaaagcgcccttttgacttattctggaagtgaaatctgaataattttgtgttactcatagcgcgtaatactggtaccgcggccgcctcgagtttaccactccctatcagtgatagagaaaagtgccttcactttagacttattaaaacacaatgagtatcgcgcattatgaccatggcgccggcggagctcaaatggtgagggatagtcactatctcttttcacaaagtcgagtttaccactccctatcagtgatagagaaaagtgaaagtcgagtttaccactccctatcagtgatagagaaaagtgaaagtcgagtttaccatttcagctcaaatggtgagggatagtcactatctcttttcactttcagctcaaatggtgagggatagtcactatctcttttcactttcagctcaaatggtctccctatcagtgatagagaaaagtgaaagtcgagtttaccactccctatcagtgatagagaaaagtgaaagtcgagtttaccactccctatcagtgatagagggatagtcactatctcttttcactttcagctcaaatggtgagggatagtcactatctcttttcactttcagctcaaatggtgagggatagtcactatgagaaaagtgaaagtcgagtttaccactccctatcagtgatagagaaaagtgaaagtcgagctcggtacccgggtcgaggtaggcgtgtacggtgggaggctcttttcactttcagctcaaatggtgagggatagtcactatctcttttcactttcagctcgagccatgggcccagctccatccgcacatgccaccctcccctatataagcagagctcgtttagtgaaccgtcagatcgcctggagacgccatccacgctgttttgacctccatagaagacaccgggaccgatccagcctggatatattcgtctcgagcaaatcacttggcagtctagcggacctctgcggtaggtgcgacaaaactggaggtatcttctgtggccctggctaggtcggaccgcggccccgaattcgagctcggtacccggggatcgatggcggatgtgtgacatacacgacgccaaaagattttgttccagctcctgccacctccgctaggcgccggggcttaagctcgagccatgggcccctagctaccgcctacacactgtatgtgctgcggttttctaaaacaaggtcgaggacggtggaggcgatcgcgagagattaaccacccacgatggccgccaaagtgcatgttgatattgaggctgacagcccattcatcaagtctttgcagaaggcatttccgtcgttcgcgctctctaattggtgggtgctaccggcggtttcacgtacaactataactccgactgtcgggtaagtagttcagaaacgtcttccgtaaaggcagcaaggaggtggagtcattgcaggtcacaccaaatgaccatgcaaatgccagagcattttcgcacctggctaccaaattgatcgagcaggagactgacaaagacactccacctcagtaacgtccagtgtggtttactggtacgtttacggtctcgtaaaagcgtggaccgatggtttaactagctcgtcctctgactgtttctgtcactcatcttggatatcggcagtgcgccttccaggagaatgatgtctacgcacaaataccactgcgtatgccctatgcgcagcgcagaagaccccgaaaggtgagtagaacctatagccgtcacgcggaaggtcctcttactacagatgcgtgtttatggtgacgcatacgggatacgcgtcgcgtcttctggggctttcgctcgatagctacgcaaagaaactggcagcggcctccgggaaggtgctggatagagagatcgcaggaaaaatcaccgacctgcagaccgtcatggctacgcgagctatcgatgcgtttctttgaccgtcgccggaggcccttccacgacctatctctctagcgtcctttttagtggctggacgtctggcagtaccgatgcccagacgctgaatctcctaccttttgcctgcatacagacgtcacgtgtcgtacggcagccgaagtggccgtataccaggacgtgtatgctgtacatgcacggtctgcgacttagaggatggaaaacggacgtatgtctgcagtgcacagcatgccgtcggcttcaccggcatatggtcctgcacatacgacatgtacgtgcaacatcgctgtaccatcaggcgatgaaaggtgtcagaacggcgtattggattgggtttgacaccaccccgtttatgtttgacgcgctagcaggcgcgtagttgtagcgacatggtagtccgctactttccacagtcttgccgcataacctaacccaaactgtggtggggcaaatacaaactgcgcgatcgtccgcgcattccaacctacgccacaaactgggccgacgagcaggtgttacaggccaggaacataggactgtgtgcagcatccttgactgagggaagactcggcaaactgaggttggatgcggtgtttgacccggctgctcgtccacaatgtccggtccttgtatcctgacacacgtcgtaggaactgactcccttctgagccgtttgactccattctccgcaagaagcaattgaaaccttgcgacacagtcatgttctcggtaggatctacattgtacactgagagcagaaagctactgaggagctggcaggtaagaggcgttcttcgttaactttggaacgctgtgtcagtacaagagccatcctagatgtaacatgtgactctcgtctttcgatgactcctcgaccgacttaccctccgtattccacctgaaaggtaaacaatcctttacctgtaggtgcgataccatcgtatcatgtgaagggtacgtagttaagaaaatcactattgaatgggaggcataaggtggactttccatttgttaggaaatggacatccacgctatggtagcatagtacacttcccatgcatcaattcttttagtgatagtgccccggcctgtacggtaaaacggtagggtacgccgtgacgtatcacgcggagggattcctagtgtgcaagaccacagacactgtcaaaggagaaagacacggggccggacatgccattttgccatcccatgcggcactgcatagtgcgcctccctaaggatcacacgttctggtgtctgtgacagtttcctctttctgtctcattccctgtatgcacctacgtcccctcaaccatctgtgatcaaatgactggcatactagcgaccgacgtcacaccggaggacgcacagaagttgtcagagtaagggacatacgtggatgcaggggagttggtagacactagtttactgaccgtatgatcgctggctgcagtgtggcctcctgcgtgtcttcaacatagtgggattgaatcagaggatagttgtgaacggaagaacacagcgaaacactaacacgatgaagaactatctgcttccgattgtggccgtcgcatttagatcaccctaacttagtctcctatcaacacttgccttcttgtgtcgctttgtgattgtgctacttcttgatagacgaaggctaacaccggcagcgtaaatccaagtgggcgagggaatacaaggcagaccttgatgatgaaaaacctctgggtgtccgagagaggtcacttacttgctgctgcttgtgggcatttaaaacggttcacccgctcccttatgttccgtctggaactactactttttggagacccacaggctctctccagtgaatgaacgacgacgaacacccgtaaattttgcaggaagatgcacaccatgtacaagaaaccagacacccagacaatagtgaaggtgccttcagagtttaactcgttcgtcatcccgagcctatggtctacagtccttctacgtgtggtacatgttctttggtctgtgggtctgttatcacttccacggaagtctcaaattgagcaagcagtagggctcggataccagatgtcgcctcgcaatcccagtcagatcacgcattaagatgcttttggccaagaagaccaagcgagagttaatacctgttctcgacgcgtcgtcagccagggatgccggagcgttagggtcagtctagtgcgtaattctacgaaaaccggttcttctggttcgctctcaattatggacaagagctgcgcagcagtcggtccctacgtgaacaagaggagaaggagaggttggaggccgagctgactagagaagccttaccacccctcgtccccatcgcgccggcggagacgggagtcgtcgacgtcacttgttctcctcttcctctccaacctccggctcgactgatctcttcggaatggtggggagcaggggtagcgcggccgcctctgccctcagcagctgcaggacgttgaagaactagagtatcacgcaggtgcaggggtcgtggaaacacctcgcagcgcgttgaaagtcaccgcacagccgaacgacgtactactaggaactgcaacttcttgatctcatagtgcgtccacgtccccagcacctttgtggagcgtcgcgcaactttcagtggcgtgtcggcttgctgcatgatgatccttattacgtagttctgtccccgcagaccgtgctcaagagctccaagttggcccccgtgcaccctctagcagagcaggtgaaaataataacacataacgggagtaatgcatcaagacaggggcgtctggcacgagttctcgaggttcaaccgggggcacgtgggagatcgtctcgtccacttttattattgtgtattgccctcggccggcggttaccaggtcgacggatatgacggcagggtcctactaccatgtggatcggccattccggtccctgagtttcaggctttgagcgagagcgccccggccgccaatggtccagctgcctatactgccgtcccaggatgatggtacacctagccggtaaggccagggactcaaagtccgaaactcgctctcgcggactatggtgtacaacgaaagggagttcgtcaacaggaaactataccatattgccgttcacggaccctcgctgaacaccgacgaggagaactacgagaaagtgataccacatgttgctttccctcaagcagttgtcctttgatatggtataacggcaagtgcctgggagcgacttgtggctgctcctcttgatgctctttctcagagctgaaagaactgacgccgagtacgtgttcgacgtagataaaaaatgctgcgtcaagagagaggaagcgtcgggtttggtgttggtgggagagctagtctcgactttcttgactgcggctcatgcacaagctgcatctattttttacgacgcagttctctctccttcgcagcccaaaccacaaccaccctctcgaaaccaaccccccgttccatgaattcgcctacgaagggctgaagatcaggccgtcggcaccatataagactacagtagtaggagtctttggggttccgggattggttggggggcaaggtacttaagcggatgcttcccgacttctagtccggcagccgtggtatattctgatgtcatcatcctcagaaaccccaaggcccttcaggcaagtctgctattattaagagcctcgtgaccaaacacgatctggtcaccagcggcaagaaggagaactgccaggaaatagttaacgacgtgaagaagtccgttcagacgataataattctcggagcactggtttgtgctagaccagtggtcgccgttcttcctcttgacggtcctttatcaattgctgcacttctagcaccgcgggaaggggacaagtagggaaaacagtgactccatcctgctaaacgggtgtcgtcgtgccgtggacatcctatatgtggacgaggctttcgctcgtggcgcccttcccctgttcatcccttttgtcactgaggtaggacgatttgcccacagcagcacggcacctgtaggatatacacctgctccgaaagcgttgccatcccggtactctgctggccctaattgctcttgttaaacctcggagcaaagtggtgttatgcggagaccccaagcaatgcggattcttcaatatgaacggtagggccatgagacgaccgggattaacgagaacaatttggagcctcgtttcaccacaatacgcctctggggttcgttacgcctaagaagttatacatgcagcttaaggtgaacttcaaccacaacatctgcactgaagtatgtcataaaagtatatccagacgttgcacgcgtccagtcacggccatcgtgtctatacgtcgaattccacttgaagttggtgttgtagacgtgacttcatacagtattttcatataggtctgcaacgtgcgcaggtcagtgccggtagcacagatcgttgcactacggaggcaagatgcgcacgaccaacccgtgcaacaaacccataatcatagacaccacaggacagaccaagcccaagccaggagacatcgtgcaacgtgatgcctccgttctacgcgtgctggttgggcacgttgtttgggtattagtatctgtggtgtcctgtctggttcgggttcggtcctctgtagcagttaacatgcttccgaggctgggcaaagcagctgcagttggactaccgtggacacgaagtcatgacagcagcagcatctcagggcctcacccgcaaagggcaattgtacgaaggctccgacccgtttcgtcgacgtcaacctgatggcacctgtgcttcagtactgtcgtcgtcgtagagtcccggagtgggcgtttcccgtatacgccgtaaggcagaaggtgaatgaaaatcccttgtatgcccctgcgtcggagcacgtgaatgtactgctgacgcgcactgaggataggctggtgtcatatgcggcattccgtcttccacttacttttagggaacatacggggacgcagcctcgtgcacttacatgacgactgcgcgtgactcctatccgaccacaggaaaacgctggccggcgatccctggattaaggtcctatcaaacattccacagggtaactttacggccacattggaagaatggcaagaagaacacgacaaccttttgcgaccggccgctagggacctaattccaggatagtttgtaaggtgtcccattgaaatgccggtgtaaccttcttaccgttcttcttgtgctgttaataatgaaggtgattgaaggaccggctgcgcctgtggacgcgttccagaacaaagcgaacgtgtgttgggcgaaaagcctggtgcctgtcctggacactttattacttccactaacttcctggccgacgcggacacctgcgcaaggtcttgtttcgcttgcacacaacccgcttttcggaccacggacaggacctgtgagccggaatcagattgacagcagaggagtggagcaccataattacagcatttaaggaggacagagcttactctccagtggtggccttgaatgaaatttgcacggccttagtctaactgtcgtctcctcacctcgtggtattaatgtcgtaaattcctcctgtctcgaatgagaggtcaccaccggaacttactttaaacgtccaagtactatggagttgacctggacagtggcctgttttctgccccgaaggtgtccctgtattacgagaacaaccactgggataacagacctggtggaagggttcatgatacctcaactggacctgtcaccggacaaaagacggggcttccacagggacataatgctcttgttggtgaccctattgtctggaccaccttcgatgtatggattcaatgccgcaacagctgccaggctggaagctagacataccttcctgaaggggcagtggcatacgggcaagcaggcagttatcgcagaactacatacctaagttacggcgttgtcgacggtccgaccttcgatctgtatggaaggacttccccgtcaccgtatgcccgttcgtccgtcaatagcgtcttagaaaaatccaaccgctttctgtgctggacaatgtaattcctatcaaccgcaggctgccgcacgccctggtggctgagtacaagacggttaaaggcagtatctttttaggttggcgaaagacacgacctgttacattaaggatagttggcgtccgacggcgtgcgggaccaccgactcatgttctgccaatttccgtcatgggttgagtggctggtcaataaagtaagagggtaccacgtcctgctggtgagtgagtacaacctggctttgcctcgacgcgacgtcacttggttgtcacccccaactcaccgaccagttatttcattctcccatggtgcaggacgaccactcactcatgttggaccgaaacggagctgcgctgcagtgaaccaacagtgggctgaatgtcacaggcgccgataggtgctacgacctaagtttaggactgccggctgacgccggcaggttcgacttggtctttgtgaacattcacacggaacgacttacagtgtccgcggctatccacgatgctggattcaaatcctgacggccgactgcggccgtccaagctgaaccagaaacacttgtaagtgtgccttttcagaatccaccactaccagcagtgtgtcgaccacgccatgaagctgcagatgcttgggggagatgcgctacgactgctaaaacccggcggcatcttgaaagtcttaggtggtgatggtcgtcacacagctggtgcggtacttcgacgtctacgaaccccctctacgcgatgctgacgattttgggccgccgtagaacttgagagcttacggatacgccgataaaatcagcgaagccgttgtttcctccttaagcagaaagttctcgtctgcaagagtgttgcgcccggattgtgtcacactctcgaatgcctatgcggctattttagtcgcttcggcaacaaaggaggaattcgtctttcaagagcagacgttctcacaacgcgggcctaacacagtgcagcaatacagaagtgttcttgctgttctccaactttgacaacggaaagagaccctctacgctacaccagatgaataccaagctgagtgccgtgtatgccgtcgttatgtcttcacaagaacgacaagaggttgaaactgttgcctttctctgggagatgcgatgtggtctacttatggttcgactcacggcacatacggggagaagccatgcacacggccgggtgtgcaccatcctacagagttaagagagcagacatagccacgtgcacagaagcggctgtggttaacgcagctaacgcctcttcggtacgtgtgccggcccacacgtggtaggatgtctcaattctctcgtctgtatcggtgcacgtgtcttcgccgacaccaattgcgtcgattgccccgtggaactgtaggggatggcgtatgcagggccgtggcgaagaaatggccgtcagcctttaagggagcagcaacaccagtgggcacaattaaaacagtgggcaccttgacatcccctaccgcatacgtcccggcaccgcttctttaccggcagtcggaaattccctcgtcgttgtggtcacccgtgttaattttgtcacatgtgcggctcgtaccccgtcatccacgctgtagcgcctaatttctctgccacgactgaagcggaaggggaccgcgaattggccgctgtctaccgggcagtacacgccgagcatggggcagtaggtgcgacatcgcggattaaagagacggtgctgacttcgccttcccctggcgcttaaccggcgacagatggcccgtgtggccgccgaagtaaacagactgtcactgagcagcgtagccatcccgctgctgtccacaggagtgttcagcggcggaagagataggctgcagcaatccccaccggcggcttcatttgtctgacagtgactcgtcgcatcggtagggcgacgacaggtgtcctcacaagtcgccgccttctctatccgacgtcgttagggtcaaccatctattcacagcaatggacgccacggacgctgacgtgaccatctactgcagagacaaaagttgggagaagaaaatccaggaagccattgacatagttggtagataagtgtcgttacctgcggtgcctgcgactgcactggtagatgacgtctctgttttcaaccctcttcttttaggtccttcggtaactgtagaggacggctgtggagttgctcaatgatgacgtggagctgaccacagacttggtgagagtgcacccggacagcagcctggtgggtcgtaagggctacagtctcctgccgacacctcaacgagttactactgcacctcgactggtgtctgaaccactctcacgtgggcctgtcgtcggaccacccagcattcccgatgtcaaccactgacgggtcgctgtactcgtactttgaaggtacgaaattcaaccaggctgctattgatatggcagagatactgacgttgtggcccagactgcaagtggtgactgcccagcgacatgagcatgaaacttccatgctttaagttggtccgacgataactataccgtctctatgactgcaacaccgggtctgacgttcaggcaaacgaacagatatgcctatacgcgctgggcgaaacaatggacaacatcagatccaaatgtccggtgaacgattccgattcatcaacacctcccagtccgtttgcttgtctatacggatatgcgcgacccgctttgttacctgttgtagtctaggtttacaggccacttgctaaggctaagtagttgtggagggtcgacagtgccctgcctgtgccgctacgcaatgacagcagaacggatcgcccgccttaggtcacaccaagttaaaagcatggtggtttgctcatcttttcccctgtcacgggacggacacggcgatgcgttactgtcgtcttgcctagcgggcggaatccagtgtggttcaattttcgtaccaccaaacgagtagaaaagggctcccgaaataccatgtagatggggtgcagaaggtaaagtgcgagaaggttctcctgttcgacccgacggtaccttcagtggttagtccgcggaagtatggagggctttatggtacatctaccccacgtcttccatttcacgctcttccaagaggacaagctgggctgccatggaagtcaccaatcaggcgccttcatacccgcatctacgacggaccactcagatcggtcgttacgagggtttgacttggactggaccaccgactcgtcttccactgccagcgataccatgtcgctaccggcgtagatgctgcctggtgagtctagccagcaatgctcccaaactgaacctgacctggtggctgagcagaaggtgacggtcgctatggtacagcgatggcagtttgcagtcgtgtgacatcgactcgatctacgagccaatggctcccatagtagtgacggctgacgtacaccctgaacccgcaggcatcgcggacctggtcaaacgtcagcacactgtagctgagctagatgctcggttaccgagggtatcatcactgccgactgcatgtgggacttgggcgtccgtagcgcctggacgcggcagatgtgcaccctgaacccgcagaccatgtggacctggagaacccgattcctccaccgcgcccgaagagagctgcataccttgcctcccgcgcggcgccgtctacacgtgggacttgggcgtctggtacacctggacctcttgggctaaggaggtggcgcgggcttctctcgacgtatggaacggagggcgcgcccggagcgaccggtgccggcgccgagaaagccgacgcctgccccaaggactgcgtttaggaacaagctgcctttgacgttcggcgactttgacgagcacgagcctcgctggccacggccgcggctctttcggctgcggacggggttcctgacgcaaatccttgttcgacggaaactgcaagccgctgaaactgctcgtgctggtcgatgcgttggcctccgggattactttcggagacttcgacgacgtcctgcgactaggccgcgcgggtgcatatattttctcctcggacactggcagcccagctacgcaaccggaggccctaatgaaagcctctgaagctgctgcaggacgctgatccggcgcgcccacgtatataaaagaggagcctgtgaccgtcgggacatttacaacaaaaatccgttaggcagcacaatctccagtgcgcacaactggatgcggtccaggaggagaaaatgtacccgccaaaattggatactgcctgtaaatgttgtttttaggcaatccgtcgtgttagaggtcacgcgtgttgacctacgccaggtcctcctcttttacatgggcggttttaacctatgacagagggagaagctgttgctgctgaaaatgcagatgcacccatcggaggctaataagagtcgataccagtctcgcaaagtggagaacatgaaagccacggttctccctcttcgacaacgacgacttttacgtctacgtgggtagcctccgattattctcagctatggtcagagcgtttcacctcttgtactttcggtgccaggtggacaggctcacatcgggggccagattgtacacgggagcggacgtaggccgcataccaacatacgcggttcggtacccccgccccgtgtactcccctccacctgtccgagtgtagcccccggtctaacatgtgccctcgcctgcatccggcgtatggttgtatgcgccaagccatgggggcggggcacatgaggggaaccgtgatcgaaagattctcaagccccgatgtagcaatcgcagcgtgcaacgaatacctatccagaaattacccaacagtggcgtcgtaccagataacagtggcactagctttctaagagttcggggctacatcgttagcgtcgcacgttgcttatggataggtctttaatgggttgtcaccgcagcatggtctattgtcatgaatacgacgcatacttggacatggttgacgggtcggatagttgcttggacagagcgacattctgcccggcgaagctccggtgctacccgaaacatcatacttatgctgcgtatgaacctgtaccaactgcccagcctatcaacgaacctgtctcgctgtaagacgggccgcttcgaggccacgatgggctttgtagttgcgtaccaccagccgactgtacgcagtgccgtcccgtcaccctttcagaacacactacagaacgtgctagcggctgccaccaagagaaactgcaacgtcacgcatggtggtcggctgacatgcgtcacggcagggcagtgggaaagtcttgtgtgatgtcttgcacgatcgccgacggtggttctctttgacgttgcagacgcaaatgcgagaactacccaccatggactcggcagtgttcaacgtggagtgcttcaagcgctatgcctgctccggagaatattgggaagaatatgctatgcgtttacgctcttgatgggtggtacctgagccgtcacaagttgcacctcacgaagttcgcgatacggacgaggcctcttataacccttcttatacgataacaacctatccggataaccactgagaacatcactacctatgtgaccaaattgaaaggcccgaaagctgctgccttgttcgctaagacccacaacttggtttgttggataggcctattggtgactcttgtagtgatggatacactggtttaactttccgggctttcgacgacggaacaagcgattctgggtgttgaaccatccgctgcaggaggttcccatggacagattcacggtcgacatgaaacgagatgtcaaagtcactccagggacgaaacacacagaggaaagacccaaagtcaggcgacgtcctccaagggtacctgtctaagtgccagctgtactttgctctacagtttcagtgaggtccctgctttgtgtgtctcctttctgggtttcagcaggtaattcaagcagcggagccattggcgaccgcttacctgtgcggcatccacagggaattagtaaggagactaaatgctgtgttacgccctaacgtgcgtccattaagttcgtcgcctcggtaaccgctggcgaatggacacgccgtaggtgtcccttaatcattcctctgatttacgacacaatgcgggattgcacgacacattgtttgatatgtcggccgaagactttgacgcgatcatcgcctctcacttccacccaggagacccggttctagagacggacattgcatcattcgatgtgtaacaaactatacagccggcttctgaaactgcgctagtagcggagagtgaaggtgggtcctctgggccaagatctctgcctgtaacgtagtaagctcaaaagccaggacgactccttggctcttacaggtttaatgatcctcgaagatctaggggtggatcagtacctgctggacttgatcgaggcagcctttggggttttcggtcctgctgaggaaccgagaatgtccaaattactaggagcttctagatccccacctagtcatggacgacctgaactagctccgtcggaaacccgaaatatccagctgtcacctaccaactggcacgcgcttcaagttcggagctatgatgaaatcgggcatgtttctgactttgtttattaacactgttttgactttataggtcgacagtggatggttgaccgtgcgcgaagttcaagcctcgatactactttagcccgtacaaagactgaaacaaataattgtgacaaaactacatcaccatagcaagcagggtactggagcagagactcactgactccgcctgtgcggccttcatcggcgacgacaacatcgttcacggagtgatctccgatgtagtggtatcgttcgtcccatgacctcgtctctgagtgactgaggcggacacgccggaagtagccgctgctgttgtagcaagtgcctcactagaggctcaagctgatggcggagaggtgcgcgtcgtgggtcaacatggaggtgaagatcattgacgctgtcatgggcgaaaaacccccatatttttgtgggggattcgttcgactaccgcctctccacgcgcagcacccagttgtacctccacttctagtaactgcgacagtacccgctttttgggggtataaaaacaccccctaagatagtttttgacagcgtcacacagaccgcctgccgtgtttcagacccacttaagcgcctgttcaagttgggtaagccgctaacagctgaagacaagcaggtatcaaaaactgtcgcagtgtgtctggcggacggcacaaagtctgggtgaattcgcggacaagttcaacccattcggcgattgtcgacttctgttcgtccacgaagacaggcgacgagcactgagtgacgaggttagcaagtggttccggacaggcttgggggccgaactggaggtggcactaacatctaggtatgaggttgcttctgtccgctgctcgtgactcactgctccaatcgttcaccaaggcctgtccgaacccccggcttgacctccaccgtgattgtagatccatactccaagagggctgcaaaagtatcctcatagccatggccaccttggcgagggacattaaggcgtttaagaaattgagaggacctgttatacacctctacggcggttctcccgacgttttcataggagtatcggtaccggtggaaccgctccctgtaattccgcaaattctttaactctcctggacaatatgtggagatgccgccacctagattggtgcgttaatacacagaattctgattggatcccggtccgaagcgcgctttcccgggaactcgagttcactagtcgatcccgcggccgctttggatctaaccacgcaattatgtgtcttaagactaacctagggccaggcttcgcgcgaaagggcccttgagctcaagtgatcagctagggcgccggcgaaacgaacctaggcaagcatgcgggcccagtgggtaattaattgaattacatccctacgcaaacgttttacggccgccggtggcgcccgcgcccggcggcccggcttggatccgttcgtacgcccgggtcacccattaattaacttaatgtagggatgcgtttgcaaaatgccggcggccaccgcgggcgcgggccgccgggctccttggccgttgcaggccactccggtggctcccgtcgtccccgacttccaggcccagcagatgcagcaactcatcagcgccgtaaatgcgctgacaatgaggaaccggcaacgtccggtgaggccaccgagggcagcaggggctgaaggtccgggtcgtctacgtcgttgagtagtcgcggcatttacgcgactgttacagacagaacgcaattgctcctgctaggagcttaattcgacgaataattggatttttattttattttgcaattggtttttaatatttccaaaaaaaaaaaatctgtcttgcgttaacgaggacgatcctcgaattaagctgcttattaacctaaaaataaaataaaacgttaaccaaaaattataaaggttttttttttttaaaaaaaaaaaaaaaaaaaaaaaaaaaaaaaaaaaaaaaaaaaaaaaaaaaaaaaaaactagaaatcgatcggcagtgcgccttccaggagaactagcgcttttttttttttttttttttttttttttttttttttttttttttttttttttttttttgatctttagctagccgtcacgcggaaggtcctcttgatcgcgggccgcatcgataagcttgtcgactcgaagatccagacatgataagatacattgatgagtttggacaaaccacaactagaatgcagtgaaaaaaatgcttccggcgtagctattcgaacagctgagcttctaggtctgtactattctatgtaactactcaaacctgtttggtgttgatcttacgtcactttttttacgaatatttgtgaaatttgtgatgctattgctttatttgtaaccattataagctgcaataaacaagttaacaacaacaattgcattcattttatgtttcaggttataaacactttaaacactacgataacgaaataaacattggtaatattcgacgttatttgttcaattgttgttgttaacgtaagtaaaatacaaagtccaacagggggaggtgtgggaggttttttcggatctgggcgtggttaagggtgggaaagaatatataaggtgggggtcttatgtagttttgtatctgttttgcagtccccctccacaccctccaaaaaagcctagacccgcaccaattcccaccctttcttatatattccacccccagaatacatcaaaacatagacaaaacgtgcagccgccgccgccatgagcaccaactcgtttgatggaagcattgtgagctcatatttgacaacgcgcatgcccccatgggccggggtgcgtcagaatgcgtcggcggcggcggtactcgtggttgagcaaactaccttcgtaacactcgagtataaactgttgcgcgtacgggggtacccggccccacgcagtcttactgatgggctccagcattgatggtcgccccgtcctgcccgcaaactctactaccttgacctacgagaccgtgtctggaacgccgttggagactgcagcctcactacccgaggtcgtaactaccagcggggcaggacgggcgtttgagatgatggaactggatgctctggcacagaccttgcggcaacctctgacgtcggagcgccgccgcttcagccgctgcagccaccgcccgcgggattgtgactgactttgctttcctgagcccgcttgcaagcagtgcagcttcccgttcatccgccgcggcggcgaagtcggcgacgtcggtggcgggcgccctaacactgactgaaacgaaaggactcgggcgaacgttcgtcacgtcgaagggcaagtaggcggcgcgatgacaagttgacggctcttttggcacaattggattctttgacccgggaacttaatgtcgtttctcagcagctgttggatctgcgccagcaggtttgcgctactgttcaactgccgagaaaaccgtgttaacctaagaaactgggcccttgaattacagcaaagagtcgtcgacaacctagacgcggtcgtccaaactgccctgaaggcttcctcccctcccaatgcggtttaaaacataaataaaaaaccagactctgtttggatttggatcaagcaagtgtcttgctgtctttagacgggacttccgaaggaggggagggttacgccaaattttgtatttattttttggtctgagacaaacctaaacctagttcgttcacagaacgacagaaattttaggggttttgcgcgcgcggtaggcccgggaccagcggtctcggtcgttgagggtcctgtgtattttttccaggacgtggtaaaggtgactctggatgaaatccccaaaacgcgcgcgccatccgggccctggtcgccagagccagcaactcccaggacacataaaaaaggtcctgcaccatttccactgagacctacttcagatacatgggcataagcccgtctctggggtggaggtagcaccactgcagagcttcatgctgcggggtggtgttgtagatgatccagtcgtagcaggaagtctatgtacccgtattcgggcagagaccccacctccatcgtggtgacgtctcgaagtacgacgccccaccacaacatctactaggtcagcatcgtccagcgctgggcgtggtgcctaaaaatgtctttcagtagcaagctgattgccaggggcaggcccttggtgtaagtgtttacaaagcggttaagctgggatggtcgcgacccgcaccacggatttttacagaaagtcatcgttcgactaacggtccccgtccgggaaccacattcacaaatgtttcgccaattcgaccctaccgtgcatacgtggggatatgagatgcatcttggactgtatttttaggttggctatgttcccagccatatccctccggggattcatgttgtgcagaaccacccacgtatgcacccctatactctacgtagaacctgacataaaaatccaaccgatacaagggtcggtatagggaggcccctaagtacaacacgtcttggtggagcacagtgtatccggtgcacttgggaaatttgtcatgtagcttagaaggaaatgcgtggaagaacttggagacgcccttgtgacctccaagattttccatcgtgtcacataggccacgtgaaccctttaaacagtacatcgaatcttcctttacgcaccttcttgaacctctgcgggaacactggaggttctaaaaggttgcattcgtccataatgatggcaatgggcccacgggcggcggcctgggcgaagatatttctgggatcactaacgtcatagttgtgttccaggatgagatcacgtaagcaggtattactaccgttacccgggtgcccgccgccggacccgcttctataaagaccctagtgattgcagtatcaacacaaggtcctactctaggtcataggccatttttacaaagcgcgggcggagggtgccagactgcggtataatggttccatccggcccaggggcgtagttaccctcacagatttgcattcagtatccggtaaaaatgtttcgcgcccgcctcccacggtctgacgccatattaccaaggtaggccgggtccccgcatcaatgggagtgtctaaacgtaatcccacgctttgagttcagatggggggatcatgtctacctgcggggcgatgaagaaaacggtttccggggtaggggagatcagctgggaagaaagcaggtagggtgcgaaactcaagtctacccccctagtacagatggacgccccgctacttcttttgccaaaggccccatcccctctagtcgacccttctttcgtccatcctgagcagctgcgacttaccgcagccggtgggcccgtaaatcacacctattaccgggtgcaactggtagttaagagagctgcagctgccgtcatccctaggactcgtcgacgctgaatggcgtcggccacccgggcatttagtgtggataatggcccacgttgaccatcaattctctcgacgtcgacggcagtagggagagcaggggggccacttcgttaagcatgtccctgactcgcatgttttccctgaccaaatccgccagaaggcgctcgccgcccagcgatagcagttcttgcctcgtccccccggtgaagcaattcgtacagggactgagcgtacaaaagggactggtttaggcggtcttccgcgagcggcgggtcgctatcgtcaagaacgaaggaagcaaagtttttcaacggtttgagaccgtccgccgtaggcatgcttttgagcgtttgaccaagcagttccaggcggtcccacagctcggtcacctttccttcgtttcaaaaagttgccaaactctggcaggcggcatccgtacgaaaactcgcaaactggttcgtcaaggtccgccagggtgtcgagccagtggagctctacggcatctcgatccagcatatctcctcgtttcgcgggttggggcggctttcgctgtacggcagtagtcggtgctcgtccagacgggccagggtccgagatgccgtagagctaggtcgtatagaggagcaaagcgcccaaccccgccgaaagcgacatgccgtcatcagccacgagcaggtctgcccggtcccagatgtctttccacgggcgcagggtcctcgtcagcgtagtctgggtcacggtgaaggggtgcgctccgggctgcgcgctggccagggtgcgcttgaggctggtacagaaaggtgcccgcgtcccaggagcagtcgcatcagacccagtgccacttccccacgcgaggcccgacgcgcgaccggtcccacgcgaactccgacctcctgctggtgctgaagcgctgccggtcttcgccctgcgcgtcggccaggtagcatttgaccatggtgtcatagtccagcccctccgcggcgtggcccttaggacgaccacgacttcgcgacggccagaagcgggacgcgcagccggtccatcgtaaactggtaccacagtatcaggtcggggaggcgccgcaccgggaaggcgcgcagcttgcccttggaggaggcgccgcacgagggagacttttgagggcgtagagcttgggcgcgagaaataccgattccggggagtaggcatccgccgcgcgtcgaacgggaacctcctccgcggcgtgctccctctgaaaactcccgcatctcgaacccgcgctctttatggctaaggcccctcatccgtaggccgccgcaggccccgcagacggtctcgcattccacgagccaggtgagctctggccgttcggggtcaaaaaccaggtttcccccatgctttttgatgcgtttgcggcgtccggggcgtctgccagagcgtaaggtgctcggtccactcgagaccggcaagccccagtttttggtccaaagggggtacgaaaaactacgcaaacttacctctggtttccatgagccggtgtccacgctcggtgacgaaaaggctgtccgtgtccccgtatacagacttgagaggcctgtcctcgagcggtgttgaatggagaccaaaggtactcggccacaggtgcgagccactgcttttccgacaggcacaggggcatatgtctgaactctccggacaggagctcgccacaaccgcggtcctcctcgtatagaaactcggaccactctgagacaaaggctcgcgtccaggccagcacgaaggaggctaagtgggaggggtagcggtcgttgtggcgccaggaggagcatatctttgagcctggtgagactctgtttccgagcgcaggtccggtcgtgcttcctccgattcaccctccccatcgccagcaacaccactagggggtccactcgctccagggtgtgaagacacatgtcgccctcttcggcatcaaggaaggtgattggtttgtaggtgtaggccacgtgaccgggggtgatcccccaggtgagcgaggtcccacacttctgtgtacagcgggagaagccgtagttccttccactaaccaaacatccacatccggtgcactggccctgttcctgaaggggggctataaaagggggtgggggcgcgttcgtcctcactctcttccgcatcgctgtctgcgagggccagctgttggggtgagtactccacaaggacttccccccgatattttcccccacccccgcgcaagcaggagtgagagaaggcgtagcgacagacgctcccggtcgacaaccccactcatgaggctctgaaaagcgggcatgacttctgcgctaagattgtcagtttccaaaaacgaggaggatttgatattcacctggcccgcggtgatgcctttgagggtgggagacttttcgcccgtactgaagacgcgattctaacagtcaaaggtttttgctcctcctaaactataagtggaccgggcgccactacggaaactcccaccccgcatccatctggtcagaaaagacaatctttttgttgtcaagcttggtggcaaacgacccgtagagggcgttggacagcaacttggcgatggagcgcagggcgtaggtagaccagtcttttctgttagaaaaacaacagttcgaaccaccgtttgctgggcatctcccgcaacctgtcgttgaaccgctacctcgcgtcggtttggtttttgtcgcgatcggcgcgctccttggccgcgatgtttagctgcacgtattcgcgcgcaacgcaccgccattcgggaaagacggtggtgcgcccaaaccaaaaacagcgctagccgcgcgaggaaccggcgctacaaatcgacgtgcataagcgcgcgttgcgtggcggtaagccctttctgccaccacgcgtcgtcgggcaccaggtgcacgcgccaaccgcggttgtgcagggtgacaaggtcaacgctggtggctacctctccgcgtaggcgctcgttggtccagcagaagcagcccgtggtccacgtgcgcggttggcgccaacacgtcccactgttccagttgcgaccaccgatggagaggcgcatccgcgagcaaccaggtcgtctggcggccgcccttgcgcgagcagaatggcggtagggggtctagctgcgtctcgtccggggggtctgcgtccacggtaaagaccccgggcagcaggcgcgcccgccggcgggaacgcgctcgtcttaccgccatcccccagatcgacgcagagcaggccccccagacgcaggtgccatttctggggcccgtcgtccgcgcggtcgaagtagtctatcttgcatccttgcaagtctagcgcctgctgccatgcgcgggcggcaagcgcgcgctcgtatgggttgagtgggggaccccatggccagcttcatcagatagaacgtaggaacgttcagatcgcggacgacggtacgcgcccgccgttcgcgcgcgagcatacccaactcaccccctggggtaccgatggggtgggtgagcgcggaggcgtacatgccgcaaatgtcgtaaacgtagaggggctctctgagtattccaagatatgtagggtagcatcttccaccgctaccccacccactcgcgcctccgcatgtacggcgtttacagcatttgcatctccccgagagactcataaggttctatacatcccatcgtagaaggtggcgggatgctggcgcgcacgtaatcgtatagttcgtgcgagggagcgaggaggtcgggaccgaggttgctacgggcgggctgctctgctcggaagactatctgcctacgaccgcgcgtgcattagcatatcaagcacgctccctcgctcctccagccctggctccaacgatgcccgcccgacgagacgagccttctgatagaccctgaagatggcatgtgagttggatgatatggttggacgctggaagacgttgaagctggcgtctgtgagacctaccgcgtcacgcacgaaggaggcgtagggacttctaccgtacactcaacctactataccaacctgcgaccttctgcaacttcgaccgcagacactctggatggcgcagtgcgtgcttcctccgcatcgagtcgcgcagcttgttgaccagctcggcggtgacctgcacgtctagggcgcagtagtccagggtttccttgatgatgtcatacttatcctgtcccttttctcagcgcgtcgaacaactggtcgagccgccactggacgtgcagatcccgcgtcatcaggtcccaaaggaactactacagtatgaataggacagggaaaattttccacagctcgcggttgaggacaaactcttcgcggtctttccagtactcttggatcggaaacccgtcggcctccgaacggtaagagcctagcatgtaaaaaggtgtcgagcgccaactcctgtttgagaagcgccagaaaggtcatgagaacctagcctttgggcagccggaggcttgccattctcggatcgtacatgaactggttgacggcctggtaggcgcagcatcccttttctacgggtagcgcgtatgcctgcgcggccttcatgaccagcatgaagggcacgagctgcttccttgaccaactgccggaccatccgcgtcgtagggaaaagatgcccatcgcgcatacggacgcgccggaagtactggtcgtacttcccgtgctcgacgaagccaaaggcccccatccaagtataggtctctacatcgtaggtgacaaagagacgctcggtgcgaggatgcgagccgatcgggaagaactggatctcccgccggtttccgggggtaggttcatatccagagatgtagcatccactgtttctctgcgagccacgctcctacgctcggctagcccttcttgacctagagggcggaccaattggaggagtggctattgatgtggtgaaagtagaagtccctgcgacgggccgaacactcgtgctggcttttgtaaaaacgtgcgcagtactggcatggttaacctcctcaccgataactacaccactttcatcttcagggacgctgcccggcttgtgagcacgaccgaaaacatttttgcacgcgtcatgaccgtgcggtgcacgggctgtacatcctgcacgaggttgacctgacgaccgcgcacaaggaagcagagtgggaatttgagcccctcgcctggcgggtttggctggcgccacgtgcccgacatgtaggacgtgctccaactggactgctggcgcgtgttccttcgtctcacccttaaactcggggagcggaccgcccaaaccgacctggtcttctacttcggctgcttgtccttgaccgtctggctgctcgaggggagttacggtggatcggaccaccacgccgcgcgagcccaaagtccagatgtaccagaagatgaagccgacgaacaggaactggcagaccgacgagctcccctcaatgccacctagcctggtggtgcggcgcgctcgggtttcaggtctacaccgcgcgcggcggtcggagcttgatgacaacatcgcgcagatgggagctgtccatggtctggagctcccgcggcgtcaggtcaggcgggagctcctgcagggcgcgcgccgccagcctcgaactactgttgtagcgcgtctaccctcgacaggtaccagacctcgagggcgccgcagtccagtccgccctcgaggacgtcgtttacctcgcatagacgggtcagggcgcgggctagatccaggtgatacctaatttccaggggctggttggtggcggcgtcgatggcttgcaagaggccgcaaatggagcgtatctgcccagtcccgcgcccgatctaggtccactatggattaaaggtccccgaccaaccaccgccgcagctaccgaacgttctccggccatccccgcggcgcgactacggtaccgcgcggcgggcggtgggccgcgggggtgtccttggatgatgcatctaaaagcggtgacgcgggcgagcccccgggtaggggcgccgcgctgatgccatggcgcgccgcccgccacccggcgcccccacaggaacctactacgtagattttcgccactgcgcccgctcgggggccaggtagggggggctccggacccgccgggagagggggcaggggcacgtcggcgccgcgcgcgggcaggagctggtgctgcgcgcgtaggttgctggcgaactccatcccccccgaggcctgggcggccctctcccccgtccccgtgcagccgcggcgcgcgcccgtcctcgaccacgacgcgcgcatccaacgaccgcttggcgacgacgcggcggttgatctcctgaatctggcgcctctgcgtgaagacgacgggcccggtgagcttgagcctgaaagagagttcgacagaatcaatttcgctgctgcgccgccaactagaggacttagaccgcggagacgcacttctgctgcccgggccactcgaactcggactttctctcaagctgtcttagttaaacggtgtcgttgacggcggcctggcgcaaaatctcctgcacgtctcctgagttgtcttgataggcgatctcggccatgaactgctcgatctcttcctcctggccacagcaactgccgccggaccgcgttttagaggacgtgcagaggactcaacagaactatccgctagagccggtacttgacgagctagagaaggaggacgagatctccgcgtccggctcgctccacggtggcggcgaggtcgttggaaatgcgggccatgagctgcgagaaggcgttgaggcctccctcgttccagacgctctagaggcgcaggccgagcgaggtgccaccgccgctccagcaacctttacgcccggtactcgacgctcttccgcaactccggagggagcaaggtctgccggctgtagaccacgcccccttcggcatcgcgggcgcgcatgaccacctgcgcgagattgagctccacgtgccgggcgaagacggcgtagtttcgcaggcgccgacatctggtgcgggggaagccgtagcgcccgcgcgtactggtggacgcgctctaactcgaggtgcacggcccgcttctgccgcatcaaagcgtccggctgaaagaggtagttgagggtggtggcggtgtgttctgccacgaagaagtacataacccagcgtcgcaacgtggattcgttgcaattgttgtgtaggtacgactttctccatcaactcccaccaccgccacacaagacggtgcttcttcatgtattgggtcgcagcgttgcacctaagcaacgttaacaacacatccatctccgccgccgagggacctgagcgagtccgcatcgaccggatcggaaaacctctcgagaaaggcgtctaaccagtcacagtcgcaaggtaggctgagcacgaggcggcggctccctggactcgctcaggcgtagctggcctagccttttggagagctctttccgcagattggtcagtgtcagcgttccatccgactcgtgcgtggcgggcggcagcgggcggcggtcggggttgtttctggcggaggtgctgctgatgatgtaattaaagtaggcggtcttgagacggcggatggtcgacgcaccgcccgccgtcgcccgccgccagccccaacaaagaccgcctccacgacgactactacattaatttcatccgccagaactctgccgcctaccagctgagaagcaccatgtccttgggtccggcctgctgaatgcgcaggcggtcggccatgccccaggcttcgttttgacatcggcgcaggtctttgtagtagtctttcttcgtggtacaggaacccaggccggacgacttacgcgtccgccagccggtacggggtccgaagcaaaactgtagccgcgtccagaaacatcatcagaagcatgagcctttctaccggcacttcttcttctccttcctcttgtcctgcatctcttgcatctatcgctgcggcggcggcggagtttggccgtaggtggcgcgtactcggaaagatggccgtgaagaagaagaggaaggagaacaggacgtagagaacgtagatagcgacgccgccgccgcctcaaaccggcatccaccgcccctcttcctcccatgcgtgtgaccccgaagcccctcatcggctgaagcagggctaggtcggcgacaacgcgctcggctaatatggcctgctgcacctgcgggagaaggagggtacgcacactggggcttcggggagtagccgacttcgtcccgatccagccgctgttgcgcgagccgattataccggacgacgtggacggtgagggtagactggaagtcatccatgtccacaaagcggtggtatgcgcccgtgttgatggtgtaagtgcagttggccataacggaccagttaacggtctcactcccatctgaccttcagtaggtacaggtgtttcgccaccatacgcgggcacaactaccacattcacgtcaaccggtattgcctggtcaattgccagaggtgacccggctgcgagagctcggtgtacctgagacgcgagtaagccctcgagtcaaatacgtagtcgttgcaagtccgcaccaggtactggtatcccacccactgggccgacgctctcgagccacatggactctgcgctcattcgggagctcagtttatgcatcagcaacgttcaggcgtggtccatgaccatagggtgcaaaaagtgcggcggcggctggcggtagaggggccagcgtagggtggccggggctccgggggcgagatcttccaacataaggcgatgatatccgtagatggtttttcacgccgccgccgaccgccatctccccggtcgcatcccaccggccccgaggcccccgctctagaaggttgtattccgctactataggcatctactacctggacatccaggtgatgccggcggcggtggtggaggcgcgcggaaagtcgcggacgcggttccagatgttgcgcagcggcaaaaagtgctccatggatggacctgtaggtccactacggccgccgccaccacctccgcgcgcctttcagcgcctgcgccaaggtctacaacgcgtcgccgtttttcacgaggtacctcgggacgctctggccggtcaggcgcgcgcaatcgttgacgctctagaccgtgcaaaaggagagcctgtaagcgggcactcttccgtggtctggtggataagccctgcgagaccggccagtccgcgcgcgttagcaactgcgagatctggcacgttttcctctcggacattcgcccgtgagaaggcaccagaccacctataattcgcaagggtatcatggcggacgaccggggttcgagccccgtatccggccgtccgccgtgatccatgcggttaccgcccgcgtgtcgaacccaggtgttaagcgttcccatagtaccgcctgctggccccaagctcggggcataggccggcaggcggcactaggtacgccaatggcgggcgcacagcttgggtccactgcgacgtcagacaacgggggagtgctccttttggcttccttccaggcgcggcggctgctgcgctagcttttttggccactggccgcgcgcagcgtaagcacgctgcagtctgttgccccctcacgaggaaaaccgaaggaaggtccgcgccgccgacgacgcgatcgaaaaaaccggtgaccggcgcgcgtcgcattcgggttaggctggaaagcgaaagcattaagtggctcgctccctgtagccggagggttattttccaagggttgagtcgcgggacccccggttcgagtctcggaccaatccgacctttcgctttcgtaattcaccgagcgagggacatcggcctcccaataaaaggttcccaactcagcgccctgggggccaagctcagagcctccggccggactgcggcgaacgggggtttgcctccccgtcatgcaagaccccgcttgcaaattcctccggaaacagggacgagccccttttttgcttttccggccggcctgacgccgcttgcccccaaacggaggggcagtacgttctggggcgaacgtttaaggaggcctttgtccctgctcggggaaaaaacgaaaaggcagatgcatccggtgctgcggcagatgcgcccccctcctcagcagcggcaagagcaagagcagcggcagacatgcagggcaccctcccctcctcctaccggtctacgtaggccacgacgccgtctacgcggggggaggagtcgtcgccgttctcgttctcgtcgccgtctgtacgtcccgtgggaggggaggaggatggccgtcaggaggggcgacatccgcggttgacgcggcagcagatggtgattacgaacccccgcggcgccgggcccggcactacctggacttggaggagggcgagcagtcctccccgctgtaggcgccaactgcgccgtcgtctaccactaatgcttgggggcgccgcggcccgggccgtgatggacctgaacctcctcccgctgggcctggcgcggctaggagcgccctctcctgagcggtacccaagggtgcagctgaagcgtgatacgcgtgaggcgtacgtgccgcggcagaacctgtttcccggaccgcgccgatcctcgcgggagaggactcgccatgggttcccacgtcgacttcgcactatgcgcactccgcatgcacggcgccgtcttggacaaacgcgaccgcgagggagaggagcccgaggagatgcgggatcgaaagttccacgcagggcgcgagctgcggcatggcctgaatcgcgagcggttgctgcgcggcgctggcgctccctctcctcgggctcctctacgccctagctttcaaggtgcgtcccgcgctcgacgccgtaccggacttagcgctcgccaacgacgcgcaggaggactttgagcccgacgcgcgaaccgggattagtcccgcgcgcgcacacgtggcggccgccgacctggtaaccgcatacgagcagacggtgaaccatcctcctgaaactcgggctgcgcgcttggccctaatcagggcgcgcgcgtgtgcaccgccggcggctggaccattggcgtatgctcgtctgccacttggtggagattaactttcaaaaaagctttaacaaccacgtgcgtacgcttgtggcgcgcgaggaggtggctataggactgatgcatctgtgggactttgtaagccctctaattgaaagttttttcgaaattgttggtgcacgcatgcgaacaccgcgcgctcctccaccgatatcctgactacgtagacaccctgaaacattcggcgctggagcaaaacccaaatagcaagccgctcatggcgcagctgttccttatagtgcagcacagcagggacaacgaggcattcagggatgcgctgctaacgcgacctcgttttgggtttatcgttcggcgagtaccgcgtcgacaaggaatatcacgtcgtgtcgtccctgttgctccgtaagtccctacgcgacgattacatagtagagcccgagggccgctggctgctcgatttgataaacatcctgcagagcatagtggtgcaggagcgcagcttgagcctggctgacaaggtggctgtatcatctcgggctcccggcgaccgacgagctaaactatttgtaggacgtctcgtatcaccacgtcctcgcgtcgaactcggaccgactgttccaccgcgccatcaactattccatgcttagcctgggcaagttttacgcccgcaagatataccataccccttacgttcccatagacaaggaggtaaagatcgagggggcggtagttgataaggtacgaatcggacccgttcaaaatgcgggcgttctatatggtatggggaatgcaagggtatctgttcctccatttctagctccccttctacatgcgcatggcgctgaaggtgcttaccttgagcgacgacctgggcgtttatcgcaacgagcgcatccacaaggccgtgagcgtgagccggcggcaagatgtacgcgtaccgcgacttccacgaatggaactcgctgctggacccgcaaatagcgttgctcgcgtaggtgttccggcactcgcactcggccgccggcgagctcagcgaccgcgagctgatgcacagcctgcaaagggccctggctggcacgggcagcggcgatagagaggccgagtcctactttgacgcgggcgccgctcgagtcgctggcgctcgactacgtgtcggacgtttcccgggaccgaccgtgcccgtcgccgctatctctccggctcaggatgaaactgcgcccgcgtgacctgcgctgggccccaagccgacgcgccctggaggcagctggggccggacctgggctggcggtggcacccgcgcgcgctggcaacgtcggcggcgtgactggacgcgacccggggttcggctgcgcgggacctccgtcgaccccggcctggacccgaccgccaccgtgggcgcgcgcgaccgttgcagccgccgcacgaggaatatgacgaggacgatgagtacgagccagaggacggcgagtactaagcggtgatgtttctgatcagatgatgcaagacgcaacggacccggcggtctccttatactgctcctgctactcatgctcggtctcctgccgctcatgattcgccactacaaagactagtctactacgttctgcgttgcctgggccgccagcgggcggcgctgcagagccagccgtccggccttaactccacggacgactggcgccaggtcatggaccgcatcatgtcgctgactgcgcgcaatcctgaccgcccgccgcgacgtctcggtcggcaggccggaattgaggtgcctgctgaccgcggtccagtacctggcgtagtacagcgactgacgcgcgttaggactggcgttccggcagcagccgcaggccaaccggctctccgcaattctggaagcggtggtcccggcgcgcgcaaaccccacgcacgagaaggtgctggcgatcgcgcaaggccgtcgtcggcgtccggttggccgagaggcgttaagaccttcgccaccagggccgcgcgcgtttggggtgcgtgctcttccacgaccgctagctaaacgcgctggccgaaaacagggccatccggcccgacgaggccggcctggtctacgacgcgctgcttcagcgcgtggctcgttacaacagcggcaacgtatttgcgcgaccggcttttgtcccggtaggccgggctgctccggccggaccagatgctgcgcgacgaagtcgcgcaccgagcaatgttgtcgccgttgcagcagaccaacctggaccggctggtgggggatgtgcgcgaggccgtggcgcagcgtgagcgcgcgcagcagcagggcaacctgggctccatggttgcactacgtctggttggacctggccgaccaccccctacacgcgctccggcaccgcgtcgcactcgcgcgcgtcgtcgtcccgttggacccgaggtaccaacgtgataacgccttcctgagtacacagcccgccaacgtgccgcggggacaggaggactacaccaactttgtgagcgcactgcggctaatggtgactgagacaccgcttgcggaaggactcatgtgtcgggcggttgcacggcgcccctgtcctcctgatgtggttgaaacactcgcgtgacgccgattaccactgactctgtggcgaaagtgaggtgtaccagtctgggccagactattttttccagaccagtagacaaggcctgcagaccgtaaacctgagccaggctttcaaaaacttgcagggtttcactccacatggtcagacccggtctgataaaaaaggtctggtcatctgttccggacgtctggcatttggactcggtccgaaagtttttgaacgtcccgctgtggggggtgcgggctcccacaggcgaccgcgcgaccgtgtctagcttgctgacgcccaactcgcgcctgttgctgctgctaatagcgcccttcacgcgacaccccccacgcccgagggtgtccgctggcgcgctggcacagatcgaacgactgcgggttgagcgcggacaacgacgacgattatcgcgggaagtgcgacagtggcagcgtgtcccgggacacatacctaggtcacttgctgacactgtaccgcgaggccataggtcaggcgcatgtggacgagcatactttccaggctgtcaccgtcgcacagggccctgtgtatggatccagtgaacgactgtgacatggcgctccggtatccagtccgcgtacacctgctcgtatgaaaggtccagattacaagtgtcagccgcgcgctggggcaggaggacacgggcagcctggaggcaaccctaaactacctgctgaccaaccggcggcagaagatcccctctctaatgttcacagtcggcgcgcgaccccgtcctcctgtgcccgtcggacctccgttgggatttgatggacgactggttggccgccgtcttctaggggaggttgcacagtttaaacagcgaggaggagcgcattttgcgctacgtgcagcagagcgtgagccttaacctgatgcgcgacggggtaacgcccagcgtggcgcaacgtgtcaaatttgtcgctcctcctcgcgtaaaacgcgatgcacgtcgtctcgcactcggaattggactacgcgctgccccattgcgggtcgcaccgcctggacatgaccgcgcgcaacatggaaccgggcatgtatgcctcaaaccggccgtttatcaaccgcctaatggactacttgcatcgcgcggccgccgtgagacctgtactggcgcgcgttgtaccttggcccgtacatacggagtttggccggcaaatagttggcggattacctgatgaacgtagcgcgccggcggcactaccccgagtatttcaccaatgccatcttgaacccgcactggctaccgccccctggtttctacaccgggggattcgaggtgcccgagggtaacgatggatttggggctcataaagtggttacggtagaacttgggcgtgaccgatggcgggggaccaaagatgtggccccctaagctccacgggctcccattgctacctaacctctgggacgacatagacgacagcgtgttttccccgcaaccgcagaccctgctagagttgcaacagcgcgagcaggcagaggcggcgctgcgaaaggaaggagaccctgctgtatctgctgtcgcacaaaaggggcgttggcgtctgggacgatctcaacgttgtcgcgctcgtccgtctccgccgcgacgctttccttagcttccgcaggccaagcagcttgtccgatctaggcgctgcggccccgcggtcagatgctagtagcccatttccaagcttgatagggtctcttaccagcatcgaaggcgtccggttcgtcgaacaggctagatccgcgacgccggggcgccagtctacgatcatcgggtaaaggttcgaactatcccagagaatggtcgtctcgcaccacccgcccgcgcctgctgggcgaggaggagtacctaaacaactcgctgctgcagccgcagcgcgaaaaaaacctgcctccggcatttcccaagagcgtggtgggcgggcgcggacgacccgctcctcctcatggatttgttgagcgacgacgtcggcgtcgcgctttttttggacggaggccgtaaagggttcaacgggatagagagcctagtggacaagatgagtagatggaagacgtacgcgcaggagcacagggacgtgccaggcccgcgcccgcccacccgtcgtcaagttgccctatctctcggatcacctgttctactcatctaccttctgcatgcgcgtcctcgtgtccctgcacggtccgggcgcgggcgggtgggcagcagttaggcacgaccgtcagcggggtctggtgtgggaggacgatgactcggcagacgacagcagcgtcctggatttgggagggagtggcaacccgtttgcgcacctccgtgctggcagtcgccccagaccacaccctcctgctactgagccgtctgctgtcgtcgcaggacctaaaccctccctcaccgttgggcaaacgcgtggttcgccccaggctggggagaatgttttaaaaaaaaaaaagcatgatgcaaaataaaaaactcaccaaggccatggcaccgagcgttggttttcttgtattaagcggggtccgacccctcttacaaaattttttttttttcgtactacgttttattttttgagtggttccggtaccgtggctcgcaaccaaaagaacataaccccttagtatgcggcgcgcggcgatgtatgaggaaggtcctcctccctcctacgagagtgtggtgagcgcggcgccagtggcggcggcgctgggttctcggggaatcatacgccgcgcgccgctacatactccttccaggaggagggaggatgctctcacaccactcgcgccgcggtcaccgccgccgcgacccaagagccttcgatgctcccctggacccgccgtttgtgcctccgcggtacctgcggcctaccggggggagaaacagcatccgttactctgagttggcacccctattggaagctacgaggggacctgggcggcaaacacggaggcgccatggacgccggatggcccccctctttgtcgtaggcaatgagactcaaccgtggggataacgacaccacccgtgtgtacctggtggacaacaagtcaacggatgtggcatccctgaactaccagaacgaccacagcaactttctgaccacggtcattcaagctgtggtgggcacacatggaccacctgttgttcagttgcctacaccgtagggacttgatggtcttgctggtgtcgttgaaagactggtgccagtaagttaacaatgactacagcccgggggaggcaagcacacagaccatcaatcttgacgaccggtcgcactggggcggcgacctgaaaaccatcctgcataccaacattgttactgatgtcgggccccctccgttcgtgtgtctggtagttagaactgctggccagcgtgaccccgccgctggacttttggtaggacgtatggttgttgccaaatgtgaacgagttcatgtttaccaataagtttaaggcgcgggtgatggtgtcgcgcttgcctactaaggacaatcaggtggagctgaaatacgaacggtttacacttgctcaagtacaaatggttattcaaattccgcgcccactaccacagcgcgaacggatgattcctgttagtccacctcgactttatgctgtgggtggagttcacgctgcccgagggcaactactccgagaccatgaccatagaccttatgaacaacgcgatcgtggagcactacttgaaagtgggcagacacccacctcaagtgcgacgggctcccgttgatgaggctctggtactggtatctggaatacttgttgcgctagcacctcgtgatgaactttcacccgtctcagaacggggttctggaaagcgacatcggggtaaagtttgacacccgcaacttcagactggggtttgaccccgtcactggtcttgtcatgcctggggtatgtcttgccccaagacctttcgctgtagccccatttcaaactgtgggcgttgaagtctgaccccaaactggggcagtgaccagaacagtacggaccccataatacaaacgaagccttccatccagacatcattttgctgccaggatgcggggtggacttcacccacagccgcctgagcaacttgttgggcatccgcaagcgtatgtttgcttcggaaggtaggtctgtagtaaaacgacggtcctacgccccacctgaagtgggtgtcggcggactcgttgaacaacccgtaggcgttcgcgcaacccttccaggagggctttaggatcacctacgatgatctggagggtggtaacattcccgcactgttggatgtggacgcctaccaggcgagcttgaaacgttgggaaggtcctcccgaaatcctagtggatgctactagacctcccaccattgtaagggcgtgacaacctacacctgcggatggtccgctcgaactttgatgacaccgaacagggcgggggtggcgcaggcggcagcaacagcagtggcagcggcgcggaagagaactccaacgcggcagccgcggcaatgcagccggctactgtggcttgtcccgcccccaccgcgtccgccgtcgttgtcgtcaccgtcgccgcgccttctcttgaggttgcgccgtcggcgccgttacgtcggcctggaggacatgaacgatcatgccattcgcggcgacacctttgccacacgggctgaggagaagcgcgctgaggccgaagcagcggccgaagctgccgccccacctcctgtacttgctagtacggtaagcgccgctgtggaaacggtgtgcccgactcctcttcgcgcgactccggcttcgtcgccggcttcgacggcggggcgctgcgcaacccgaggtcgagaagcctcagaagaaaccggtgatcaaacccctgacagaggacagcaagaaacgcagttacaacctaataagcaatgacgcgacgcgttgggctccagctcttcggagtcttctttggccactagtttggggactgtctcctgtcgttctttgcgtcaatgttggattattcgttactgagcaccttcacccagtaccgcagctggtaccttgcatacaactacggcgaccctcagaccggaatccgctcatggaccctgctttgcactcctgacgtaatcgtggaagtgggtcatggcgtcgaccatggaacgtatgttgatgccgctgggagtctggccttaggcgagtacctgggacgaaacgtgaggactgcattcctgcggctcggagcaggtctactggtcgttgccagacatgatgcaagaccccgtgaccttccgctccacgcgccagatcagcaactttccggtggtgggggacgccgagcctcgtccagatgaccagcaacggtctgtactacgttctggggcactggaaggcgaggtgcgcggtctagtcgttgaaaggccaccaccccgccgagctgttgcccgtgcactccaagagcttctacaacgaccaggccgtctactcccaactcatccgccagtttacctctctgacccacgtgttcaatgcggctcgacaacgggcacgtgaggttctcgaagatgttgctggtccggcagatgagggttgagtaggcggtcaaatggagagactgggtgcacaagttacgctttcccgagaaccagattttggcgcgcccgccagcccccaccatcaccaccgtcagtgaaaacgttcctgctctcacagatcacgggacgctaccgcgcgaaagggctcttggtctaaaaccgcgcgggcggtcgggggtggtagtggtggcagtcacttttgcaaggacgagagtgtctagtgccctgcgatggcgtgcgcaacagcatcggaggagtccagcgagtgaccattactgacgccagacgccgcacctgcccctacgtttacaaggccctgggcatagtctcgccgcgacgcgttgtcgtagcctcctcaggtcgctcactggtaatgactgcggtctgcggcgtggacggggatgcaaatgttccgggacccgtatcagagcggcgccgtcctatcgagccgcactttttgagcaagcatgtccatccttatatcgcccagcaataacacaggctggggcctgcgcttcccaagcaagatgtttggcgcaggatagctcggcgtgaaaaactcgttcgtacaggtaggaatatagcgggtcgttattgtgtccgaccccggacgcgaagggttcgttctacaaaccgggggccaagaagcgctccgaccaacacccagtgcgcgtgcgcgggcactaccgcgcgccctggggcgcgcacaaacgcggccgcactgggcgcaccaccgccccggttcttcgcgaggctggttgtgggtcacgcgcacgcgcccgtgatggcgcgcgggaccccgcgcgtgtttgcgccggcgtgacccgcgtggtggctcgatgacgccatcgacgcggtggtggaggaggcgcgcaactacacgcccacgccgccaccagtgtccacagtggacgcggccattcagaccgtggtgcgagctactgcggtagctgcgccaccacctcctccgcgcgttgatgtgcgggtgcggcggtggtcacaggtgtcacctgcgccggtaagtctggcaccacgccggagcccggcgctatgctaaaatgaagagacggcggaggcgcgtagcacgtcgccaccgccgccgacccggcactgccgcccaacgcgcggcggcggccgcctcgggccgcgatacgattttacttctctgccgcctccgcgcatcgtgcagcggtggcggcggctgggccgtgacggcgggttgcgcgccgccgccggctgcttaaccgcgcacgtcgcaccggccgacgggcggccatgcgggccgctcgaaggctggccgcgggtattgtcactgtgccccccaggtccaggcgacgacgaattggcgcgtgcagcgtggccggctgcccgccggtacgcccggcgagcttccgaccggcgcccataacagtgacacggggggtccaggtccgctggagcggccgccgcagcagccgcggccattagtgctatgactcagggtcgcaggggcaacgtgtattgggtgcgcgactcggttagcggcctgcgcgtgccctcgccggcggcgtcgtcggcgccggtaatcacgatactgagtcccagcgtccccgttgcacataacccacgcgctgagccaatcgccggacgcgcacggcgtgcgcacccgccccccgcgcaactagattgcaagaaaaaactacttagactcgtactgttgtatgtatccagcggcggcggcgcgcaacgaagctatggcacgcgtgggcggggggcgcgttgatctaacgttcttttttgatgaatctgagcatgacaacatacataggtcgccgccgccgcgcgttgcttcgatactccaagcgcaaaatcaaagaagagatgctccaggtcatcgcgccggagatctatggccccccgaagaaggaagagcaggattacaagccccgaaagctaaaggttcgcgttttagtttcttctctacgaggtccagtagcgcggcctctagataccggggggcttcttccttctcgtcctaatgttcggggctttcgattagcgggtcaaaaagaaaaagaaagatgatgatgatgaacttgacgacgaggtggaactgctgcacgctaccgcgcccaggcgacgggtacagtggaaaggtcgcccagtttttctttttctttctactactactacttgaactgctgctccaccttgacgacgtgcgatggcgcgggtccgctgcccatgtcacctttcctcgacgcgtaaaacgtgttttgcgacccggcaccaccgtagtctttacgcccggtgagcgctccacccgcacctacaagcgcgtgtatgatgaggtgtacagctgcgcattttgcacaaaacgctgggccgtggtggcatcagaaatgcgggccactcgcgaggtgggcgtggatgttcgcgcacatactactccacatgggcgacgaggacctgcttgagcaggccaacgagcgcctcggggagtttgcctacggaaagcggcataaggacatgctggcgttgccgctggacgagggcaccgctgctcctggacgaactcgtccggttgctcgcggagcccctcaaacggatgcctttcgccgtattcctgtacgaccgcaacggcgacctgctcccgtacccaacacctagcctaaagcccgtaacactgcagcaggtgctgcccgcgcttgcaccgtccgaagaaaagcgcggcctaaagcgcgagtctggtgactttgggttgtggatcggatttcgggcattgtgacgtcgtccacgacgggcgcgaacgtggcaggcttcttttcgcgccggatttcgcgctcagaccactgaaggcacccaccgtgcagctgatggtacccaagcgccagcgactggaagatgtcttggaaaaaatgaccgtggaacctgggctggagcccgaggtccgcgtgccgtgggtggcacgtcgactaccatgggttcgcggtcgctgaccttctacagaaccttttttactggcaccttggacccgacctcgggctccaggcgcaccggccaatcaagcaggtggcgccgggactgggcgtgcagaccgtggacgttcagatacccactaccagtagcaccagtattgccaccgccacagagggcagccggttagttcgtccaccgcggccctgacccgcacgtctggcacctgcaagtctatgggtgatggtcatcgtggtcataacggtggcggtgtctcccgttggagacacaaacgtccccggttgcctcagcggtggcggatgccgcggtgcaggcggtcgctgcggccgcgtccaagacctctacggaggtgcaaacggaacctctgtgtttgcaggggccaacggagtcgccaccgcctacggcgccacgtccgccagcgacgccggcgcaggttctggagatgcctccacgtttgcctcccgtggatgtttcgcgtttcagccccccggcgcccgcgcggttcgaggaagtacggcgccgccagcgcgctactgcccgaatatgccctacatccttccgggcacctacaaagcgcaaagtcggggggccgcgggcgcgccaagctccttcatgccgcggcggtcgcgcgatgacgggcttatacgggatgtaggaaggattgcgcctacccccggctatcgtggctacacctaccgccccagaagacgagcaactacccgacgccgaaccaccactggaacccgccgccgccgtcgcctaacgcggatgggggccgatagcaccgatgtggatggcggggtcttctgctcgttgatgggctgcggcttggtggtgaccttgggcggcggcggcagcgggtcgccagcccgtgctggccccgatttccgtgcgcagggtggctcgcgaaggaggcaggaccctggtgctgccaacagcgcgctaccaccccagcatcgtcagcggtcgggcacgaccggggctaaaggcacgcgtcccaccgagcgcttcctccgtcctgggaccacgacggttgtcgcgcgatggtggggtcgtagcattaaaagccggtctttgtggttcttgcagatatggccctcacctgccgcctccgtttcccggtgccgggattccgaggaagaatgcaccgtaggaggggcaattttcggccagaaacaccaagaacgtctataccgggagtggacggcggaggcaaagggccacggccctaaggctccttcttacgtggcatcctccccgatggccggccacggcctgacgggcggcatgcgtcgtgcgcaccaccggcggcggcgcgcgtcgcaccgtcgcatgcgcggcggtatcctgcccctccttataccggccggtgccggactgcccgccgtacgcagcacgcgtggtggccgccgccgcgcgcagcgtggcagcgtacgcgccgccataggacggggaggaatttccactgatcgccgcggcgattggcgccgtgcccggaattgcatccgtggccttgcaggcgcagagacactgattaaaaacaagttgcatgtggaaaaaaaggtgactagcggcgccgctaaccgcggcacgggccttaacgtaggcaccggaacgtccgcgtctctgtgactaatttttgttcaacgtacaccttttttcaaaataaaaagtctggactctcacgctcgcttggtcctgtaactattttgtagaatggaagacatcaactttgcgtctctggccccgcgacacggctcagttttatttttcagacctgagagtgcgagcgaaccaggacattgataaaacatcttaccttctgtagttgaaacgcagagaccggggcgctgtgccgaggcgcccgttcatgggaaactggcaagatatcggcaccagcaatatgagcggtggcgccttcagctggggctcgctgtggagcggcattaaaaatttcggtcgcgggcaagtaccctttgaccgttctatagccgtggtcgttatactcgccaccgcggaagtcgaccccgagcgacacctcgccgtaatttttaaagccatccaccgttaagaactatggcagcaaggcctggaacagcagcacaggccagatgctgagggataagttgaaagagcaaaatttccaacaaaaggtggtagaggtggcaattcttgataccgtcgttccggaccttgtcgtcgtgtccggtctacgactccctattcaactttctcgttttaaaggttgttttccaccatcatggcctggcctctggcattagcggggtggtggacctggccaaccaggcagtgcaaaataagattaacagtaagcttgatccccgccctcccgtagaggataccggaccggagaccgtaatcgccccaccacctggaccggttggtccgtcacgttttattctaattgtcattcgaactaggggcgggagggcatctcctgcctccaccggccgtggagacagtgtctccagaggggcgtggcgaaaagcgtccgcgccccgacagggaagaaactctggtgacgcaaatagacgagcctcggaggtggccggcacctctgtcacagaggtctccccgcaccgcttttcgcaggcgcggggctgtcccttctttgagaccactgcgtttatctgctcggaccctcgtacgaggaggcactaaagcaaggcctgcccaccacccgtcccatcgcgcccatggctaccggagtgctgggccagcacacacccgtaacgctgggggagcatgctcctccgtgatttcgttccggacgggtggtgggcagggtagcgcgggtaccgatggcctcacgacccggtcgtgtgtgggcattgcgaccacctgcctccccccgccgacacccagcagaaacctgtgctgccaggcccgaccgccgttgttgtaacccgtcctagccgcgcgtccctgcgccgcgccgctggacggaggggggcggctgtgggtcgtctttggacacgacggtccgggctggcggcaacaacattgggcaggatcggcgcgcagggacgcggcgcggcgcagcggtccgcgatcgttgcggcccgtagccagtggcaactggcaaagcacactgaacagcatcgtgggtctgggggtgcaatccctgaagcgccgacgagtcgccaggcgctagcaacgccgggcatcggtcaccgttgaccgtttcgtgtgacttgtcgtagcacccagacccccacgttagggacttcgcggctgcttgcttctgaatagctaacgtgtcgtatgtgtgtcatgtatgcgtccatgtcgccgccagaggagctgctgagccgccgcgcgcccgctttccaagatggcacgaagacttatcgattgcacagcatacacacagtacatacgcaggtacagcggcggtctcctcgacgactcggcggcgcgcgggcgaaaggttctaccgtaccccttcgatgatgccgcagtggtcttacatgcacatctcgggccaggacgcctcggagtacctgagccccgggctggtgcagtttgcccgcgccaccatggggaagctactacggcgtcaccagaatgtacgtgtagagcccggtcctgcggagcctcatggactcggggcccgaccacgtcaaacgggcgcggtgggagacgtacttcagcctgaataacaagtttagaaaccccacggtggcgcctacgcacgacgtgaccacagaccggtcccagcgtttgacgctgcggttcactctgcatgaagtcggacttattgttcaaatctttggggtgccaccgcggatgcgtgctgcactggtgtctggccagggtcgcaaactgcgacgccaagttccctgtggaccgtgaggatactgcgtactcgtacaaggcgcggttcaccctagctgtgggtgataaccgtgtgctggacatggcttccacgtactttgaagggacacctggcactcctatgacgcatgagcatgttccgcgccaagtgggatcgacacccactattggcacacgacctgtaccgaaggtgcatgaaactcatccgcggcgtgctggacaggggccctacttttaagccctactctggcactgcctacaacgccctggctcccaagggtgccccaaatccttgcgaatgggtaggcgccgcacgacctgtccccgggatgaaaattcgggatgagaccgtgacggatgttgcgggaccgagggttcccacggggtttaggaacgcttaccgatgaagctgctactgctcttgaaataaacctagaagaagaggacgatgacaacgaagacgaagtagacgagcaagctgagcagcaaaaaactcacgtatctacttcgacgatgacgagaactttatttggatcttcttctcctgctactgttgcttctgcttcatctgctcgttcgactcgtcgttttttgagtgcatattgggcaggcgccttattctggtataaatattacaaaggagggtattcaaataggtgtcgaaggtcaaacacctaaatatgccgataaaacatttcaaccaacccgtccgcggaataagaccatatttataatgtttcctcccataagtttatccacagcttccagtttgtggatttatacggctattttgtaaagttggtgaacctcaaataggagaatctcagtggtacgaaactgaaattaatcatgcagctgggagagtccttaaaaagactaccccaatgaaaccatgttacggtacttggagtttatcctcttagagtcaccatgctttgactttaattagtacgtcgaccctctcaggaatttttctgatggggttactttggtacaatgccatcatatgcaaaacccacaaatgaaaatggagggcaaggcattcttgtaaagcaacaaaatggaaagctagaaagtcaagtggaaatgcaatttttctcaaagtatacgttttgggtgtttacttttacctcccgttccgtaagaacatttcgttgttttacctttcgatctttcagttcacctttacgttaaaaagagttctactgaggcgaccgcaggcaatggtgataacttgactcctaaagtggtattgtacagtgaagatgtagatatagaaaccccagacactcatatttcttagatgactccgctggcgtccgttaccactattgaactgaggatttcaccataacatgtcacttctacatctatatctttggggtctgtgagtataaagaatcatgcccactattaaggaaggtaactcacgagaactaatgggccaacaatctatgcccaacaggcctaattacattgcttttagggacaattttattggtgtacgggtgataattccttccattgagtgctcttgattacccggttgttagatacgggttgtccggattaatgtaacgaaaatccctgttaaaataaccactaatgtattacaacagcacgggtaatatgggtgttctggcgggccaagcatcgcagttgaatgctgttgtagatttgcaagacagaaacacagagctttgattacataatgttgtcgtgcccattatacccacaagaccgcccggttcgtagcgtcaacttacgacaacatctaaacgttctgtctttgtgtctcgaaacataccagcttttgcttgattccattggtgatagaaccaggtacttttctatgtggaatcaggctgttgacagctatgatccagatgttagaattattgagtatggtcgaaaacgaactaaggtaaccactatcttggtccatgaaaagatacaccttagtccgacaactgtcgatactaggtctacaatcttaataactaaatcatggaactgaagatgaacttccaaattactgctttccactgggaggtgtgattaatacagagactcttaccaaggtaaaacctaaaacaggtcagtttagtaccttgacttctacttgaaggtttaatgacgaaaggtgaccctccacactaattatgtctctgagaatggttccattttggattttgtccagtcgaaaatggatgggaaaaagatgctacagaattttcagataaaaatgaaataagagttggaaataattttgccatggaaatcaatctaaatgccaacctgtcttttacctaccctttttctacgatgtcttaaaagtctatttttactttattctcaacctttattaaaacggtacctttagttagatttacggttggacaggagaaatttcctgtactccaacatagcgctgtatttgcccgacaagctaaagtacagtccttccaacgtaaaaatttctgataacccaaacacctacgacctctttaaaggacatgaggttgtatcgcgacataaacgggctgttcgatttcatgtcaggaaggttgcatttttaaagactattgggtttgtggatgctctacatgaacaagcgagtggtggctcccgggttagtggactgctacattaaccttggagcacgctggtcccttgactatatggacaacgtcaacccatttgatgtacttgttcgctcaccaccgagggcccaatcacctgacgatgtaattggaacctcgtgcgaccagggaactgatatacctgttgcagttgggtaaaaaccaccaccgcaatgctggcctgcgctaccgctcaatgttgctgggcaatggtcgctatgtgcccttccacatccaggtgcctcagaagttctttgccattggtggtggcgttacgaccggacgcgatggcgagttacaacgacccgttaccagcgatacacgggaaggtgtaggtccacggagtcttcaagaaacggtttaaaaacctccttctcctgccgggctcatacacctacgagtggaacttcaggaaggatgttaacatggttctgcagagctccctaggaaatgacctaagaatttttggaggaagaggacggcccgagtatgtggatgctcaccttgaagtccttcctacaattgtaccaagacgtctcgagggatcctttactggattcggttgacggagccagcattaagtttgatagcatttgcctttacgccaccttcttccccatggcccacaacaccgcctccacgcttgaggccatgcttagaccaactgcctcggtcgtaattcaaactatcgtaaacggaaatgcggtggaagaaggggtaccgggtgttgtggcggaggtgcgaactccggtacgaatctaacgacaccaacgaccagtcctttaacgactatctctccgccgccaacatgctctaccctatacccgccaacgctaccaacgtgcccatatccatcccctttgctgtggttgctggtcaggaaattgctgatagagaggcggcggttgtacgagatgggatatgggcggttgcgatggttgcacgggtataggtaggggacccgcaactgggcggctttccgcggctgggccttcacgcgccttaagactaaggaaaccccatcactgggctcgggctacgacccttattacacctactcgggcgttgacccgccgaaaggcgccgacccggaagtgcgcggaattctgattcctttggggtagtgacccgagcccgatgctgggaataatgtggatgagtggctctataccctacctagatggaaccttttacctcaaccacacctttaagaaggtggccattacctttgactcttctgtcagctggcctggcaatgacaccgagatatgggatggatctaccttggaaaatggagttggtgtggaaattcttccaccggtaatggaaactgagaagacagtcgaccggaccgttactgcgcctgcttacccccaacgagtttgaaattaagcgctcagttgacggggagggttacaacgttgcccagtgtaacatgaccaaagactggttcctggtacgcggacgaatgggggttgctcaaactttaattcgcgagtcaactgcccctcccaatgttgcaacgggtcacattgtactggtttctgaccaaggaccatgaaatgctagctaactacaacattggctaccagggcttctatatcccagagagctacaaggaccgcatgtactccttctttagaaacttccagcccatgagtttacgatcgattgatgttgtaaccgatggtcccgaagatatagggtctctcgatgttcctggcgtacatgaggaagaaatctttgaaggtcgggtactcccgtcaggtggtggatgatactaaatacaaggactaccaacaggtgggcatcctacaccaacacaacaactctggatttgttggctaccttgcccccaccggcagtccaccacctactatgatttatgttcctgatggttgtccacccgtaggatgtggttgtgttgttgagacctaaacaaccgatggaacgggggtggatgcgcgaaggacaggcctaccctgctaacttcccctatccgcttataggcaagaccgcagttgacagcattacccagaaaaagtttctttgcgatcgcatacgcgcttcctgtccggatgggacgattgaaggggataggcgaatatccgttctggcgtcaactgtcgtaatgggtctttttcaaagaaacgctagcgtccctttggcgcatcccattctccagtaactttatgtccatgggcgcactcacagacctgggccaaaaccttctctacgccaactccgcccacgcgctagagggaaaccgcgtagggtaagaggtcattgaaatacaggtacccgcgtgagtgtctggacccggttttggaagagatgcggttgaggcgggtgcgcgatctcatgacttttgaggtggatcccatggacgagcccacccttctttatgttttgtttgaagtctttgacgtggtccgtgtgcaccggccgcaccgcggcgtcgtactgaaaactccacctagggtacctgctcgggtgggaagaaatacaaaacaaacttcagaaactgcaccaggcacacgtggccggcgtggcgccgcagatcgaaaccgtgtacctgcgcacgcccttctcggccggcaacgccacaacataaagaagcaagcaacatcaacaacagctgccgccatgggctccagtgatagctttggcacatggacgcgtgcgggaagagccggccgttgcggtgttgtatttcttcgttcgttgtagttgttgtcgacggcggtacccgaggtcactgcaggaactgaaagccattgtcaaagatcttggttgtgggccatattttttgggcacctatgacaagcgctttccaggctttgtttctccacacaagctccgtccttgactttcggtaacagtttctagaaccaacacccggtataaaaaacccgtggatactgttcgcgaaaggtccgaaacaaagaggtgtgttcgaggcctgcgccatagtcaatacggccggtcgcgagactgggggcgtacactggatggcctttgcctggaacccgcactcaaaaacatgctacctctttgagccggacgcggtatcagttatgccggccagcgctctgacccccgcatgtgacctaccggaaacggaccttgggcgtgagtttttgtacgatggagaaactcgcctttggcttttctgaccagcgactcaagcaggtttaccagtttgagtacgagtcactcctgcgccgtagcgccattgcttcttcccccgaccgctgtatggaaaccgaaaagactggtcgctgagttcgtccaaatggtcaaactcatgctcagtgaggacgcggcatcgcggtaacgaagaagggggctggcgacataaacgctggaaaagtccacccaaagcgtacaggggcccaactcggccgcctgtggactattctgctgcatgtttctccacgcctttgccaactggccccaattgcgaccttttcaggtgggtttcgcatgtccccgggttgagccggcggacacctgataagacgacgtacaaagaggtgcggaaacggttgaccggggttactcccatggatcacaaccccaccatgaaccttattaccggggtacccaactccatgctcaacagtccccaggtacagcccaccctgcgtcgcaaccaggtgagggtacctagtgttggggtggtacttggaataatggccccatgggttgaggtacgagttgtcaggggtccatgtcgggtgggacgcagcgttggtccaacagctctacagcttcctggagcgccactcgccctacttccgcagccacagtgcgcagattaggagcgccacttctttttgtcacttgaaaaacatgtattgtcgagatgtcgaaggacctcgcggtgagcgggatgaaggcgtcggtgtcacgcgtctaatcctcgcggtgaagaaaaacagtgaactttttgtacataaaataatgtactagagacactttcaataaaggcaaatgcttttatttgtacactctcgggtgattatttacccccacccttgccgtctgcgccgtttaattttattacatgatctctgtgaaagttatttccgtttacgaaaataaacatgtgagagcccactaataaatgggggtgggaacggcagacgcggcaaattaaatcaaaggggttctgccgcgcatcgctatgcgccactggcagggacacgttgcgatactggtgtttagtgctccacttaaactcaggcacaaccatcctttagtttccccaagacggcgcgtagcgatacgcggtgaccgtccctgtgcaacgctatgaccacaaatcacgaggtgaatttgagtccgtgttggtagggcggcagctcggtgaagttttcactccacaggctgcgcaccatcaccaacgcgtttagcaggtcgggcgccgatatcttgaagtcgcagttggggcctcccgccgtcgagccacttcaaaagtgaggtgtccgacgcgtggtagtggttgcgcaaatcgtccagcccgcggctatagaacttcagcgtcaaccccggagggccctgcgcgcgcgagttgcgatacacagggttgcagcactggaacactatcagcgccgggtggtgcacgctggccagcacgctcttgtcggagatcagacgggacgcgcgcgctcaacgctatgtgtcccaacgtcgtgaccttgtgatagtcgcggcccaccacgtgcgaccggtcgtgcgagaacagcctctagtcttccgcgtccaggtcctccgcgttgctcagggcgaacggagtcaactttggtagctgccttcccaaaaagggcgcgtgcccaggctttgagttgcactcgcaggcgcaggtccaggaggcgcaacgagtcccgcttgcctcagttgaaaccatcgacggaagggtttttcccgcgcacgggtccgaaactcaacgtgagcgaccgtagtggcatcaaaaggtgaccgtgcccggtctgggcgttaggatacagcgcctgcataaaagccttgatctgcttaaaagccacctgagcctttgctggcatcaccgtagttttccactggcacgggccagacccgcaatcctatgtcgcggacgtattttcggaactagacgaattttcggtggactcggaaacggccttcagagaagaacatgccgcaagacttgccggaaaactgattggccggacaggccgcgtcgtgcacgcagcaccttgcgtcggtgttggagatctgccggaagtctcttcttgtacggcgttctgaacggccttttgactaaccggcctgtccggcgcagcacgtgcgtcgtggaacgcagccacaacctctagacgaccacatttcggccccaccggttcttcacgatcttggccttgctagactgctccttcagcgcgcgctgcccgttttcgctcgtcacatccatttcaatcatggtgtaaagccggggtggccaagaagtgctagaaccggaacgatctgacgaggaagtcgcgcgcgacgggcaaaagcgagcagtgtaggtaaagttagtcgtgctccttatttatcataatgcttccgtgtagacacttaagctcgccttcgatctcagcgcagcggtgcagccacaacgcgcagcccgtgggctcgtggcacgaggaataaatagtattacgaaggcacatctgtgaattcgagcggaagctagagtcgcgtcgccacgtcggtgttgcgcgtcgggcacccgagcacatgcttgtaggtcacctctgcaaacgactgcaggtacgcctgcaggaatcgccccatcatcgtcacaaaggtcttgttgctggtgaaggtcagctgcaactacgaacatccagtggagacgtttgctgacgtccatgcggacgtccttagcggggtagtagcagtgtttccagaacaacgaccacttccagtcgacgttgccgcggtgctcctcgttcagccaggtcttgcatacggccgccagagcttccacttggtcaggcagtagtttgaagttcgcctttagatcgttatccacgtggcgccacgaggagcaagtcggtccagaacgtatgccggcggtctcgaaggtgaaccagtccgtcatcaaacttcaagcggaaatctagcaataggtgcaggtacttgtccatcagcgcgcgcgcagcctccatgcccttctcccacgcagacacgatcggcacactcagcgggttcatcaccgtaatttcactttccgcccatgaacaggtagtcgcgcgcgcgtcggaggtacgggaagagggtgcgtctgtgctagccgtgtgagtcgcccaagtagtggcattaaagtgaaaggcgttcgctgggctcttcctcttcctcttgcgtccgcataccacgcgccactgggtcgtcttcattcagccgccgcactgtgcgcttacctcctttgccatgcaagcgacccgagaaggagaaggagaacgcaggcgtatggtgcgcggtgacccagcagaagtaagtcggcggcgtgacacgcgaatggaggaaacggtacgttgattagcaccggtgggttgctgaaacccaccatttgtagcgccacatcttctctttcttcctcgctgtccacgattacctctggtgatggcgggcgctaactaatcgtggccacccaacgactttgggtggtaaacatcgcggtgtagaagagaaagaaggagcgacaggtgctaatggagaccactaccgcccgcgacgggcttgggagaagggcgcttctttttcttcttgggcgcaatggccaaatccgccgccgaggtcgatggccgcgggctgggtgtgcgcggcaccagcgcgcccgaaccctcttcccgcgaagaaaaagaagaacccgcgttaccggtttaggcggcggctccagctaccggcgcccgacccacacgcgccgtggtcgcggtcttgtgatgagtcttcctcgtcctcggactcgatacgccgcctcatccgcttttttgggggcgcccggggaggcggcggcgacggggacggggacgaccagaacactactcagaaggagcaggagcctgagctatgcggcggagtaggcgaaaaaacccccgcgggcccctccgccgccgctgcccctgcccctgctgacgtcctccatggttgggggacgtcgcgccgcaccgcgtccgcgctcgggggtggtttcgcgctgctcctcttcccgactggccatttccttctcctatatgcaggaggtaccaaccccctgcagcgcggcgtggcgcaggcgcgagcccccaccaaagcgcgacgaggagaagggctgaccggtaaaggaagaggatatggcagaaaaagatcatggagtcagtcgagaagaaggacagcctaaccgccccctctgagttcgccaccaccgcctccaccgatgccgccaacgcgcctacccgtctttttctagtacctcagtcagctcttcttcctgtcggattggcgggggagactcaagcggtggtggcggaggtggctacggcggttgcgcggatgcaccttccccgtcgaggcacccccgcttgaggaggaggaagtgattatcgagcaggacccaggttttgtaagcgaagacgacgaggaccgctcagtaccagtggaaggggcagctccgtgggggcgaactcctcctccttcactaatagctcgtcctgggtccaaaacattcgcttctgctgctcctggcgagtcatggtacagaggataaaaagcaagaccaggacaacgcagaggcaaacgaggaacaagtcgggcggggggacgaaaggcatggcgactacctagatgtgggagacgtgtctcctatttttcgttctggtcctgttgcgtctccgtttgctccttgttcagcccgcccccctgctttccgtaccgctgatggatctacaccctctgcacgtgctgttgaagcatctgcagcgccagtgcgccattatctgcgacgcgttgcaagagcgcagcgatgtgcccctcgccatagcggatgtcagccttgctgcacgacaacttcgtagacgtcgcggtcacgcggtaatagacgctgcgcaacgttctcgcgtcgctacacggggagcggtatcgcctacagtcggaacgctacgaacgccacctattctcaccgcgcgtaccccccaaacgccaagaaaacggcacatgcgagcccaacccgcgcctcaacttctaccccgtatttgccgatgcttgcggtggataagagtggcgcgcatggggggtttgcggttcttttgccgtgtacgctcgggttgggcgcggagttgaagatggggcataaacgggtgccagaggtgcttgccacctatcacatctttttccaaaactgcaagatacccctatcctgccgtgccaaccgcagccgagcggacaagcagctggcctcacggtctccacgaacggtggatagtgtagaaaaaggttttgacgttctatggggataggacggcacggttggcgtcggctcgcctgttcgtcgaccggatgcggcagggcgctgtcatacctgatatcgcctcgctcaacgaagtgccaaaaatctttgagggtcttggacgcgacgagaagcgcgcggcaaacgctctacgccgtcccgcgacagtatggactatagcggagcgagttgcttcacggtttttagaaactcccagaacctgcgctgctcttcgcgcgccgtttgcgagagcaacaggaaaacagcgaaaatgaaagtcactctggagtgttggtggaactcgagggtgacaacgcgcgcctagccgtactaaaacgcagcatcgaggtccgttgtccttttgtcgcttttactttcagtgagacctcacaaccaccttgagctcccactgttgcgcgcggatcggcatgattttgcgtcgtagctccagacccactttgcctacccggcacttaacctaccccccaaggtcatgagcacagtcatgagtgagctgatcgtgcgccgtgcgcagcccctggagagggatgtgggtgaaacggatgggccgtgaattggatggggggttccagtactcgtgtcagtactcactcgactagcacgcggcacgcgtcggggacctctccctaccaaatttgcaagaacaaacagaggagggcctacccgcagttggcgacgagcagctagcgcgctggcttcaaacgcgcgagcctgccgacttggaggagcggtttaaacgttcttgtttgtctcctcccggatgggcgtcaaccgctgctcgtcgatcgcgcgaccgaagtttgcgcgctcggacggctgaacctcctcgcacgcaaactaatgatggccgcagtgctcgttaccgtggagcttgagtgcatgcagcggttctttgctgacccggagatgcagcgcaagctagaggaaacatgcgtttgattactaccggcgtcacgagcaatggcacctcgaactcacgtacgtcgccaagaaacgactgggcctctacgtcgcgttcgatctcctttgtttgcactacacctttcgacagggctacgtacgccaggcctgcaagatctccaacgtggagctctgcaacctggtctcctaccttggaattttgcacgaaaaacgtgatgtggaaagctgtcccgatgcatgcggtccggacgttctagaggttgcacctcgagacgttggaccagaggatggaaccttaaaacgtgctttaccgccttgggcaaaacgtgcttcattccacgctcaagggcgaggcgcgccgcgactacgtccgcgactgcgtttacttatttctatgctacacctggcatggcggaacccgttttgcacgaagtaaggtgcgagttcccgctccgcgcggcgctgatgcaggcgctgacgcaaatgaataaagatacgatgtggaccgtgacggccatgggcgtttggcagcagtgcttggaggagtgcaacctcaaggagctgcagaaactgctaaagcaaaacttgaaggacctatggacggccttcctgccggtacccgcaaaccgtcgtcacgaacctcctcacgttggagttcctcgacgtctttgacgatttcgttttgaacttcctggatacctgccggaagaacgagcgctccgtggccgcgcacctggcggacatcattttccccgaacgcctgcttaaaaccctgcaacagggtctgccagacttcaccagtcaaagcattgctcgcgaggcaccggcgcgtggaccgcctgtagtaaaaggggcttgcggacgaattttgggacgttgtcccagacggtctgaagtggtcagtttcgttgttgcagaactttaggaactttatcctagagcgctcaggaatcttgcccgccacctgctgtgcacttcctagcgactttgtgcccattaagtaccgcgaacaacgtcttgaaatccttgaaataggatctcgcgagtccttagaacgggcggtggacgacacgtgaaggatcgctgaaacacgggtaattcatggcgctatgccctccgccgctttggggccactgctaccttctgcagctagccaactaccttgcctaccactctgacataatggaagacgtgagcggtgacggtctatacgggaggcggcgaaaccccggtgacgatggaagacgtcgatcggttgatggaacggatggtgagactgtattaccttctgcactcgccactgccagatctggagtgtcactgtcgctgcaacctatgcaccccgcaccgctccctggtttgcaattcgcagctgcttaacgaaagtcaaattatcggtacctttgagcgacctcacagtgacagcgacgttggatacgtggggcgtggcgagggaccaaacgttaagcgtcgacgaattgctttcagtttaatagccatggaaactcgtgcagggtccctcgcctgacgaaaagtccgcggctccggggttgaaactcactccggggctgtggacgtcggcttaccttcgcaaatttgtacctgaggaacgtcccagggagcggactgcttttcaggcgccgaggccccaactttgagtgaggccccgacacctgcagccgaatggaagcgtttaaacatggactcctctaccacgcccacgagattaggttctacgaagaccaatcccgcccgccaaatgcggagcttaccgcctgcgtcattacccagggccacattcttggccaagatggtgcgggtgctctaatccaagatgcttctggttagggcgggcggtttacgcctcgaatggcggacgcagtaatgggtcccggtgtaagaaccggttttgcaagccatcaacaaagcccgccaagagtttctgctacgaaagggacggggggtttacttggacccccagtccggcgaggagctcaacccaatcccccaacgttcggtagttgtttcgggcggttctcaaagacgatgctttccctgccccccaaatgaacctgggggtcaggccgctcctcgagttgggttagggggcgccgccgcagccctatcagcagcagccgcgggcccttgcttcccaggatggcacccaaaaagaagctgcagctgccgccgccacccacggacgaggagggcggcggcgtcgggatagtcgtcgtcggcgcccgggaacgaagggtcctaccgtgggtttttcttcgacgtcgacggcggcggtgggtgcctgctcctccaatactgggacagtcaggcagaggaggttttggacgaggaggaggaggacatgatggaagactgggagagcctagacgaggaagcttccgaggtcgaagattatgaccctgtcagtccgtctcctccaaaacctgctcctcctcctcctgtactaccttctgaccctctcggatctgctccttcgaaggctccagcttctggtgtcagacgaaacaccgtcaccctcggtcgcattcccctcgccggcgccccagaaatcggcaaccggttccagcatggctacaacctccgctcctcagccacagtctgctttgtggcagtgggagccagcgtaaggggagcggccgcggggtctttagccgttggccaaggtcgtaccgatgttggaggcgaggagtcgcgccgccggcactgcccgttcgccgacccaaccgtagatgggacaccactggaaccagggccggtaagtccaagcagccgccgccgttagcccaagagccgcggcggccgtgacgggcaagcggctgggttggcatctaccctgtggtgaccttggtcccggccattcaggttcgtcggcggcggcaatcgggttctcgaacaacagcgccaaggctaccgctcatggcgcgggcacaagaacgccatagttgcttgcttgcaagactgtgggggcaacatctccttcgcccgccgcttttgttgtcgcggttccgatggcgagtaccgcgcccgtgttcttgcggtatcaacgaacgaacgttctgacacccccgttgtagaggaagcgggcggcgaatcttctctaccatcacggcgtggccttcccccgtaacatcctgcattactaccgtcatctctacagcccatactgcaccggcggcagcggcagcggcagcagaagagatggtagtgccgcaccggaagggggcattgtaggacgtaatgatggcagtagagatgtcgggtatgacgtggccgccgtcgccgtcgccgtcgaacagcagcggccacacagaagcaaaggcgaccggatagcaagactctgacaaagcccaagaaatccacagcggcggcagcagcaggaggaggagcgctgttgtcgtcgccggtgtgtcttcgtttccgctggcctatcgttctgagactgtttcgggttctttaggtgtcgccgccgtcgtcgtcctcctcctcgcgaccgtctggcgcccaacgaacccgtatcgacccgcgagcttagaaacaggatttttcccactctgtatgctatatttcaacagagcaggggccaagaacaaggcagaccgcgggttgcttgggcatagctgggcgctcgaatctttgtcctaaaaagggtgagacatacgatataaagttgtctcgtccccggttcttgttcagctgaaaataaaaaacaggtctctgcgatccctcacccgcagctgcctgtatcacaaaagcgaagatcagcttcggcgcacgctggaagacgcggaggctcgacttttattttttgtccagagacgctagggagtgggcgtcgacggacatagtgttttcgcttctagtcgaagccgcgtgcgaccttctgcgcctccgtctcttcagtaaatactgcgcgctgactcttaaggactagtttcgcgccctttctcaaatttaagcgcgaaaactacgtcatctccagcggccacacccgagagaagtcatttatgacgcgcgactgagaattcctgatcaaagcgcgggaaagagtttaaattcgcgcttttgatgcagtagaggtcgccggtgtgggcgcgccagcacctgtcgtcagcgccattatgagcaaggaaattcccacgccctacatgtggagttaccagccacaaatgggacttgcggctggagctgccccgcggtcgtggacagcagtcgcggtaatactcgttcctttaagggtgcgggatgtacacctcaatggtcggtgtttaccctgaacgccgacctcgacgggaagactactcaacccgaataaactacatgagcgcgggaccccacatgatatcccgggtcaacggaatccgcgcccaccgaaaccgaattctcttggaacattctgatgagttgggcttatttgatgtactcgcgccctggggtgtactatagggcccagttgccttaggcgcgggtggctttggcttaagagaaccttgtggcggctattaccaccacacctcgtaataaccttaatccccgtagttggcccgctgccctggtgtaccaggaaagtcccgctcccaccactgtggtacttccgccgataatggtggtgtggagcattattggaattaggggcatcaaccgggcgacgggaccacatggtcctttcagggcgagggtggtgacaccatgaacccagagacgcccaggccgaagttcagatgactaactcaggggcgcagcttgcgggcggctttcgtcacagggtgcggtcgcccgggcagggtataactcgggtctctgcgggtccggcttcaagtctactgattgagtccccgcgtcgaacgcccgccgaaagcagtgtcccacgccagcgggcccgtcccatattgagacctgacaatcagagggcgaggtattcagctcaacgacgagtcggtgagctcctcgcttggtctccgtccggacgggacatttcagatcggcggcgccggtggactgttagtctcccgctccataagtcgagttgctgctcagccactcgaggagcgaaccagaggcaggcctgccctgtaaagtctagccgccgcggccccgtccttcattcacgcctcgtcaggcaatcctaactctgcagacctcgtcctctgagccgcgctctggaggcattggaactctgcaatttattgaggagggcaggaagtaagtgcggagcagtccgttaggattgagacgtctggagcaggagactcggcgcgagacctccgtaaccttgagacgttaaataactcctctttgtgccatcggtctactttaaccccttctcgggacctcccggccactatccggatcaatttattcctaactttgacgcggtaaaggactcggcggacgaaacacggtagccagatgaaattggggaagagccctggagggccggtgataggcctagttaaataaggattgaaactgcgccatttcctgagccgcctgcgctacgactgaatgttaagtggagaggcagagcaactgcgcctgaaacacctggtccactgtcgccgccacaagtgctttgcccgcgactccggtgagttcgatgctgacttacaattcacctctccgtctcgttgacgcggactttgtggaccaggtgacagcggcggtgttcacgaaacgggcgctgaggccactcaattgctactttgaattgcccgaggatcatatcgagggcccggcgcacggcgtccggcttaccgcccagggagagcttgcccgtagcctgattcgggagtttaacgatgaaacttaacgggctcctagtatagctcccgggccgcgtgccgcaggccgaatggcgggtccctctcgaacgggcatcggactaagccctcaaaacccagcgccccctgctagttgagcgggacaggggaccctgtgttctcactgtgatttgcaactgtcctaaccttggattacatcaagatcctctagttatgggtcgcgggggacgatcaactcgccctgtcccctgggacacaagagtgacactaaacgttgacaggattggaacctaatgtagttctaggagatcaattaactagagtacccggggatcttattccctttaactaataaaaaaaaataataaagcatcacttacttaaaatcagttagcaaatttctgtccagtttatattgatctcatgggcccctagaataagggaaattgattatttttttttattatttcgtagtgaatgaattttagtcaatcgtttaaagacaggtcaaatatcagcagcacctccttgccctcctcccagctctggtattgcagcttcctcctggctgcaaactttctccacaatctaaatggaatgtcagtttcctcctgagtcgtcgtggaggaacgggaggagggtcgagaccataacgtcgaaggaggaccgacgtttgaaagaggtgttagatttaccttacagtcaaaggaggacttcctgtccatccgcacccactatcttcatgttgttgcagatgaagcgcgcaagaccgtctgaagataccttcaaccccgtgtatccatatgacacggaaaaggacaggtaggcgtgggtgatagaagtacaacaacgtctacttcgcgcgttctggcagacttctatggaagttggggcacataggtatactgtgccttaccggtcctccaactgtgccttttcttactcctccctttgtatcccccaatgggtttcaagagagtccccctggggtactctctttgcgcctatccgaactggccaggaggttgacacggaaaagaatgaggagggaaacatagggggttacccaaagttctctcagggggaccccatgagagaaacgcggataggcttgctctagttacctccaatggcatgcttgcgctcaaaatgggcaacggcctctctctggacgaggccggcaaccttacctcccaaaatgtaaccactgtgaggagatcaatggaggttaccgtacgaacgcgagttttacccgttgccggagagagacctgctccggccgttggaatggagggttttacattggtgacactccccacctctcaaaaaaaccaagtcaaacataaacctggaaatatctgcacccctcacagttacctcagaagccctaactgtggctgccgccgcacctctagggtggagagtttttttggttcagtttgtatttggacctttatagacgtggggagtgtcaatggagtcttcgggattgacaccgacggcggcgtggagatatggtcgcgggcaacacactcaccatgcaatcacaggccccgctaaccgtgcacgactccaaacttagcattgccacccaaggacccctcacagtgtcagtaccagcgcccgttgtgtgagtggtacgttagtgtccggggcgattggcacgtgctgaggtttgaatcgtaacggtgggttcctggggagtgtcacagtcaaggaaagctagccctgcaaacatcaggccccctcaccaccaccgatagcagtacccttactatcactgcctcaccccctctaactactgccactggtagttcctttcgatcgggacgtttgtagtccgggggagtggtggtggctatcgtcatgggaatgatagtgacggagtgggggagattgatgacggtgaccatccttgggcattgacttgaaagagcccatttatacacaaaatggaaaactaggactaaagtacggggctcctttgcatgtaacagacgacctaaacactttggaacccgtaactgaactttctcgggtaaatatgtgttttaccttttgatcctgatttcatgccccgaggaaacgtacattgtctgctggatttgtgaaacaccgtagcaactggtccaggtgtgactattaataatacttccttgcaaactaaagttactggagccttgggttttgattcacaaggcaatatgcaacttatggcatcgttgaccaggtccacactgataattattatgaaggaacgtttgatttcaatgacctcggaacccaaaactaagtgttccgttatacgttgaatatgtagcaggaggactaaggattgattctcaaaacagacgccttatacttgatgttagttatccgtttgatgctcaaaaccaactaaatctaagactaggtacatcgtcctcctgattcctaactaagagttttgtctgcggaatatgaactacaatcaataggcaaactacgagttttggttgatttagattctgatccacagggccctctttttataaactcagcccacaacttggatattaactacaacaaaggcctttacttgtttacagcttcaaacaattccaaaaagcttgagtgtcccgggagaaaaatatttgagtcgggtgttgaacctataattgatgttgtttccggaaatgaacaaatgtcgaagtttgttaaggtttttcgaactcgttaacctaagcactgccaaggggttgatgtttgacgctacagccatagccattaatgcaggagatgggcttgaatttggttcacctaatgcaccaaacacaattggattcgtgacggttccccaactacaaactgcgatgtcggtatcggtaattacgtcctctacccgaacttaaaccaagtggattacgtggtttgtcaaatcccctcaaaacaaaaattggccatggcctagaatttgattcaaacaaggctatggttcctaaactaggaactggccttagttttgacagcacagggtttaggggagttttgtttttaaccggtaccggatcttaaactaagtttgttccgataccaaggatttgatccttgaccggaatcaaaactgtcgtgtcctgccattacagtaggaaacaaaaataatgataagctaactttgtggaccacaccagctccatctcctaactgtagactaaatgcagagaaagatgctaaaacggtaatgtcatcctttgtttttattactattcgattgaaacacctggtgtggtcgaggtagaggattgacatctgatttacgtctctttctacgatttctcactttggtcttaacaaaatgtggcagtcaaatacttgctacagtttcagttttggctgttaaaggcagtttggctccaatatctggaacagttcaaagagtgaaaccagaattgttttacaccgtcagtttatgaacgatgtcaaagtcaaaaccgacaatttccgtcaaaccgaggttatagaccttgtcaagtttgtgctcatcttattataagatttgacgaaaatggagtgctactaaacaattccttcctggacccagaatattggaactttagaaatggagatcttactgacacgagtagaataatattctaaactgcttttacctcacgatgatttgttaaggaaggacctgggtcttataaccttgaaatctttacctctagaatgactaggcacagcctatacaaacgctgttggatttatgcctaacctatcagcttatccaaaatctcacggtaaaactgccaaaagtaacattgtcagtcaagtttccgtgtcggatatgtttgcgacaacctaaatacggattggatagtcgaataggttttagagtgccattttgacggttttcattgtaacagtcagttcaatacttaaacggagacaaaactaaacctgtaacactaaccattacactaaacggtacacaggaaacaggagacacaactccaagtgcatactctatgtcatatgaatttgcctctgttttgatttggacattgtgattggtaatgtgatttgccatgtgtcctttgtcctctgtgttgaggttcacgtatgagatacagtatttcatgggactggtctggccacaactacattaatgaaatatttgccacatcctcttacactttttcatacattgcccaagaataaagaatcgtttgtgtaaagtaccctgaccagaccggtgttgatgtaattactttataaacggtgtaggagaatgtgaaaaagtatgtaacgggttcttatttcttagcaaacacatatgtttcaacgtgtttatttttcaattgcagaaaatttcaagtcatttttcattcagtagtatagccccaccaccacatagcttatacagatcaccgtaatacaaagttgcacaaataaaaagttaacgtcttttaaagttcagtaaaaagtaagtcatcatatcggggtggtggtgtatcgaatatgtctagtggcatccttaatcaaactcacagaaccctagtattcaacctgccacctccctcccaacacacagagtacacagtcctttctccccggctggccttaaaaagcatcggaattagtttgagtgtcttgggatcataagttggacggtggagggagggttgtgtgtctcatgtgtcaggaaagaggggccgaccggaatttttcgtagatatcatgggtaacagacatattcttaggtgttatattccacacggtttcctgtcgagccaaacgctcatcagtgatattaataaactccccgggcagcttatagtacccattgtctgtataagaatccacaatataaggtgtgccaaaggacagctcggtttgcgagtagtcactataattatttgaggggcccgtcgacacttaagttcatgtcgctgtccagctgctgagccacaggctgctgtccaacttgcggttgcttaacgggcggcgaaggagaagtccacgcctacatggggtgaattcaagtacagcgacaggtcgacgactcggtgtccgacgacaggttgaacgccaacgaattgcccgccgcttcctcttcaggtgcggatgtacccggtagagtcataatcgtgcatcaggatagggcggtggtgctgcagcagcgcgcgaataaactgctgccgccgccgctccgtcctgcaggaatacaacatgccatctcagtattagcacgtagtcctatcccgccaccacgacgtcgtcgcgcgcttatttgacgacggcggcggcgaggcaggacgtccttatgttgtacgcagtggtctcctcagcgatgattcgcaccgcccgcagcataaggcgccttgtcctccgggcacagcagcgcaccctgatctcacttaaatcagcacagtcgtcaccagaggagtcgctactaagcgtggcgggcgtcgtattccgcggaacaggaggcccgtgtcgtcgcgtgggactagagtgaatttagtcgtgtcaaactgcagcacagcaccacaatattgttcaaaatcccacagtgcaaggcgctgtatccaaagctcatggcggggaccacagaacccacgtggccatcatattgacgtcgtgtcgtggtgttataacaagttttagggtgtcacgttccgcgacataggtttcgagtaccgcccctggtgtcttgggtgcaccggtagtatccacaagcgcaggtagattaagtggcgacccctcataaacacgctggacataaacattacctcttttggcatgttgtaattcaccacctcccggtaccatggtgttcgcgtccatctaattcaccgctggggagtatttgtgcgacctgtatttgtaatggagaaaaccgtacaacattaagtggtggagggccatggtaataaacctctgattaaacatggcgccatccaccaccatcctaaaccagctggccaaaacctgcccgccggctatacactgcagggaaccgggactggaactatttggagactaatttgtaccgcggtaggtggtggtaggatttggtcgaccggttttggacgggcggccgatatgtgacgtcccttggccctgaccttgaatgacagtggagagcccaggactcgtaaccatggatcatcatgctcgtcatgatatcaatgttggcacaacacaggcacacgtgcatacacttcctcagttactgtcacctctcgggtcctgagcattggtacctagtagtacgagcagtactatagttacaaccgtgttgtgtccgtgtgcacgtatgtgaaggagtcgattacaagctcctcccgcgttagaaccatatcccagggaacaacccattcctgaatcagcgtaaatcccacactgcagggaagacctcgcacgtaactcctaatgttcgaggagggcgcaatcttggtatagggtcccttgttgggtaaggacttagtcgcatttagggtgtgacgtcccttctggagcgtgcattgagacgttgtgcattgtcaaagtgttacattcgggcagcagcggatgatcctccagtatggtagcgcgggtttctgtctcaaaaggaggtagacgatccctactgcaacacgtaacagtttcacaatgtaagcccgtcgtcgcctactaggaggtcataccatcgcgcccaaagacagagttttcctccatctgctagggatgtgtacggagtgcgccgagacaaccgagatcgtgttggtcgtagtgtcatgccaaatggaacgccggacgtagtcatatttcctgaagcaaaaccaggtgcacatgcctcacgcggctctgttggctctagcacaaccagcatcacagtacggtttaccttgcggcctgcatcagtataaaggacttcgttttggtccacggggcgtgacaaacagatctgcgtctccggtctcgccgcttagatcgctctgtgtagtagttgtagtatatccactctctcaaagcatccaggcgccccctcccgcactgtttgtctagacgcagaggccagagcggcgaatctagcgagacacatcatcaacatcatataggtgagagagtttcgtaggtccgcgggggaggcttcgggttctatgtaaactccttcatgcgccgctgccctgataacatccaccaccgcagaataagccacacccagccaacctacacattcgttctgcccgaagcccaagatacatttgaggaagtacgcggcgacgggactattgtaggtggtggcgtcttattcggtgtgggtcggttggatgtgtaagcaagacggagtcacacacgggaggagcgggaagagctggaagaaccatgtttttttttttattccaaaagattatccaaaacctcaaaatgaagatctattaagtgactcagtgtgtgccctcctcgcccttctcgaccttcttggtacaaaaaaaaaaataaggttttctaataggttttggagttttacttctagataattcactacgcgctcccctccggtggcgtggtcaaactctacagccaaagaacagataatggcatttgtaagatgttgcacaatggcttccaaaaggcaaacggccctgcgcgaggggaggccaccgcaccagtttgagatgtcggtttcttgtctattaccgtaaacattctacaacgtgttaccgaaggttttccgtttgccgggtcacgtccaagtggacgtaaaggctaaacccttcagggtgaatctcctctataaacattccagcaccttcaaccatgcccaaataattctcatctcgccaagtgcaggttcacctgcatttccgatttgggaagtcccacttagaggagatatttgtaaggtcgtggaagttggtacgggtttattaagagtagagcggtccttctcaatatatctctaagcaaatcccgaatattaagtccggccattgtaaaaatctgctccagagcgccctccaccttcagcctcaagcagcgaatcggaagagttatatagagattcgtttagggcttataattcaggccggtaacatttttagacgaggtctcgcgggaggtggaagtcggagttcgtcgcttagatgattgcaaaaattcaggttcctcacagacctgtataagattcaaaagcggaacattaacaaaaataccgcgatcccgtaggtcccttcgcagggccagtactaacgtttttaagtccaaggagtgtctggacatattctaagttttcgccttgtaattgtttttatggcgctagggcatccagggaagcgtcccggtcctgaacataatcgtgcaggtctgcacggaccagcgcggccacttccccgccaggaaccttgacaaaagaacccacactgattatgacacgcatactcggagacttgtattagcacgtccagacgtgcctggtcgcgccggtgaaggggcggtccttggaactgttttcttgggtgtgactaatactgtgcgtatgagcctgctatgctaaccagcgtagccccgatgtaagctttgttgcatgggcggcgatataaaatgcaaggtagcttgttgcatgggcggcgatataaaatgcaagcgatacgattggtcgcatcggggctacattcgaaacaacgtacccgccgctatattttacgttccatcgaacaacgtacccgccgctatattttacgttcgtgctgctcaaaaaatcaggcaaagcctcgcgcaaaaaagaaagcacatcgtagtcatgctcatgcagataaaggcaggtaagctccggaaccaccacagcacgacgagttttttagtccgtttcggagcgcgttttttctttcgtgtagcatcagtacgagtacgtctatttccgtccattcgaggccttggtggtgtcaaaaagacaccatttttctctcaaacatgtctgcgggtttctgcataaacacaaaataaaataacaaaaaaacatttaaacattagaagcctgtcttacatttttctgtggtaaaaagagagtttgtacagacgcccaaagacgtatttgtgttttattttattgtttttttgtaaatttgtaatcttcggacagaatgtacaggaaaaacaacccttataagcataagacggactacggccatgccggcgtgaccgtaaaaaaactggtcaccgtgattaaaaagcaccaccgacagcttgtcctttttgttgggaatattcgtattctgcctgatgccggtacggccgcactggcatttttttgaccagtggcactaatttttcgtggtggctgtcgacctcggtcatgtccggagtcataatgtaagactcggtaaacacatcaggttgattcatcggtcagtgctaaaaagcgaccgaaatagcccgggggaatacggagccagtacaggcctcagtattacattctgagccatttgtgtagtccaactaagtagccagtcacgatttttcgctggctttatcgggcccccttatgatacccgcaggcgtagagacaacattacagcccccataggaggtataacaaaattaataggagagaaaaacacataaacacctgaaaaaccctcctgccttatgggcgtccgcatctctgttgtaatgtcgggggtatcctccatattgttttaattatcctctctttttgtgtatttgtggactttttgggaggacggaaggcaaaatagcaccctcccgctccagaacaacatacagcgcttcacagcggcagcctaacagtcagccttaccagtaaaaaagaaaacctattaaaaaatccgttttatcgtgggagggcgaggtcttgttgtatgtcgcgaagtgtcgccgtcggattgtcagtcggaatggtcattttttcttttggataattttttacaccactcgacacggcaccagctcaatcagtcacagtgtaaaaaagggccaagtgcagagcgagtatatataggactaaaaaatgacgtaacggttaaatgtggtgagctgtgccgtggtcgagttagtcagtgtcacattttttcccggttcacgtctcgctcatatatatcctgattttttactgcattgccaatttgtccacaaaaaacacccagaaaaccgcacgcgaacctacgcccagaaacgaaagccaaaaaacccacaacttcctcaaatcgtcacttccgttttcccaccaggtgttttttgtgggtcttttggcgtgcgcttggatgcgggtctttgctttcggttttttgggtgttgaaggagtttagcagtgaaggcaaaagggtggttacgtaacttcccattttaagaaaactacaattcccaacacatacaagttactccgccctaaaacctacgtcacccgccccgttcccacgccccgcgccaatgcattgaagggtaaaattcttttgatgttaagggttgtgtatgttcaatgaggcgggattttggatgcagtgggcggggcaagggtgcggggcgcgcacgtcacaaactccaccccctcattatcatattggcttcaatccaaaataaggtatattattgatgatgttaatgtgcagtgtttgaggtgggggagtaatagtataaccgaagttaggttttattccatataataactactacaatta

SEQ ID NO: 2 - DNA, 72118 bp, Artificial
  /note = Sequence of Hybrid Ad-SFV vector containing E1, E3 and E4 deletions from the adenovirus backbone
taacatcatcaataatataccttattttggattgaagccaatatgataatgagggggtggagtttgtgacgtggcgcggggcgtgggaacggggcgggtgattgtagtagttattatatggaataaaacctaacttcggttatactattactcccccacctcaaacactgcaccgcgccccgcacccttgccccgcccacacgtagtagtgtggcggaagtgtgatgttgcaagtgtggcggaacacatgtaagcgacggatgtggcaaaagtgacgtttttggtgtgcgccggtgtacatgcatcatcacaccgccttcacactacaacgttcacaccgccttgtgtacattcgctgcctacaccgttttcactgcaaaaaccacacgcggccacatgtcaggaagtgacaattttcgcgcggttttaggcggatgttgtagtaaatttgggcgtaaccgagtaagatttggccattttcgcgggaaaactgaataagagtccttcactgttaaaagcgcgccaaaatccgcctacaacatcatttaaacccgcattggctcattctaaaccggtaaaagcgcccttttgacttattctggaagtgaaatctgaataattttgtgttactcatagcgcgtaatactggtaccgcggccgcctcgagtttaccactccctatcagtgatagagaaaagtgccttcactttagacttattaaaacacaatgagtatcgcgcattatgaccatggcgccggcggagctcaaatggtgagggatagtcactatctcttttcacaaagtcgagtttaccactccctatcagtgatagagaaaagtgaaagtcgagtttaccactccctatcagtgatagagaaaagtgaaagtcgagtttaccatttcagctcaaatggtgagggatagtcactatctcttttcactttcagctcaaatggtgagggatagtcactatctcttttcactttcagctcaaatggtctccctatcagtgatagagaaaagtgaaagtcgagtttaccactccctatcagtgatagagaaaagtgaaagtcgagtttaccactccctatcagtgatagagggatagtcactatctcttttcactttcagctcaaatggtgagggatagtcactatctcttttcactttcagctcaaatggtgagggatagtcactatgagaaaagtgaaagtcgagtttaccactccctatcagtgatagagaaaagtgaaagtcgagctcggtacccgggtcgaggtaggcgtgtacggtgggaggctcttttcactttcagctcaaatggtgagggatagtcactatctcttttcactttcagctcgagccatgggcccagctccatccgcacatgccaccctcccctatataagcagagctcgtttagtgaaccgtcagatcgcctggagacgccatccacgctgttttgacctccatagaagacaccgggaccgatccagcctggatatattcgtctcgagcaaatcacttggcagtctagcggacctctgcggtaggtgcgacaaaactggaggtatcttctgtggccctggctaggtcggaccgcggccccgaattcgagctcggtacccggggatcgatggcggatgtgtgacatacacgacgccaaaagattttgttccagctcctgccacctccgctaggcgccggggcttaagctcgagccatgggcccctagctaccgcctacacactgtatgtgctgcggttttctaaaacaaggtcgaggacggtggaggcgatcgcgagagattaaccacccacgatggccgccaaagtgcatgttgatattgaggctgacagcccattcatcaagtctttgcagaaggcatttccgtcgttcgcgctctctaattggtgggtgctaccggcggtttcacgtacaactataactccgactgtcgggtaagtagttcagaaacgtcttccgtaaaggcagcaaggaggtggagtcattgcaggtcacaccaaatgaccatgcaaatgccagagcattttcgcacctggctaccaaattgatcgagcaggagactgacaaagacactccacctcagtaacgtccagtgtggtttactggtacgtttacggtctcgtaaaagcgtggaccgatggtttaactagctcgtcctctgactgtttctgtcactcatcttggatatcggcagtgcgccttccaggagaatgatgtctacgcacaaataccactgcgtatgccctatgcgcagcgcagaagaccccgaaaggtgagtagaacctatagccgtcacgcggaaggtcctcttactacagatgcgtgtttatggtgacgcatacgggatacgcgtcgcgtcttctggggctttcgctcgatagctacgcaaagaaactggcagcggcctccgggaaggtgctggatagagagatcgcaggaaaaatcaccgacctgcagaccgtcatggctacgcgagctatcgatgcgtttctttgaccgtcgccggaggcccttccacgacctatctctctagcgtcctttttagtggctggacgtctggcagtaccgatgcccagacgctgaatctcctaccttttgcctgcatacagacgtcacgtgtcgtacggcagccgaagtggccgtataccaggacgtgtatgctgtacatgcacggtctgcgacttagaggatggaaaacggacgtatgtctgcagtgcacagcatgccgtcggcttcaccggcatatggtcctgcacatacgacatgtacgtgcaacatcgctgtaccatcaggcgatgaaaggtgtcagaacggcgtattggattgggtttgacaccaccccgtttatgtttgacgcgctagcaggcgcgtagttgtagcgacatggtagtccgctactttccacagtcttgccgcataacctaacccaaactgtggtggggcaaatacaaactgcgcgatcgtccgcgcattccaacctacgccacaaactgggccgacgagcaggtgttacaggccaggaacataggactgtgtgcagcatccttgactgagggaagactcggcaaactgaggttggatgcggtgtttgacccggctgctcgtccacaatgtccggtccttgtatcctgacacacgtcgtaggaactgactcccttctgagccgtttgactccattctccgcaagaagcaattgaaaccttgcgacacagtcatgttctcggtaggatctacattgtacactgagagcagaaagctactgaggagctggcaggtaagaggcgttcttcgttaactttggaacgctgtgtcagtacaagagccatcctagatgtaacatgtgactctcgtctttcgatgactcctcgaccgacttaccctccgtattccacctgaaaggtaaacaatcctttacctgtaggtgcgataccatcgtatcatgtgaagggtacgtagttaagaaaatcactattgaatgggaggcataaggtggactttccatttgttaggaaatggacatccacgctatggtagcatagtacacttcccatgcatcaattcttttagtgatagtgccccggcctgtacggtaaaacggtagggtacgccgtgacgtatcacgcggagggattcctagtgtgcaagaccacagacactgtcaaaggagaaagacacggggccggacatgccattttgccatcccatgcggcactgcatagtgcgcctccctaaggatcacacgttctggtgtctgtgacagtttcctctttctgtctcattccctgtatgcacctacgtcccctcaaccatctgtgatcaaatgactggcatactagcgaccgacgtcacaccggaggacgcacagaagttgtcagagtaagggacatacgtggatgcaggggagttggtagacactagtttactgaccgtatgatcgctggctgcagtgtggcctcctgcgtgtcttcaacatagtgggattgaatcagaggatagttgtgaacggaagaacacagcgaaacactaacacgatgaagaactatctgcttccgattgtggccgtcgcatttagatcaccctaacttagtctcctatcaacacttgccttcttgtgtcgctttgtgattgtgctacttcttgatagacgaaggctaacaccggcagcgtaaatccaagtgggcgagggaatacaaggcagaccttgatgatgaaaaacctctgggtgtccgagagaggtcacttacttgctgctgcttgtgggcatttaaaacggttcacccgctcccttatgttccgtctggaactactactttttggagacccacaggctctctccagtgaatgaacgacgacgaacacccgtaaattttgcaggaagatgcacaccatgtacaagaaaccagacacccagacaatagtgaaggtgccttcagagtttaactcgttcgtcatcccgagcctatggtctacagtccttctacgtgtggtacatgttctttggtctgtgggtctgttatcacttccacggaagtctcaaattgagcaagcagtagggctcggataccagatgtcgcctcgcaatcccagtcagatcacgcattaagatgcttttggccaagaagaccaagcgagagttaatacctgttctcgacgcgtcgtcagccagggatgccggagcgttagggtcagtctagtgcgtaattctacgaaaaccggttcttctggttcgctctcaattatggacaagagctgcgcagcagtcggtccctacgtgaacaagaggagaaggagaggttggaggccgagctgactagagaagccttaccacccctcgtccccatcgcgccggcggagacgggagtcgtcgacgtcacttgttctcctcttcctctccaacctccggctcgactgatctcttcggaatggtggggagcaggggtagcgcggccgcctctgccctcagcagctgcaggacgttgaagaactagagtatcacgcaggtgcaggggtcgtggaaacacctcgcagcgcgttgaaagtcaccgcacagccgaacgacgtactactaggaactgcaacttcttgatctcatagtgcgtccacgtccccagcacctttgtggagcgtcgcgcaactttcagtggcgtgtcggcttgctgcatgatgatccttattacgtagttctgtccccgcagaccgtgctcaagagctccaagttggcccccgtgcaccctctagcagagcaggtgaaaataataacacataacgggagtaatgcatcaagacaggggcgtctggcacgagttctcgaggttcaaccgggggcacgtgggagatcgtctcgtccacttttattattgtgtattgccctcggccggcggttaccaggtcgacggatatgacggcagggtcctactaccatgtggatcggccattccggtccctgagtttcaggctttgagcgagagcgccccggccgccaatggtccagctgcctatactgccgtcccaggatgatggtacacctagccggtaaggccagggactcaaagtccgaaactcgctctcgcggactatggtgtacaacgaaagggagttcgtcaacaggaaactataccatattgccgttcacggaccctcgctgaacaccgacgaggagaactacgagaaagtgataccacatgttgctttccctcaagcagttgtcctttgatatggtataacggcaagtgcctgggagcgacttgtggctgctcctcttgatgctctttctcagagctgaaagaactgacgccgagtacgtgttcgacgtagataaaaaatgctgcgtcaagagagaggaagcgtcgggtttggtgttggtgggagagctagtctcgactttcttgactgcggctcatgcacaagctgcatctattttttacgacgcagttctctctccttcgcagcccaaaccacaaccaccctctcgaaaccaaccccccgttccatgaattcgcctacgaagggctgaagatcaggccgtcggcaccatataagactacagtagtaggagtctttggggttccgggattggttggggggcaaggtacttaagcggatgcttcccgacttctagtccggcagccgtggtatattctgatgtcatcatcctcagaaaccccaaggcccttcaggcaagtctgctattattaagagcctcgtgaccaaacacgatctggtcaccagcggcaagaaggagaactgccaggaaatagttaacgacgtgaagaagtccgttcagacgataataattctcggagcactggtttgtgctagaccagtggtcgccgttcttcctcttgacggtcctttatcaattgctgcacttctagcaccgcgggaaggggacaagtagggaaaacagtgactccatcctgctaaacgggtgtcgtcgtgccgtggacatcctatatgtggacgaggctttcgctcgtggcgcccttcccctgttcatcccttttgtcactgaggtaggacgatttgcccacagcagcacggcacctgtaggatatacacctgctccgaaagcgttgccatcccggtactctgctggccctaattgctcttgttaaacctcggagcaaagtggtgttatgcggagaccccaagcaatgcggattcttcaatatgaacggtagggccatgagacgaccgggattaacgagaacaatttggagcctcgtttcaccacaatacgcctctggggttcgttacgcctaagaagttatacatgcagcttaaggtgaacttcaaccacaacatctgcactgaagtatgtcataaaagtatatccagacgttgcacgcgtccagtcacggccatcgtgtctatacgtcgaattccacttgaagttggtgttgtagacgtgacttcatacagtattttcatataggtctgcaacgtgcgcaggtcagtgccggtagcacagatcgttgcactacggaggcaagatgcgcacgaccaacccgtgcaacaaacccataatcatagacaccacaggacagaccaagcccaagccaggagacatcgtgcaacgtgatgcctccgttctacgcgtgctggttgggcacgttgtttgggtattagtatctgtggtgtcctgtctggttcgggttcggtcctctgtagcagttaacatgcttccgaggctgggcaaagcagctgcagttggactaccgtggacacgaagtcatgacagcagcagcatctcagggcctcacccgcaaagggcaattgtacgaaggctccgacccgtttcgtcgacgtcaacctgatggcacctgtgcttcagtactgtcgtcgtcgtagagtcccggagtgggcgtttcccgtatacgccgtaaggcagaaggtgaatgaaaatcccttgtatgcccctgcgtcggagcacgtgaatgtactgctgacgcgcactgaggataggctggtgtcatatgcggcattccgtcttccacttacttttagggaacatacggggacgcagcctcgtgcacttacatgacgactgcgcgtgactcctatccgaccacaggaaaacgctggccggcgatccctggattaaggtcctatcaaacattccacagggtaactttacggccacattggaagaatggcaagaagaacacgacaaccttttgcgaccggccgctagggacctaattccaggatagtttgtaaggtgtcccattgaaatgccggtgtaaccttcttaccgttcttcttgtgctgttaataatgaaggtgattgaaggaccggctgcgcctgtggacgcgttccagaacaaagcgaacgtgtgttgggcgaaaagcctggtgcctgtcctggacactttattacttccactaacttcctggccgacgcggacacctgcgcaaggtcttgtttcgcttgcacacaacccgcttttcggaccacggacaggacctgtgagccggaatcagattgacagcagaggagtggagcaccataattacagcatttaaggaggacagagcttactctccagtggtggccttgaatgaaatttgcacggccttagtctaactgtcgtctcctcacctcgtggtattaatgtcgtaaattcctcctgtctcgaatgagaggtcaccaccggaacttactttaaacgtccaagtactatggagttgacctggacagtggcctgttttctgccccgaaggtgtccctgtattacgagaacaaccactgggataacagacctggtggaagggttcatgatacctcaactggacctgtcaccggacaaaagacggggcttccacagggacataatgctcttgttggtgaccctattgtctggaccaccttcgatgtatggattcaatgccgcaacagctgccaggctggaagctagacataccttcctgaaggggcagtggcatacgggcaagcaggcagttatcgcagaactacatacctaagttacggcgttgtcgacggtccgaccttcgatctgtatggaaggacttccccgtcaccgtatgcccgttcgtccgtcaatagcgtcttagaaaaatccaaccgctttctgtgctggacaatgtaattcctatcaaccgcaggctgccgcacgccctggtggctgagtacaagacggttaaaggcagtatctttttaggttggcgaaagacacgacctgttacattaaggatagttggcgtccgacggcgtgcgggaccaccgactcatgttctgccaatttccgtcatgggttgagtggctggtcaataaagtaagagggtaccacgtcctgctggtgagtgagtacaacctggctttgcctcgacgcgacgtcacttggttgtcacccccaactcaccgaccagttatttcattctcccatggtgcaggacgaccactcactcatgttggaccgaaacggagctgcgctgcagtgaaccaacagtgggctgaatgtcacaggcgccgataggtgctacgacctaagtttaggactgccggctgacgccggcaggttcgacttggtctttgtgaacattcacacggaacgacttacagtgtccgcggctatccacgatgctggattcaaatcctgacggccgactgcggccgtccaagctgaaccagaaacacttgtaagtgtgccttttcagaatccaccactaccagcagtgtgtcgaccacgccatgaagctgcagatgcttgggggagatgcgctacgactgctaaaacccggcggcatcttgaaagtcttaggtggtgatggtcgtcacacagctggtgcggtacttcgacgtctacgaaccccctctacgcgatgctgacgattttgggccgccgtagaacttgagagcttacggatacgccgataaaatcagcgaagccgttgtttcctccttaagcagaaagttctcgtctgcaagagtgttgcgcccggattgtgtcacactctcgaatgcctatgcggctattttagtcgcttcggcaacaaaggaggaattcgtctttcaagagcagacgttctcacaacgcgggcctaacacagtgcagcaatacagaagtgttcttgctgttctccaactttgacaacggaaagagaccctctacgctacaccagatgaataccaagctgagtgccgtgtatgccgtcgttatgtcttcacaagaacgacaagaggttgaaactgttgcctttctctgggagatgcgatgtggtctacttatggttcgactcacggcacatacggggagaagccatgcacacggccgggtgtgcaccatcctacagagttaagagagcagacatagccacgtgcacagaagcggctgtggttaacgcagctaacgcctcttcggtacgtgtgccggcccacacgtggtaggatgtctcaattctctcgtctgtatcggtgcacgtgtcttcgccgacaccaattgcgtcgattgccccgtggaactgtaggggatggcgtatgcagggccgtggcgaagaaatggccgtcagcctttaagggagcagcaacaccagtgggcacaattaaaacagtgggcaccttgacatcccctaccgcatacgtcccggcaccgcttctttaccggcagtcggaaattccctcgtcgttgtggtcacccgtgttaattttgtcacatgtgcggctcgtaccccgtcatccacgctgtagcgcctaatttctctgccacgactgaagcggaaggggaccgcgaattggccgctgtctaccgggcagtacacgccgagcatggggcagtaggtgcgacatcgcggattaaagagacggtgctgacttcgccttcccctggcgcttaaccggcgacagatggcccgtgtggccgccgaagtaaacagactgtcactgagcagcgtagccatcccgctgctgtccacaggagtgttcagcggcggaagagataggctgcagcaatccccaccggcggcttcatttgtctgacagtgactcgtcgcatcggtagggcgacgacaggtgtcctcacaagtcgccgccttctctatccgacgtcgttagggtcaaccatctattcacagcaatggacgccacggacgctgacgtgaccatctactgcagagacaaaagttgggagaagaaaatccaggaagccattgacatagttggtagataagtgtcgttacctgcggtgcctgcgactgcactggtagatgacgtctctgttttcaaccctcttcttttaggtccttcggtaactgtagaggacggctgtggagttgctcaatgatgacgtggagctgaccacagacttggtgagagtgcacccggacagcagcctggtgggtcgtaagggctacagtctcctgccgacacctcaacgagttactactgcacctcgactggtgtctgaaccactctcacgtgggcctgtcgtcggaccacccagcattcccgatgtcaaccactgacgggtcgctgtactcgtactttgaaggtacgaaattcaaccaggctgctattgatatggcagagatactgacgttgtggcccagactgcaagtggtgactgcccagcgacatgagcatgaaacttccatgctttaagttggtccgacgataactataccgtctctatgactgcaacaccgggtctgacgttcaggcaaacgaacagatatgcctatacgcgctgggcgaaacaatggacaacatcagatccaaatgtccggtgaacgattccgattcatcaacacctcccagtccgtttgcttgtctatacggatatgcgcgacccgctttgttacctgttgtagtctaggtttacaggccacttgctaaggctaagtagttgtggagggtcgacagtgccctgcctgtgccgctacgcaatgacagcagaacggatcgcccgccttaggtcacaccaagttaaaagcatggtggtttgctcatcttttcccctgtcacgggacggacacggcgatgcgttactgtcgtcttgcctagcgggcggaatccagtgtggttcaattttcgtaccaccaaacgagtagaaaagggctcccgaaataccatgtagatggggtgcagaaggtaaagtgcgagaaggttctcctgttcgacccgacggtaccttcagtggttagtccgcggaagtatggagggctttatggtacatctaccccacgtcttccatttcacgctcttccaagaggacaagctgggctgccatggaagtcaccaatcaggcgccttcatacccgcatctacgacggaccactcagatcggtcgttacgagggtttgacttggactggaccaccgactcgtcttccactgccagcgataccatgtcgctaccggcgtagatgctgcctggtgagtctagccagcaatgctcccaaactgaacctgacctggtggctgagcagaaggtgacggtcgctatggtacagcgatggcagtttgcagtcgtgtgacatcgactcgatctacgagccaatggctcccatagtagtgacggctgacgtacaccctgaacccgcaggcatcgcggacctggtcaaacgtcagcacactgtagctgagctagatgctcggttaccgagggtatcatcactgccgactgcatgtgggacttgggcgtccgtagcgcctggacgcggcagatgtgcaccctgaacccgcagaccatgtggacctggagaacccgattcctccaccgcgcccgaagagagctgcataccttgcctcccgcgcggcgccgtctacacgtgggacttgggcgtctggtacacctggacctcttgggctaaggaggtggcgcgggcttctctcgacgtatggaacggagggcgcgcccggagcgaccggtgccggcgccgagaaagccgacgcctgccccaaggactgcgtttaggaacaagctgcctttgacgttcggcgactttgacgagcacgagcctcgctggccacggccgcggctctttcggctgcggacggggttcctgacgcaaatccttgttcgacggaaactgcaagccgctgaaactgctcgtgctggtcgatgcgttggcctccgggattactttcggagacttcgacgacgtcctgcgactaggccgcgcgggtgcatatattttctcctcggacactggcagcccagctacgcaaccggaggccctaatgaaagcctctgaagctgctgcaggacgctgatccggcgcgcccacgtatataaaagaggagcctgtgaccgtcgggacatttacaacaaaaatccgttaggcagcacaatctccagtgcgcacaactggatgcggtccaggaggagaaaatgtacccgccaaaattggatactgcctgtaaatgttgtttttaggcaatccgtcgtgttagaggtcacgcgtgttgacctacgccaggtcctcctcttttacatgggcggttttaacctatgacagagggagaagctgttgctgctgaaaatgcagatgcacccatcggaggctaataagagtcgataccagtctcgcaaagtggagaacatgaaagccacggttctccctcttcgacaacgacgacttttacgtctacgtgggtagcctccgattattctcagctatggtcagagcgtttcacctcttgtactttcggtgccaggtggacaggctcacatcgggggccagattgtacacgggagcggacgtaggccgcataccaacatacgcggttcggtacccccgccccgtgtactcccctccacctgtccgagtgtagcccccggtctaacatgtgccctcgcctgcatccggcgtatggttgtatgcgccaagccatgggggcggggcacatgaggggaaccgtgatcgaaagattctcaagccccgatgtagcaatcgcagcgtgcaacgaatacctatccagaaattacccaacagtggcgtcgtaccagataacagtggcactagctttctaagagttcggggctacatcgttagcgtcgcacgttgcttatggataggtctttaatgggttgtcaccgcagcatggtctattgtcatgaatacgacgcatacttggacatggttgacgggtcggatagttgcttggacagagcgacattctgcccggcgaagctccggtgctacccgaaacatcatacttatgctgcgtatgaacctgtaccaactgcccagcctatcaacgaacctgtctcgctgtaagacgggccgcttcgaggccacgatgggctttgtagttgcgtaccaccagccgactgtacgcagtgccgtcccgtcaccctttcagaacacactacagaacgtgctagcggctgccaccaagagaaactgcaacgtcacgcatggtggtcggctgacatgcgtcacggcagggcagtgggaaagtcttgtgtgatgtcttgcacgatcgccgacggtggttctctttgacgttgcagacgcaaatgcgagaactacccaccatggactcggcagtgttcaacgtggagtgcttcaagcgctatgcctgctccggagaatattgggaagaatatgctatgcgtttacgctcttgatgggtggtacctgagccgtcacaagttgcacctcacgaagttcgcgatacggacgaggcctcttataacccttcttatacgataacaacctatccggataaccactgagaacatcactacctatgtgaccaaattgaaaggcccgaaagctgctgccttgttcgctaagacccacaacttggtttgttggataggcctattggtgactcttgtagtgatggatacactggtttaactttccgggctttcgacgacggaacaagcgattctgggtgttgaaccatccgctgcaggaggttcccatggacagattcacggtcgacatgaaacgagatgtcaaagtcactccagggacgaaacacacagaggaaagacccaaagtcaggcgacgtcctccaagggtacctgtctaagtgccagctgtactttgctctacagtttcagtgaggtccctgctttgtgtgtctcctttctgggtttcagcaggtaattcaagcagcggagccattggcgaccgcttacctgtgcggcatccacagggaattagtaaggagactaaatgctgtgttacgccctaacgtgcgtccattaagttcgtcgcctcggtaaccgctggcgaatggacacgccgtaggtgtcccttaatcattcctctgatttacgacacaatgcgggattgcacgacacattgtttgatatgtcggccgaagactttgacgcgatcatcgcctctcacttccacccaggagacccggttctagagacggacattgcatcattcgatgtgtaacaaactatacagccggcttctgaaactgcgctagtagcggagagtgaaggtgggtcctctgggccaagatctctgcctgtaacgtagtaagctcaaaagccaggacgactccttggctcttacaggtttaatgatcctcgaagatctaggggtggatcagtacctgctggacttgatcgaggcagcctttggggttttcggtcctgctgaggaaccgagaatgtccaaattactaggagcttctagatccccacctagtcatggacgacctgaactagctccgtcggaaacccgaaatatccagctgtcacctaccaactggcacgcgcttcaagttcggagctatgatgaaatcgggcatgtttctgactttgtttattaacactgttttgactttataggtcgacagtggatggttgaccgtgcgcgaagttcaagcctcgatactactttagcccgtacaaagactgaaacaaataattgtgacaaaactacatcaccatagcaagcagggtactggagcagagactcactgactccgcctgtgcggccttcatcggcgacgacaacatcgttcacggagtgatctccgatgtagtggtatcgttcgtcccatgacctcgtctctgagtgactgaggcggacacgccggaagtagccgctgctgttgtagcaagtgcctcactagaggctcaagctgatggcggagaggtgcgcgtcgtgggtcaacatggaggtgaagatcattgacgctgtcatgggcgaaaaacccccatatttttgtgggggattcgttcgactaccgcctctccacgcgcagcacccagttgtacctccacttctagtaactgcgacagtacccgctttttgggggtataaaaacaccccctaagatagtttttgacagcgtcacacagaccgcctgccgtgtttcagacccacttaagcgcctgttcaagttgggtaagccgctaacagctgaagacaagcaggtatcaaaaactgtcgcagtgtgtctggcggacggcacaaagtctgggtgaattcgcggacaagttcaacccattcggcgattgtcgacttctgttcgtccacgaagacaggcgacgagcactgagtgacgaggttagcaagtggttccggacaggcttgggggccgaactggaggtggcactaacatctaggtatgaggttgcttctgtccgctgctcgtgactcactgctccaatcgttcaccaaggcctgtccgaacccccggcttgacctccaccgtgattgtagatccatactccaagagggctgcaaaagtatcctcatagccatggccaccttggcgagggacattaaggcgtttaagaaattgagaggacctgttatacacctctacggcggttctcccgacgttttcataggagtatcggtaccggtggaaccgctccctgtaattccgcaaattctttaactctcctggacaatatgtggagatgccgccacctagattggtgcgttaatacacagaattctgattggatcccggtccgaagcgcgctttcccgggaactcgagttcactagtcgatcccgcggccgctttggatctaaccacgcaattatgtgtcttaagactaacctagggccaggcttcgcgcgaaagggcccttgagctcaagtgatcagctagggcgccggcgaaacgaacctaggcaagcatgcgggcccagtgggtaattaattgaattacatccctacgcaaacgttttacggccgccggtggcgcccgcgcccggcggcccggcttggatccgttcgtacgcccgggtcacccattaattaacttaatgtagggatgcgtttgcaaaatgccggcggccaccgcgggcgcgggccgccgggctccttggccgttgcaggccactccggtggctcccgtcgtccccgacttccaggcccagcagatgcagcaactcatcagcgccgtaaatgcgctgacaatgaggaaccggcaacgtccggtgaggccaccgagggcagcaggggctgaaggtccgggtcgtctacgtcgttgagtagtcgcggcatttacgcgactgttacagacagaacgcaattgctcctgctaggagcttaattcgacgaataattggatttttattttattttgcaattggtttttaatatttccaaaaaaaaaaaatctgtcttgcgttaacgaggacgatcctcgaattaagctgcttattaacctaaaaataaaataaaacgttaaccaaaaattataaaggttttttttttttaaaaaaaaaaaaaaaaaaaaaaaaaaaaaaaaaaaaaaaaaaaaaaaaaaaaaaaaaactagaaatcgatcggcagtgcgccttccaggagaactagcgcttttttttttttttttttttttttttttttttttttttttttttttttttttttttttgatctttagctagccgtcacgcggaaggtcctcttgatcgcgggccgcatcgataagcttgtcgactcgaagatccagacatgataagatacattgatgagtttggacaaaccacaactagaatgcagtgaaaaaaatgcttccggcgtagctattcgaacagctgagcttctaggtctgtactattctatgtaactactcaaacctgtttggtgttgatcttacgtcactttttttacgaatatttgtgaaatttgtgatgctattgctttatttgtaaccattataagctgcaataaacaagttaacaacaacaattgcattcattttatgtttcaggttataaacactttaaacactacgataacgaaataaacattggtaatattcgacgttatttgttcaattgttgttgttaacgtaagtaaaatacaaagtccaacagggggaggtgtgggaggttttttcggatctgggcgtggttaagggtgggaaagaatatataaggtgggggtcttatgtagttttgtatctgttttgcagtccccctccacaccctccaaaaaagcctagacccgcaccaattcccaccctttcttatatattccacccccagaatacatcaaaacatagacaaaacgtgcagccgccgccgccatgagcaccaactcgtttgatggaagcattgtgagctcatatttgacaacgcgcatgcccccatgggccggggtgcgtcagaatgcgtcggcggcggcggtactcgtggttgagcaaactaccttcgtaacactcgagtataaactgttgcgcgtacgggggtacccggccccacgcagtcttactgatgggctccagcattgatggtcgccccgtcctgcccgcaaactctactaccttgacctacgagaccgtgtctggaacgccgttggagactgcagcctcactacccgaggtcgtaactaccagcggggcaggacgggcgtttgagatgatggaactggatgctctggcacagaccttgcggcaacctctgacgtcggagcgccgccgcttcagccgctgcagccaccgcccgcgggattgtgactgactttgctttcctgagcccgcttgcaagcagtgcagcttcccgttcatccgccgcggcggcgaagtcggcgacgtcggtggcgggcgccctaacactgactgaaacgaaaggactcgggcgaacgttcgtcacgtcgaagggcaagtaggcggcgcgatgacaagttgacggctcttttggcacaattggattctttgacccgggaacttaatgtcgtttctcagcagctgttggatctgcgccagcaggtttgcgctactgttcaactgccgagaaaaccgtgttaacctaagaaactgggcccttgaattacagcaaagagtcgtcgacaacctagacgcggtcgtccaaactgccctgaaggcttcctcccctcccaatgcggtttaaaacataaataaaaaaccagactctgtttggatttggatcaagcaagtgtcttgctgtctttagacgggacttccgaaggaggggagggttacgccaaattttgtatttattttttggtctgagacaaacctaaacctagttcgttcacagaacgacagaaattttaggggttttgcgcgcgcggtaggcccgggaccagcggtctcggtcgttgagggtcctgtgtattttttccaggacgtggtaaaggtgactctggatgaaatccccaaaacgcgcgcgccatccgggccctggtcgccagagccagcaactcccaggacacataaaaaaggtcctgcaccatttccactgagacctacttcagatacatgggcataagcccgtctctggggtggaggtagcaccactgcagagcttcatgctgcggggtggtgttgtagatgatccagtcgtagcaggaagtctatgtacccgtattcgggcagagaccccacctccatcgtggtgacgtctcgaagtacgacgccccaccacaacatctactaggtcagcatcgtccagcgctgggcgtggtgcctaaaaatgtctttcagtagcaagctgattgccaggggcaggcccttggtgtaagtgtttacaaagcggttaagctgggatggtcgcgacccgcaccacggatttttacagaaagtcatcgttcgactaacggtccccgtccgggaaccacattcacaaatgtttcgccaattcgaccctaccgtgcatacgtggggatatgagatgcatcttggactgtatttttaggttggctatgttcccagccatatccctccggggattcatgttgtgcagaaccacccacgtatgcacccctatactctacgtagaacctgacataaaaatccaaccgatacaagggtcggtatagggaggcccctaagtacaacacgtcttggtggagcacagtgtatccggtgcacttgggaaatttgtcatgtagcttagaaggaaatgcgtggaagaacttggagacgcccttgtgacctccaagattttccatcgtgtcacataggccacgtgaaccctttaaacagtacatcgaatcttcctttacgcaccttcttgaacctctgcgggaacactggaggttctaaaaggttgcattcgtccataatgatggcaatgggcccacgggcggcggcctgggcgaagatatttctgggatcactaacgtcatagttgtgttccaggatgagatcacgtaagcaggtattactaccgttacccgggtgcccgccgccggacccgcttctataaagaccctagtgattgcagtatcaacacaaggtcctactctaggtcataggccatttttacaaagcgcgggcggagggtgccagactgcggtataatggttccatccggcccaggggcgtagttaccctcacagatttgcattcagtatccggtaaaaatgtttcgcgcccgcctcccacggtctgacgccatattaccaaggtaggccgggtccccgcatcaatgggagtgtctaaacgtaatcccacgctttgagttcagatggggggatcatgtctacctgcggggcgatgaagaaaacggtttccggggtaggggagatcagctgggaagaaagcaggtagggtgcgaaactcaagtctacccccctagtacagatggacgccccgctacttcttttgccaaaggccccatcccctctagtcgacccttctttcgtccatcctgagcagctgcgacttaccgcagccggtgggcccgtaaatcacacctattaccgggtgcaactggtagttaagagagctgcagctgccgtcatccctaggactcgtcgacgctgaatggcgtcggccacccgggcatttagtgtggataatggcccacgttgaccatcaattctctcgacgtcgacggcagtagggagagcaggggggccacttcgttaagcatgtccctgactcgcatgttttccctgaccaaatccgccagaaggcgctcgccgcccagcgatagcagttcttgcctcgtccccccggtgaagcaattcgtacagggactgagcgtacaaaagggactggtttaggcggtcttccgcgagcggcgggtcgctatcgtcaagaacgaaggaagcaaagtttttcaacggtttgagaccgtccgccgtaggcatgcttttgagcgtttgaccaagcagttccaggcggtcccacagctcggtcacctttccttcgtttcaaaaagttgccaaactctggcaggcggcatccgtacgaaaactcgcaaactggttcgtcaaggtccgccagggtgtcgagccagtggagctctacggcatctcgatccagcatatctcctcgtttcgcgggttggggcggctttcgctgtacggcagtagtcggtgctcgtccagacgggccagggtccgagatgccgtagagctaggtcgtatagaggagcaaagcgcccaaccccgccgaaagcgacatgccgtcatcagccacgagcaggtctgcccggtcccagatgtctttccacgggcgcagggtcctcgtcagcgtagtctgggtcacggtgaaggggtgcgctccgggctgcgcgctggccagggtgcgcttgaggctggtacagaaaggtgcccgcgtcccaggagcagtcgcatcagacccagtgccacttccccacgcgaggcccgacgcgcgaccggtcccacgcgaactccgacctcctgctggtgctgaagcgctgccggtcttcgccctgcgcgtcggccaggtagcatttgaccatggtgtcatagtccagcccctccgcggcgtggcccttaggacgaccacgacttcgcgacggccagaagcgggacgcgcagccggtccatcgtaaactggtaccacagtatcaggtcggggaggcgccgcaccgggaaggcgcgcagcttgcccttggaggaggcgccgcacgagggggcagtgcagacttttgagggcgtagagcttgggcgcgagaaataccgattccggggagtaccgcgcgtcgaacgggaacctcctccgcggcgtgctcccccgtcacgtctgaaaactcccgcatctcgaacccgcgctctttatggctaaggcccctcatggcatccgcgccgcaggccccgcagacggtctcgcattccacgagccaggtgagctctggccgttcggggtcaaaaaccaggtttcccccatgctttttgccgtaggcgcggcgtccggggcgtctgccagagcgtaaggtgctcggtccactcgagaccggcaagccccagtttttggtccaaagggggtacgaaaaacatgcgtttcttacctctggtttccatgagccggtgtccacgctcggtgacgaaaaggctgtccgtgtccccgtatacagacttgagaggcctgtcctcgatacgcaaagaatggagaccaaaggtactcggccacaggtgcgagccactgcttttccgacaggcacaggggcatatgtctgaactctccggacaggagctgcggtgttccgcggtcctcctcgtatagaaactcggaccactctgagacaaaggctcgcgtccaggccagcacgaaggaggctaagtgggaggggtagcgcgccacaaggcgccaggaggagcatatctttgagcctggtgagactctgtttccgagcgcaggtccggtcgtgcttcctccgattcaccctccccatcgcgtcgttgtccactagggggtccactcgctccagggtgtgaagacacatgtcgccctcttcggcatcaaggaaggtgattggtttgtaggtgtaggccacgcagcaacaggtgatcccccaggtgagcgaggtcccacacttctgtgtacagcgggagaagccgtagttccttccactaaccaaacatccacatccggtgctgaccgggtgttcctgaaggggggctataaaagggggtgggggcgcgttcgtcctcactctcttccgcatcgctgtctgcgagggccagctgttggggtgactggcccacaaggacttccccccgatattttcccccacccccgcgcaagcaggagtgagagaaggcgtagcgacagacgctcccggtcgacaaccccacagtactccctctgaaaagcgggcatgacttctgcgctaagattgtcagtttccaaaaacgaggaggatttgatattcacctggcccgcggtgatgccttttcatgagggagacttttcgcccgtactgaagacgcgattctaacagtcaaaggtttttgctcctcctaaactataagtggaccgggcgccactacggaaagagggtggccgcatccatctggtcagaaaagacaatctttttgttgtcaagcttggtggcaaacgacccgtagagggcgttggacagcaacttggcgatgctcccaccggcgtaggtagaccagtcttttctgttagaaaaacaacagttcgaaccaccgtttgctgggcatctcccgcaacctgtcgttgaaccgctacgagcgcagggtttggtttttgtcgcgatcggcgcgctccttggccgcgatgtttagctgcacgtattcgcgcgcaacgcaccgccattcgggaaagacggctcgcgtcccaaaccaaaaacagcgctagccgcgcgaggaaccggcgctacaaatcgacgtgcataagcgcgcgttgcgtggcggtaagccctttctgcctggtgcgctcgtcgggcaccaggtgcacgcgccaaccgcggttgtgcagggtgacaaggtcaacgctggtggctacctctccgcgtaggcgctcgttggtaccacgcgagcagcccgtggtccacgtgcgcggttggcgccaacacgtcccactgttccagttgcgaccaccgatggagaggcgcatccgcgagcaaccaccagcagaggcggccgcccttgcgcgagcagaatggcggtagggggtctagctgcgtctcgtccggggggtctgcgtccacggtaaagaccccgggcagcggtcgtctccgccggcgggaacgcgctcgtcttaccgccatcccccagatcgacgcagagcaggccccccagacgcaggtgccatttctggggcccgtcgaggcgcgcgtcgaagtagtctatcttgcatccttgcaagtctagcgcctgctgccatgcgcgggcggcaagcgcgcgctcgtatgggttgagtgggggactccgcgcgcagcttcatcagatagaacgtaggaacgttcagatcgcggacgacggtacgcgcccgccgttcgcgcgcgagcatacccaactcaccccctgcccatggcatggggtgggtgagcgcggaggcgtacatgccgcaaatgtcgtaaacgtagaggggctctctgagtattccaagatatgtagggtagcatctgggtaccgtaccccacccactcgcgcctccgcatgtacggcgtttacagcatttgcatctccccgagagactcataaggttctatacatcccatcgtagatccaccgcggatgctggcgcgcacgtaatcgtatagttcgtgcgagggagcgaggaggtcgggaccgaggttgctacgggcgggctgctctgctcggaagaggtggcgcctacgaccgcgcgtgcattagcatatcaagcacgctccctcgctcctccagccctggctccaacgatgcccgcccgacgagacgagccttcactatctgcctgaagatggcatgtgagttggatgatatggttggacgctggaagacgttgaagctggcgtctgtgagacctaccgcgtcacgcacgaaggtgatagacggacttctaccgtacactcaacctactataccaacctgcgaccttctgcaacttcgaccgcagacactctggatggcgcagtgcgtgcttccaggcgtaggagtcgcgcagcttgttgaccagctcggcggtgacctgcacgtctagggcgcagtagtccagggtttccttgatgatgtcatacttatcctgtccgcatcctcagcgcgtcgaacaactggtcgagccgccactggacgtgcagatcccgcgtcatcaggtcccaaaggaactactacagtatgaataggactcccttttttttccacagctcgcggttgaggacaaactcttcgcggtctttccagtactcttggatcggaaacccgtcggcctccgaacggtaagagcctagggaaaaaaaaggtgtcgagcgccaactcctgtttgagaagcgccagaaaggtcatgagaacctagcctttgggcagccggaggcttgccattctcggaagcatgtagaactggttgacggcctggtaggcgcagcatcccttttctacgggtagcgcgtatgcctgcgcggccttccggagcgaggtgtgggtgagcgtcgtacatcttgaccaactgccggaccatccgcgtcgtagggaaaagatgcccatcgcgcatacggacgcgccggaaggcctcgctccacacccactcgccaaaggtgtccctgaccatgactttgaggtactggtatttgaagtcagtgtcgtcgcatccgccctgctcccagagcaaaaagtccgtgcgctttttggagtttccacagggactggtactgaaactccatgaccataaacttcagtcacagcagcgtaggcgggacgagggtctcgtttttcaggcacgcgaaaaacctacgcggatttggcagggcgaaggtgacatcgttgaagagtatctttcccgcgcgaggcataaagttgcgtgtgatgcggaagggtcccggcacctcggaatgcgcctaaaccgtcccgcttccactgtagcaacttctcatagaaagggcgcgctccgtatttcaacgcacactacgccttcccagggccgtggagccttcggttgttaattacctgggcggcgagcacgatctcgtcaaagccgttgatgttgtggcccacaatgtaaagttccaagaagcgcgggatgcccttgatgggccaacaattaatggacccgccgctcgtgctagagcagtttcggcaactacaacaccgggtgttacatttcaaggttcttcgcgccctacgggaactaccaaggcaattttttaagttcctcgtaggtgagctcttcaggggagctgagcccgtgctctgaaagggcccagtctgcaagatgagggttggaagcgacgaattccgttaaaaaattcaaggagcatccactcgagaagtcccctcgactcgggcacgagactttcccgggtcagacgttctactcccaaccttcgctgctttgagctccacaggtcacgggccattagcatttgcaggtggtcgcgaaaggtcctaaactggcgacctatggccattttttctggggtgatgcagtagaagactcgaggtgtccagtgcccggtaatcgtaaacgtccaccagcgctttccaggatttgaccgctggataccggtaaaaaagaccccactacgtcatcttcgtaagcgggtcttgttcccagcggtcccatccaaggttcgcggctaggtctcgcgcggcagtcactagaggctcatctccgccgaacttcatgaccagcacattcgcccagaacaagggtcgccagggtaggttccaagcgccgatccagagcgcgccgtcagtgatctccgagtagaggcggcttgaagtactggtcgttgaagggcacgagctgcttcccaaaggcccccatccaagtataggtctctacatcgtaggtgacaaagagacgctcggtgcgaggatgcgagccgatcggacttcccgtgctcgacgaagggtttccgggggtaggttcatatccagagatgtagcatccactgtttctctgcgagccacgctcctacgctcggctagccgaagaactggatctcccgccaccaattggaggagtggctattgatgtggtgaaagtagaagtccctgcgacgggccgaacactcgtgctggcttttgtaacttcttgacctagagggcggtggttaacctcctcaccgataactacaccactttcatcttcagggacgctgcccggcttgtgagcacgaccgaaaacattaaacgtgcgcagtactggcagcggtgcacgggctgtacatcctgcacgaggttgacctgacgaccgcgcacaaggaagcagagtgggaatttgagccccttttgcacgcgtcatgaccgtcgccacgtgcccgacatgtaggacgtgctccaactggactgctggcgcgtgttccttcgtctcacccttaaactcggggacgcctggcgggtttggctggtggtcttctacttcggctgcttgtccttgaccgtctggctgctcgaggggagttacggtggatcggaccaccacgccgcggcggaccgcccaaaccgaccaccagaagatgaagccgacgaacaggaactggcagaccgacgagctcccctcaatgccacctagcctggtggtgcggcgccgagcccaaagtccagatgtccgcgcgcggcggtcggagcttgatgacaacatcgcgcagatgggagctgtccatggtctggagctcccgcggcgtcagggctcgggtttcaggtctacaggcgcgcgccgccagcctcgaactactgttgtagcgcgtctaccctcgacaggtaccagacctcgagggcgccgcagtcctcaggcgggagctcctgcaggtttacctcgcatagacgggtcagggcgcgggctagatccaggtgatacctaatttccaggggctggttggtggcggcgtagtccgccctcgaggacgtccaaatggagcgtatctgcccagtcccgcgcccgatctaggtccactatggattaaaggtccccgaccaaccaccgccgcacgatggcttgcaagaggccgcatccccgcggcgcgactacggtaccgcgcggcgggcggtgggccgcgggggtgtccttggatgatgcatctaaaagcgggctaccgaacgttctccggcgtaggggcgccgcgctgatgccatggcgcgccgcccgccacccggcgcccccacaggaacctactacgtagattttcgcctgacgcgggcgagcccccggaggtagggggggctccggacccgccgggagagggggcaggggcacgtcggcgccgcgcgcgggcaggagctggtgctgcgactgcgcccgctcgggggcctccatcccccccgaggcctgggcggccctctcccccgtccccgtgcagccgcggcgcgcgcccgtcctcgaccacgacgccgcgtaggttgctggcgaacgcgacgacgcggcggttgatctcctgaatctggcgcctctgcgtgaagacgacgggcccggtgagcttgagcctgaaagagcgcatccaacgaccgcttgcgctgctgcgccgccaactagaggacttagaccgcggagacgcacttctgctgcccgggccactcgaactcggactttctgagttcgacagaatcaatttcggtgtcgttgacggcggcctggcgcaaaatctcctgcacgtctcctgagttgtcttgataggcgatctcggccatgaacctcaagctgtcttagttaaagccacagcaactgccgccggaccgcgttttagaggacgtgcagaggactcaacagaactatccgctagagccggtacttgtgctcgatctcttcctcctggagatctccgcgtccggctcgctccacggtggcggcgaggtcgttggaaatgcgggccatgagctgcgagaaggcgttgaacgagctagagaaggaggacctctagaggcgcaggccgagcgaggtgccaccgccgctccagcaacctttacgcccggtactcgacgctcttccgcaactggcctccctcgttccagacgcggctgtagaccacgcccccttcggcatcgcgggcgcgcatgaccacctgcgcgagattgagctccacgtgccgggcgaaccggagggagcaaggtctgcgccgacatctggtgcgggggaagccgtagcgcccgcgcgtactggtggacgcgctctaactcgaggtgcacggcccgcttgacggcgtagtttcgcaggcgctgaaagaggtagttgagggtggtggcggtgtgttctgccacgaagaagtacataacccagcgtcgcaacgtggattcgctgccgcatcaaagcgtccgcgactttctccatcaactcccaccaccgccacacaagacggtgcttcttcatgtattgggtcgcagcgttgcacctaagcttgatatcccccaaggcctcaaggcgctccatggcctcgtagaagtccacggcgaagttgaaaaactgggagttgcgcgccgacacggttaactcctcctaactatagggggttccggagttccgcgaggtaccggagcatcttcaggtgccgcttcaactttttgaccctcaacgcgcggctgtgccaattgaggaggaccagaagacggatgagctcggcgacagtgtcgcgcacctcgcgctcaaaggctacaggggcctcttcttcttcttcaatctcctcttccataagggcctcggtcttctgcctactcgagccgctgtcacagcgcgtggagcgcgagtttccgatgtccccggagaagaagaagaagttagaggagaaggtattcccggagcccttcttcttcttctggcggcggtgggggaggggggacacggcggcgacgacggcgcaccgggaggcggtcgacaaagcgctcgatcatctccccgcgggggaagaagaagaagaccgccgccaccccctcccccctgtgccgccgctgctgccgcgtggccctccgccagctgtttcgcgagctagtagaggggcgcccgacggcgcatggtctcggtgacggcgcggccgttctcgcgggggcgcagttggaagacgccgcccgtcatgtcccggttatgggttggcggggggctgcgctgccgcgtaccagagccactgccgcgccggcaagagcgcccccgcgtcaaccttctgcggcgggcagtacagggccaatacccaaccgccccccgacgcatgcggcagggatacggcgctaacgatgcatctcaacaattgttgtgtaggtactccgccgccgagggacctgagcgagtccgcatcgaccggatcggagtacgccgtccctatgccgcgattgctacgtagagttgttaacaacacatccatgaggcggcggctccctggactcgctcaggcgtagctggcctagcctaaacctctcgagaaaggcgtctaaccagtcacagtcgcaaggtaggctgagcaccgtggcgggcggcagcgggcggcggtcggggttgtttctggcggagtttggagagctctttccgcagattggtcagtgtcagcgttccatccgactcgtggcaccgcccgccgtcgcccgccgccagccccaacaaagaccgcctcgtgctgctgatgatgtaattaaagtaggcggtcttgagacggcggatggtcgacagaagcaccatgtccttgggtccggcctgctgaatgcgcaggcggtcacgacgactactacattaatttcatccgccagaactctgccgcctaccagctgtcttcgtggtacaggaacccaggccggacgacttacgcgtccgccacggccatgccccaggcttcgttttgacatcggcgcaggtctttgtagtagtcttgcatgagcctttctaccggcacttcttcttctccttcctcttgtccgccggtacggggtccgaagcaaaactgtagccgcgtccagaaacatcatcagaacgtactcggaaagatggccgtgaagaagaagaggaaggagaacaggtgcatctcttgcatctatcgctgcggcggcggcggagtttggccgtaggtggcgccctcttcctcccatgcgtgtgaccccgaagcccctcatcggctgaacgtagagaacgtagatagcgacgccgccgccgcctcaaaccggcatccaccgcgggagaaggagggtacgcacactggggcttcggggagtagccgactagcagggctaggtcggcgacaacgcgctcggctaatatggcctgctgcacctgcgtgagggtagactggaagtcatccatgtccacaaagcggtggtatgtcgtcccgatccagccgctgttgcgcgagccgattataccggacgacgtggacgcactcccatctgaccttcagtaggtacaggtgtttcgccaccataccgcccgtgttgatggtgtaagtgcagttggccataacggaccagttaacggtctggtgacccggctgcgagagctcggtgtacctgagacgcgagtaagcgcgggcacaactaccacattcacgtcaaccggtattgcctggtcaattgccagaccactgggccgacgctctcgagccacatggactctgcgctcattcgcctcgagtcaaatacgtagtcgttgcaagtccgcaccaggtactggtatcccaccaaaaagtgcggcggcggctggcggtagaggggccagcgtagggtgggagctcagtttatgcatcagcaacgttcaggcgtggtccatgaccatagggtggtttttcacgccgccgccgaccgccatctccccggtcgcatcccacgccggggctccgggggcgagatcttccaacataaggcgatgatatccgtagatgtacctggacatccaggtgatgccggcggcggtggtggaggcgcgcgcggccccgaggcccccgctctagaaggttgtattccgctactataggcatctacatggacctgtaggtccactacggccgccgccaccacctccgcgcgcgaaagtcgcggacgcggttccagatgttgcgcagcggcaaaaagtgctccatggtcgggacgctctggccggtcaggcgcgcgcaatcgttgacgctctactttcagcgcctgcgccaaggtctacaacgcgtcgccgtttttcacgaggtaccagccctgcgagaccggccagtccgcgcgcgttagcaactgcgagatccgtgcaaaaggagagcctgtaagcgggcactcttccgtggtctggtggataaattcgcaagggtatcatggcggacgaccggggttcgagccccgtatcggcacgttttcctctcggacattcgcccgtgagaaggcaccagaccacctatttaagcgttcccatagtaccgcctgctggccccaagctcggggcatagcggccgtccgccgtgatccatgcggttaccgcccgcgtgtcgaacccaggtgtgcgacgtcagacaacgggggagtgctccttttggcttccttccaggcgccggcaggcggcactaggtacgccaatggcgggcgcacagcttgggtccacacgctgcagtctgttgccccctcacgaggaaaaccgaaggaaggtccggcggcggctgctgcgctagcttttttggccactggccgcgcgcagcgtaagcggttaggctggaaagcgaaagcattaagtggctcgctccctgtagccgcgccgccgacgacgcgatcgaaaaaaccggtgaccggcgcgcgtcgcattcgccaatccgacctttcgctttcgtaattcaccgagcgagggacatcggcgagggttattttccaagggttgagtcgcgggacccccggttcgagtctcggaccggccggactgcggcgaacgggggtttgcctccccgtcatgcaagacctcccaataaaaggttcccaactcagcgccctgggggccaagctcagagcctggccggcctgacgccgcttgcccccaaacggaggggcagtacgttctgcccgcttgcaaattcctccggaaacagggacgagccccttttttgcttttcccagatgcatccggtgctgcggcagatgcgcccccctcctcagcagcgggggcgaacgtttaaggaggcctttgtccctgctcggggaaaaaacgaaaagggtctacgtaggccacgacgccgtctacgcggggggaggagtcgtcgcccaagagcaagagcagcggcagacatgcagggcaccctcccctcctcctaccgcgtcaggaggggcgacatccgcggttgacgcggcagcagatggtgattgttctcgttctcgtcgccgtctgtacgtcccgtgggaggggaggaggatggcgcagtcctccccgctgtaggcgccaactgcgccgtcgtctaccactaaacgaacccccgcggcgccgggcccggcactacctggacttggaggagggcgagggcctggcgcggctaggagcgccctctcctgagcggtacccaagggttgcttgggggcgccgcggcccgggccgtgatggacctgaacctcctcccgctcccggaccgcgccgatcctcgcgggagaggactcgccatgggttcccagcagctgaagcgtgatacgcgtgaggcgtacgtgccgcggcagaacctgtttcgcgaccgcgagggagaggagcccgaggagatgcgggatcgaaagttccgtcgacttcgcactatgcgcactccgcatgcacggcgccgtcttggacaaagcgctggcgctccctctcctcgggctcctctacgccctagctttcaagcacgcagggcgcgagctgcggcatggcctgaatcgcgagcggttgctgcgcgaggaggactttgagcccgacgcgcgaaccgggattagtcccgcgcgcggtgcgtcccgcgctcgacgccgtaccggacttagcgctcgccaacgacgcgctcctcctgaaactcgggctgcgcgcttggccctaatcagggcgcgcgccacacgtggcggccgccgacctggtaaccgcatacgagcagacggtgaaccaggagattaactttcaaaaaagctttaacaaccacgtgcgtacgcttgtgtgtgcaccgccggcggctggaccattggcgtatgctcgtctgccacttggtcctctaattgaaagttttttcgaaattgttggtgcacgcatgcgaacaggcgcgcgaggaggtggctataggactgatgcatctgtgggactttgtaagcgcgctggagcaaaacccaaatagcaagccgctcatggcgcagctgttcccgcgcgctcctccaccgatatcctgactacgtagacaccctgaaacattcgcgcgacctcgttttgggtttatcgttcggcgagtaccgcgtcgacaagcttatagtgcagcacagcagggacaacgaggcattcagggatgcgctgctaaacatagtagagcccgagggccgctggctgctcgatttgataaacatccgaatatcacgtcgtgtcgtccctgttgctccgtaagtccctacgcgacgatttgtatcatctcgggctcccggcgaccgacgagctaaactatttgtaggtgcagagcatagtggtgcaggagcgcagcttgagcctggctgacaaggtggccgccatcaactattccatgcttagcctgggcaagttttacgcccgcaaacgtctcgtatcaccacgtcctcgcgtcgaactcggaccgactgttccaccggcggtagttgataaggtacgaatcggacccgttcaaaatgcgggcgttgatataccataccccttacgttcccatagacaaggaggtaaagatcgaggggttctacatgcgcatggcgctgaaggtgcttaccttgagcgacgacctgctatatggtatggggaatgcaagggtatctgttcctccatttctagctccccaagatgtacgcgtaccgcgacttccacgaatggaactcgctgctggacggcgtttatcgcaacgagcgcatccacaaggccgtgagcgtgagccggcggcgcgagctcagcgaccgcgagctgatgcacagcctgcaaagggccctggccgcaaatagcgttgctcgcgtaggtgttccggcactcgcactcggccgccgcgctcgagtcgctggcgctcgactacgtgtcggacgtttcccgggaccctggcacgggcagcggcgatagagaggccgagtcctactttgacgcgggcgctgacctgcgctgggccccaagccgacgcgccctggaggcagctggggcgaccgtgcccgtcgccgctatctctccggctcaggatgaaactgcgcccgcgactggacgcgacccggggttcggctgcgcgggacctccgtcgaccccgcggacctgggctggcggtggcacccgcgcgcgctggcaacgtcggcggcgtggaggaatatgacgaggacgatgagtacgagccagaggacggcgagtacgcctggacccgaccgccaccgtgggcgcgcgcgaccgttgcagccgccgcacctccttatactgctcctgctactcatgctcggtctcctgccgctcatgtaagcggtgatgtttctgatcagatgatgcaagacgcaacggacccggcggtgcgggcggcgctgcagagccagccgtccggccttaactccacggacgaattcgccactacaaagactagtctactacgttctgcgttgcctgggccgccacgcccgccgcgacgtctcggtcggcaggccggaattgaggtgcctgctctggcgccaggtcatggaccgcatcatgtcgctgactgcgcgcaatcctgacgcgttccggcagcagccgcaggccaaccggctctccgcaattctggaagaccgcggtccagtacctggcgtagtacagcgactgacgcgcgttaggactgcgcaaggccgtcgtcggcgtccggttggccgagaggcgttaagaccttgcggtggtcccggcgcgcgcaaaccccacgcacgagaaggtgctggcgatcgtaaacgcgctggccgaaaacagggccatccggcccgacgaggccggcccgccaccagggccgcgcgcgtttggggtgcgtgctcttccacgaccgctagcatttgcgcgaccggcttttgtcccggtaggccgggctgctccggccggtggtctacgacgcgctgcttcagcgcgtggctcgttacaacagcggcaacgtgcagaccaacctggaccggctggtgggggatgtgcgcgaggccgtggcaccagatgctgcgcgacgaagtcgcgcaccgagcaatgttgtcgccgttgcacgtctggttggacctggccgaccaccccctacacgcgctccggcaccggcagcgtgagcgcgcgcagcagcagggcaacctgggctccatggttgcactaaacgccttcctgagtacacagcccgccaacgtgccgcggggacaggagcgtcgcactcgcgcgcgtcgtcgtcccgttggacccgaggtaccaacgtgatttgcggaaggactcatgtgtcgggcggttgcacggcgcccctgtcctcgactacaccaactttgtgagcgcactgcggctaatggtgactgagacaccgcaaagtgaggtgtaccagtctgggccagactattttttccagaccagtactgatgtggttgaaacactcgcgtgacgccgattaccactgactctgtggcgtttcactccacatggtcagacccggtctgataaaaaaggtctggtcatgacaaggcctgcagaccgtaaacctgagccaggctttcaaaaacttgcaggggctgtggggggtgcgggctcccacaggcgaccgcgcgaccgtgtctagctgttccggacgtctggcatttggactcggtccgaaagtttttgaacgtccccgacaccccccacgcccgagggtgtccgctggcgcgctggcacagatccttgctgacgcccaactcgcgcctgttgctgctgctaatagcgcccttcacggacagtggcagcgtgtcccgggacacatacctaggtcacttgctgacagaacgactgcgggttgagcgcggacaacgacgacgattatcgcgggaagtgcctgtcaccgtcgcacagggccctgtgtatggatccagtgaacgactgtctgtaccgcgaggccataggtcaggcgcatgtggacgagcatactttccaggagattacaagtgtcagccgcgcgctggggcaggaggacacgggcagccgacatggcgctccggtatccagtccgcgtacacctgctcgtatgaaaggtcctctaatgttcacagtcggcgcgcgaccccgtcctcctgtgcccgtcggtggaggcaaccctaaactacctgctgaccaaccggcggcagaagatcccctcgttgcacagtttaaacagcgaggaggagcgcattttgcgctacgtgcaacctccgttgggatttgatggacgactggttggccgccgtcttctaggggagcaacgtgtcaaatttgtcgctcctcctcgcgtaaaacgcgatgcacgtgcagagcgtgagccttaacctgatgcgcgacggggtaacgcccagcgtggcgctggacatgaccgcgcgcaacatggaaccgggcatgtatgcctcaaaccgtctcgcactcggaattggactacgcgctgccccattgcgggtcgcaccgcgacctgtactggcgcgcgttgtaccttggcccgtacatacggagtttgcggccgtttatcaaccgcctaatggactacttgcatcgcgcggccgccgtgaaccccgagtatttcaccaatgccatcttgaacccgcactggctaccgcgccggcaaatagttggcggattacctgatgaacgtagcgcgccggcggcacttggggctcataaagtggttacggtagaacttgggcgtgaccgatggcgcccctggtttctacaccgggggattcgaggtgcccgagggtaacgatggattcctctgggacgacatagacgacagcgtgttttccccgcaaccgcagacggggaccaaagatgtggccccctaagctccacgggctcccattgctacctaaggagaccctgctgtatctgctgtcgcacaaaaggggcgttggcgtctgcctgctagagttgcaacagcgcgagcaggcagaggcggcgctgcgaaaggaaagcttccgcaggccaagcagcttgtccgatctaggcgctgcggccccgggacgatctcaacgttgtcgcgctcgtccgtctccgccgcgacgctttcctttcgaaggcgtccggttcgtcgaacaggctagatccgcgacgccggggccggtcagatgctagtagcccatttccaagcttgatagggtctcttaccagcactcgcaccacccgcccgcgcctgctgggcgaggaggagtacctaaacagccagtctacgatcatcgggtaaaggttcgaactatcccagagaatggtcgtgagcgtggtgggcgggcgcggacgacccgctcctcctcatggatttgtactcgctgctgcagccgcagcgcgaaaaaaacctgcctccggcatttcccaacaacgggatagagagcctagtggacaagatgagtagatggaagacgtatgagcgacgacgtcggcgtcgcgctttttttggacggaggccgtaaagggttgttgccctatctctcggatcacctgttctactcatctaccttctgcatcgcgcaggagcacagggacgtgccaggcccgcgcccgcccacccgtcgtcaaaggcacgaccgtcagcggggtctggtgtgggaggacgatgactcggcagcgcgtcctcgtgtccctgcacggtccgggcgcgggcgggtgggcagcagtttccgtgctggcagtcgccccagaccacaccctcctgctactgagccgtgacgacagcagcgtcctggatttgggagggagtggcaacccgtttgcgcaccttcgccccaggctggggagaatgttttaaaaaaaaaaaagcatgatgcctgctgtcgtcgcaggacctaaaccctccctcaccgttgggcaaacgcgtggaagcggggtccgacccctcttacaaaattttttttttttcgtactacgaaaataaaaaactcaccaaggccatggcaccgagcgttggttttcttgtattccccttagtatgcggcgcgcggcgatgtatgaggaaggtcctcctcccttttattttttgagtggttccggtaccgtggctcgcaaccaaaagaacataaggggaatcatacgccgcgcgccgctacatactccttccaggaggagggtcctacgagagtgtggtgagcgcggcgccagtggcggcggcgctgggttctcccttcgatgctcccctggacccgccgtttgtgcctccgcggtacctgcaggatgctctcacaccactcgcgccgcggtcaccgccgccgcgacccaagagggaagctacgaggggacctgggcggcaaacacggaggcgccatggacgggcctaccggggggagaaacagcatccgttactctgagttggcacccctattcgacaccacccgtgtgtacctggtggacaacaagtcaacggatgtggcccggatggcccccctctttgtcgtaggcaatgagactcaaccgtggggataagctgtggtgggcacacatggaccacctgttgttcagttgcctacaccgatccctgaactaccagaacgaccacagcaactttctgaccacggtcattcaaaacaatgactacagcccgggggaggcaagcacacagaccatcaatctttagggacttgatggtcttgctggtgtcgttgaaagactggtgccagtaagttttgttactgatgtcgggccccctccgttcgtgtgtctggtagttagaagacgaccggtcgcactggggcggcgacctgaaaaccatcctgcataccaacatgccaaatgtgaacgagttcatgtttaccaataagtttaaggcgcgggctgctggccagcgtgaccccgccgctggacttttggtaggacgtatggttgtacggtttacacttgctcaagtacaaatggttattcaaattccgcgccctgatggtgtcgcgcttgcctactaaggacaatcaggtggagctgaaatacgagtgggtggagttcacgctgcccgagggcaactactccgagaccatgacactaccacagcgcgaacggatgattcctgttagtccacctcgactttatgctcacccacctcaagtgcgacgggctcccgttgatgaggctctggtactgcatagaccttatgaacaacgcgatcgtggagcactacttgaaagtgggcagacagaacggggttctggaaagcgacatcggggtaaagtttgacacccgcgtatctggaatacttgttgcgctagcacctcgtgatgaactttcacccgtctgtcttgccccaagacctttcgctgtagccccatttcaaactgtgggcgaacttcagactggggtttgaccccgtcactggtcttgtcatgcctggggtatatacaaacgaagccttccatccagacatcattttgctgccaggatgcgttgaagtctgaccccaaactggggcagtgaccagaacagtacggaccccatatatgtttgcttcggaaggtaggtctgtagtaaaacgacggtcctacgcgggtggacttcacccacagccgcctgagcaacttgttgggcatccgcaagcggcaacccttccaggagggctttaggatcacctacgatgatctggagggcccacctgaagtgggtgtcggcggactcgttgaacaacccgtaggcgttcgccgttgggaaggtcctcccgaaatcctagtggatgctactagacctccctggtaacattcccgcactgttggatgtggacgcctaccaggcgagcttgaaagatgacaccgaacagggcgggggtggcgcaggcggcagcaacagcagtaccattgtaagggcgtgacaacctacacctgcggatggtccgctcgaactttctactgtggcttgtcccgcccccaccgcgtccgccgtcgttgtcgtcaggcagcggcgcggaagagaactccaacgcggcagccgcggcaatgcagccggtggaggacatgaacgatcatgccattcgcggcgacacctttgccacacccgtcgccgcgccttctcttgaggttgcgccgtcggcgccgttacgtcggccacctcctgtacttgctagtacggtaagcgccgctgtggaaacggtgtggggctgaggagaagcgcgctgaggccgaagcagcggccgaagctgccgcccccgctgcgcaacccgaggtcgagaagcctcagaagaaaccggtgatcaacccgactcctcttcgcgcgactccggcttcgtcgccggcttcgacggcgggggcgacgcgttgggctccagctcttcggagtcttctttggccactagttacccctgacagaggacagcaagaaacgcagttacaacctaataagcaatgacagcaccttcacccagtaccgcagctggtaccttgcatacaactacggctggggactgtctcctgtcgttctttgcgtcaatgttggattattcgttactgtcgtggaagtgggtcatggcgtcgaccatggaacgtatgttgatgccggaccctcagaccggaatccgctcatggaccctgctttgcactcctgacgtaacctgcggctcggagcaggtctactggtcgttgccagacatgatgcaagctgggagtctggccttaggcgagtacctgggacgaaacgtgaggactgcattggacgccgagcctcgtccagatgaccagcaacggtctgtactacgttcaccccgtgaccttccgctccacgcgccagatcagcaactttccggtggtgggcgccgagctgttgcccgtgcactccaagagcttctacaacgaccaggctggggcactggaaggcgaggtgcgcggtctagtcgttgaaaggccaccacccgcggctcgacaacgggcacgtgaggttctcgaagatgttgctggtccgcgtctactcccaactcatccgccagtttacctctctgacccacgtgttcaatcgctttcccgagaaccagattttggcgcgcccgccagcccccaccatcgcagatgagggttgagtaggcggtcaaatggagagactgggtgcacaagttagcgaaagggctcttggtctaaaaccgcgcgggcggtcgggggtggtagaccaccgtcagtgaaaacgttcctgctctcacagatcacgggacgctaccgctgcgcaacagcatcggaggagtccagcgagtgaccattactgacgccatggtggcagtcacttttgcaaggacgagagtgtctagtgccctgcgatggcgacgcgttgtcgtagcctcctcaggtcgctcactggtaatgactgcggtgacgccgcacctgcccctacgtttacaaggccctgggcatagtctcgccgcgcgtcctatcgagccgcactttttgagcaagcatgtccatccttatatcctgcggcgtggacggggatgcaaatgttccgggacccgtatcagagcggcgcgcaggatagctcggcgtgaaaaactcgttcgtacaggtaggaatataggcccagcaataacacaggctggggcctgcgcttcccaagcaagatgtttggcggggccaagaagcgctccgaccaacacccagtgcgcgtgcgcgggcaccgggtcgttattgtgtccgaccccggacgcgaagggttcgttctacaaaccgccccggttcttcgcgaggctggttgtgggtcacgcgcacgcgcccgtgtaccgcgcgccctggggcgcgcacaaacgcggccgcactgggcgcaccaccgtcgatgacgccatcgacgcggtggtggaggaggcgcgcaactacacgcatggcgcgcgggaccccgcgcgtgtttgcgccggcgtgacccgcgtggtggcagctactgcggtagctgcgccaccacctcctccgcgcgttgatgtgcgccacgccgccaccagtgtccacagtggacgcggccattcagaccgtggtgcgcggagcccggcgctatgctaaaatgaagagacggcggaggcgcgtagcggtgcggcggtggtcacaggtgtcacctgcgccggtaagtctggcaccacgcgcctcgggccgcgatacgattttacttctctgccgcctccgcgcatcgacgtcgccaccgccgccgacccggcactgccgcccaacgcgcggcggcggccctgcttaaccgcgcacgtcgcaccggccgacgggcggccatgcgggcctgcagcggtggcggcggctgggccgtgacggcgggttgcgcgccgccgccgggacgaattggcgcgtgcagcgtggccggctgcccgccggtacgcccgggctcgaaggctggccgcggtattgtcactgtgccccccaggtccaggcgacgagcggccgccgcagcagccgcggccattagtgctatgactcagggtcgcgagcttccgaccggcgccataacagtgacacggggggtccaggtccgctgctcgccggcggcgtcgtcggcgccggtaatcacgatactgagtcccagccaggggcaacgtgtattgggtgcgcgactcggttagcggcctgcgcgtgcccgtgcgcacccgccccccgcgcaactagattgcaagaaaaaactacttagtccccgttgcacataacccacgcgctgagccaatcgccggacgcgcacgggcacgcgtgggcggggggcgcgttgatctaacgttcttttttgatgaatgactcgtactgttgtatgtatccagcggcggcggcgcgcaacgaagctatgtccaagcgcaaaatcaaagaagagatgctccaggtcatcgcgccggagactgagcatgacaacatacataggtcgccgccgccgcgcgttgcttcgatacaggttcgcgttttagtttcttctctacgaggtccagtagcgcggcctcttctatggccccccgaagaaggaagagcaggattacaagccccgaaagctaaagcgggtcaaaaagaaaaagaaagatgatgatgatgaacttgacgacgaagataccggggggcttcttccttctcgtcctaatgttcggggctttcgatttcgcccagtttttctttttctttctactactactacttgaactgctgctggtggaactgctgcacgctaccgcgcccaggcgacgggtacagtggaaaggtcgacgcgtaaaacgtgttttgcgacccggcaccaccgtagtctttacgccaccttgacgacgtgcgatggcgcgggtccgctgcccatgtcacctttccagctgcgcattttgcacaaaacgctgggccgtggtggcatcagaaatgccccggtgagcgctccacccgcacctacaagcgcgtgtatgatgaggtgtacggcgacgaggacctgcttgagcaggccaacgagcgcctcggggagtttggggccactcgcgaggtgggcgtggatgttcgcgcacatactactccacatgccgctgctcctggacgaactcgtccggttgctcgcggagcccctcaaaccctacggaaagcggcataaggacatgctggcgttgccgctggacgagggcaacccaacacctagcctaaagcccgtaacactgcagcaggtgctgcccgcggatgcctttcgccgtattcctgtacgaccgcaacggcgacctgctcccgttgggttgtggatcggatttcgggcattgtgacgtcgtccacgacgggcggcttgcaccgtccgaagaaaagcgcggcctaaagcgcgagtctggtgacttggcacccaccgtgcagctgatggtacccaagcgccagcgactggaagatcgaacgtggcaggcttcttttcgcgccggatttcgcgctcagaccactgaaccgtgggtggcacgtcgactaccatgggttcgcggtcgctgaccttctagtcttggaaaaaatgaccgtggaacctgggctggagcccgaggtccgcgtgcggccaatcaagcaggtggcgccgggactgggcgtgcagaccgtggacgcagaaccttttttactggcaccttggacccgacctcgggctccaggcgcacgccggttagttcgtccaccgcggccctgacccgcacgtctggcacctgcttcagatacccactaccagtagcaccagtattgccaccgccacagagggcatggagacacaaacgtccccggttgcctcagcggtggcggatgccgcggtaagtctatgggtgatggtcatcgtggtcataacggtggcggtgtctcccgtacctctgtgtttgcaggggccaacggagtcgccaccgcctacggcgccagcaggcggtcgctgcggccgcgtccaagacctctacggaggtgcaaacggacccgtggatgtttcgcgtttcagccccccggcgcccgcgcggttcgaggcgtccgccagcgacgccggcgcaggttctggagatgcctccacgtttgcctgggcacctacaaagcgcaaagtcggggggccgcgggcgcgccaagctccaagtacggcgccgccagcgcgctactgcccgaatatgccctacatccttccattgcgcctacccccggctatcgtggctacacctaccgccccagaagacttcatgccgcggcggtcgcgcgatgacgggcttatacgggatgtaggaaggtaacgcggatgggggccgatagcaccgatgtggatggcggggtcttctggagcaactacccgacgccgaaccaccactggaacccgccgccgccgtcgccgtcgccagcccgtgctggccccgatttccgtgcgcagggtggctcgcgactcgttgatgggctgcggcttggtggtgaccttgggcggcggcggcagcggcagcggtcgggcacgaccggggctaaaggcacgcgtcccaccgagcgctaggaggcaggaccctggtgctgccaacagcgcgctaccaccccagcatcgtttaaaagccggtctttgtggttcttgcagatatggccctcacctgccgctcctccgtcctgggaccacgacggttgtcgcgcgatggtggggtcgtagcaaattttcggccagaaacaccaagaacgtctataccgggagtggacggcgctccgtttcccggtgccgggattccgaggaagaatgcaccgtaggaggggcatggccggccacggcctgacgggcggcatgcgtcgtgcgcaccaccggcgaggcaaagggccacggccctaaggctccttcttacgtggcatcctccccgtaccggccggtgccggactgcccgccgtacgcagcacgcgtggtggccgggcggcgcgcgtcgcaccgtcgcatgcgcggcggtatcctgcccctccttattccactgatcgccgcggcgattggcgccgtgcccggaattgcatccgtccgccgcgcgcagcgtggcagcgtacgcgccgccataggacggggaggaataaggtgactagcggcgccgctaaccgcggcacgggccttaacgtaggcaggccttgcaggcgcagagacactgattaaaaacaagttgcatgtggaaaaatcaaaataaaaagtctggactctcacgctcgcttggtcctgtaactattccggaacgtccgcgtctctgtgactaatttttgttcaacgtacacctttttagttttatttttcagacctgagagtgcgagcgaaccaggacattgataattgtagaatggaagacatcaactttgcgtctctggccccgcgacacggctcgcgcccgttcatgggaaactggcaagatatcggcaccagcaatatgagcaacatcttaccttctgtagttgaaacgcagagaccggggcgctgtgccgagcgcgggcaagtaccctttgaccgttctatagccgtggtcgttatactcgggtggcgccttcagctggggctcgctgtggagcggcattaaaaatttcggttccaccgttaagaactatggcagcaaggcctggaacagcagcacaggccccaccgcggaagtcgaccccgagcgacacctcgccgtaatttttaaagccaaggtggcaattcttgataccgtcgttccggaccttgtcgtcgtgtccggagatgctgagggataagttgaaagagcaaaatttccaacaaaaggtggtagatggcctggcctctggcattagcggggtggtggacctggccaaccaggctctacgactccctattcaactttctcgttttaaaggttgttttccaccatctaccggaccggagaccgtaatcgccccaccacctggaccggttggtccgagtgcaaaataagattaacagtaagcttgatccccgccctcccgtagaggagcctccaccggccgtggagacagtgtctccagaggggcgtggcgaaaagtcacgttttattctaattgtcattcgaactaggggcgggagggcatctcctcggaggtggccggcacctctgtcacagaggtctccccgcaccgcttttccgtccgcgccccgacagggaagaaactctggtgacgcaaatagacgagcctccctcgtacgaggaggcactaaagcaaggcctgcccaccacccgtcccagcaggcgcggggctgtcccttctttgagaccactgcgtttatctgctcggagggagcatgctcctccgtgatttcgttccggacgggtggtgggcagggttcgcgcccatggctaccggagtgctgggccagcacacacccgtaacgctggacctgcctccccccgccgacacccagcagaaacctgtgctgccaggcccagcgcgggtaccgatggcctcacgacccggtcgtgtgtgggcattgcgacctggacggaggggggcggctgtgggtcgtctttggacacgacggtccggggaccgccgttgttgtaacccgtcctagccgcgcgtccctgcgccgcgccgccagcggtccgcgatcgttgcggcccgtagccagtggcaactggcaaagcctggcggcaacaacattgggcaggatcggcgcgcagggacgcggcgcggcggtcgccaggcgctagcaacgccgggcatcggtcaccgttgaccgtttcgacactgaacagcatcgtgggtctgggggtgcaatccctgaagcgccgacgatgcttctgaatagctaacgtgtcgtatgtgtgtcatgtatgcgtccatgtgtgacttgtcgtagcacccagacccccacgttagggacttcgcggctgctacgaagacttatcgattgcacagcatacacacagtacatacgcaggtactcgccgccagaggagctgctgagccgccgcgcgcccgctttccaagatggctaccccttcgatgatgccgcagtggtcttacatgcacatctcgggccagagcggcggtctcctcgacgactcggcggcgcgcgggcgaaaggttctaccgatggggaagctactacggcgtcaccagaatgtacgtgtagagcccggtcgacgcctcggagtacctgagccccgggctggtgcagtttgcccgcgccaccgagacgtacttcagcctgaataacaagtttagaaaccccacggtggcgcctgcggagcctcatggactcggggcccgaccacgtcaaacgggcgcggtggctctgcatgaagtcggacttattgttcaaatctttggggtgccaccgcgctacgcacgacgtgaccacagaccggtcccagcgtttgacgctgcggttcatccctgtggaccgtgaggatactgcgtactcgtacaaggcgcggttcacgatgcgtgctgcactggtgtctggccagggtcgcaaactgcgacgccaagtagggacacctggcactcctatgacgcatgagcatgttccgcgccaagtgcctagctgtgggtgataaccgtgtgctggacatggcttccacgtactttgacatccgcggcgtgctggacaggggccctacttttaagccctactctggcggatcgacacccactattggcacacgacctgtaccgaaggtgcatgaaactgtaggcgccgcacgacctgtccccgggatgaaaattcgggatgagaccgactgcctacaacgccctggctcccaagggtgccccaaatccttgcgaatgggatgaagctgctactgctcttgaaataaacctagaagaagaggacgatgtgacggatgttgcgggaccgagggttcccacggggtttaggaacgcttaccctacttcgacgatgacgagaactttatttggatcttcttctcctgctacacaacgaagacgaagtagacgagcaagctgagcagcaaaaaactcacgtatttgggcaggcgccttattctggtataaatattacaaaggagggtattcatgttgcttctgcttcatctgctcgttcgactcgtcgttttttgagtgcataaacccgtccgcggaataagaccatatttataatgtttcctcccataagtaataggtgtcgaaggtcaaacacctaaatatgccgataaaacatttcaacctgaacctcaaataggagaatctcagtggtacgaaactgaaattaatcatttatccacagcttccagtttgtggatttatacggctattttgtaaagttggacttggagtttatcctcttagagtcaccatgctttgactttaattagtagcagctgggagagtccttaaaaagactaccccaatgaaaccatgttacggttcatatgcaaaacccacaaatgaaaatggagggcaaggcattcttgtaacgtcgaccctctcaggaatttttctgatggggttactttggtacaatgccaagtatacgttttgggtgtttacttttacctcccgttccgtaagaacattagcaacaaaatggaaagctagaaagtcaagtggaaatgcaatttttctcaactactgaggcgaccgcaggcaatggtgataacttgactcctaaagtggttcgttgttttacctttcgatctttcagttcacctttacgttaaaaagagttgatgactccgctggcgtccgttaccactattgaactgaggatttcaccaattgtacagtgaagatgtagatatagaaaccccagacactcatatttcttacatgcccactattaaggaaggtaactcacgagaactaatgggccaacaataacatgtcacttctacatctatatctttggggtctgtgagtataaagaatgtacgggtgataattccttccattgagtgctcttgattacccggttgtttctatgcccaacaggcctaattacattgcttttagggacaattttattggtctaatgtattacaacagcacgggtaatatgggtgttctggcgggccaagagatacgggttgtccggattaatgtaacgaaaatccctgttaaaataaccagattacataatgttgtcgtgcccattatacccacaagaccgcccggttccatcgcagttgaatgctgttgtagatttgcaagacagaaacacagagctttcataccagcttttgcttgattccattggtgatagaaccaggtacttttcgtagcgtcaacttacgacaacatctaaacgttctgtctttgtgtctcgaaagtatggtcgaaaacgaactaaggtaaccactatcttggtccatgaaaagtatgtggaatcaggctgttgacagctatgatccagatgttagaattattgaaaatcatggaactgaagatgaacttccaaattactgctttccactgggaatacaccttagtccgacaactgtcgatactaggtctacaatcttaataacttttagtaccttgacttctacttgaaggtttaatgacgaaaggtgaccctggtgtgattaatacagagactcttaccaaggtaaaacctaaaacaggtcaggaaaatggatgggaaaaagatgctacagaattttcagataaaaatgaaaccacactaattatgtctctgagaatggttccattttggattttgtccagtccttttacctaccctttttctacgatgtcttaaaagtctatttttacttttaagagttggaaataattttgccatggaaatcaatctaaatgccaacctgtggagaaatttcctgtactccaacatagcgctgtatttgcccgacaagctattctcaacctttattaaaacggtacctttagttagatttacggttggacacctctttaaaggacatgaggttgtatcgcgacataaacgggctgttcgaaaagtacagtccttccaacgtaaaaatttctgataacccaaacacctacgactacatgaacaagcgagtggtggctcccgggttagtggactgctacatttttcatgtcaggaaggttgcatttttaaagactattgggtttgtggatgctgatgtacttgttcgctcaccaccgagggcccaatcacctgacgatgtaaaaccttggagcacgctggtcccttgactatatggacaacgtcaacccatttaaccaccaccgcaatgctggcctgcgctaccgctcaatgttgctgggcattggaacctcgtgcgaccagggaactgatatacctgttgcagttgggtaaattggtggtggcgttacgaccggacgcgatggcgagttacaacgacccgtatggtcgctatgtgcccttccacatccaggtgcctcagaagttctttgccattaaaaacctccttctcctgccgggctcatacacctacgagtggaactttaccagcgatacacgggaaggtgtaggtccacggagtcttcaagaaacggtaatttttggaggaagaggacggcccgagtatgtggatgctcaccttgaacaggaaggatgttaacatggttctgcagagctccctaggaaatgacctaagggttgacggagccagcattaagtttgatagcatttgcctttacgccaccgtccttcctacaattgtaccaagacgtctcgagggatcctttactggattcccaactgcctcggtcgtaattcaaactatcgtaaacggaaatgcggtggttcttccccatggcccacaacaccgcctccacgcttgaggccatgcttagaaacgacaccaacgaccagtcctttaacgactatctctccgccgccaacaaagaaggggtaccgggtgttgtggcggaggtgcgaactccggtacgaatctttgctgtggttgctggtcaggaaattgctgatagagaggcggcggttgttgctctaccctatacccgccaacgctaccaacgtgcccatatccatcccctcccgcaactgggcggctttccgcggctgggccttcacgcgccttaagacacgagatgggatatgggcggttgcgatggttgcacgggtataggtaggggagggcgttgacccgccgaaaggcgccgacccggaagtgcgcggaattctgtaaggaaaccccatcactgggctcgggctacgacccttattacacctactctggctctataccctacctagatggaaccttttacctcaaccacacctttattcctttggggtagtgacccgagcccgatgctgggaataatgtggatgagaccgagatatgggatggatctaccttggaaaatggagttggtgtggaaaaagaaggtggccattacctttgactcttctgtcagctggcctggcaatgaccgcctgcttacccccaacgagtttgaaattaagcgctcagttgacggggttcttccaccggtaatggaaactgagaagacagtcgaccggaccgttactggcggacgaatgggggttgctcaaactttaattcgcgagtcaactgccccagggttacaacgttgcccagtgtaacatgaccaaagactggttcctggtacaaatgctagctaactacaacattggctaccagggcttctatatcccagatcccaatgttgcaacgggtcacattgtactggtttctgaccaaggaccatgtttacgatcgattgatgttgtaaccgatggtcccgaagatatagggtctgagctacaaggaccgcatgtactccttctttagaaacttccagcccatgagccgtcaggtggtggatgatactaaatacaaggactaccaacaggtgggcctcgatgttcctggcgtacatgaggaagaaatctttgaaggtcgggtactcggcagtccaccacctactatgatttatgttcctgatggttgtccacccgatcctacaccaacacaacaactctggatttgttggctaccttgcccccaccatgcgcgaaggacaggcctaccctgctaacttcccctatccgcttatagtaggatgtggttgtgttgttgagacctaaacaaccgatggaacgggggtggtacgcgcttcctgtccggatgggacgattgaaggggataggcgaatatcgcaagaccgcagttgacagcattacccagaaaaagtttctttgcgatcgcaccctttggcgcatcccattctccagtaactttatgtccatgggcgcactcgttctggcgtcaactgtcgtaatgggtctttttcaaagaaacgctagcgtgggaaaccgcgtagggtaagaggtcattgaaatacaggtacccgcgtgacacagacctgggccaaaaccttctctacgccaactccgcccacgcgctagacatgacttttgaggtggatcccatggacgagcccacccttctttatgttgtgtctggacccggttttggaagagatgcggttgaggcgggtgcgcgatctgtactgaaaactccacctagggtacctgctcgggtgggaagaaatacaattgtttgaagtctttgacgtggtccgtgtgcaccggccgcaccgcggcgtcatcgaaaccgtgtacctgcgcacgcccttctcggccggcaacgccacaaaacaaacttcagaaactgcaccaggcacacgtggccggcgtggcgccgcagtagctttggcacatggacgcgtgcgggaagagccggccgttgcggtgttcataaagaagcaagcaacatcaacaacagctgccgccatgggctccagtgagcaggaactgaaagccattgtcaaagatcttggttgtgggccatattttgtatttcttcgttcgttgtagttgttgtcgacggcggtacccgaggtcactcgtccttgactttcggtaacagtttctagaaccaacacccggtataaaattgggcacctatgacaagcgctttccaggctttgtttctccacacaagctcgcctgcgccatagtcaatacggccggtcgcgagactgggggcgtacactaacccgtggatactgttcgcgaaaggtccgaaacaaagaggtgtgttcgagcggacgcggtatcagttatgccggccagcgctctgacccccgcatgtgaggatggcctttgcctggaacccgcactcaaaaacatgctacctctttgagccctttggcttttctgaccagcgactcaagcaggtttaccagtttgagtacctaccggaaacggaccttgggcgtgagtttttgtacgatggagaaactcgggaaaccgaaaagactggtcgctgagttcgtccaaatggtcaaactcatcgagtcactcctgcgccgtagcgccattgcttcttcccccgaccgctgtataacgctggaaaagtccacccaaagcgtacaggggcccaactcggccgccgctcagtgaggacgcggcatcgcggtaacgaagaagggggctggcgacatattgcgaccttttcaggtgggtttcgcatgtccccgggttgagccggcggtgtggactattctgctgcatgtttctccacgcctttgccaactggccccaaactcccatggatcacaaccccaccatgaaccttattaccggggtacccaacacctgataagacgacgtacaaagaggtgcggaaacggttgaccggggtttgagggtacctagtgttggggtggtacttggaataatggccccatgggtactccatgctcaacagtccccaggtacagcccaccctgcgtcgcaaccaggaacagctctacagcttcctggagcgccactcgccctacttccgcagccatgaggtacgagttgtcaggggtccatgtcgggtgggacgcagcgttggtccttgtcgagatgtcgaaggacctcgcggtgagcgggatgaaggcgtcggtcagtgcgcagattaggagcgccacttctttttgtcacttgaaaaacatgtaaaaataatgtactagagacactttcaataaaggcaaatgcttttatttggtcacgcgtctaatcctcgcggtgaagaaaaacagtgaactttttgtacatttttattacatgatctctgtgaaagttatttccgtttacgaaaataaactacactctcgggtgattatttacccccacccttgccgtctgcgccgtttaaaaatcaaaggggttctgccgcgcatcgctatgcgccactggcagggacaatgtgagagcccactaataaatgggggtgggaacggcagacgcggcaaatttttagtttccccaagacggcgcgtagcgatacgcggtgaccgtccctgtcgttgcgatactggtgtttagtgctccacttaaactcaggcacaaccatccgcggcagctcggtgaagttttcactccacaggctgcgcaccatcaccaagcaacgctatgaccacaaatcacgaggtgaatttgagtccgtgttggtaggcgccgtcgagccacttcaaaagtgaggtgtccgacgcgtggtagtggttcgcgtttagcaggtcgggcgccgatatcttgaagtcgcagttggggcctccgccctgcgcgcgcgagttgcgatacacagggttgcagcactggaacactgcgcaaatcgtccagcccgcggctatagaacttcagcgtcaaccccggaggcgggacgcgcgcgctcaacgctatgtgtcccaacgtcgtgaccttgtgaatcagcgccgggtggtgcacgctggccagcacgctcttgtcggagatcagatccgcgtccaggtcctccgcgttgctcagggcgaacggagtcaactttgtagtcgcggcccaccacgtgcgaccggtcgtgcgagaacagcctctagtctaggcgcaggtccaggaggcgcaacgagtcccgcttgcctcagttgaaacgtagctgccttcccaaaaagggcgcgtgcccaggctttgagttgcactcgcaccgtagtggcatcaaaaggtgaccgtgcccggtctgggcgttaggatacatcgacggaagggtttttcccgcgcacgggtccgaaactcaacgtgagcgtggcatcaccgtagttttccactggcacgggccagacccgcaatcctatcagcgcctgcataaaagccttgatctgcttaaaagccacctgagcctttgcgccttcagagaagaacatgccgcaagacttgccggaaaactgattggccgtcgcggacgtattttcggaactagacgaattttcggtggactcggaaacgcggaagtctcttcttgtacggcgttctgaacggccttttgactaaccggggacaggccgcgtcgtgcacgcagcaccttgcgtcggtgttggagatctgcaccacatttcggccccaccggttcttcacgatcttggccttgctagactcctgtccggcgcagcacgtgcgtcgtggaacgcagccacaacctctagacgtggtgtaaagccggggtggccaagaagtgctagaaccggaacgatctgagctccttcagcgcgcgctgcccgttttcgctcgtcacatccatttcaatcacgtgctccttatttatcataatgcttccgtgtagacacttaagctcgcccgaggaagtcgcgcgcgacgggcaaaagcgagcagtgtaggtaaagttagtgcacgaggaataaatagtattacgaaggcacatctgtgaattcgagcggttcgatctcagcgcagcggtgcagccacaacgcgcagcccgtgggctcgtgatgcttgtaggtcacctctgcaaacgactgcaggtacgcctgcaggaataagctagagtcgcgtcgccacgtcggtgttgcgcgtcgggcacccgagcactacgaacatccagtggagacgtttgctgacgtccatgcggacgtccttacgccccatcatcgtcacaaaggtcttgttgctggtgaaggtcagctgcaacccgcggtgctcctcgttcagccaggtcttgcatacggccgccagagcttgcggggtagtagcagtgtttccagaacaacgaccacttccagtcgacgttgggcgccacgaggagcaagtcggtccagaacgtatgccggcggtctcgaaccacttggtcaggcagtagtttgaagttcgcctttagatcgttatccacgtggtacttgtccatcagcgcgcgcgcagcctccatgcccttctcccacgcggtgaaccagtccgtcatcaaacttcaagcggaaatctagcaataggtgcaccatgaacaggtagtcgcgcgcgcgtcggaggtacgggaagagggtgcgagacacgatcggcacactcagcgggttcatcaccgtaatttcactttccgcttcgctgggctcttcctcttcctcttgcgtccgcataccacgcgccacttctgtgctagccgtgtgagtcgcccaagtagtggcattaaagtgaaaggcgaagcgacccgagaaggagaaggagaacgcaggcgtatggtgcgcggtgagggtcgtcttcattcagccgccgcactgtgcgcttacctcctttgccatgcttgattagcaccggtgggttgctgaaacccaccatttgtagcgccacatcccagcagaagtaagtcggcggcgtgacacgcgaatggaggaaacggtacgaactaatcgtggccacccaacgactttgggtggtaaacatcgcggtgtacttctctttcttcctcgctgtccacgattacctctggtgatggcgggcgctcgggcttgggagaagggcgcttctttttcttcttgggcgcaatggccaagaagagaaagaaggagcgacaggtgctaatggagaccactaccgcccgcgagcccgaaccctcttcccgcgaagaaaaagaagaacccgcgttaccggttatccgccgccgaggtcgatggccgcgggctgggtgtgcgcggcaccagcgcgtcttgtgatgagtcttcctcgtcctcggactcgatacgccgcctcatctaggcggcggctccagctaccggcgcccgacccacacgcgccgtggtcgcgcagaacactactcagaaggagcaggagcctgagctatgcggcggagtagcgcttttttgggggcgcccggggaggcggcggcgacggggacggggacgacacgtcctccatggttgggggacgtcgcgccgcaccgcgtccgcgctcgggcgaaaaaacccccgcgggcccctccgccgccgctgcccctgcccctgctgtgcaggaggtaccaaccccctgcagcgcggcgtggcgcaggcgcgagccgggtggtttcgcgctgctcctcttcccgactggccatttccttctcctataggcagaaaaagatcatggagtcagtcgagaagaaggacagcctaaccgccccaccaaagcgcgacgaggagaagggctgaccggtaaaggaagaggatatccgtctttttctagtacctcagtcagctcttcttcctgtcggattggcgcccctctgagttcgccaccaccgcctccaccgatgccgccaacgcgcctaccaccttccccgtcgaggcacccccgcttgaggaggaggaagtgattatcggggagactcaagcggtggtggcggaggtggctacggcggttgcgcggatggtggaaggggcagctccgtgggggcgaactcctcctccttcactaataggagcaggacccaggttttgtaagcgaagacgacgaggaccgctcagtaccaacagaggataaaaagcaagaccaggacaacgcagaggcaaacgaggaacctcgtcctgggtccaaaacattcgcttctgctgctcctggcgagtcatggttgtctcctatttttcgttctggtcctgttgcgtctccgtttgctccttgaagtcgggcggggggacgaaaggcatggcgactacctagatgtgggagacgacgtgctgttgaagcatctgcagcgccagtgcgccattatctgcgacgcttcagcccgcccccctgctttccgtaccgctgatggatctacaccctctgctgcacgacaacttcgtagacgtcgcggtcacgcggtaatagacgctgcggttgcaagagcgcagcgatgtgcccctcgccatagcggatgtcagccttgcctacgaacgccacctattctcaccgcgcgtaccccccaaacgccaagaacaacgttctcgcgtcgctacacggggagcggtatcgcctacagtcggaacggatgcttgcggtggataagagtggcgcgcatggggggtttgcggttcttaacggcacatgcgagcccaacccgcgcctcaacttctaccccgtatttgccgtgccagaggtgcttgccacctatcacatctttttccaaaactgcaagattgccgtgtacgctcgggttgggcgcggagttgaagatggggcataaacggcacggtctccacgaacggtggatagtgtagaaaaaggttttgacgttcttacccctatcctgccgtgccaaccgcagccgagcggacaagcagctggccttgcggcagggcgctgtcatacctgatatcgcctcgctcaacgaagtgccatggggataggacggcacggttggcgtcggctcgcctgttcgtcgaccggaacgccgtcccgcgacagtatggactatagcggagcgagttgcttcacggaaaaatctttgagggtcttggacgcgacgagaagcgcgcggcaaacgctctgcaacaggaaaacagcgaaaatgaaagtcactctggagtgttggtggaatttttagaaactcccagaacctgcgctgctcttcgcgcgccgtttgcgagacgttgtccttttgtcgcttttactttcagtgagacctcacaaccaccttctcgagggtgacaacgcgcgcctagccgtactaaaacgcagcatcgaggtcacccactttgcctacccggcacttaacctaccccccaaggtcatgagcagagctcccactgttgcgcgcggatcggcatgattttgcgtcgtagctccagtgggtgaaacggatgggccgtgaattggatggggggttccagtactcgtcagtcatgagtgagctgatcgtgcgccgtgcgcagcccctggagagggatgcaaatttgcaagaacaaacagaggagggcctacccgcagttggcgacgagtcagtactcactcgactagcacgcggcacgcgtcggggacctctccctacgtttaaacgttcttgtttgtctcctcccggatgggcgtcaaccgctgctgcagctagcgcgctggcttcaaacgcgcgagcctgccgacttggaggagcgacgcaaactaatgatggccgcagtgctcgttaccgtggagcttgagtgccgtcgatcgcgcgaccgaagtttgcgcgctcggacggctgaacctcctcgctgcgtttgattactaccggcgtcacgagcaatggcacctcgaactcacgatgcagcggttctttgctgacccggagatgcagcgcaagctagaggaaacattgcactacacctttcgacagggctacgtacgccaggcctgcaagatcttacgtcgccaagaaacgactgggcctctacgtcgcgttcgatctcctttgtaacgtgatgtggaaagctgtcccgatgcatgcggtccggacgttctagaccaacgtggagctctgcaacctggtctcctaccttggaattttgcacgaaaaccgccttgggcaaaacgtgcttcattccacgctcaagggcgaggcgcgggttgcacctcgagacgttggaccagaggatggaaccttaaaacgtgcttttggcggaacccgttttgcacgaagtaaggtgcgagttcccgctccgcgcccgcgactacgtccgcgactgcgtttacttatttctatgctacacctggcagacggccatgggcgtttggcagcagtgcttggaggagtgcaacctcaagggcgctgatgcaggcgctgacgcaaatgaataaagatacgatgtggaccgtctgccggtacccgcaaaccgtcgtcacgaacctcctcacgttggagttcgagctgcagaaactgctaaagcaaaacttgaaggacctatggacggccttcaacgagcgctccgtggccgcgcacctggcggacatcattttccccgaacctcgacgtctttgacgatttcgttttgaacttcctggatacctgccggaagttgctcgcgaggcaccggcgcgtggaccgcctgtagtaaaaggggcttggcctgcttaaaaccctgcaacagggtctgccagacttcaccagtcaaagcatgttgcagaactttaggaactttatcctagagcgctcaggaatcttgcccggacgaattttgggacgttgtcccagacggtctgaagtggtcagtttcgtacaacgtcttgaaatccttgaaataggatctcgcgagtccttagaacggcgccacctgctgtgcacttcctagcgactttgtgcccattaagtaccgcgaatgccctccgccgctttggggccactgctaccttctgcagctagccaacgcggtggacgacacgtgaaggatcgctgaaacacgggtaattcatggcgcttacgggaggcggcgaaaccccggtgacgatggaagacgtcgatcggttgtaccttgcctaccactctgacataatggaagacgtgagcggtgacggtctactggagtgtcactgtcgctgcaacctatgcaccccgcaccgctccctggatggaacggatggtgagactgtattaccttctgcactcgccactgccagatgacctcacagtgacagcgacgttggatacgtggggcgtggcgagggacctttgcaattcgcagctgcttaacgaaagtcaaattatcggtacctttgagctgcagggtccctcgcctgacgaaaagtccgcggctccggggttgaaactaaacgttaagcgtcgacgaattgctttcagtttaatagccatggaaactcgacgtcccagggagcggactgcttttcaggcgccgaggccccaactttgacactccggggctgtggacgtcggcttaccttcgcaaatttgtacctgaggactaccacgcccacgagattaggttctacgaagaccaatcccgcccgccagtgaggccccgacacctgcagccgaatggaagcgtttaaacatggactcctgatggtgcgggtgctctaatccaagatgcttctggttagggcgggcggtaatgcggagcttaccgcctgcgtcattacccagggccacattcttggccaattgcaagccatcaacaaagcccgccaagagtttctgctacgaaagggacttacgcctcgaatggcggacgcagtaatgggtcccggtgtaagaaccggttaacgttcggtagttgtttcgggcggttctcaaagacgatgctttccctgggggggtttacttggacccccagtccggcgaggagctcaacccaatccccccgccgccgcagccctatcagcagcagccgcgggcccttgcttcccaggaccccccaaatgaacctgggggtcaggccgctcctcgagttgggttaggggggcggcggcgtcgggatagtcgtcgtcggcgcccgggaacgaagggtccttggcacccaaaaagaagctgcagctgccgccgccacccacggacgaggaggaatactgggacagtcaggcagaggaggttttggacgaggaggaggaggaaccgtgggtttttcttcgacgtcgacggcggcggtgggtgcctgctcctccttatgaccctgtcagtccgtctcctccaaaacctgctcctcctcctcctcatgatggaagactgggagagcctagacgaggaagcttccgaggtcgaagaggtgtcagacgaaacaccgtcaccctcggtcgcattcccctcgccggcggtactaccttctgaccctctcggatctgctccttcgaaggctccagcttctccacagtctgctttgtggcagtgggagccagcgtaaggggagcggccgcccccagaaatcggcaaccggttccagcatggctacaacctccgctcctcaggcgccgccggcactgcccgttcgccgacccaaccgtagatgggacaccaggggtctttagccgttggccaaggtcgtaccgatgttggaggcgaggagtccgcggcggccgtgacgggcaagcggctgggttggcatctaccctgtggtctggaaccagggccggtaagtccaagcagccgccgccgttagcccaagagcaacaacagcgccaaggctaccgctcatggcgcgggcacaagaacgccatgaccttggtcccggccattcaggttcgtcggcggcggcaatcgggttctcgttgttgtcgcggttccgatggcgagtaccgcgcccgtgttcttgcggtaagttgcttgcttgcaagactgtgggggcaacatctccttcgcccgccgctttcttctctaccatcacggcgtggccttcccccgtaacatcctgcattactcaacgaacgaacgttctgacacccccgttgtagaggaagcgggcggcgaaagaagagatggtagtgccgcaccggaagggggcattgtaggacgtaatgtaccgtcatctctacagcccatactgcaccggcggcagcggcagcggcagcaacagcagcggccacacagaagcaaaggcgaccggatagcaagactctgatggcagtagagatgtcgggtatgacgtggccgccgtcgccgtcgccgtcgttgtcgtcgccggtgtgtcttcgtttccgctggcctatcgttctgagacacaaagcccaagaaatccacagcggcggcagcagcaggaggaggagcgctgcgtctggcgcccaacgaacccgtatcgacccgcgagcttagaaacaggatgtttcgggttctttaggtgtcgccgccgtcgtcgtcctcctcctcgcgacgcagaccgcgggttgcttgggcatagctgggcgctcgaatctttgtccttttttcccactctgtatgctatatttcaacagagcaggggccaagaacaagagctgaaaataaaaaacaggtctctgcgatccctcacccgcagctgcctaaaaagggtgagacatacgatataaagttgtctcgtccccggttcttgttctcgacttttattttttgtccagagacgctagggagtgggcgtcgacggagtatcacaaaagcgaagatcagcttcggcgcacgctggaagacgcggaggctctcttcagtaaatactgcgcgctgactcttaaggactagtttcgcgcccatagtgttttcgcttctagtcgaagccgcgtgcgaccttctgcgcctccgagagaagtcatttatgacgcgcgactgagaattcctgatcaaagcgcggctttctcaaatttaagcgcgaaaactacgtcatctccagcggccacacccggcgccagcacctgtcgtcagcgccattatgagcaaggaaattcccacgcgaaagagtttaaattcgcgcttttgatgcagtagaggtcgccggtgtgggccgcggtcgtggacagcagtcgcggtaatactcgttcctttaagggtgcgcctacatgtggagttaccagccacaaatgggacttgcggctggagctgcccaagactactcaacccgaataaactacatgagcgcgggaccccacatgatggatgtacacctcaatggtcggtgtttaccctgaacgccgacctcgacgggttctgatgagttgggcttatttgatgtactcgcgccctggggtgtactaatcccgggtcaacggaatccgcgcccaccgaaaccgaattctcttggaacaggcggctattaccaccacacctcgtaataaccttaatccccgtagttggtagggcccagttgccttaggcgcgggtggctttggcttaagagaaccttgtccgccgataatggtggtgtggagcattattggaattaggggcatcaacccccgctgccctggtgtaccaggaaagtcccgctcccaccactgtggtacttcccagagacgcccaggccgaagttcagatgactaactcaggggcgcagcgggcgacgggaccacatggtcctttcagggcgagggtggtgacaccatgaagggtctctgcgggtccggcttcaagtctactgattgagtccccgcgtcgttgcgggcggctttcgtcacagggtgcggtcgcccgggcagggtataactcacctgacaatcagagggcgaggtattcagctcaacgacgagtcggtgagaacgcccgccgaaagcagtgtcccacgccagcgggcccgtcccatattgagtggactgttagtctcccgctccataagtcgagttgctgctcagccactcctcctcgcttggtctccgtccggacgggacatttcagatcggcggcgccggccgtccttcattcacgcctcgtcaggcaatcctaactctgcagacctcggaggagcgaaccagaggcaggcctgccctgtaaagtctagccgccgcggccggcaggaagtaagtgcggagcagtccgttaggattgagacgtctggagctcctctgagccgcgctctggaggcattggaactctgcaatttattgaggagtttgtgccatcggtctactttaaccccttctcgggacctcccggccactaggagactcggcgcgagacctccgtaaccttgagacgttaaataactcctcaaacacggtagccagatgaaattggggaagagccctggagggccggtgaatccggatcaatttattcctaactttgacgcggtaaaggactcggcggacggctacgactgaatgttaagtggagaggcagagcaactgcgcctgaaacataggcctagttaaataaggattgaaactgcgccatttcctgagccgcctgccgatgctgacttacaattcacctctccgtctcgttgacgcggactttgtcctggtccactgtcgccgccacaagtgctttgcccgcgactccggtgagttttgctactttgaattgcccgaggatcatatcgagggcccggcgcacggcggaccaggtgacagcggcggtgttcacgaaacgggcgctgaggccactcaaaacgatgaaacttaacgggctcctagtatagctcccgggccgcgtgccggtccggcttaccgcccagggagagcttgcccgtagcctgattcgggagtttacccagcgccccctgctagttgagcgggacaggggaccctgtgttctcacaggccgaatggcgggtccctctcgaacgggcatcggactaagccctcaaatgggtcgcgggggacgatcaactcgccctgtcccctgggacacaagagtctgtgatttgcaactgtcctaaccttggattacatcaagatcctctagttataactagagtacccggggatcttattccctttaactaataaaaaaaaatgacactaaacgttgacaggattggaacctaatgtagttctaggagatcaatattgatctcatgggcccctagaataagggaaattgattatttttttttaaataaagcatcacttacttaaaatcagttagcaaatttctgtccagtttattcagcagcacctccttgccctcctcccagctctggtattgcagcttcctttatttcgtagtgaatgaattttagtcaatcgtttaaagacaggtcaaataagtcgtcgtggaggaacgggaggagggtcgagaccataacgtcgaaggacctggctgcaaactttctccacaatctaaatggaatgtcagtttcctcctgttcctgtccatccgcacccactatcttcatgttgttgcagatgaagcgcggaccgacgtttgaaagaggtgttagatttaccttacagtcaaaggaggacaaggacaggtaggcgtgggtgatagaagtacaacaacgtctacttcgcggcaagaccgtctgaagataccttcaaccccgtgtatccatatgacacggaaaccggtcctccaactgtgccttttcttactcctccctttgtatcccccacgttctggcagacttctatggaagttggggcacataggtatactgtgcctttggccaggaggttgacacggaaaagaatgaggagggaaacatagggggtatgggtttcaagagagtccccctggggtactctctttgcgcctatccgaacctctagttacctccaatggcatgcttgcgctcaaaatgggcaacggccttacccaaagttctctcagggggaccccatgagagaaacgcggataggcttggagatcaatggaggttaccgtacgaacgcgagttttacccgttgccggactctctggacgaggccggcaaccttacctcccaaaatgtaaccactgtgagcccacctctcaaaaaaaccaagtcaaacataaacctggaaatatctgcagagagacctgctccggccgttggaatggagggttttacattggtgacactcgggtggagagtttttttggttcagtttgtatttggacctttatagacgtcccctcacagttacctcagaagccctaactgtggctgccgccgcacctctaatggtcgcgggcaacacactcaccatgcaatcacaggccccgctaaccgggggagtgtcaatggagtcttcgggattgacaccgacggcggcgtggagattaccagcgcccgttgtgtgagtggtacgttagtgtccggggcgattggctgcacgactccaaacttagcattgccacccaaggacccctcacagtgtcagaaggaaagctagccctgcaaacatcaggccccctcaccaccaccgatagacgtgctgaggtttgaatcgtaacggtgggttcctggggagtgtcacagtcttcctttcgatcgggacgtttgtagtccgggggagtggtggtggctatccagtacccttactatcactgcctcaccccctctaactactgccactggtagcttgggcattgacttgaaagagcccatttatacacaaaatggaaaactagtcatgggaatgatagtgacggagtgggggagattgatgacggtgaccatcgaacccgtaactgaactttctcgggtaaatatgtgttttaccttttgatggactaaagtacggggctcctttgcatgtaacagacgacctaaacactttgaccgtagcaactggtccaggtgtgactattaataatacttccttgcaaacctgatttcatgccccgaggaaacgtacattgtctgctggatttgtgaaactggcatcgttgaccaggtccacactgataattattatgaaggaacgtttctaaagttactggagccttgggttttgattcacaaggcaatatgcaacttaatgtagcaggaggactaaggattgattctcaaaacagacgccttatactgatttcaatgacctcggaacccaaaactaagtgttccgttatacgttgaattacatcgtcctcctgattcctaactaagagttttgtctgcggaatatgatgatgttagttatccgtttgatgctcaaaaccaactaaatctaagactaggacagggccctctttttataaactcagcccacaacttggatattaactacactacaatcaataggcaaactacgagttttggttgatttagattctgatcctgtcccgggagaaaaatatttgagtcgggtgttgaacctataattgatgaacaaaggcctttacttgtttacagcttcaaacaattccaaaaagcttgaggttaacctaagcactgccaaggggttgatgtttgacgctacagccatagttgtttccggaaatgaacaaatgtcgaagtttgttaaggtttttcgaactccaattggattcgtgacggttccccaactacaaactgcgatgtcggtatcccattaatgcaggagatgggcttgaatttggttcacctaatgcaccaaacacaaatcccctcaaaacaaaaattggccatggcctagaatttgattcaaaggtaattacgtcctctacccgaacttaaaccaagtggattacgtggtttgtgtttaggggagttttgtttttaaccggtaccggatcttaaactaagtttcaaggctatggttcctaaactaggaactggccttagttttgacagcacaggtgccattacagtaggaaacaaaaataatgataagctaactttgtggaccgttccgataccaaggatttgatccttgaccggaatcaaaactgtcgtgtccacggtaatgtcatcctttgtttttattactattcgattgaaacacctggacaccagctccatctcctaactgtagactaaatgcagagaaagatgctaaactcactttggtcttaacaaaatgtggcagtcaaatacttgctacagttttgtggtcgaggtagaggattgacatctgatttacgtctctttctacgatttgagtgaaaccagaattgttttacaccgtcagtttatgaacgatgtcaaacagttttggctgttaaaggcagtttggctccaatatctggaacagttcaaagtgctcatcttattataagatttgacgaaaatggagtgctactaaacaagtcaaaaccgacaatttccgtcaaaccgaggttatagaccttgtcaagtttcacgagtagaataatattctaaactgcttttacctcacgatgatttgttttccttcctggacccagaatattggaactttagaaatggagatcttactgaaggcacagcctatacaaacgctgttggatttatgcctaacctatcagctaaggaaggacctgggtcttataaccttgaaatctttacctctagaatgacttccgtgtcggatatgtttgcgacaacctaaatacggattggatagtcgatatccaaaatctcacggtaaaactgccaaaagtaacattgtcagtcaagtttacttaaacggagacaaaactaaacctgtaacactaaccattacactaaataggttttagagtgccattttgacggttttcattgtaacagtcagttcaaatgaatttgcctctgttttgatttggacattgtgattggtaatgtgattacggtacacaggaaacaggagacacaactccaagtgcatactctatgtcattttcatgggactggtctggccacaactacattaatgaaatatttgccactgccatgtgtcctttgtcctctgtgttgaggttcacgtatgagatacagtaaaagtaccctgaccagaccggtgttgatgtaattactttataaacggtgatcctcttacactttttcatacattgcccaagaataaagaatcgtttgtgttatgtttcaacgtgtttatttttcannnnccctaggcaaaatagcaccctaggagaatgtgaaaaagtatgtaacgggttcttatttcttagcaaacacaatacaaagttgcacaaataaaaagtnnnngggatccgttttatcgtgggtcccgctccagaacaacatacagcgcttcacagcggcagcctaacagtcagccttaccagtaaaaaagaaaacctattaaaaaaacaccactcgacacggagggcgaggtcttgttgtatgtcgcgaagtgtcgccgtcggattgtcagtcggaatggtcattttttcttttggataatttttttgtggtgagctgtgcccaccagctcaatcagtcacagtgtaaaaaagggccaagtgcagagcgagtatatataggactaaaaaatgacgtaacggttaaagtccacaaaaaacaccgtggtcgagttagtcagtgtcacattttttcccggttcacgtctcgctcatatatatcctgattttttactgcattgccaatttcaggtgttttttgtggcagaaaaccgcacgcgaacctacgcccagaaacgaaagccaaaaaacccacaacttcctcaaatcgtcacttccgttttcccacgttacgtaacttcccagtcttttggcgtgcgcttggatgcgggtctttgctttcggttttttgggtgttgaaggagtttagcagtgaaggcaaaagggtgcaatgcattgaagggtttttaagaaaactacaattcccaacacatacaagttactccgccctaaaacctacgtcacccgccccgttcccacgccccgcgccacgtcacaaactccaaaaattcttttgatgttaagggttgtgtatgttcaatgaggcgggattttggatgcagtgggcggggcaagggtgcggggcgcggtgcagtgtttgaggtccccctcattatcatattggcttcaatccaaaataaggtatattattgatgatgttaatgggggagtaatagtataaccgaagttaggttttattccatataataactactacaatta

SEQ ID NO: 3 - DNA, 70016 bp, Artificial
  /note = Sequence of Hybrid Ad-SFV vector containing E1, E2, E3and E4 deletions from the adenovirus backbone
taacatcatcaataatataccttattttggattgaagccaatatgataatgagggggtggagtttgtgacgtggcgcggggcgtgggaacggggcgggtgattgtagtagttattatatggaataaaacctaacttcggttatactattactcccccacctcaaacactgcaccgcgccccgcacccttgccccgcccacacgtagtagtgtggcggaagtgtgatgttgcaagtgtggcggaacacatgtaagcgacggatgtggcaaaagtgacgtttttggtgtgcgccggtgtacatgcatcatcacaccgccttcacactacaacgttcacaccgccttgtgtacattcgctgcctacaccgttttcactgcaaaaaccacacgcggccacatgtcaggaagtgacaattttcgcgcggttttaggcggatgttgtagtaaatttgggcgtaaccgagtaagatttggccattttcgcgggaaaactgaataagagtccttcactgttaaaagcgcgccaaaatccgcctacaacatcatttaaacccgcattggctcattctaaaccggtaaaagcgcccttttgacttattctggaagtgaaatctgaataattttgtgttactcatagcgcgtaatactggtaccgcggccgcctcgagtttaccactccctatcagtgatagagaaaagtgccttcactttagacttattaaaacacaatgagtatcgcgcattatgaccatggcgccggcggagctcaaatggtgagggatagtcactatctcttttcacaaagtcgagtttaccactccctatcagtgatagagaaaagtgaaagtcgagtttaccactccctatcagtgatagagaaaagtgaaagtcgagtttaccatttcagctcaaatggtgagggatagtcactatctcttttcactttcagctcaaatggtgagggatagtcactatctcttttcactttcagctcaaatggtctccctatcagtgatagagaaaagtgaaagtcgagtttaccactccctatcagtgatagagaaaagtgaaagtcgagtttaccactccctatcagtgatagagggatagtcactatctcttttcactttcagctcaaatggtgagggatagtcactatctcttttcactttcagctcaaatggtgagggatagtcactatgagaaaagtgaaagtcgagtttaccactccctatcagtgatagagaaaagtgaaagtcgagctcggtacccgggtcgaggtaggcgtgtacggtgggaggctcttttcactttcagctcaaatggtgagggatagtcactatctcttttcactttcagctcgagccatgggcccagctccatccgcacatgccaccctcccctatataagcagagctcgtttagtgaaccgtcagatcgcctggagacgccatccacgctgttttgacctccatagaagacaccgggaccgatccagcctggatatattcgtctcgagcaaatcacttggcagtctagcggacctctgcggtaggtgcgacaaaactggaggtatcttctgtggccctggctaggtcggaccgcggccccgaattcgagctcggtacccggggatcgatggcggatgtgtgacatacacgacgccaaaagattttgttccagctcctgccacctccgctaggcgccggggcttaagctcgagccatgggcccctagctaccgcctacacactgtatgtgctgcggttttctaaaacaaggtcgaggacggtggaggcgatcgcgagagattaaccacccacgatggccgccaaagtgcatgttgatattgaggctgacagcccattcatcaagtctttgcagaaggcatttccgtcgttcgcgctctctaattggtgggtgctaccggcggtttcacgtacaactataactccgactgtcgggtaagtagttcagaaacgtcttccgtaaaggcagcaaggaggtggagtcattgcaggtcacaccaaatgaccatgcaaatgccagagcattttcgcacctggctaccaaattgatcgagcaggagactgacaaagacactccacctcagtaacgtccagtgtggtttactggtacgtttacggtctcgtaaaagcgtggaccgatggtttaactagctcgtcctctgactgtttctgtcactcatcttggatatcggcagtgcgccttccaggagaatgatgtctacgcacaaataccactgcgtatgccctatgcgcagcgcagaagaccccgaaaggtgagtagaacctatagccgtcacgcggaaggtcctcttactacagatgcgtgtttatggtgacgcatacgggatacgcgtcgcgtcttctggggctttcgctcgatagctacgcaaagaaactggcagcggcctccgggaaggtgctggatagagagatcgcaggaaaaatcaccgacctgcagaccgtcatggctacgcgagctatcgatgcgtttctttgaccgtcgccggaggcccttccacgacctatctctctagcgtcctttttagtggctggacgtctggcagtaccgatgcccagacgctgaatctcctaccttttgcctgcatacagacgtcacgtgtcgtacggcagccgaagtggccgtataccaggacgtgtatgctgtacatgcacggtctgcgacttagaggatggaaaacggacgtatgtctgcagtgcacagcatgccgtcggcttcaccggcatatggtcctgcacatacgacatgtacgtgcaacatcgctgtaccatcaggcgatgaaaggtgtcagaacggcgtattggattgggtttgacaccaccccgtttatgtttgacgcgctagcaggcgcgtagttgtagcgacatggtagtccgctactttccacagtcttgccgcataacctaacccaaactgtggtggggcaaatacaaactgcgcgatcgtccgcgcattccaacctacgccacaaactgggccgacgagcaggtgttacaggccaggaacataggactgtgtgcagcatccttgactgagggaagactcggcaaactgaggttggatgcggtgtttgacccggctgctcgtccacaatgtccggtccttgtatcctgacacacgtcgtaggaactgactcccttctgagccgtttgactccattctccgcaagaagcaattgaaaccttgcgacacagtcatgttctcggtaggatctacattgtacactgagagcagaaagctactgaggagctggcaggtaagaggcgttcttcgttaactttggaacgctgtgtcagtacaagagccatcctagatgtaacatgtgactctcgtctttcgatgactcctcgaccgacttaccctccgtattccacctgaaaggtaaacaatcctttacctgtaggtgcgataccatcgtatcatgtgaagggtacgtagttaagaaaatcactattgaatgggaggcataaggtggactttccatttgttaggaaatggacatccacgctatggtagcatagtacacttcccatgcatcaattcttttagtgatagtgccccggcctgtacggtaaaacggtagggtacgccgtgacgtatcacgcggagggattcctagtgtgcaagaccacagacactgtcaaaggagaaagacacggggccggacatgccattttgccatcccatgcggcactgcatagtgcgcctccctaaggatcacacgttctggtgtctgtgacagtttcctctttctgtctcattccctgtatgcacctacgtcccctcaaccatctgtgatcaaatgactggcatactagcgaccgacgtcacaccggaggacgcacagaagttgtcagagtaagggacatacgtggatgcaggggagttggtagacactagtttactgaccgtatgatcgctggctgcagtgtggcctcctgcgtgtcttcaacatagtgggattgaatcagaggatagttgtgaacggaagaacacagcgaaacactaacacgatgaagaactatctgcttccgattgtggccgtcgcatttagatcaccctaacttagtctcctatcaacacttgccttcttgtgtcgctttgtgattgtgctacttcttgatagacgaaggctaacaccggcagcgtaaatccaagtgggcgagggaatacaaggcagaccttgatgatgaaaaacctctgggtgtccgagagaggtcacttacttgctgctgcttgtgggcatttaaaacggttcacccgctcccttatgttccgtctggaactactactttttggagacccacaggctctctccagtgaatgaacgacgacgaacacccgtaaattttgcaggaagatgcacaccatgtacaagaaaccagacacccagacaatagtgaaggtgccttcagagtttaactcgttcgtcatcccgagcctatggtctacagtccttctacgtgtggtacatgttctttggtctgtgggtctgttatcacttccacggaagtctcaaattgagcaagcagtagggctcggataccagatgtcgcctcgcaatcccagtcagatcacgcattaagatgcttttggccaagaagaccaagcgagagttaatacctgttctcgacgcgtcgtcagccagggatgccggagcgttagggtcagtctagtgcgtaattctacgaaaaccggttcttctggttcgctctcaattatggacaagagctgcgcagcagtcggtccctacgtgaacaagaggagaaggagaggttggaggccgagctgactagagaagccttaccacccctcgtccccatcgcgccggcggagacgggagtcgtcgacgtcacttgttctcctcttcctctccaacctccggctcgactgatctcttcggaatggtggggagcaggggtagcgcggccgcctctgccctcagcagctgcaggacgttgaagaactagagtatcacgcaggtgcaggggtcgtggaaacacctcgcagcgcgttgaaagtcaccgcacagccgaacgacgtactactaggaactgcaacttcttgatctcatagtgcgtccacgtccccagcacctttgtggagcgtcgcgcaactttcagtggcgtgtcggcttgctgcatgatgatccttattacgtagttctgtccccgcagaccgtgctcaagagctccaagttggcccccgtgcaccctctagcagagcaggtgaaaataataacacataacgggagtaatgcatcaagacaggggcgtctggcacgagttctcgaggttcaaccgggggcacgtgggagatcgtctcgtccacttttattattgtgtattgccctcggccggcggttaccaggtcgacggatatgacggcagggtcctactaccatgtggatcggccattccggtccctgagtttcaggctttgagcgagagcgccccggccgccaatggtccagctgcctatactgccgtcccaggatgatggtacacctagccggtaaggccagggactcaaagtccgaaactcgctctcgcggactatggtgtacaacgaaagggagttcgtcaacaggaaactataccatattgccgttcacggaccctcgctgaacaccgacgaggagaactacgagaaagtgataccacatgttgctttccctcaagcagttgtcctttgatatggtataacggcaagtgcctgggagcgacttgtggctgctcctcttgatgctctttctcagagctgaaagaactgacgccgagtacgtgttcgacgtagataaaaaatgctgcgtcaagagagaggaagcgtcgggtttggtgttggtgggagagctagtctcgactttcttgactgcggctcatgcacaagctgcatctattttttacgacgcagttctctctccttcgcagcccaaaccacaaccaccctctcgaaaccaaccccccgttccatgaattcgcctacgaagggctgaagatcaggccgtcggcaccatataagactacagtagtaggagtctttggggttccgggattggttggggggcaaggtacttaagcggatgcttcccgacttctagtccggcagccgtggtatattctgatgtcatcatcctcagaaaccccaaggcccttcaggcaagtctgctattattaagagcctcgtgaccaaacacgatctggtcaccagcggcaagaaggagaactgccaggaaatagttaacgacgtgaagaagtccgttcagacgataataattctcggagcactggtttgtgctagaccagtggtcgccgttcttcctcttgacggtcctttatcaattgctgcacttctagcaccgcgggaaggggacaagtagggaaaacagtgactccatcctgctaaacgggtgtcgtcgtgccgtggacatcctatatgtggacgaggctttcgctcgtggcgcccttcccctgttcatcccttttgtcactgaggtaggacgatttgcccacagcagcacggcacctgtaggatatacacctgctccgaaagcgttgccatcccggtactctgctggccctaattgctcttgttaaacctcggagcaaagtggtgttatgcggagaccccaagcaatgcggattcttcaatatgaacggtagggccatgagacgaccgggattaacgagaacaatttggagcctcgtttcaccacaatacgcctctggggttcgttacgcctaagaagttatacatgcagcttaaggtgaacttcaaccacaacatctgcactgaagtatgtcataaaagtatatccagacgttgcacgcgtccagtcacggccatcgtgtctatacgtcgaattccacttgaagttggtgttgtagacgtgacttcatacagtattttcatataggtctgcaacgtgcgcaggtcagtgccggtagcacagatcgttgcactacggaggcaagatgcgcacgaccaacccgtgcaacaaacccataatcatagacaccacaggacagaccaagcccaagccaggagacatcgtgcaacgtgatgcctccgttctacgcgtgctggttgggcacgttgtttgggtattagtatctgtggtgtcctgtctggttcgggttcggtcctctgtagcagttaacatgcttccgaggctgggcaaagcagctgcagttggactaccgtggacacgaagtcatgacagcagcagcatctcagggcctcacccgcaaagggcaattgtacgaaggctccgacccgtttcgtcgacgtcaacctgatggcacctgtgcttcagtactgtcgtcgtcgtagagtcccggagtgggcgtttcccgtatacgccgtaaggcagaaggtgaatgaaaatcccttgtatgcccctgcgtcggagcacgtgaatgtactgctgacgcgcactgaggataggctggtgtcatatgcggcattccgtcttccacttacttttagggaacatacggggacgcagcctcgtgcacttacatgacgactgcgcgtgactcctatccgaccacaggaaaacgctggccggcgatccctggattaaggtcctatcaaacattccacagggtaactttacggccacattggaagaatggcaagaagaacacgacaaccttttgcgaccggccgctagggacctaattccaggatagtttgtaaggtgtcccattgaaatgccggtgtaaccttcttaccgttcttcttgtgctgttaataatgaaggtgattgaaggaccggctgcgcctgtggacgcgttccagaacaaagcgaacgtgtgttgggcgaaaagcctggtgcctgtcctggacactttattacttccactaacttcctggccgacgcggacacctgcgcaaggtcttgtttcgcttgcacacaacccgcttttcggaccacggacaggacctgtgagccggaatcagattgacagcagaggagtggagcaccataattacagcatttaaggaggacagagcttactctccagtggtggccttgaatgaaatttgcacggccttagtctaactgtcgtctcctcacctcgtggtattaatgtcgtaaattcctcctgtctcgaatgagaggtcaccaccggaacttactttaaacgtccaagtactatggagttgacctggacagtggcctgttttctgccccgaaggtgtccctgtattacgagaacaaccactgggataacagacctggtggaagggttcatgatacctcaactggacctgtcaccggacaaaagacggggcttccacagggacataatgctcttgttggtgaccctattgtctggaccaccttcgatgtatggattcaatgccgcaacagctgccaggctggaagctagacataccttcctgaaggggcagtggcatacgggcaagcaggcagttatcgcagaactacatacctaagttacggcgttgtcgacggtccgaccttcgatctgtatggaaggacttccccgtcaccgtatgcccgttcgtccgtcaatagcgtcttagaaaaatccaaccgctttctgtgctggacaatgtaattcctatcaaccgcaggctgccgcacgccctggtggctgagtacaagacggttaaaggcagtatctttttaggttggcgaaagacacgacctgttacattaaggatagttggcgtccgacggcgtgcgggaccaccgactcatgttctgccaatttccgtcatgggttgagtggctggtcaataaagtaagagggtaccacgtcctgctggtgagtgagtacaacctggctttgcctcgacgcgacgtcacttggttgtcacccccaactcaccgaccagttatttcattctcccatggtgcaggacgaccactcactcatgttggaccgaaacggagctgcgctgcagtgaaccaacagtgggctgaatgtcacaggcgccgataggtgctacgacctaagtttaggactgccggctgacgccggcaggttcgacttggtctttgtgaacattcacacggaacgacttacagtgtccgcggctatccacgatgctggattcaaatcctgacggccgactgcggccgtccaagctgaaccagaaacacttgtaagtgtgccttttcagaatccaccactaccagcagtgtgtcgaccacgccatgaagctgcagatgcttgggggagatgcgctacgactgctaaaacccggcggcatcttgaaagtcttaggtggtgatggtcgtcacacagctggtgcggtacttcgacgtctacgaaccccctctacgcgatgctgacgattttgggccgccgtagaacttgagagcttacggatacgccgataaaatcagcgaagccgttgtttcctccttaagcagaaagttctcgtctgcaagagtgttgcgcccggattgtgtcacactctcgaatgcctatgcggctattttagtcgcttcggcaacaaaggaggaattcgtctttcaagagcagacgttctcacaacgcgggcctaacacagtgcagcaatacagaagtgttcttgctgttctccaactttgacaacggaaagagaccctctacgctacaccagatgaataccaagctgagtgccgtgtatgccgtcgttatgtcttcacaagaacgacaagaggttgaaactgttgcctttctctgggagatgcgatgtggtctacttatggttcgactcacggcacatacggggagaagccatgcacacggccgggtgtgcaccatcctacagagttaagagagcagacatagccacgtgcacagaagcggctgtggttaacgcagctaacgcctcttcggtacgtgtgccggcccacacgtggtaggatgtctcaattctctcgtctgtatcggtgcacgtgtcttcgccgacaccaattgcgtcgattgccccgtggaactgtaggggatggcgtatgcagggccgtggcgaagaaatggccgtcagcctttaagggagcagcaacaccagtgggcacaattaaaacagtgggcaccttgacatcccctaccgcatacgtcccggcaccgcttctttaccggcagtcggaaattccctcgtcgttgtggtcacccgtgttaattttgtcacatgtgcggctcgtaccccgtcatccacgctgtagcgcctaatttctctgccacgactgaagcggaaggggaccgcgaattggccgctgtctaccgggcagtacacgccgagcatggggcagtaggtgcgacatcgcggattaaagagacggtgctgacttcgccttcccctggcgcttaaccggcgacagatggcccgtgtggccgccgaagtaaacagactgtcactgagcagcgtagccatcccgctgctgtccacaggagtgttcagcggcggaagagataggctgcagcaatccccaccggcggcttcatttgtctgacagtgactcgtcgcatcggtagggcgacgacaggtgtcctcacaagtcgccgccttctctatccgacgtcgttagggtcaaccatctattcacagcaatggacgccacggacgctgacgtgaccatctactgcagagacaaaagttgggagaagaaaatccaggaagccattgacatagttggtagataagtgtcgttacctgcggtgcctgcgactgcactggtagatgacgtctctgttttcaaccctcttcttttaggtccttcggtaactgtagaggacggctgtggagttgctcaatgatgacgtggagctgaccacagacttggtgagagtgcacccggacagcagcctggtgggtcgtaagggctacagtctcctgccgacacctcaacgagttactactgcacctcgactggtgtctgaaccactctcacgtgggcctgtcgtcggaccacccagcattcccgatgtcaaccactgacgggtcgctgtactcgtactttgaaggtacgaaattcaaccaggctgctattgatatggcagagatactgacgttgtggcccagactgcaagtggtgactgcccagcgacatgagcatgaaacttccatgctttaagttggtccgacgataactataccgtctctatgactgcaacaccgggtctgacgttcaggcaaacgaacagatatgcctatacgcgctgggcgaaacaatggacaacatcagatccaaatgtccggtgaacgattccgattcatcaacacctcccagtccgtttgcttgtctatacggatatgcgcgacccgctttgttacctgttgtagtctaggtttacaggccacttgctaaggctaagtagttgtggagggtcgacagtgccctgcctgtgccgctacgcaatgacagcagaacggatcgcccgccttaggtcacaccaagttaaaagcatggtggtttgctcatcttttcccctgtcacgggacggacacggcgatgcgttactgtcgtcttgcctagcgggcggaatccagtgtggttcaattttcgtaccaccaaacgagtagaaaagggctcccgaaataccatgtagatggggtgcagaaggtaaagtgcgagaaggttctcctgttcgacccgacggtaccttcagtggttagtccgcggaagtatggagggctttatggtacatctaccccacgtcttccatttcacgctcttccaagaggacaagctgggctgccatggaagtcaccaatcaggcgccttcatacccgcatctacgacggaccactcagatcggtcgttacgagggtttgacttggactggaccaccgactcgtcttccactgccagcgataccatgtcgctaccggcgtagatgctgcctggtgagtctagccagcaatgctcccaaactgaacctgacctggtggctgagcagaaggtgacggtcgctatggtacagcgatggcagtttgcagtcgtgtgacatcgactcgatctacgagccaatggctcccatagtagtgacggctgacgtacaccctgaacccgcaggcatcgcggacctggtcaaacgtcagcacactgtagctgagctagatgctcggttaccgagggtatcatcactgccgactgcatgtgggacttgggcgtccgtagcgcctggacgcggcagatgtgcaccctgaacccgcagaccatgtggacctggagaacccgattcctccaccgcgcccgaagagagctgcataccttgcctcccgcgcggcgccgtctacacgtgggacttgggcgtctggtacacctggacctcttgggctaaggaggtggcgcgggcttctctcgacgtatggaacggagggcgcgcccggagcgaccggtgccggcgccgagaaagccgacgcctgccccaaggactgcgtttaggaacaagctgcctttgacgttcggcgactttgacgagcacgagcctcgctggccacggccgcggctctttcggctgcggacggggttcctgacgcaaatccttgttcgacggaaactgcaagccgctgaaactgctcgtgctggtcgatgcgttggcctccgggattactttcggagacttcgacgacgtcctgcgactaggccgcgcgggtgcatatattttctcctcggacactggcagcccagctacgcaaccggaggccctaatgaaagcctctgaagctgctgcaggacgctgatccggcgcgcccacgtatataaaagaggagcctgtgaccgtcgggacatttacaacaaaaatccgttaggcagcacaatctccagtgcgcacaactggatgcggtccaggaggagaaaatgtacccgccaaaattggatactgcctgtaaatgttgtttttaggcaatccgtcgtgttagaggtcacgcgtgttgacctacgccaggtcctcctcttttacatgggcggttttaacctatgacagagggagaagctgttgctgctgaaaatgcagatgcacccatcggaggctaataagagtcgataccagtctcgcaaagtggagaacatgaaagccacggttctccctcttcgacaacgacgacttttacgtctacgtgggtagcctccgattattctcagctatggtcagagcgtttcacctcttgtactttcggtgccaggtggacaggctcacatcgggggccagattgtacacgggagcggacgtaggccgcataccaacatacgcggttcggtacccccgccccgtgtactcccctccacctgtccgagtgtagcccccggtctaacatgtgccctcgcctgcatccggcgtatggttgtatgcgccaagccatgggggcggggcacatgaggggaaccgtgatcgaaagattctcaagccccgatgtagcaatcgcagcgtgcaacgaatacctatccagaaattacccaacagtggcgtcgtaccagataacagtggcactagctttctaagagttcggggctacatcgttagcgtcgcacgttgcttatggataggtctttaatgggttgtcaccgcagcatggtctattgtcatgaatacgacgcatacttggacatggttgacgggtcggatagttgcttggacagagcgacattctgcccggcgaagctccggtgctacccgaaacatcatacttatgctgcgtatgaacctgtaccaactgcccagcctatcaacgaacctgtctcgctgtaagacgggccgcttcgaggccacgatgggctttgtagttgcgtaccaccagccgactgtacgcagtgccgtcccgtcaccctttcagaacacactacagaacgtgctagcggctgccaccaagagaaactgcaacgtcacgcatggtggtcggctgacatgcgtcacggcagggcagtgggaaagtcttgtgtgatgtcttgcacgatcgccgacggtggttctctttgacgttgcagacgcaaatgcgagaactacccaccatggactcggcagtgttcaacgtggagtgcttcaagcgctatgcctgctccggagaatattgggaagaatatgctatgcgtttacgctcttgatgggtggtacctgagccgtcacaagttgcacctcacgaagttcgcgatacggacgaggcctcttataacccttcttatacgataacaacctatccggataaccactgagaacatcactacctatgtgaccaaattgaaaggcccgaaagctgctgccttgttcgctaagacccacaacttggtttgttggataggcctattggtgactcttgtagtgatggatacactggtttaactttccgggctttcgacgacggaacaagcgattctgggtgttgaaccatccgctgcaggaggttcccatggacagattcacggtcgacatgaaacgagatgtcaaagtcactccagggacgaaacacacagaggaaagacccaaagtcaggcgacgtcctccaagggtacctgtctaagtgccagctgtactttgctctacagtttcagtgaggtccctgctttgtgtgtctcctttctgggtttcagcaggtaattcaagcagcggagccattggcgaccgcttacctgtgcggcatccacagggaattagtaaggagactaaatgctgtgttacgccctaacgtgcgtccattaagttcgtcgcctcggtaaccgctggcgaatggacacgccgtaggtgtcccttaatcattcctctgatttacgacacaatgcgggattgcacgacacattgtttgatatgtcggccgaagactttgacgcgatcatcgcctctcacttccacccaggagacccggttctagagacggacattgcatcattcgatgtgtaacaaactatacagccggcttctgaaactgcgctagtagcggagagtgaaggtgggtcctctgggccaagatctctgcctgtaacgtagtaagctcaaaagccaggacgactccttggctcttacaggtttaatgatcctcgaagatctaggggtggatcagtacctgctggacttgatcgaggcagcctttggggttttcggtcctgctgaggaaccgagaatgtccaaattactaggagcttctagatccccacctagtcatggacgacctgaactagctccgtcggaaacccgaaatatccagctgtcacctaccaactggcacgcgcttcaagttcggagctatgatgaaatcgggcatgtttctgactttgtttattaacactgttttgactttataggtcgacagtggatggttgaccgtgcgcgaagttcaagcctcgatactactttagcccgtacaaagactgaaacaaataattgtgacaaaactacatcaccatagcaagcagggtactggagcagagactcactgactccgcctgtgcggccttcatcggcgacgacaacatcgttcacggagtgatctccgatgtagtggtatcgttcgtcccatgacctcgtctctgagtgactgaggcggacacgccggaagtagccgctgctgttgtagcaagtgcctcactagaggctcaagctgatggcggagaggtgcgcgtcgtgggtcaacatggaggtgaagatcattgacgctgtcatgggcgaaaaacccccatatttttgtgggggattcgttcgactaccgcctctccacgcgcagcacccagttgtacctccacttctagtaactgcgacagtacccgctttttgggggtataaaaacaccccctaagatagtttttgacagcgtcacacagaccgcctgccgtgtttcagacccacttaagcgcctgttcaagttgggtaagccgctaacagctgaagacaagcaggtatcaaaaactgtcgcagtgtgtctggcggacggcacaaagtctgggtgaattcgcggacaagttcaacccattcggcgattgtcgacttctgttcgtccacgaagacaggcgacgagcactgagtgacgaggttagcaagtggttccggacaggcttgggggccgaactggaggtggcactaacatctaggtatgaggttgcttctgtccgctgctcgtgactcactgctccaatcgttcaccaaggcctgtccgaacccccggcttgacctccaccgtgattgtagatccatactccaagagggctgcaaaagtatcctcatagccatggccaccttggcgagggacattaaggcgtttaagaaattgagaggacctgttatacacctctacggcggttctcccgacgttttcataggagtatcggtaccggtggaaccgctccctgtaattccgcaaattctttaactctcctggacaatatgtggagatgccgccacctagattggtgcgttaatacacagaattctgattggatcccggtccgaagcgcgctttcccgggaactcgagttcactagtcgatcccgcggccgctttggatctaaccacgcaattatgtgtcttaagactaacctagggccaggcttcgcgcgaaagggcccttgagctcaagtgatcagctagggcgccggcgaaacgaacctaggcaagcatgcgggcccagtgggtaattaattgaattacatccctacgcaaacgttttacggccgccggtggcgcccgcgcccggcggcccggcttggatccgttcgtacgcccgggtcacccattaattaacttaatgtagggatgcgtttgcaaaatgccggcggccaccgcgggcgcgggccgccgggctccttggccgttgcaggccactccggtggctcccgtcgtccccgacttccaggcccagcagatgcagcaactcatcagcgccgtaaatgcgctgacaatgaggaaccggcaacgtccggtgaggccaccgagggcagcaggggctgaaggtccgggtcgtctacgtcgttgagtagtcgcggcatttacgcgactgttacagacagaacgcaattgctcctgctaggagcttaattcgacgaataattggatttttattttattttgcaattggtttttaatatttccaaaaaaaaaaaatctgtcttgcgttaacgaggacgatcctcgaattaagctgcttattaacctaaaaataaaataaaacgttaaccaaaaattataaaggttttttttttttaaaaaaaaaaaaaaaaaaaaaaaaaaaaaaaaaaaaaaaaaaaaaaaaaaaaaaaaaactagaaatcgatcggcagtgcgccttccaggagaactagcgcttttttttttttttttttttttttttttttttttttttttttttttttttttttttttgatctttagctagccgtcacgcggaaggtcctcttgatcgcgggccgcatcgataagcttgtcgactcgaagatccagacatgataagatacattgatgagtttggacaaaccacaactagaatgcagtgaaaaaaatgcttccggcgtagctattcgaacagctgagcttctaggtctgtactattctatgtaactactcaaacctgtttggtgttgatcttacgtcactttttttacgaatatttgtgaaatttgtgatgctattgctttatttgtaaccattataagctgcaataaacaagttaacaacaacaattgcattcattttatgtttcaggttataaacactttaaacactacgataacgaaataaacattggtaatattcgacgttatttgttcaattgttgttgttaacgtaagtaaaatacaaagtccaacagggggaggtgtgggaggttttttcggatctgggcgtggttaagggtgggaaagaatatataaggtgggggtcttatgtagttttgtatctgttttgcagtccccctccacaccctccaaaaaagcctagacccgcaccaattcccaccctttcttatatattccacccccagaatacatcaaaacatagacaaaacgtgcagccgccgccgccatgagcaccaactcgtttgatggaagcattgtgagctcatatttgacaacgcgcatgcccccatgggccggggtgcgtcagaatgcgtcggcggcggcggtactcgtggttgagcaaactaccttcgtaacactcgagtataaactgttgcgcgtacgggggtacccggccccacgcagtcttactgatgggctccagcattgatggtcgccccgtcctgcccgcaaactctactaccttgacctacgagaccgtgtctggaacgccgttggagactgcagcctcactacccgaggtcgtaactaccagcggggcaggacgggcgtttgagatgatggaactggatgctctggcacagaccttgcggcaacctctgacgtcggagcgccgccgcttcagccgctgcagccaccgcccgcgggattgtgactgactttgctttcctgagcccgcttgcaagcagtgcagcttcccgttcatccgccgcggcggcgaagtcggcgacgtcggtggcgggcgccctaacactgactgaaacgaaaggactcgggcgaacgttcgtcacgtcgaagggcaagtaggcggcgcgatgacaagttgacggctcttttggcacaattggattctttgacccgggaacttaatgtcgtttctcagcagctgttggatctgcgccagcaggtttgcgctactgttcaactgccgagaaaaccgtgttaacctaagaaactgggcccttgaattacagcaaagagtcgtcgacaacctagacgcggtcgtccaaactgccctgaaggcttcctcccctcccaatgcggtttaaaacataaataaaaaaccagactctgtttggatttggatcaagcaagtgtcttgctgtctttagacgggacttccgaaggaggggagggttacgccaaattttgtatttattttttggtctgagacaaacctaaacctagttcgttcacagaacgacagaaattttaggggttttgcgcgcgcggtaggcccgggaccagcggtctcggtcgttgagggtcctgtgtattttttccaggacgtggtaaaggtgactctggatgaaatccccaaaacgcgcgcgccatccgggccctggtcgccagagccagcaactcccaggacacataaaaaaggtcctgcaccatttccactgagacctacttcagatacatgggcataagcccgtctctggggtggaggtagcaccactgcagagcttcatgctgcggggtggtgttgtagatgatccagtcgtagcaggaagtctatgtacccgtattcgggcagagaccccacctccatcgtggtgacgtctcgaagtacgacgccccaccacaacatctactaggtcagcatcgtccagcgctgggcgtggtgcctaaaaatgtctttcagtagcaagctgattgccaggggcaggcccttggtgtaagtgtttacaaagcggttaagctgggatggtcgcgacccgcaccacggatttttacagaaagtcatcgttcgactaacggtccccgtccgggaaccacattcacaaatgtttcgccaattcgaccctaccgtgcatacgtggggatatgagatgcatcttggactgtatttttaggttggctatgttcccagccatatccctccggggattcatgttgtgcagaaccacccacgtatgcacccctatactctacgtagaacctgacataaaaatccaaccgatacaagggtcggtatagggaggcccctaagtacaacacgtcttggtggagcacagtgtatccggtgcacttgggaaatttgtcatgtagcttagaaggaaatgcgtggaagaacttggagacgcccttgtgacctccaagattttccatcgtgtcacataggccacgtgaaccctttaaacagtacatcgaatcttcctttacgcaccttcttgaacctctgcgggaacactggaggttctaaaaggttgcattcgtccataatgatggcaatgggcccacgggcggcggcctgggcgaagatatttctgggatcactaacgtcatagttgtgttccaggatgagatcacgtaagcaggtattactaccgttacccgggtgcccgccgccggacccgcttctataaagaccctagtgattgcagtatcaacacaaggtcctactctaggtcataggccatttttacaaagcgcgggcggagggtgccagactgcggtataatggttccatccggcccaggggcgtagttaccctcacagatttgcattcagtatccggtaaaaatgtttcgcgcccgcctcccacggtctgacgccatattaccaaggtaggccgggtccccgcatcaatgggagtgtctaaacgtaatcccacgctttgagttcagatggggggatcatgtctacctgcggggcgatgaagaaaacggtttccggggtaggggagatcagctgggaagaaagcaggtagggtgcgaaactcaagtctacccccctagtacagatggacgccccgctacttcttttgccaaaggccccatcccctctagtcgacccttctttcgtccatcctgagcagctgcgacttaccgcagccggtgggcccgtaaatcacacctattaccgggtgcaactggtagttaagagagctgcagctgccgtcatccctaggactcgtcgacgctgaatggcgtcggccacccgggcatttagtgtggataatggcccacgttgaccatcaattctctcgacgtcgacggcagtagggagagcaggggggccacttcgttaagcatgtccctgactcgcatgttttccctgaccaaatccgccagaaggcgctcgccgcccagcgatagcagttcttgcctcgtccccccggtgaagcaattcgtacagggactgagcgtacaaaagggactggtttaggcggtcttccgcgagcggcgggtcgctatcgtcaagaacgaaggaagcaaagtttttcaacggtttgagaccgtccgccgtaggcatgcttttgagcgtttgaccaagcagttccaggcggtcccacagctcggtcacctttccttcgtttcaaaaagttgccaaactctggcaggcggcatccgtacgaaaactcgcaaactggttcgtcaaggtccgccagggtgtcgagccagtggagctctacggcatctcgatccagcatatctcctcgtttcgcgggttggggcggctttcgctgtacggcagtagtcggtgctcgtccagacgggccagggtccgagatgccgtagagctaggtcgtatagaggagcaaagcgcccaaccccgccgaaagcgacatgccgtcatcagccacgagcaggtctgcccggtcccagatgtctttccacgggcgcagggtcctcgtcagcgtagtctgggtcacggtgaaggggtgcgctccgggctgcgcgctggccagggtgcgcttgaggctggtacagaaaggtgcccgcgtcccaggagcagtcgcatcagacccagtgccacttccccacgcgaggcccgacgcgcgaccggtcccacgcgaactccgacctcctgctggtgctgaagcgctgccggtcttcgccctgcgcgtcggccaggtagcatttgaccatggtgtcatagtccagcccctccgcggcgtggcccttaggacgaccacgacttcgcgacggccagaagcgggacgcgcagccggtccatcgtaaactggtaccacagtatcaggtcggggaggcgccgcaccgggaaggcgcgcagcttgcccttggaggaggcgccgcacgagggagacttttgagggcgtagagcttgggcgcgagaaataccgattccggggagtaggcatccgccgcgcgtcgaacgggaacctcctccgcggcgtgctccctctgaaaactcccgcatctcgaacccgcgctctttatggctaaggcccctcatccgtaggccgccgcaggccccgcagacggtctcgcattccacgagccaggtgagctctggccgttcggggtcaaaaaccaggtttcccccatgctttttgatgcgtttgcggcgtccggggcgtctgccagagcgtaaggtgctcggtccactcgagaccggcaagccccagtttttggtccaaagggggtacgaaaaactacgcaaacttacctctggtttccatgagccggtgtccacgctcggtgacgaaaaggctgtccgtgtccccgtatacagacttgagaggcctgtcctcgagcggtgttgaatggagaccaaaggtactcggccacaggtgcgagccactgcttttccgacaggcacaggggcatatgtctgaactctccggacaggagctcgccacaaccgcggtcctcctcgtatagaaactcggaccactctgagacaaaggctcgcgtccaggccagcacgaaggaggctaagtgggaggggtagcggtcgttgtggcgccaggaggagcatatctttgagcctggtgagactctgtttccgagcgcaggtccggtcgtgcttcctccgattcaccctccccatcgccagcaacaccactagggggtccactcgctccagggtgtgaagacacatgtcgccctcttcggcatcaaggaaggtgattggtttgtaggtgtaggccacgtgaccgggggtgatcccccaggtgagcgaggtcccacacttctgtgtacagcgggagaagccgtagttccttccactaaccaaacatccacatccggtgcactggccctgttcctgaaggggggctataaaagggggtgggggcgcgttcgtcctcactctcttccgcatcgctgtctgcgagggccagctgttggggtgagtactccacaaggacttccccccgatattttcccccacccccgcgcaagcaggagtgagagaaggcgtagcgacagacgctcccggtcgacaaccccactcatgaggctctgaaaagcgggcatgacttctgcgctaagattgtcagtttccaaaaacgaggaggatttgatattcacctggcccgcggtgatgcctttgagggtgggagacttttcgcccgtactgaagacgcgattctaacagtcaaaggtttttgctcctcctaaactataagtggaccgggcgccactacggaaactcccaccccgcatccatctggtcagaaaagacaatctttttgttgtcaagcttggtggcaaacgacccgtagagggcgttggacagcaacttggcgatggagcgcagggcgtaggtagaccagtcttttctgttagaaaaacaacagttcgaaccaccgtttgctgggcatctcccgcaacctgtcgttgaaccgctacctcgcgtcggtttggtttttgtcgcgatcggcgcgctccttggccgcgatgtttagctgcacgtattcgcgcgcaacgcaccgccattcgggaaagacggtggtgcgcccaaaccaaaaacagcgctagccgcgcgaggaaccggcgctacaaatcgacgtgcataagcgcgcgttgcgtggcggtaagccctttctgccaccacgcgtcgtcgggcaccaggtgcacgcgccaaccgcggttgtgcagggtgacaaggtcaacgctggtggctacctctccgcgtaggcgctcgttggtccagcagaagcagcccgtggtccacgtgcgcggttggcgccaacacgtcccactgttccagttgcgaccaccgatggagaggcgcatccgcgagcaaccaggtcgtctggcggccgcccttgcgcgagcagaatggcggtagggggtctagctgcgtctcgtccggggggtctgcgtccacggtaaagaccccgggcagcaggcgcgcccgccggcgggaacgcgctcgtcttaccgccatcccccagatcgacgcagagcaggccccccagacgcaggtgccatttctggggcccgtcgtccgcgcggtcgaagtagtctatcttgcatccttgcaagtctagcgcctgctgccatgcgcgggcggcaagcgcgcgctcgtatgggttgagtgggggaccccatggccagcttcatcagatagaacgtaggaacgttcagatcgcggacgacggtacgcgcccgccgttcgcgcgcgagcatacccaactcaccccctggggtaccgatggggtgggtgagcgcggaggcgtacatgccgcaaatgtcgtaaacgtagaggggctctctgagtattccaagatatgtagggtagcatcttccaccgctaccccacccactcgcgcctccgcatgtacggcgtttacagcatttgcatctccccgagagactcataaggttctatacatcccatcgtagaaggtggcgggatgctggcgcgcacgtaatcgtatagttcgtgcgagggagcgaggaggtcgggaccgaggttgctacgggcgggctgctctgctcggaagactatctgcctacgaccgcgcgtgcattagcatatcaagcacgctccctcgctcctccagccctggctccaacgatgcccgcccgacgagacgagccttctgatagaccctgaagatggcatgtgagttggatgatatggttggacgctggaagacgttgaagctggcgtctgtgagacctaccgcgtcacgcacgaaggaggcgtagggacttctaccgtacactcaacctactataccaacctgcgaccttctgcaacttcgaccgcagacactctggatggcgcagtgcgtgcttcctccgcatcgagtcgcgcagcttgttgaccagctcggcggtgacctgcacgtctagggcgcagtagtccagggtttccttgatgatgtcatacttatcctgtcccttttctcagcgcgtcgaacaactggtcgagccgccactggacgtgcagatcccgcgtcatcaggtcccaaaggaactactacagtatgaataggacagggaaaattttccacagctcgcggttgaggacaaactcttcgcggtctttccagtactcttggatcggaaacccgtcggcctccgaacggtaagagcctagcatgtaaaaaggtgtcgagcgccaactcctgtttgagaagcgccagaaaggtcatgagaacctagcctttgggcagccggaggcttgccattctcggatcgtacatgaactggttgacggcctggtaggcgcagcatcccttttctacgggtagcgcgtatgcctgcgcggccttcatgaccagcatgaagggcacgagctgcttccttgaccaactgccggaccatccgcgtcgtagggaaaagatgcccatcgcgcatacggacgcgccggaagtactggtcgtacttcccgtgctcgacgaagccaaaggcccccatccaagtataggtctctacatcgtaggtgacaaagagacgctcggtgcgaggatgcgagccgatcgggaagaactggatctcccgccggtttccgggggtaggttcatatccagagatgtagcatccactgtttctctgcgagccacgctcctacgctcggctagcccttcttgacctagagggcggaccaattggaggagtggctattgatgtggtgaaagtagaagtccctgcgacgggccgaacactcgtgctggcttttgtaaaaacgtgcgcagtactggcatggttaacctcctcaccgataactacaccactttcatcttcagggacgctgcccggcttgtgagcacgaccgaaaacatttttgcacgcgtcatgaccgtgcggtgcacgggctgtacatcctgcacgaggttgacctgacgaccgcgcacaaggaagcagagtgggaatttgagcccctcgcctggcgggtttggctggcgccacgtgcccgacatgtaggacgtgctccaactggactgctggcgcgtgttccttcgtctcacccttaaactcggggagcggaccgcccaaaccgacctggtcttctacttcggctgcttgtccttgaccgtctggctgctcgaggggagttacggtggatcggaccaccacgccgcgcgagcccaaagtccagatgtaccagaagatgaagccgacgaacaggaactggcagaccgacgagctcccctcaatgccacctagcctggtggtgcggcgcgctcgggtttcaggtctacaccgcgcgcggcggtcggagcttgatgacaacatcgcgcagatgggagctgtccatggtctggagctcccgcggcgtcaggtcaggcgggagctcctgcagggcgcgcgccgccagcctcgaactactgttgtagcgcgtctaccctcgacaggtaccagacctcgagggcgccgcagtccagtccgccctcgaggacgtcgtttacctcgcatagacgggtcagggcgcgggctagatccaggtgatacctaatttccaggggctggttggtggcggcgtcgatggcttgcaagaggccgcaaatggagcgtatctgcccagtcccgcgcccgatctaggtccactatggattaaaggtccccgaccaaccaccgccgcagctaccgaacgttctccggccatccccgcggcgcgactacggtaccgcgcggcgggcggtgggccgcgggggtgtccttggatgatgcatctaaaagcggtgacgcgggcgagcccccgggtaggggcgccgcgctgatgccatggcgcgccgcccgccacccggcgcccccacaggaacctactacgtagattttcgccactgcgcccgctcgggggccaggtagggggggctccggacccgccgggagagggggcaggggcacgtcggcgccgcgcgcgggcaggagctggtgctgcgcgcgtaggttgctggcgaactccatcccccccgaggcctgggcggccctctcccccgtccccgtgcagccgcggcgcgcgcccgtcctcgaccacgacgcgcgcatccaacgaccgcttggcgacgacgcggcggttgatctcctgaatctggcgcctctgcgtgaagacgacgggcccggtgagcttgagcctgaaagagagttcgacagaatcaatttcgctgctgcgccgccaactagaggacttagaccgcggagacgcacttctgctgcccgggccactcgaactcggactttctctcaagctgtcttagttaaacggtgtcgttgacggcggcctggcgcaaaatctcctgcacgtctcctgagttgtcttgataggcgatctcggccatgaactgctcgatctcttcctcctggccacagcaactgccgccggaccgcgttttagaggacgtgcagaggactcaacagaactatccgctagagccggtacttgacgagctagagaaggaggacgagatctccgcgtccggctcgctccacggtggcggcgaggtcgttggaaatgcgggccatgagctgcgagaaggcgttgaggcctccctcgttccagacgctctagaggcgcaggccgagcgaggtgccaccgccgctccagcaacctttacgcccggtactcgacgctcttccgcaactccggagggagcaaggtctgccggctgtagaccacgcccccttcggcatcgcgggcgcgcatgaccacctgcgcgagattgagctccacgtgccgggcgaagacggcgtagtttcgcaggcgccgacatctggtgcgggggaagccgtagcgcccgcgcgtactggtggacgcgctctaactcgaggtgcacggcccgcttctgccgcatcaaagcgtccggctgaaagaggtagttgagggtggtggcggtgtgttctgccacgaagaagtacataacccagcgtcgcaacgtggattcgttgcaattgttgtgtaggtacgactttctccatcaactcccaccaccgccacacaagacggtgcttcttcatgtattgggtcgcagcgttgcacctaagcaacgttaacaacacatccatctccgccgccgagggacctgagcgagtccgcatcgaccggatcggaaaacctctcgagaaaggcgtctaaccagtcacagtcgcaaggtaggctgagcacgaggcggcggctccctggactcgctcaggcgtagctggcctagccttttggagagctctttccgcagattggtcagtgtcagcgttccatccgactcgtgcgtggcgggcggcagcgggcggcggtcggggttgtttctggcggaggtgctgctgatgatgtaattaaagtaggcggtcttgagacggcggatggtcgacgcaccgcccgccgtcgcccgccgccagccccaacaaagaccgcctccacgacgactactacattaatttcatccgccagaactctgccgcctaccagctgagaagcaccatgtccttgggtccggcctgctgaatgcgcaggcggtcggccatgccccaggcttcgttttgacatcggcgcaggtctttgtagtagtctttcttcgtggtacaggaacccaggccggacgacttacgcgtccgccagccggtacggggtccgaagcaaaactgtagccgcgtccagaaacatcatcagaagcatgagcctttctaccggcacttcttcttctccttcctcttgtcctgcatctcttgcatctatcgctgcggcggcggcggagtttggccgtaggtggcgcgtactcggaaagatggccgtgaagaagaagaggaaggagaacaggacgtagagaacgtagatagcgacgccgccgccgcctcaaaccggcatccaccgcccctcttcctcccatgcgtgtgaccccgaagcccctcatcggctgaagcagggctaggtcggcgacaacgcgctcggctaatatggcctgctgcacctgcgggagaaggagggtacgcacactggggcttcggggagtagccgacttcgtcccgatccagccgctgttgcgcgagccgattataccggacgacgtggacggtgagggtagactggaagtcatccatgtccacaaagcggtggtatgcgcccgtgttgatggtgtaagtgcagttggccataacggaccagttaacggtctcactcccatctgaccttcagtaggtacaggtgtttcgccaccatacgcgggcacaactaccacattcacgtcaaccggtattgcctggtcaattgccagaggtgacccggctgcgagagctcggtgtacctgagacgcgagtaagccctcgagtcaaatacgtagtcgttgcaagtccgcaccaggtactggtatcccacccactgggccgacgctctcgagccacatggactctgcgctcattcgggagctcagtttatgcatcagcaacgttcaggcgtggtccatgaccatagggtgcaaaaagtgcggcggcggctggcggtagaggggccagcgtagggtggccggggctccgggggcgagatcttccaacataaggcgatgatatccgtagatggtttttcacgccgccgccgaccgccatctccccggtcgcatcccaccggccccgaggcccccgctctagaaggttgtattccgctactataggcatctactacctggacatccaggtgatgccggcggcggtggtggaggcgcgcggaaagtcgcggacgcggttccagatgttgcgcagcggcaaaaagtgctccatggatggacctgtaggtccactacggccgccgccaccacctccgcgcgcctttcagcgcctgcgccaaggtctacaacgcgtcgccgtttttcacgaggtacctcgggacgctctggccggtcaggcgcgcgcaatcgttgacgctctagaccgtgcaaaaggagagcctgtaagcgggcactcttccgtggtctggtggataagccctgcgagaccggccagtccgcgcgcgttagcaactgcgagatctggcacgttttcctctcggacattcgcccgtgagaaggcaccagaccacctataattcgcaagggtatcatggcggacgaccggggttcgagccccgtatccggccgtccgccgtgatccatgcggttaccgcccgcgtgtcgaacccaggtgttaagcgttcccatagtaccgcctgctggccccaagctcggggcataggccggcaggcggcactaggtacgccaatggcgggcgcacagcttgggtccactgcgacgtcagacaacgggggagtgctccttttggcttccttccaggcgcggcggctgctgcgctagcttttttggccactggccgcgcgcagcgtaagcacgctgcagtctgttgccccctcacgaggaaaaccgaaggaaggtccgcgccgccgacgacgcgatcgaaaaaaccggtgaccggcgcgcgtcgcattcgggttaggctggaaagcgaaagcattaagtggctcgctccctgtagccggagggttattttccaagggttgagtcgcgggacccccggttcgagtctcggaccaatccgacctttcgctttcgtaattcaccgagcgagggacatcggcctcccaataaaaggttcccaactcagcgccctgggggccaagctcagagcctccggccggactgcggcgaacgggggtttgcctccccgtcatgcaagaccccgcttgcaaattcctccggaaacagggacgagccccttttttgcttttccggccggcctgacgccgcttgcccccaaacggaggggcagtacgttctggggcgaacgtttaaggaggcctttgtccctgctcggggaaaaaacgaaaaggcagatgcatccggtgctgcggcagatgcgcccccctcctcagcagcggcaagagcaagagcagcggcagacatgcagggcaccctcccctcctcctaccggtctacgtaggccacgacgccgtctacgcggggggaggagtcgtcgccgttctcgttctcgtcgccgtctgtacgtcccgtgggaggggaggaggatggccgtcaggaggggcgacatccgcggttgacgcggcagcagatggtgattacgaacccccgcggcgccgggcccggcactacctggacttggaggagggcgagcagtcctccccgctgtaggcgccaactgcgccgtcgtctaccactaatgcttgggggcgccgcggcccgggccgtgatggacctgaacctcctcccgctgggcctggcgcggctaggagcgccctctcctgagcggtacccaagggtgcagctgaagcgtgatacgcgtgaggcgtacgtgccgcggcagaacctgtttcccggaccgcgccgatcctcgcgggagaggactcgccatgggttcccacgtcgacttcgcactatgcgcactccgcatgcacggcgccgtcttggacaaacgcgaccgcgagggagaggagcccgaggagatgcgggatcgaaagttccacgcagggcgcgagctgcggcatggcctgaatcgcgagcggttgctgcgcggcgctggcgctccctctcctcgggctcctctacgccctagctttcaaggtgcgtcccgcgctcgacgccgtaccggacttagcgctcgccaacgacgcgcaggaggactttgagcccgacgcgcgaaccgggattagtcccgcgcgcgcacacgtggcggccgccgacctggtaaccgcatacgagcagacggtgaaccatcctcctgaaactcgggctgcgcgcttggccctaatcagggcgcgcgcgtgtgcaccgccggcggctggaccattggcgtatgctcgtctgccacttggtggagattaactttcaaaaaagctttaacaaccacgtgcgtacgcttgtggcgcgcgaggaggtggctataggactgatgcatctgtgggactttgtaagccctctaattgaaagttttttcgaaattgttggtgcacgcatgcgaacaccgcgcgctcctccaccgatatcctgactacgtagacaccctgaaacattcggcgctggagcaaaacccaaatagcaagccgctcatggcgcagctgttccttatagtgcagcacagcagggacaacgaggcattcagggatgcgctgctaacgcgacctcgttttgggtttatcgttcggcgagtaccgcgtcgacaaggaatatcacgtcgtgtcgtccctgttgctccgtaagtccctacgcgacgattacatagtagagcccgagggccgctggctgctcgatttgataaacatcctgcagagcatagtggtgcaggagcgcagcttgagcctggctgacaaggtggctgtatcatctcgggctcccggcgaccgacgagctaaactatttgtaggacgtctcgtatcaccacgtcctcgcgtcgaactcggaccgactgttccaccgcgccatcaactattccatgcttagcctgggcaagttttacgcccgcaagatataccataccccttacgttcccatagacaaggaggtaaagatcgagggggcggtagttgataaggtacgaatcggacccgttcaaaatgcgggcgttctatatggtatggggaatgcaagggtatctgttcctccatttctagctccccttctacatgcgcatggcgctgaaggtgcttaccttgagcgacgacctgggcgtttatcgcaacgagcgcatccacaaggccgtgagcgtgagccggcggcaagatgtacgcgtaccgcgacttccacgaatggaactcgctgctggacccgcaaatagcgttgctcgcgtaggtgttccggcactcgcactcggccgccggcgagctcagcgaccgcgagctgatgcacagcctgcaaagggccctggctggcacgggcagcggcgatagagaggccgagtcctactttgacgcgggcgccgctcgagtcgctggcgctcgactacgtgtcggacgtttcccgggaccgaccgtgcccgtcgccgctatctctccggctcaggatgaaactgcgcccgcgtgacctgcgctgggccccaagccgacgcgccctggaggcagctggggccggacctgggctggcggtggcacccgcgcgcgctggcaacgtcggcggcgtgactggacgcgacccggggttcggctgcgcgggacctccgtcgaccccggcctggacccgaccgccaccgtgggcgcgcgcgaccgttgcagccgccgcacgaggaatatgacgaggacgatgagtacgagccagaggacggcgagtactaagcggtgatgtttctgatcagatgatgcaagacgcaacggacccggcggtctccttatactgctcctgctactcatgctcggtctcctgccgctcatgattcgccactacaaagactagtctactacgttctgcgttgcctgggccgccagcgggcggcgctgcagagccagccgtccggccttaactccacggacgactggcgccaggtcatggaccgcatcatgtcgctgactgcgcgcaatcctgaccgcccgccgcgacgtctcggtcggcaggccggaattgaggtgcctgctgaccgcggtccagtacctggcgtagtacagcgactgacgcgcgttaggactggcgttccggcagcagccgcaggccaaccggctctccgcaattctggaagcggtggtcccggcgcgcgcaaaccccacgcacgagaaggtgctggcgatcgcgcaaggccgtcgtcggcgtccggttggccgagaggcgttaagaccttcgccaccagggccgcgcgcgtttggggtgcgtgctcttccacgaccgctagctaaacgcgctggccgaaaacagggccatccggcccgacgaggccggcctggtctacgacgcgctgcttcagcgcgtggctcgttacaacagcggcaacgtatttgcgcgaccggcttttgtcccggtaggccgggctgctccggccggaccagatgctgcgcgacgaagtcgcgcaccgagcaatgttgtcgccgttgcagcagaccaacctggaccggctggtgggggatgtgcgcgaggccgtggcgcagcgtgagcgcgcgcagcagcagggcaacctgggctccatggttgcactacgtctggttggacctggccgaccaccccctacacgcgctccggcaccgcgtcgcactcgcgcgcgtcgtcgtcccgttggacccgaggtaccaacgtgataacgccttcctgagtacacagcccgccaacgtgccgcggggacaggaggactacaccaactttgtgagcgcactgcggctaatggtgactgagacaccgcttgcggaaggactcatgtgtcgggcggttgcacggcgcccctgtcctcctgatgtggttgaaacactcgcgtgacgccgattaccactgactctgtggcgaaagtgaggtgtaccagtctgggccagactattttttccagaccagtagacaaggcctgcagaccgtaaacctgagccaggctttcaaaaacttgcagggtttcactccacatggtcagacccggtctgataaaaaaggtctggtcatctgttccggacgtctggcatttggactcggtccgaaagtttttgaacgtcccgctgtggggggtgcgggctcccacaggcgaccgcgcgaccgtgtctagcttgctgacgcccaactcgcgcctgttgctgctgctaatagcgcccttcacgcgacaccccccacgcccgagggtgtccgctggcgcgctggcacagatcgaacgactgcgggttgagcgcggacaacgacgacgattatcgcgggaagtgcgacagtggcagcgtgtcccgggacacatacctaggtcacttgctgacactgtaccgcgaggccataggtcaggcgcatgtggacgagcatactttccaggctgtcaccgtcgcacagggccctgtgtatggatccagtgaacgactgtgacatggcgctccggtatccagtccgcgtacacctgctcgtatgaaaggtccagattacaagtgtcagccgcgcgctggggcaggaggacacgggcagcctggaggcaaccctaaactacctgctgaccaaccggcggcagaagatcccctctctaatgttcacagtcggcgcgcgaccccgtcctcctgtgcccgtcggacctccgttgggatttgatggacgactggttggccgccgtcttctaggggaggttgcacagtttaaacagcgaggaggagcgcattttgcgctacgtgcagcagagcgtgagccttaacctgatgcgcgacggggtaacgcccagcgtggcgcaacgtgtcaaatttgtcgctcctcctcgcgtaaaacgcgatgcacgtcgtctcgcactcggaattggactacgcgctgccccattgcgggtcgcaccgcctggacatgaccgcgcgcaacatggaaccgggcatgtatgcctcaaaccggccgtttatcaaccgcctaatggactacttgcatcgcgcggccgccgtgagacctgtactggcgcgcgttgtaccttggcccgtacatacggagtttggccggcaaatagttggcggattacctgatgaacgtagcgcgccggcggcactaccccgagtatttcaccaatgccatcttgaacccgcactggctaccgccccctggtttctacaccgggggattcgaggtgcccgagggtaacgatggatttggggctcataaagtggttacggtagaacttgggcgtgaccgatggcgggggaccaaagatgtggccccctaagctccacgggctcccattgctacctaacctctgggacgacatagacgacagcgtgttttccccgcaaccgcagaccctgctagagttgcaacagcgcgagcaggcagaggcggcgctgcgaaaggaaggagaccctgctgtatctgctgtcgcacaaaaggggcgttggcgtctgggacgatctcaacgttgtcgcgctcgtccgtctccgccgcgacgctttccttagcttccgcaggccaagcagcttgtccgatctaggcgctgcggccccgcggtcagatgctagtagcccatttccaagcttgatagggtctcttaccagcatcgaaggcgtccggttcgtcgaacaggctagatccgcgacgccggggcgccagtctacgatcatcgggtaaaggttcgaactatcccagagaatggtcgtctcgcaccacccgcccgcgcctgctgggcgaggaggagtacctaaacaactcgctgctgcagccgcagcgcgaaaaaaacctgcctccggcatttcccaagagcgtggtgggcgggcgcggacgacccgctcctcctcatggatttgttgagcgacgacgtcggcgtcgcgctttttttggacggaggccgtaaagggttcaacgggatagagagcctagtggacaagatgagtagatggaagacgtacgcgcaggagcacagggacgtgccaggcccgcgcccgcccacccgtcgtcaagttgccctatctctcggatcacctgttctactcatctaccttctgcatgcgcgtcctcgtgtccctgcacggtccgggcgcgggcgggtgggcagcagttaggcacgaccgtcagcggggtctggtgtgggaggacgatgactcggcagacgacagcagcgtcctggatttgggagggagtggcaacccgtttgcgcacctccgtgctggcagtcgccccagaccacaccctcctgctactgagccgtctgctgtcgtcgcaggacctaaaccctccctcaccgttgggcaaacgcgtggttcgccccaggctggggagaatgttttaaaaaaaaaaaagcatgatgcaaaataaaaaactcaccaaggccatggcaccgagcgttggttttcttgtattaagcggggtccgacccctcttacaaaattttttttttttcgtactacgttttattttttgagtggttccggtaccgtggctcgcaaccaaaagaacataaccccttagtatgcggcgcgcggcgatgtatgaggaaggtcctcctccctcctacgagagtgtggtgagcgcggcgccagtggcggcggcgctgggttctcggggaatcatacgccgcgcgccgctacatactccttccaggaggagggaggatgctctcacaccactcgcgccgcggtcaccgccgccgcgacccaagagccttcgatgctcccctggacccgccgtttgtgcctccgcggtacctgcggcctaccggggggagaaacagcatccgttactctgagttggcacccctattggaagctacgaggggacctgggcggcaaacacggaggcgccatggacgccggatggcccccctctttgtcgtaggcaatgagactcaaccgtggggataacgacaccacccgtgtgtacctggtggacaacaagtcaacggatgtggcatccctgaactaccagaacgaccacagcaactttctgaccacggtcattcaagctgtggtgggcacacatggaccacctgttgttcagttgcctacaccgtagggacttgatggtcttgctggtgtcgttgaaagactggtgccagtaagttaacaatgactacagcccgggggaggcaagcacacagaccatcaatcttgacgaccggtcgcactggggcggcgacctgaaaaccatcctgcataccaacattgttactgatgtcgggccccctccgttcgtgtgtctggtagttagaactgctggccagcgtgaccccgccgctggacttttggtaggacgtatggttgttgccaaatgtgaacgagttcatgtttaccaataagtttaaggcgcgggtgatggtgtcgcgcttgcctactaaggacaatcaggtggagctgaaatacgaacggtttacacttgctcaagtacaaatggttattcaaattccgcgcccactaccacagcgcgaacggatgattcctgttagtccacctcgactttatgctgtgggtggagttcacgctgcccgagggcaactactccgagaccatgaccatagaccttatgaacaacgcgatcgtggagcactacttgaaagtgggcagacacccacctcaagtgcgacgggctcccgttgatgaggctctggtactggtatctggaatacttgttgcgctagcacctcgtgatgaactttcacccgtctcagaacggggttctggaaagcgacatcggggtaaagtttgacacccgcaacttcagactggggtttgaccccgtcactggtcttgtcatgcctggggtatgtcttgccccaagacctttcgctgtagccccatttcaaactgtgggcgttgaagtctgaccccaaactggggcagtgaccagaacagtacggaccccataatacaaacgaagccttccatccagacatcattttgctgccaggatgcggggtggacttcacccacagccgcctgagcaacttgttgggcatccgcaagcgtatgtttgcttcggaaggtaggtctgtagtaaaacgacggtcctacgccccacctgaagtgggtgtcggcggactcgttgaacaacccgtaggcgttcgcgcaacccttccaggagggctttaggatcacctacgatgatctggagggtggtaacattcccgcactgttggatgtggacgcctaccaggcgagcttgaaacgttgggaaggtcctcccgaaatcctagtggatgctactagacctcccaccattgtaagggcgtgacaacctacacctgcggatggtccgctcgaactttgatgacaccgaacagggcgggggtggcgcaggcggcagcaacagcagtggcagcggcgcggaagagaactccaacgcggcagccgcggcaatgcagccggctactgtggcttgtcccgcccccaccgcgtccgccgtcgttgtcgtcaccgtcgccgcgccttctcttgaggttgcgccgtcggcgccgttacgtcggcctggaggacatgaacgatcatgccattcgcggcgacacctttgccacacgggctgaggagaagcgcgctgaggccgaagcagcggccgaagctgccgccccacctcctgtacttgctagtacggtaagcgccgctgtggaaacggtgtgcccgactcctcttcgcgcgactccggcttcgtcgccggcttcgacggcggggcgctgcgcaacccgaggtcgagaagcctcagaagaaaccggtgatcaaacccctgacagaggacagcaagaaacgcagttacaacctaataagcaatgacgcgacgcgttgggctccagctcttcggagtcttctttggccactagtttggggactgtctcctgtcgttctttgcgtcaatgttggattattcgttactgagcaccttcacccagtaccgcagctggtaccttgcatacaactacggcgaccctcagaccggaatccgctcatggaccctgctttgcactcctgacgtaatcgtggaagtgggtcatggcgtcgaccatggaacgtatgttgatgccgctgggagtctggccttaggcgagtacctgggacgaaacgtgaggactgcattcctgcggctcggagcaggtctactggtcgttgccagacatgatgcaagaccccgtgaccttccgctccacgcgccagatcagcaactttccggtggtgggggacgccgagcctcgtccagatgaccagcaacggtctgtactacgttctggggcactggaaggcgaggtgcgcggtctagtcgttgaaaggccaccaccccgccgagctgttgcccgtgcactccaagagcttctacaacgaccaggccgtctactcccaactcatccgccagtttacctctctgacccacgtgttcaatgcggctcgacaacgggcacgtgaggttctcgaagatgttgctggtccggcagatgagggttgagtaggcggtcaaatggagagactgggtgcacaagttacgctttcccgagaaccagattttggcgcgcccgccagcccccaccatcaccaccgtcagtgaaaacgttcctgctctcacagatcacgggacgctaccgcgcgaaagggctcttggtctaaaaccgcgcgggcggtcgggggtggtagtggtggcagtcacttttgcaaggacgagagtgtctagtgccctgcgatggcgtgcgcaacagcatcggaggagtccagcgagtgaccattactgacgccagacgccgcacctgcccctacgtttacaaggccctgggcatagtctcgccgcgacgcgttgtcgtagcctcctcaggtcgctcactggtaatgactgcggtctgcggcgtggacggggatgcaaatgttccgggacccgtatcagagcggcgccgtcctatcgagccgcactttttgagcaagcatgtccatccttatatcgcccagcaataacacaggctggggcctgcgcttcccaagcaagatgtttggcgcaggatagctcggcgtgaaaaactcgttcgtacaggtaggaatatagcgggtcgttattgtgtccgaccccggacgcgaagggttcgttctacaaaccgggggccaagaagcgctccgaccaacacccagtgcgcgtgcgcgggcactaccgcgcgccctggggcgcgcacaaacgcggccgcactgggcgcaccaccgccccggttcttcgcgaggctggttgtgggtcacgcgcacgcgcccgtgatggcgcgcgggaccccgcgcgtgtttgcgccggcgtgacccgcgtggtggctcgatgacgccatcgacgcggtggtggaggaggcgcgcaactacacgcccacgccgccaccagtgtccacagtggacgcggccattcagaccgtggtgcgagctactgcggtagctgcgccaccacctcctccgcgcgttgatgtgcgggtgcggcggtggtcacaggtgtcacctgcgccggtaagtctggcaccacgccggagcccggcgctatgctaaaatgaagagacggcggaggcgcgtagcacgtcgccaccgccgccgacccggcactgccgcccaacgcgcggcggcggccgcctcgggccgcgatacgattttacttctctgccgcctccgcgcatcgtgcagcggtggcggcggctgggccgtgacggcgggttgcgcgccgccgccggctgcttaaccgcgcacgtcgcaccggccgacgggcggccatgcgggccgctcgaaggctggccgcgggtattgtcactgtgccccccaggtccaggcgacgacgaattggcgcgtgcagcgtggccggctgcccgccggtacgcccggcgagcttccgaccggcgcccataacagtgacacggggggtccaggtccgctggagcggccgccgcagcagccgcggccattagtgctatgactcagggtcgcaggggcaacgtgtattgggtgcgcgactcggttagcggcctgcgcgtgccctcgccggcggcgtcgtcggcgccggtaatcacgatactgagtcccagcgtccccgttgcacataacccacgcgctgagccaatcgccggacgcgcacggcgtgcgcacccgccccccgcgcaactagattgcaagaaaaaactacttagactcgtactgttgtatgtatccagcggcggcggcgcgcaacgaagctatggcacgcgtgggcggggggcgcgttgatctaacgttcttttttgatgaatctgagcatgacaacatacataggtcgccgccgccgcgcgttgcttcgatactccaagcgcaaaatcaaagaagagatgctccaggtcatcgcgccggagatctatggccccccgaagaaggaagagcaggattacaagccccgaaagctaaaggttcgcgttttagtttcttctctacgaggtccagtagcgcggcctctagataccggggggcttcttccttctcgtcctaatgttcggggctttcgattagcgggtcaaaaagaaaaagaaagatgatgatgatgaacttgacgacgaggtggaactgctgcacgctaccgcgcccaggcgacgggtacagtggaaaggtcgcccagtttttctttttctttctactactactacttgaactgctgctccaccttgacgacgtgcgatggcgcgggtccgctgcccatgtcacctttcctcgacgcgtaaaacgtgttttgcgacccggcaccaccgtagtctttacgcccggtgagcgctccacccgcacctacaagcgcgtgtatgatgaggtgtacagctgcgcattttgcacaaaacgctgggccgtggtggcatcagaaatgcgggccactcgcgaggtgggcgtggatgttcgcgcacatactactccacatgggcgacgaggacctgcttgagcaggccaacgagcgcctcggggagtttgcctacggaaagcggcataaggacatgctggcgttgccgctggacgagggcaccgctgctcctggacgaactcgtccggttgctcgcggagcccctcaaacggatgcctttcgccgtattcctgtacgaccgcaacggcgacctgctcccgtacccaacacctagcctaaagcccgtaacactgcagcaggtgctgcccgcgcttgcaccgtccgaagaaaagcgcggcctaaagcgcgagtctggtgactttgggttgtggatcggatttcgggcattgtgacgtcgtccacgacgggcgcgaacgtggcaggcttcttttcgcgccggatttcgcgctcagaccactgaaggcacccaccgtgcagctgatggtacccaagcgccagcgactggaagatgtcttggaaaaaatgaccgtggaacctgggctggagcccgaggtccgcgtgccgtgggtggcacgtcgactaccatgggttcgcggtcgctgaccttctacagaaccttttttactggcaccttggacccgacctcgggctccaggcgcaccggccaatcaagcaggtggcgccgggactgggcgtgcagaccgtggacgttcagatacccactaccagtagcaccagtattgccaccgccacagagggcagccggttagttcgtccaccgcggccctgacccgcacgtctggcacctgcaagtctatgggtgatggtcatcgtggtcataacggtggcggtgtctcccgttggagacacaaacgtccccggttgcctcagcggtggcggatgccgcggtgcaggcggtcgctgcggccgcgtccaagacctctacggaggtgcaaacggaacctctgtgtttgcaggggccaacggagtcgccaccgcctacggcgccacgtccgccagcgacgccggcgcaggttctggagatgcctccacgtttgcctcccgtggatgtttcgcgtttcagccccccggcgcccgcgcggttcgaggaagtacggcgccgccagcgcgctactgcccgaatatgccctacatccttccgggcacctacaaagcgcaaagtcggggggccgcgggcgcgccaagctccttcatgccgcggcggtcgcgcgatgacgggcttatacgggatgtaggaaggattgcgcctacccccggctatcgtggctacacctaccgccccagaagacgagcaactacccgacgccgaaccaccactggaacccgccgccgccgtcgcctaacgcggatgggggccgatagcaccgatgtggatggcggggtcttctgctcgttgatgggctgcggcttggtggtgaccttgggcggcggcggcagcgggtcgccagcccgtgctggccccgatttccgtgcgcagggtggctcgcgaaggaggcaggaccctggtgctgccaacagcgcgctaccaccccagcatcgtcagcggtcgggcacgaccggggctaaaggcacgcgtcccaccgagcgcttcctccgtcctgggaccacgacggttgtcgcgcgatggtggggtcgtagcattaaaagccggtctttgtggttcttgcagatatggccctcacctgccgcctccgtttcccggtgccgggattccgaggaagaatgcaccgtaggaggggcaattttcggccagaaacaccaagaacgtctataccgggagtggacggcggaggcaaagggccacggccctaaggctccttcttacgtggcatcctccccgatggccggccacggcctgacgggcggcatgcgtcgtgcgcaccaccggcggcggcgcgcgtcgcaccgtcgcatgcgcggcggtatcctgcccctccttataccggccggtgccggactgcccgccgtacgcagcacgcgtggtggccgccgccgcgcgcagcgtggcagcgtacgcgccgccataggacggggaggaatttccactgatcgccgcggcgattggcgccgtgcccggaattgcatccgtggccttgcaggcgcagagacactgattaaaaacaagttgcatgtggaaaaaaaggtgactagcggcgccgctaaccgcggcacgggccttaacgtaggcaccggaacgtccgcgtctctgtgactaatttttgttcaacgtacaccttttttcaaaataaaaagtctggactctcacgctcgcttggtcctgtaactattttgtagaatggaagacatcaactttgcgtctctggccccgcgacacggctcagttttatttttcagacctgagagtgcgagcgaaccaggacattgataaaacatcttaccttctgtagttgaaacgcagagaccggggcgctgtgccgaggcgcccgttcatgggaaactggcaagatatcggcaccagcaatatgagcggtggcgccttcagctggggctcgctgtggagcggcattaaaaatttcggtcgcgggcaagtaccctttgaccgttctatagccgtggtcgttatactcgccaccgcggaagtcgaccccgagcgacacctcgccgtaatttttaaagccatccaccgttaagaactatggcagcaaggcctggaacagcagcacaggccagatgctgagggataagttgaaagagcaaaatttccaacaaaaggtggtagaggtggcaattcttgataccgtcgttccggaccttgtcgtcgtgtccggtctacgactccctattcaactttctcgttttaaaggttgttttccaccatcatggcctggcctctggcattagcggggtggtggacctggccaaccaggcagtgcaaaataagattaacagtaagcttgatccccgccctcccgtagaggataccggaccggagaccgtaatcgccccaccacctggaccggttggtccgtcacgttttattctaattgtcattcgaactaggggcgggagggcatctcctgcctccaccggccgtggagacagtgtctccagaggggcgtggcgaaaagcgtccgcgccccgacagggaagaaactctggtgacgcaaatagacgagcctcggaggtggccggcacctctgtcacagaggtctccccgcaccgcttttcgcaggcgcggggctgtcccttctttgagaccactgcgtttatctgctcggaccctcgtacgaggaggcactaaagcaaggcctgcccaccacccgtcccatcgcgcccatggctaccggagtgctgggccagcacacacccgtaacgctgggggagcatgctcctccgtgatttcgttccggacgggtggtgggcagggtagcgcgggtaccgatggcctcacgacccggtcgtgtgtgggcattgcgaccacctgcctccccccgccgacacccagcagaaacctgtgctgccaggcccgaccgccgttgttgtaacccgtcctagccgcgcgtccctgcgccgcgccgctggacggaggggggcggctgtgggtcgtctttggacacgacggtccgggctggcggcaacaacattgggcaggatcggcgcgcagggacgcggcgcggcgcagcggtccgcgatcgttgcggcccgtagccagtggcaactggcaaagcacactgaacagcatcgtgggtctgggggtgcaatccctgaagcgccgacgagtcgccaggcgctagcaacgccgggcatcggtcaccgttgaccgtttcgtgtgacttgtcgtagcacccagacccccacgttagggacttcgcggctgcttgcttctgaatagctaacgtgtcgtatgtgtgtcatgtatgcgtccatgtcgccgccagaggagctgctgagccgccgcgcgcccgctttccaagatggcacgaagacttatcgattgcacagcatacacacagtacatacgcaggtacagcggcggtctcctcgacgactcggcggcgcgcgggcgaaaggttctaccgtaccccttcgatgatgccgcagtggtcttacatgcacatctcgggccaggacgcctcggagtacctgagccccgggctggtgcagtttgcccgcgccaccatggggaagctactacggcgtcaccagaatgtacgtgtagagcccggtcctgcggagcctcatggactcggggcccgaccacgtcaaacgggcgcggtgggagacgtacttcagcctgaataacaagtttagaaaccccacggtggcgcctacgcacgacgtgaccacagaccggtcccagcgtttgacgctgcggttcactctgcatgaagtcggacttattgttcaaatctttggggtgccaccgcggatgcgtgctgcactggtgtctggccagggtcgcaaactgcgacgccaagttccctgtggaccgtgaggatactgcgtactcgtacaaggcgcggttcaccctagctgtgggtgataaccgtgtgctggacatggcttccacgtactttgaagggacacctggcactcctatgacgcatgagcatgttccgcgccaagtgggatcgacacccactattggcacacgacctgtaccgaaggtgcatgaaactcatccgcggcgtgctggacaggggccctacttttaagccctactctggcactgcctacaacgccctggctcccaagggtgccccaaatccttgcgaatgggtaggcgccgcacgacctgtccccgggatgaaaattcgggatgagaccgtgacggatgttgcgggaccgagggttcccacggggtttaggaacgcttaccgatgaagctgctactgctcttgaaataaacctagaagaagaggacgatgacaacgaagacgaagtagacgagcaagctgagcagcaaaaaactcacgtatctacttcgacgatgacgagaactttatttggatcttcttctcctgctactgttgcttctgcttcatctgctcgttcgactcgtcgttttttgagtgcatattgggcaggcgccttattctggtataaatattacaaaggagggtattcaaataggtgtcgaaggtcaaacacctaaatatgccgataaaacatttcaaccaacccgtccgcggaataagaccatatttataatgtttcctcccataagtttatccacagcttccagtttgtggatttatacggctattttgtaaagttggtgaacctcaaataggagaatctcagtggtacgaaactgaaattaatcatgcagctgggagagtccttaaaaagactaccccaatgaaaccatgttacggtacttggagtttatcctcttagagtcaccatgctttgactttaattagtacgtcgaccctctcaggaatttttctgatggggttactttggtacaatgccatcatatgcaaaacccacaaatgaaaatggagggcaaggcattcttgtaaagcaacaaaatggaaagctagaaagtcaagtggaaatgcaatttttctcaaagtatacgttttgggtgtttacttttacctcccgttccgtaagaacatttcgttgttttacctttcgatctttcagttcacctttacgttaaaaagagttctactgaggcgaccgcaggcaatggtgataacttgactcctaaagtggtattgtacagtgaagatgtagatatagaaaccccagacactcatatttcttagatgactccgctggcgtccgttaccactattgaactgaggatttcaccataacatgtcacttctacatctatatctttggggtctgtgagtataaagaatcatgcccactattaaggaaggtaactcacgagaactaatgggccaacaatctatgcccaacaggcctaattacattgcttttagggacaattttattggtgtacgggtgataattccttccattgagtgctcttgattacccggttgttagatacgggttgtccggattaatgtaacgaaaatccctgttaaaataaccactaatgtattacaacagcacgggtaatatgggtgttctggcgggccaagcatcgcagttgaatgctgttgtagatttgcaagacagaaacacagagctttgattacataatgttgtcgtgcccattatacccacaagaccgcccggttcgtagcgtcaacttacgacaacatctaaacgttctgtctttgtgtctcgaaacataccagcttttgcttgattccattggtgatagaaccaggtacttttctatgtggaatcaggctgttgacagctatgatccagatgttagaattattgagtatggtcgaaaacgaactaaggtaaccactatcttggtccatgaaaagatacaccttagtccgacaactgtcgatactaggtctacaatcttaataactaaatcatggaactgaagatgaacttccaaattactgctttccactgggaggtgtgattaatacagagactcttaccaaggtaaaacctaaaacaggtcagtttagtaccttgacttctacttgaaggtttaatgacgaaaggtgaccctccacactaattatgtctctgagaatggttccattttggattttgtccagtcgaaaatggatgggaaaaagatgctacagaattttcagataaaaatgaaataagagttggaaataattttgccatggaaatcaatctaaatgccaacctgtcttttacctaccctttttctacgatgtcttaaaagtctatttttactttattctcaacctttattaaaacggtacctttagttagatttacggttggacaggagaaatttcctgtactccaacatagcgctgtatttgcccgacaagctaaagtacagtccttccaacgtaaaaatttctgataacccaaacacctacgacctctttaaaggacatgaggttgtatcgcgacataaacgggctgttcgatttcatgtcaggaaggttgcatttttaaagactattgggtttgtggatgctctacatgaacaagcgagtggtggctcccgggttagtggactgctacattaaccttggagcacgctggtcccttgactatatggacaacgtcaacccatttgatgtacttgttcgctcaccaccgagggcccaatcacctgacgatgtaattggaacctcgtgcgaccagggaactgatatacctgttgcagttgggtaaaaaccaccaccgcaatgctggcctgcgctaccgctcaatgttgctgggcaatggtcgctatgtgcccttccacatccaggtgcctcagaagttctttgccattggtggtggcgttacgaccggacgcgatggcgagttacaacgacccgttaccagcgatacacgggaaggtgtaggtccacggagtcttcaagaaacggtttaaaaacctccttctcctgccgggctcatacacctacgagtggaacttcaggaaggatgttaacatggttctgcagagctccctaggaaatgacctaagaatttttggaggaagaggacggcccgagtatgtggatgctcaccttgaagtccttcctacaattgtaccaagacgtctcgagggatcctttactggattcggttgacggagccagcattaagtttgatagcatttgcctttacgccaccttcttccccatggcccacaacaccgcctccacgcttgaggccatgcttagaccaactgcctcggtcgtaattcaaactatcgtaaacggaaatgcggtggaagaaggggtaccgggtgttgtggcggaggtgcgaactccggtacgaatctaacgacaccaacgaccagtcctttaacgactatctctccgccgccaacatgctctaccctatacccgccaacgctaccaacgtgcccatatccatcccctttgctgtggttgctggtcaggaaattgctgatagagaggcggcggttgtacgagatgggatatgggcggttgcgatggttgcacgggtataggtaggggacccgcaactgggcggctttccgcggctgggccttcacgcgccttaagactaaggaaaccccatcactgggctcgggctacgacccttattacacctactcgggcgttgacccgccgaaaggcgccgacccggaagtgcgcggaattctgattcctttggggtagtgacccgagcccgatgctgggaataatgtggatgagtggctctataccctacctagatggaaccttttacctcaaccacacctttaagaaggtggccattacctttgactcttctgtcagctggcctggcaatgacaccgagatatgggatggatctaccttggaaaatggagttggtgtggaaattcttccaccggtaatggaaactgagaagacagtcgaccggaccgttactgcgcctgcttacccccaacgagtttgaaattaagcgctcagttgacggggagggttacaacgttgcccagtgtaacatgaccaaagactggttcctggtacgcggacgaatgggggttgctcaaactttaattcgcgagtcaactgcccctcccaatgttgcaacgggtcacattgtactggtttctgaccaaggaccatgaaatgctagctaactacaacattggctaccagggcttctatatcccagagagctacaaggaccgcatgtactccttctttagaaacttccagcccatgagtttacgatcgattgatgttgtaaccgatggtcccgaagatatagggtctctcgatgttcctggcgtacatgaggaagaaatctttgaaggtcgggtactcccgtcaggtggtggatgatactaaatacaaggactaccaacaggtgggcatcctacaccaacacaacaactctggatttgttggctaccttgcccccaccggcagtccaccacctactatgatttatgttcctgatggttgtccacccgtaggatgtggttgtgttgttgagacctaaacaaccgatggaacgggggtggatgcgcgaaggacaggcctaccctgctaacttcccctatccgcttataggcaagaccgcagttgacagcattacccagaaaaagtttctttgcgatcgcatacgcgcttcctgtccggatgggacgattgaaggggataggcgaatatccgttctggcgtcaactgtcgtaatgggtctttttcaaagaaacgctagcgtccctttggcgcatcccattctccagtaactttatgtccatgggcgcactcacagacctgggccaaaaccttctctacgccaactccgcccacgcgctagagggaaaccgcgtagggtaagaggtcattgaaatacaggtacccgcgtgagtgtctggacccggttttggaagagatgcggttgaggcgggtgcgcgatctcatgacttttgaggtggatcccatggacgagcccacccttctttatgttttgtttgaagtctttgacgtggtccgtgtgcaccggccgcaccgcggcgtcgtactgaaaactccacctagggtacctgctcgggtgggaagaaatacaaaacaaacttcagaaactgcaccaggcacacgtggccggcgtggcgccgcagatcgaaaccgtgtacctgcgcacgcccttctcggccggcaacgccacaacataaagaagcaagcaacatcaacaacagctgccgccatgggctccagtgatagctttggcacatggacgcgtgcgggaagagccggccgttgcggtgttgtatttcttcgttcgttgtagttgttgtcgacggcggtacccgaggtcactgcaggaactgaaagccattgtcaaagatcttggttgtgggccatattttttgggcacctatgacaagcgctttccaggctttgtttctccacacaagctccgtccttgactttcggtaacagtttctagaaccaacacccggtataaaaaacccgtggatactgttcgcgaaaggtccgaaacaaagaggtgtgttcgaggcctgcgccatagtcaatacggccggtcgcgagactgggggcgtacactggatggcctttgcctggaacccgcactcaaaaacatgctacctctttgagccggacgcggtatcagttatgccggccagcgctctgacccccgcatgtgacctaccggaaacggaccttgggcgtgagtttttgtacgatggagaaactcgcctttggcttttctgaccagcgactcaagcaggtttaccagtttgagtacgagtcactcctgcgccgtagcgccattgcttcttcccccgaccgctgtatggaaaccgaaaagactggtcgctgagttcgtccaaatggtcaaactcatgctcagtgaggacgcggcatcgcggtaacgaagaagggggctggcgacataaacgctggaaaagtccacccaaagcgtacaggggcccaactcggccgcctgtggactattctgctgcatgtttctccacgcctttgccaactggccccaattgcgaccttttcaggtgggtttcgcatgtccccgggttgagccggcggacacctgataagacgacgtacaaagaggtgcggaaacggttgaccggggttactcccatggatcacaaccccaccatgaaccttattaccggggtacccaactccatgctcaacagtccccaggtacagcccaccctgcgtcgcaaccaggtgagggtacctagtgttggggtggtacttggaataatggccccatgggttgaggtacgagttgtcaggggtccatgtcgggtgggacgcagcgttggtccaacagctctacagcttcctggagcgccactcgccctacttccgcagccacagtgcgcagattaggagcgccacttctttttgtcacttgaaaaacatgtattgtcgagatgtcgaaggacctcgcggtgagcgggatgaaggcgtcggtgtcacgcgtctaatcctcgcggtgaagaaaaacagtgaactttttgtacataaaataatgtactagagacactttcaataaaggcaaatgcttttatttgtacactctcgggtgattatttacccccacccttgccgtctgcgccgtttaattttattacatgatctctgtgaaagttatttccgtttacgaaaataaacatgtgagagcccactaataaatgggggtgggaacggcagacgcggcaaattaaatcaaaggggttctgccgcgcatcgctatgcgccactggcagggacacgttgcgatactggtgtttagtgctccacttaaactcaggcacaaccatcctttagtttccccaagacggcgcgtagcgatacgcggtgaccgtccctgtgcaacgctatgaccacaaatcacgaggtgaatttgagtccgtgttggtagggcggcagctcggtgaagttttcactccacaggctgcgcaccatcaccaacgcgtttagcaggtcgggcgccgatatcttgaagtcgcagttggggcctcccgccgtcgagccacttcaaaagtgaggtgtccgacgcgtggtagtggttgcgcaaatcgtccagcccgcggctatagaacttcagcgtcaaccccggagggccctgcgcgcgcgagttgcgatacacagggttgcagcactggaacactatcagcgccgggtggtgcacgctggccagcacgctcttgtcggagatcagacgggacgcgcgcgctcaacgctatgtgtcccaacgtcgtgaccttgtgatagtcgcggcccaccacgtgcgaccggtcgtgcgagaacagcctctagtcttccgcgtccaggtcctccgcgttgctcagggcgaacggagtcaactttggtagctgccttcccaaaaagggcgcgtgcccaggctttgagttgcactcgcaggcgcaggtccaggaggcgcaacgagtcccgcttgcctcagttgaaaccatcgacggaagggtttttcccgcgcacgggtccgaaactcaacgtgagcgaccgtagtggcatcaaaaggtgaccgtgcccggtctgggcgttaggatacagcgcctgcataaaagccttgatctgcttaaaagccacctgagcctttgctggcatcaccgtagttttccactggcacgggccagacccgcaatcctatgtcgcggacgtattttcggaactagacgaattttcggtggactcggaaacggccttcagagaagaacatgccgcaagacttgccggaaaactgattggccggacaggccgcgtcgtgcacgcagcaccttgcgtcggtgttggagatctgccggaagtctcttcttgtacggcgttctgaacggccttttgactaaccggcctgtccggcgcagcacgtgcgtcgtggaacgcagccacaacctctagacgaccacatttcggccccaccggttcttcacgatcttggccttgctagactgctccttcagcgcgcgctgcccgttttcgctcgtcacatccatttcaatcatggtgtaaagccggggtggccaagaagtgctagaaccggaacgatctgacgaggaagtcgcgcgcgacgggcaaaagcgagcagtgtaggtaaagttagtcgtgctccttatttatcataatgcttccgtgtagacacttaagctcgccttcgatctcagcgcagcggtgcagccacaacgcgcagcccgtgggctcgtggcacgaggaataaatagtattacgaaggcacatctgtgaattcgagcggaagctagagtcgcgtcgccacgtcggtgttgcgcgtcgggcacccgagcacatgcttgtaggtcacctctgcaaacgactgcaggtacgcctgcaggaatcgccccatcatcgtcacaaaggtcttgttgctggtgaaggtcagctgcaactacgaacatccagtggagacgtttgctgacgtccatgcggacgtccttagcggggtagtagcagtgtttccagaacaacgaccacttccagtcgacgttgccgcggtgctcctcgttcagccaggtcttgcatacggccgccagagcttccacttggtcaggcagtagtttgaagttcgcctttagatcgttatccacgtggcgccacgaggagcaagtcggtccagaacgtatgccggcggtctcgaaggtgaaccagtccgtcatcaaacttcaagcggaaatctagcaataggtgcaggtacttgtccatcagcgcgcgcgcagcctccatgcccttctcccacgcagacacgatcggcacactcagcgggttcatcaccgtaatttcactttccgcccatgaacaggtagtcgcgcgcgcgtcggaggtacgggaagagggtgcgtctgtgctagccgtgtgagtcgcccaagtagtggcattaaagtgaaaggcgttcgctgggctcttcctcttcctcttgcgtccgcataccacgcgccactgggtcgtcttcattcagccgccgcactgtgcgcttacctcctttgccatgcaagcgacccgagaaggagaaggagaacgcaggcgtatggtgcgcggtgacccagcagaagtaagtcggcggcgtgacacgcgaatggaggaaacggtacgttgattagcaccggtgggttgctgaaacccaccatttgtagcgccacatcttctctttcttcctcgctgtccacgattacctctggtgatggcgggcgctaactaatcgtggccacccaacgactttgggtggtaaacatcgcggtgtagaagagaaagaaggagcgacaggtgctaatggagaccactaccgcccgcgacgggcttgggagaagggcgcttctttttcttcttgggcgcaatggccaaatccgccgccgaggtcgatggccgcgggctgggtgtgcgcggcaccagcgcgcccgaaccctcttcccgcgaagaaaaagaagaacccgcgttaccggtttaggcggcggctccagctaccggcgcccgacccacacgcgccgtggtcgcggtcttgtgatgagtcttcctcgtcctcggactcgatacgccgcctcatccgcttttttgggggcgcccggggaggcggcggcgacggggacggggacgaccagaacactactcagaaggagcaggagcctgagctatgcggcggagtaggcgaaaaaacccccgcgggcccctccgccgccgctgcccctgcccctgctgacgtcctccatggttgggggacgtcgcgccgcaccgcgtccgcgctcgggggtggtttcgcgctgctcctcttcccgactggccatttccttctcctatatgcaggaggtaccaaccccctgcagcgcggcgtggcgcaggcgcgagcccccaccaaagcgcgacgaggagaagggctgaccggtaaaggaagaggatatggcagaaaaagatcatggagtcagtcgagaagaaggacagcctaaccgccccctctgagttcgccaccaccgcctccaccgatgccgccaacgcgcctacccgtctttttctagtacctcagtcagctcttcttcctgtcggattggcgggggagactcaagcggtggtggcggaggtggctacggcggttgcgcggatgcaccttccccgtcgaggcacccccgcttgaggaggaggaagtgattatcgagcaggacccaggttttgtaagcgaagacgacgaggaccgctcagtaccagtggaaggggcagctccgtgggggcgaactcctcctccttcactaatagctcgtcctgggtccaaaacattcgcttctgctgctcctggcgagtcatggtacagaggataaaaagcaagaccaggacaacgcagaggcaaacgaggaacaagtcgggcggggggacgaaaggcatggcgactacctagatgtgggagacgtgtctcctatttttcgttctggtcctgttgcgtctccgtttgctccttgttcagcccgcccccctgctttccgtaccgctgatggatctacaccctctgcacgtgctgttgaagcatctgcagcgccagtgcgccattatctgcgacgcgttgcaagagcgcagcgatgtgcccctcgccatagcggatgtcagccttgctgcacgacaacttcgtagacgtcgcggtcacgcggtaatagacgctgcgcaacgttctcgcgtcgctacacggggagcggtatcgcctacagtcggaacgctacgaacgccacctattctcaccgcgcgtaccccccaaacgccaagaaaacggcacatgcgagcccaacccgcgcctcaacttctaccccgtatttgccgatgcttgcggtggataagagtggcgcgcatggggggtttgcggttcttttgccgtgtacgctcgggttgggcgcggagttgaagatggggcataaacgggtgccagaggtgcttgccacctatcacatctttttccaaaactgcaagatacccctatcctgccgtgccaaccgcagccgagcggacaagcagctggcctcacggtctccacgaacggtggatagtgtagaaaaaggttttgacgttctatggggataggacggcacggttggcgtcggctcgcctgttcgtcgaccggatgcggcagggcgctgtcatacctgatatcgcctcgctcaacgaagtgccaaaaatctttgagggtcttggacgcgacgagaagcgcgcggcaaacgctctacgccgtcccgcgacagtatggactatagcggagcgagttgcttcacggtttttagaaactcccagaacctgcgctgctcttcgcgcgccgtttgcgagagcaacaggaaaacagcgaaaatgaaagtcactctggagtgttggtggaactcgagggtgacaacgcgcgcctagccgtactaaaacgcagcatcgaggtccgttgtccttttgtcgcttttactttcagtgagacctcacaaccaccttgagctcccactgttgcgcgcggatcggcatgattttgcgtcgtagctccagacccactttgcctacccggcacttaacctaccccccaaggtcatgagcacagtcatgagtgagctgatcgtgcgccgtgcgcagcccctggagagggatgtgggtgaaacggatgggccgtgaattggatggggggttccagtactcgtgtcagtactcactcgactagcacgcggcacgcgtcggggacctctccctaccaaatttgcaagaacaaacagaggagggcctacccgcagttggcgacgagcagctagcgcgctggcttcaaacgcgcgagcctgccgacttggaggagcggtttaaacgttcttgtttgtctcctcccggatgggcgtcaaccgctgctcgtcgatcgcgcgaccgaagtttgcgcgctcggacggctgaacctcctcgcacgcaaactaatgatggccgcagtgctcgttaccgtggagcttgagtgcatgcagcggttctttgctgacccggagatgcagcgcaagctagaggaaacatgcgtttgattactaccggcgtcacgagcaatggcacctcgaactcacgtacgtcgccaagaaacgactgggcctctacgtcgcgttcgatctcctttgtttgcactacacctttcgacagggctacgtacgccaggcctgcaagatctccaacgtggagctctgcaacctggtctcctaccttggaattttgcacgaaaaacgtgatgtggaaagctgtcccgatgcatgcggtccggacgttctagaggttgcacctcgagacgttggaccagaggatggaaccttaaaacgtgctttaccgccttgggcaaaacgtgcttcattccacgctcaagggcgaggcgcgccgcgactacgtccgcgactgcgtttacttatttctatgctacacctggcatggcggaacccgttttgcacgaagtaaggtgcgagttcccgctccgcgcggcgctgatgcaggcgctgacgcaaatgaataaagatacgatgtggaccgtgacggccatgggcgtttggcagcagtgcttggaggagtgcaacctcaaggagctgcagaaactgctaaagcaaaacttgaaggacctatggacggccttcctgccggtacccgcaaaccgtcgtcacgaacctcctcacgttggagttcctcgacgtctttgacgatttcgttttgaacttcctggatacctgccggaagaacgagcgctccgtggccgcgcacctggcggacatcattttccccgaacgcctgcttaaaaccctgcaacagggtctgccagacttcaccagtcaaagcattgctcgcgaggcaccggcgcgtggaccgcctgtagtaaaaggggcttgcggacgaattttgggacgttgtcccagacggtctgaagtggtcagtttcgttgttgcagaactttaggaactttatcctagagcgctcaggaatcttgcccgccacctgctgtgcacttcctagcgactttgtgcccattaagtaccgcgaacaacgtcttgaaatccttgaaataggatctcgcgagtccttagaacgggcggtggacgacacgtgaaggatcgctgaaacacgggtaattcatggcgctatgccctccgccgctttggggccactgctaccttctgcagctagccaactaccttgcctaccactctgacataatggaagacgtgagcggtgacggtctatacgggaggcggcgaaaccccggtgacgatggaagacgtcgatcggttgatggaacggatggtgagactgtattaccttctgcactcgccactgccagatctggagtgtcactgtcgctgcaacctatgcaccccgcaccgctccctggtttgcaattcgcagctgcttaacgaaagtcaaattatcggtacctttgagcgacctcacagtgacagcgacgttggatacgtggggcgtggcgagggaccaaacgttaagcgtcgacgaattgctttcagtttaatagccatggaaactcgtgcagggtccctcgcctgacgaaaagtccgcggctccggggttgaaactcactccggggctgtggacgtcggcttaccttcgcaaatttgtacctgaggaacgtcccagggagcggactgcttttcaggcgccgaggccccaactttgagtgaggccccgacacctgcagccgaatggaagcgtttaaacatggactcctctaccacgcccacgagattaggttctacgaagaccaatcccgcccgccaaatgcggagcttaccgcctgcgtcattacccagggccacattcttggccaagatggtgcgggtgctctaatccaagatgcttctggttagggcgggcggtttacgcctcgaatggcggacgcagtaatgggtcccggtgtaagaaccggttttgcaagccatcaacaaagcccgccaagagtttctgctacgaaagggacggggggtttacttggacccccagtccggcgaggagctcaacccaatcccccaacgttcggtagttgtttcgggcggttctcaaagacgatgctttccctgccccccaaatgaacctgggggtcaggccgctcctcgagttgggttagggggcgccgccgcagccctatcagcagcagccgcgggcccttgcttcccaggatggcacccaaaaagaagctgcagctgccgccgccacccacggacgaggagggcggcggcgtcgggatagtcgtcgtcggcgcccgggaacgaagggtcctaccgtgggtttttcttcgacgtcgacggcggcggtgggtgcctgctcctccaatactgggacagtcaggcagaggaggttttggacgaggaggaggaggacatgatggaagactgggagagcctagacgaggaagcttccgaggtcgaagattatgaccctgtcagtccgtctcctccaaaacctgctcctcctcctcctgtactaccttctgaccctctcggatctgctccttcgaaggctccagcttctggtgtcagacgaaacaccgtcaccctcggtcgcattcccctcgccggcgccccagaaatcggcaaccggttccagcatggctacaacctccgctcctcagccacagtctgctttgtggcagtgggagccagcgtaaggggagcggccgcggggtctttagccgttggccaaggtcgtaccgatgttggaggcgaggagtcgcgccgccggcactgcccgttcgccgacccaaccgtagatgggacaccactggaaccagggccggtaagtccaagcagccgccgccgttagcccaagagccgcggcggccgtgacgggcaagcggctgggttggcatctaccctgtggtgaccttggtcccggccattcaggttcgtcggcggcggcaatcgggttctcgaacaacagcgccaaggctaccgctcatggcgcgggcacaagaacgccatagttgcttgcttgcaagactgtgggggcaacatctccttcgcccgccgcttttgttgtcgcggttccgatggcgagtaccgcgcccgtgttcttgcggtatcaacgaacgaacgttctgacacccccgttgtagaggaagcgggcggcgaatcttctctaccatcacggcgtggccttcccccgtaacatcctgcattactaccgtcatctctacagcccatactgcaccggcggcagcggcagcggcagcagaagagatggtagtgccgcaccggaagggggcattgtaggacgtaatgatggcagtagagatgtcgggtatgacgtggccgccgtcgccgtcgccgtcgaacagcagcggccacacagaagcaaaggcgaccggatagcaagactctgacaaagcccaagaaatccacagcggcggcagcagcaggaggaggagcgctgttgtcgtcgccggtgtgtcttcgtttccgctggcctatcgttctgagactgtttcgggttctttaggtgtcgccgccgtcgtcgtcctcctcctcgcgaccgtctggcgcccaacgaacccgtatcgacccgcgagcttagaaacaggatttttcccactctgtatgctatatttcaacagagcaggggccaagaacaaggcagaccgcgggttgcttgggcatagctgggcgctcgaatctttgtcctaaaaagggtgagacatacgatataaagttgtctcgtccccggttcttgttcagctgaaaataaaaaacaggtctctgcgatccctcacccgcagctgcctgtatcacaaaagcgaagatcagcttcggcgcacgctggaagacgcggaggctcgacttttattttttgtccagagacgctagggagtgggcgtcgacggacatagtgttttcgcttctagtcgaagccgcgtgcgaccttctgcgcctccgtctcttcagtaaatactgcgcgctgactcttaaggactagtttcgcgccctttctcaaatttaagcgcgaaaactacgtcatctccagcggccacacccgagagaagtcatttatgacgcgcgactgagaattcctgatcaaagcgcgggaaagagtttaaattcgcgcttttgatgcagtagaggtcgccggtgtgggcgcgccagcacctgtcgtcagcgccattatgagcaaggaaattcccacgccctacatgtggagttaccagccacaaatgggacttgcggctggagctgccccgcggtcgtggacagcagtcgcggtaatactcgttcctttaagggtgcgggatgtacacctcaatggtcggtgtttaccctgaacgccgacctcgacgggaagactactcaacccgaataaactacatgagcgcgggaccccacatgatatcccgggtcaacggaatccgcgcccaccgaaaccgaattctcttggaacattctgatgagttgggcttatttgatgtactcgcgccctggggtgtactatagggcccagttgccttaggcgcgggtggctttggcttaagagaaccttgtggcggctattaccaccacacctcgtaataaccttaatccccgtagttggcccgctgccctggtgtaccaggaaagtcccgctcccaccactgtggtacttccgccgataatggtggtgtggagcattattggaattaggggcatcaaccgggcgacgggaccacatggtcctttcagggcgagggtggtgacaccatgaacccagagacgcccaggccgaagttcagatgactaactcaggggcgcagcttgcgggcggctttcgtcacagggtgcggtcgcccgggcagggtataactcgggtctctgcgggtccggcttcaagtctactgattgagtccccgcgtcgaacgcccgccgaaagcagtgtcccacgccagcgggcccgtcccatattgagacctgacaatcagagggcgaggtattcagctcaacgacgagtcggtgagctcctcgcttggtctccgtccggacgggacatttcagatcggcggcgccggtggactgttagtctcccgctccataagtcgagttgctgctcagccactcgaggagcgaaccagaggcaggcctgccctgtaaagtctagccgccgcggccccgtccttcattcacgcctcgtcaggcaatcctaactctgcagacctcgtcctctgagccgcgctctggaggcattggaactctgcaatttattgaggagggcaggaagtaagtgcggagcagtccgttaggattgagacgtctggagcaggagactcggcgcgagacctccgtaaccttgagacgttaaataactcctctttgtgccatcggtctactttaaccccttctcgggacctcccggccactatccggatcaatttattcctaactttgacgcggtaaaggactcggcggacgaaacacggtagccagatgaaattggggaagagccctggagggccggtgataggcctagttaaataaggattgaaactgcgccatttcctgagccgcctgcgctacgactgaatgttaagtggagaggcagagcaactgcgcctgaaacacctggtccactgtcgccgccacaagtgctttgcccgcgactccggtgagttcgatgctgacttacaattcacctctccgtctcgttgacgcggactttgtggaccaggtgacagcggcggtgttcacgaaacgggcgctgaggccactcaattgctactttgaattgcccgaggatcatatcgagggcccggcgcacggcgtccggcttaccgcccagggagagcttgcccgtagcctgattcgggagtttaacgatgaaacttaacgggctcctagtatagctcccgggccgcgtgccgcaggccgaatggcgggtccctctcgaacgggcatcggactaagccctcaaaacccagcgccccctgctagttgagcgggacaggggaccctgtgttctcactgtgatttgcaactgtcctaaccttggattacatcaagatcctctagttatgggtcgcgggggacgatcaactcgccctgtcccctgggacacaagagtgacactaaacgttgacaggattggaacctaatgtagttctaggagatcaattaactagagtacccggggatcttattccctttaactaataaaaaaaaataataaagcatcacttacttaaaatcagttagcaaatttctgtccagtttatattgatctcatgggcccctagaataagggaaattgattatttttttttattatttcgtagtgaatgaattttagtcaatcgtttaaagacaggtcaaatatcagcagcacctccttgccctcctcccagctctggtattgcagcttcctcctggctgcaaactttctccacaatctaaatggaatgtcagtttcctcctgagtcgtcgtggaggaacgggaggagggtcgagaccataacgtcgaaggaggaccgacgtttgaaagaggtgttagatttaccttacagtcaaaggaggacttcctgtccatccgcacccactatcttcatgttgttgcagatgaagcgcgcaagaccgtctgaagataccttcaaccccgtgtatccatatgacacggaaaaggacaggtaggcgtgggtgatagaagtacaacaacgtctacttcgcgcgttctggcagacttctatggaagttggggcacataggtatactgtgccttaccggtcctccaactgtgccttttcttactcctccctttgtatcccccaatgggtttcaagagagtccccctggggtactctctttgcgcctatccgaactggccaggaggttgacacggaaaagaatgaggagggaaacatagggggttacccaaagttctctcagggggaccccatgagagaaacgcggataggcttgctctagttacctccaatggcatgcttgcgctcaaaatgggcaacggcctctctctggacgaggccggcaaccttacctcccaaaatgtaaccactgtgaggagatcaatggaggttaccgtacgaacgcgagttttacccgttgccggagagagacctgctccggccgttggaatggagggttttacattggtgacactccccacctctcaaaaaaaccaagtcaaacataaacctggaaatatctgcacccctcacagttacctcagaagccctaactgtggctgccgccgcacctctagggtggagagtttttttggttcagtttgtatttggacctttatagacgtggggagtgtcaatggagtcttcgggattgacaccgacggcggcgtggagatatggtcgcgggcaacacactcaccatgcaatcacaggccccgctaaccgtgcacgactccaaacttagcattgccacccaaggacccctcacagtgtcagtaccagcgcccgttgtgtgagtggtacgttagtgtccggggcgattggcacgtgctgaggtttgaatcgtaacggtgggttcctggggagtgtcacagtcaaggaaagctagccctgcaaacatcaggccccctcaccaccaccgatagcagtacccttactatcactgcctcaccccctctaactactgccactggtagttcctttcgatcgggacgtttgtagtccgggggagtggtggtggctatcgtcatgggaatgatagtgacggagtgggggagattgatgacggtgaccatccttgggcattgacttgaaagagcccatttatacacaaaatggaaaactaggactaaagtacggggctcctttgcatgtaacagacgacctaaacactttggaacccgtaactgaactttctcgggtaaatatgtgttttaccttttgatcctgatttcatgccccgaggaaacgtacattgtctgctggatttgtgaaacaccgtagcaactggtccaggtgtgactattaataatacttccttgcaaactaaagttactggagccttgggttttgattcacaaggcaatatgcaacttatggcatcgttgaccaggtccacactgataattattatgaaggaacgtttgatttcaatgacctcggaacccaaaactaagtgttccgttatacgttgaatatgtagcaggaggactaaggattgattctcaaaacagacgccttatacttgatgttagttatccgtttgatgctcaaaaccaactaaatctaagactaggtacatcgtcctcctgattcctaactaagagttttgtctgcggaatatgaactacaatcaataggcaaactacgagttttggttgatttagattctgatccacagggccctctttttataaactcagcccacaacttggatattaactacaacaaaggcctttacttgtttacagcttcaaacaattccaaaaagcttgagtgtcccgggagaaaaatatttgagtcgggtgttgaacctataattgatgttgtttccggaaatgaacaaatgtcgaagtttgttaaggtttttcgaactcgttaacctaagcactgccaaggggttgatgtttgacgctacagccatagccattaatgcaggagatgggcttgaatttggttcacctaatgcaccaaacacaattggattcgtgacggttccccaactacaaactgcgatgtcggtatcggtaattacgtcctctacccgaacttaaaccaagtggattacgtggtttgtcaaatcccctcaaaacaaaaattggccatggcctagaatttgattcaaacaaggctatggttcctaaactaggaactggccttagttttgacagcacagggtttaggggagttttgtttttaaccggtaccggatcttaaactaagtttgttccgataccaaggatttgatccttgaccggaatcaaaactgtcgtgtcctgccattacagtaggaaacaaaaataatgataagctaactttgtggaccacaccagctccatctcctaactgtagactaaatgcagagaaagatgctaaaacggtaatgtcatcctttgtttttattactattcgattgaaacacctggtgtggtcgaggtagaggattgacatctgatttacgtctctttctacgatttctcactttggtcttaacaaaatgtggcagtcaaatacttgctacagtttcagttttggctgttaaaggcagtttggctccaatatctggaacagttcaaagagtgaaaccagaattgttttacaccgtcagtttatgaacgatgtcaaagtcaaaaccgacaatttccgtcaaaccgaggttatagaccttgtcaagtttgtgctcatcttattataagatttgacgaaaatggagtgctactaaacaattccttcctggacccagaatattggaactttagaaatggagatcttactgacacgagtagaataatattctaaactgcttttacctcacgatgatttgttaaggaaggacctgggtcttataaccttgaaatctttacctctagaatgactaggcacagcctatacaaacgctgttggatttatgcctaacctatcagcttatccaaaatctcacggtaaaactgccaaaagtaacattgtcagtcaagtttccgtgtcggatatgtttgcgacaacctaaatacggattggatagtcgaataggttttagagtgccattttgacggttttcattgtaacagtcagttcaatacttaaacggagacaaaactaaacctgtaacactaaccattacactaaacggtacacaggaaacaggagacacaactccaagtgcatactctatgtcatatgaatttgcctctgttttgatttggacattgtgattggtaatgtgatttgccatgtgtcctttgtcctctgtgttgaggttcacgtatgagatacagtatttcatgggactggtctggccacaactacattaatgaaatatttgccacatcctcttacactttttcatacattgcccaagaataaagaatcgtttgtgtaaagtaccctgaccagaccggtgttgatgtaattactttataaacggtgtaggagaatgtgaaaaagtatgtaacgggttcttatttcttagcaaacacatatgtttcaacgtgtttatttttcannnnccctaggcaaaatagcaccctcccgctccagaacaacatacagcgcttcacagcggcagcctaacagtcagatacaaagttgcacaaataaaaagtnnnngggatccgttttatcgtgggagggcgaggtcttgttgtatgtcgcgaagtgtcgccgtcggattgtcagtcccttaccagtaaaaaagaaaacctattaaaaaaacaccactcgacacggcaccagctcaatcagtcacagtgtaaaaaagggccaagtgcagagcgagtaggaatggtcattttttcttttggataatttttttgtggtgagctgtgccgtggtcgagttagtcagtgtcacattttttcccggttcacgtctcgctcattatataggactaaaaaatgacgtaacggttaaagtccacaaaaaacacccagaaaaccgcacgcgaacctacgcccagaaacgaaagccaaaaaacccacatatatcctgattttttactgcattgccaatttcaggtgttttttgtgggtcttttggcgtgcgcttggatgcgggtctttgctttcggttttttgggtgaacttcctcaaatcgtcacttccgttttcccacgttacgtaacttcccattttaagaaaactacaattcccaacacatacaagttactccgccctaaaacttgaaggagtttagcagtgaaggcaaaagggtgcaatgcattgaagggtaaaattcttttgatgttaagggttgtgtatgttcaatgaggcgggattttgctacgtcacccgccccgttcccacgccccgcgccacgtcacaaactccaccccctcattatcatattggcttcaatccaaaataaggtatattattgatggatgcagtgggcggggcaagggtgcggggcgcggtgcagtgtttgaggtgggggagtaatagtataaccgaagttaggttttattccatataataactacatgttaattacaatta